(19) United States
(12) Patent Application Publication
(10) Pub. No.: US 2025/0270332 A1
(43) Pub. Date: Aug. 28, 2025

(54) Tetravalent FZD and WNT Co-Receptor Binding Antibody Molecules and Uses Thereof

(71) Applicant: AntlerA Therapeutics Inc., Toronto, Ontario (CA)

(72) Inventors: Stephane ANGERS, Mississauga, Ontario (CA); Sachdev SIDHU, Toronto, Ontario (CA); Levi BLAZER, Etobicoke, Ontario (CA); Jarrett ADAMS, Toronto, Ontario (CA); Somasekar SESHAGIRI, Toronto, Ontario (CA)

(21) Appl. No.: 18/267,914

(22) PCT Filed: Dec. 17, 2021
(86) PCT No.: PCT/IB2021/061971

§ 371 Date: Jun. 16, 2023

Related U.S. Application Data
(60) Provisional application No. 63/127,408, filed on Dec. 18, 2020.

(51) Int. Cl. C07K 16/28

(52) U.S. Cl. CPC C07K 16/2863; C07K 16/28; C07K 2317/31; C07K 2317/35; C07K 2317/622

(57) ABSTRACT

Described herein are tetravalent binding antibody molecules comprising a FZD receptor binding domain and an LRP5/6 co-receptor binding domain on opposite termini of an Fc domain that activate a Wnt beta-catenin signaling pathway and methods for their use.

### SEQUENCE LISTING

The instant application contains a Sequence Listing which has been submitted electronically in ASCII format and is hereby incorporated by reference in its entirety. Said ASCII copy, created on Dec. 16, 2021, is named 117946_PD606WO_FINAL.txt and is 989,432 bytes in size.

### BACKGROUND

Wnt signaling pathways are critical for embryonic development and tissue homeostasis in adults. Wnt signaling is initiated when a Frizzled (FZD) receptor on the cell surface membrane binds with a Wnt ligand. Wnt ligands are secreted growth factors that regulate various cellular processes such as proliferation, differentiation, survival and migration.

Nineteen Wnt ligands exist in humans that interact with a network of ten Frizzled cell surface receptors (FZD) and one of several co-receptors that guide the selective engagement of different intracellular signaling branches (Wodarz, A. and Nusse, R. Annu. Rev. Cell Dev. Biol. 14, 59-88 (1998); Angers, S and Moon, R. T., transduction. Nat. Rev. Mol. Cell Biol. 10, 468-477 (2009)). FZDs have conserved structural features including seven hydrophobic transmembrane domains and a cysteine-rich ligand-binding domain. FZDs are known to function in three distinct signaling pathways, known as the Wnt planar cell polarity (PCP) pathway, the canonical Wnt/β-catenin pathway, and the Wnt/calcium pathway. The presence of Wnt co-receptors is also required to direct the differential engagement of the intracellular signaling cascades listed above. For example, Wnt ligands bind to a Frizzled receptor and a member of the low-density lipoprotein receptor-related proteins 5 and 6 (LRP5/6) co-receptor family to activate the Wnt/β-catenin pathway, or with a receptor tyrosine kinase-like orphan receptors 1 and 2 (ROR1/2), related to receptor tyrosine kinase (RYK) or protein tyrosine kinase 7 (PTK7) co-receptor to activate alternate β-catenin-independent signaling pathways.

Wnt ligands are universally important for the control of tissue stem cells self-renewal and regulation of many progenitor cell populations, but the hydrophobicity and sensitive tertiary structure of Wnt proteins makes their biochemical purification challenging and their use in vitro and in vivo inefficient. Described herein are tetravalent binding antibody molecules that activate a Wnt signaling pathway and methods for their use.

### SUMMARY OF THE INVENTION

Described herein are tetravalent binding antibody molecules that activate a Wnt signaling pathway and methods for their use. The tetravalent binding antibody molecules bind to both an FZD receptor, e.g., Frizzled Class Receptor 1 (FZD1), Frizzled Class Receptor 2 (FZD2), Frizzled Class Receptor 3 (FZD3), Frizzled Class Receptor 4 (FZD4), Frizzled Class Receptor 5 (FZD5), Frizzled Class Receptor 6 (FZD6), Frizzled Class Receptor 7 (FZD7), Frizzled Class Receptor 8 (FZD8), Frizzled Class Receptor 9 (FZD9), or Frizzled Class Receptor 10 (FZD10) and a Wnt co-receptor, e.g., LRP5 or LRP6 (LRP5/6), thereby activating a Wnt signaling pathway. In an embodiment, the tetravalent binding antibody molecules bind to both a FZD4 receptor and LRP5 and/or LRP6 and activate the Wnt/β-catenin signaling pathway. The tetravalent binding antibody molecules of this invention are also referred herein as “FZD Agonists”, Frizzled and LRP5/6 Agonist (FLAg), and in some embodiments as “ANTs”.

The tetravalent binding antibody molecules include an Fc domain comprised of CH2 and CH3 domains or fragment thereof comprising the CH3 domain, and a first bivalent binding domain that interacts with one or more FZD receptor, e.g., one or more of FZD1, FZD2, FZD3, FZD4, FZD5, FZD6, FZD7, FZD8, FZD9, and FZD10, and a second bivalent binding domain that binds a WNT co-receptor, e.g., LRP5 or LRP6, wherein the FZD binding domain is linked to one terminus of the Fc domain and the co-receptor binding domain is linked to the other terminus of the Fc domain. Thus, the binding domain for the FZD receptor and the binding domain for the WNT co-receptor are not directly linked rather they are separated by the Fc domain, or fragment thereof comprising the CH3 domain.

The Fc domain of the FZD Agonists may be an Fc domain of an immunoglobulin with or without effector function. The immunoglobulin may be an IgG, e.g., an IgG₁. In an embodiment of this invention the tetravalent binding antibody molecule comprises two polypeptides containing an Fc region that dimerize via the intrinsic ability of the Fc region in each polypeptide to dimerize or via a knob-in-holes configuration within the Fc. Thus, the Fc dimer may be a heterodimer or a homodimer. Methods for dimerizing peptides via a knob-in-hole configuration are described in WO2018/026942, inventors Van Dyk et al., Carter P. (2001) J. Immunol. Methods 248, 7-15; Ridgway et al. (1996) Protein Eng. 9, 617-621; Merchant, et al. (1998) Nat. Biotechnol. 16, 677-681, and; Atwell et al., (1997) J. Mol. Biol. 270, 26-35, all incorporated herein by reference.

In an embodiment, each of the binding domains of the FZD Agonists described herein are bivalent and each may be monospecific, having two binding sites for the same epitope of an FZD receptor, e.g., FZD4, or Wnt co-receptor, e.g. LRP5/6, or bispecific having two binding sites with each site binding a different epitope on an FZD or Wnt co-receptor, e.g., a Wnt1 binding (domain E1-E2 within the extracellular domain of LRP5/6) site and a Wnt3 binding site (domain E3-E4 within the extracellular domain of LRP5/6) within the LRP5/6 co-receptor. In an embodiment, the LRP5/6 binding domain binds to a Wnt3A site (domain E3-E4) on LRP5 and binds to a Wnt3A site (domain E3-E4) on LRP6.

In embodiments of this invention the FZD binding domain linked to the Fc domain of the FZD Agonist comprises one or more immunoglobulin heavy-chain variable domain (VH) fragments and/or one or more immunoglobulin light-chain variable domain (VL) fragments that bind the FZD, e.g., FZD4. In an embodiment of this invention the FZD binding domain may comprise Fabs, a diabody or single chain variable fragments (scFv) single-domain antibody fragments, e.g., V_{H}H, or combinations thereof that bind to the same or different epitopes on the FZD.

In an embodiment of this invention the VHs and/or VLs of the FZD binding domain binds FZD4 or FZD5 and comprise the light chain CDRs and the heavy chain CDRs of a FZD4 or FZD5 binding antibody of Table 1, Table 2, or Table 6, and/or comprise light chain CDRs and heavy chain CDRs that are 50%, 55%, 60%, 75%, 80%, 85%, 90%, 95%, 96%, 97%, 98% or 99% identical to the CDRs of an FZD4 antibody of Table 1, Table 2 or Table 6, and still retain binding to the FZD4 or FZD5 receptor. For example, in an embodiment of the invention, the FZD binding domain may comprise a first heavy chain (CDR-H1), a second heavy chain (CDR-H2), and/or a third heavy chain (CDR-H3), wherein the VH that binds FZD may comprise CDR-H1 of SEQ ID NO: 24, SEQ ID NO: 365, or SEQ ID NO: 893, a CDR-H2 of SEQ ID NO: 51, SEQ ID NO: 61, SEQ ID NO: 462, or SEQ ID NO: 894 and/or CDR-H3 of SEQ ID NO: 79, SEQ ID NO: 90, SEQ ID NO: 484, or SEQ ID NO: 895 and a first light chain (CDR-L1), a second light chain (CDR-L2), and/or a third light chain (CDR-L3), wherein the VL that binds FZD may comprise CDR-L1 of SEQ ID NO: 1, SEQ ID NO: 3 or SEQ ID NO: 12, a CDR-L2 of SEQ ID NO: 2, SEQ ID NO: 3 or SEQ ID NO: 12 and/or a CDR-L3 of SEQ ID NO: 3, SEQ ID NO: 12, SEQ ID NO: 285, or SEQ ID NO: 896.

In an embodiment of this invention the co-receptor (LRP5/6) binding domain linked to the Fc domain of the FZD Agonist comprises one or more immunoglobulin heavy-chain variable domain (VH) fragments and/or one or more immunoglobulin light-chain variable domain (VL) fragments that bind to the Wnt co-receptor, e.g., LRP5 and/or LRP6. For example, in an embodiment of the invention, the LRP binding domain may comprise a first heavy chain (CDR-H1), a second heavy chain (CDR-H2), and/or a third heavy chain (CDR-H3), wherein the VH that binds LRP may comprise a CDR-H1 of SEQ ID NO: 527, SEQ ID NO: 528, SEQ ID NO: 536, SEQ ID NO: 716, or SEQ ID NO: 720, a CDR-H2 of SEQ ID NO: 552, SEQ ID NO: 553, or SEQ ID NO: 566, SEQ ID NO: 785, or SEQ ID NO: 791 and/or a CDR-H3 of SEQ ID NO: 584, SEQ ID NO: 585, SEQ ID NO: 586 or SEQ ID NO: 603, SEQ ID NO: 856 or SEQ ID NO: 862 CDR-H3 and a first light chain (CDR-L1), a second light chain (CDR-L2), and/or a third light chain (CDR-L3), wherein the VL that binds LRP may comprise CDR-L1 of SEQ ID NO: 1, a CDR-L2 of SEQ ID NO: 2, or SEQ ID NO: 491 and/or a CDR-L3 of SEQ ID NO: 130, SEQ ID NO: 492, SEQ ID NO: 493, SEQ ID NO: 510, SEQ ID NO: 623 or SEQ ID NO: 665.

In an embodiment of this invention, the Wnt co-receptor binding domain is bivalent and may comprise a diabody, or may comprise a Fab, a single chain variable fragment (scFv) or a single domain antibody fragments (V_{H}H) or combinations thereof for binding to the same or different epitopes on the co-receptor. In embodiments of this invention the VHs and VLs of the Wnt coreceptor binding domain comprise the light chain CDRs and/or the heavy chain CDRs of a LRP5 and/or LRP6 binding antibody of Table 3, Table 4 or Table 6, or comprise light chain CDRs and/or heavy chain CDRs that are 50%, 55%, 60%, 75%, 80%, 85%, 90%, 95%, 96%, 97%, 98% or 99% identical to the CDRs of an LRP5 and/or LRP6 antibody of Table 3, Table 4 or Table 6, and still retain binding to the LRP5 and/or LRP6 co-receptor.

In an embodiment of this invention the Wnt co-receptor binding domain linked to the Fc domain of the FZD Agonists described herein comprises a diabody, formed by two peptides each peptide comprising a heavy-chain variable domain (VH or VH domain) linked to a light-chain variable domain (VL or VL domain) wherein the VH and the VL from one peptide pair with the VL and VH of the other peptide forming the diabody. In this configuration, the binding domain has two binding sites that bind to the Wnt co-receptor, e.g., LRP5 or LRP6. The diabody may be monospecific binding the same site on the co-receptor or may be bispecific (bs) binding two different sites on the co-receptor. By using a knobs-in-holes Fc configuration, the peptides comprising the VH and VL linked to Fc regions, can be non-identical but will still pair to form a bispecific binding domain capable of binding to two different sites on the Wnt co-receptor (e.g. LRP5 or LRP6).

The peptides forming the diabodies, the V_{H}H, the scFv, and the Fabs that form the binding domains may be derived from an antibody selected for its binding to a desired target, a “source antibody”. For the FZD binding domain, the “FZD source antibody” may be an antibody that binds to one or more of the FZD receptor(s), e.g., one or more of FZD1, FZD2, FZD3, FZD4, FZD5, FZD6, FZD7, FZD8, FZD9, and FZD10, and antagonizes Wnt signaling or inhibits Wnt binding to the given FZD receptor(s). Alternatively, the FZD source antibody may be an antibody that binds to the FZD receptor(s) without antagonizing Wnt signaling or without inhibiting Wnt binding to the FZD receptor. Likewise, for the co-receptor binding domain, the “co-receptor source antibody” may be an antibody that binds to the Wnt co-receptor, e.g., LRP5/6, and antagonizes Wnt signaling or inhibits Wnt binding to the Wnt co-receptor. Alternatively, the co-receptor source antibody may be an antibody that binds to a co-receptor, e.g., LRP5/6, without antagonizing Wnt signaling or without inhibiting Wnt binding to the co-receptor.

In an embodiment of this invention the FZD binding domain of the FZD Agonist may bind specifically to a specific FZD, e.g., FZD4, with a higher affinity than to other FZDs, i.e., FZD1, FZD2, FZD3, FZD5, FZD6, FZD7, FZD8, FZD9, and FZD10, or may be pan-specific, binding to one or more other members of the FZD receptor family. In an embodiment the FZD binding domain binds specifically to one FZD with an affinity greater than 10-fold over the binding to any other Frizzled family member.

In an embodiment of this invention the FZD Agonist binds to FZD4, a “FZD4 Agonist”. The FZD4 binding domain of the FZD4 Agonist may bind specifically to FZD4, binding with a higher affinity to FZD4 over other FZDs, or may be pan-specific, binding to FZD4 and one or more other members of the FZD receptor family, e.g., Frizzled Class Receptor 1 (FZD1), Frizzled Class Receptor 2 (FZD2), Frizzled Class Receptor 3 (FZD3), Frizzled Class Receptor 5 (FZD5), Frizzled Class Receptor 6 (FZD6), Frizzled Class Receptor 7 (FZD7), Frizzled Class Receptor 8 (FZD8), Frizzled Class Receptor 9 (FZD9), or Frizzled Class Receptor 10 (FZD10). In an embodiment the FZD binding domain binds specifically to FZD4 with an affinity greater than 10-fold over any other Frizzled family member listed above.

In an embodiment of this invention the FZD Agonist binds to FZD5, a “FZD5 Agonist.” The FZD5 binding domain of the FZD5 Agonist may bind specifically to FZD5, binding with a higher affinity to FZD5 over other FZDs, or may be panspecific, binding to FZD5 and one or more other members of the FZD receptor family, e.g., FZD1, FZD2, FZD3, FZD4, FZD6, FZD7, FZD8, FZD9, or FZD10. In an embodiment the FZD binding domain binds specifically to FZD5 with an affinity greater than 10-fold over any other Frizzled family member listed above.

In an embodiment of the FZD Agonists of this invention the Wnt co-receptor binding domain is a monospecific bivalent LRP5/6 co-receptor binding domain and binds to a single epitope on the LRP5 and/or LRP6 co-receptor, e.g., an epitope of the LRP5 and/or LRP6 coreceptor that binds to Wnt1 (E1-E2 domain of LRP5 or LRP6) or binds Wnt3a (E3-E4 domain of LRP5 or LRP6). In an embodiment of this invention the co-receptor binding domain is a bispecific bivalent LRP5/6 binding domain that binds to two epitopes within the LRP5 and/or LRP6 co-receptor extracellular domain, e.g., the co-receptor binding domain interacts with the Wnt1 (E1-E2) and Wnt3 (E3-E4) epitopes of the LRP5 and/or LRP6 co-receptor. In an embodiment of this invention the co-receptor binding domain is a bispecific bivalent binding domain that binds to an extracellular domain of LRP5 and LPR6, e.g., the domain interacts with the Wnt1 (E1-E2) epitope of the LRP5 co-receptor and the Wnt1 (E1-E2) epitope of the LRP6 co-receptor LRP5, or the domain interacts with the Wnt3a (E3-E4) epitope of the LRP5 co-receptor and the Wnt3a (E3-E4) epitope of the LRP6 co-receptor or alternatively the domain interacts with a Wnt1 (E1-E2) epitope or LRP5 co-receptor and a Wnt3a (E3-E4) epitope of LPR6 co-receptor or vis versa.

Various formats of tetravalent binding antibody molecules described herein are depicted in FIG. 6. In a particular format, Diabody-Fc-Fab, an LRP5/6 binding diabody is linked to the N-terminus of an Fc domain and two Fabs are linked to the C-terminus of the Fc domain wherein the Fab is linked to the CH3 of the Fc domain via the Fab heavy chain (VH) variable domain. Alternatively, the Fab is linked to the CH3 of the Fc domain via the variable region (VL) of the light chain.

We previously reported multivalent binding molecules comprising an Fc domain, a FZD binding domain and a Wnt co-receptor (LRP5/6) binding domain on opposite termini of the Fc domain, e.g., a molecule having a FZD4 diabody linked one terminus of an Fc domain and a LRP5/6-binding diabody linked to the other terminus of the Fc domain, see PCT/IB2019/051174 inventors Angers et al. and PCT/IB2020/055463 inventors Angers et al., both incorporated in their entirety by reference.

It has been reported that Wnt-βcatenin signaling, specifically through activation of FZD4, is important for vasculature development and for adult vasculature homeostasis. More specifically, it is critical for barrier function at the blood-retina and blood-brain barriers (BRB and BBB). Defects in FZD4 signaling can lead to endothelial cell permeability defects and genetic mutations within this pathway are known to lead to vascular defects (e.g. Norrie disease, FEVR). At the blood-retina barrier, the extracellular ligand Norrin predominantly activates a FZD4-TSPAN12-LRP5 complex to regulate endothelial cell-cell interactions, barrier functions and permeability (Wang et al. (2012) Norrin/Frizzled4 signaling in retinal vascular development and blood brain barrier plasticity. Cell. 151:1332-1344). At the blood-brain barrier the secreted Wnt7a/b growth factor chiefly activates the FZD4-GPR124-LRP6 receptor complex (Chang et al., (2017). GPR124 is essential for blood-brain barrier integrity in central nervous system disease. (Nat. Med. 23:450-460). The FZD4 Agonists described herein, e.g., the configurations having a diabody binding domain for a LRP5/6 and an FZD4 binding domain comprised of two Fab fragments that bind FZD4, wherein the binding domains are on opposite termini of an Fc domain, produce a particularly stable and homogenous molecule with an unexpectedly high level of Wnt-βcatenin signaling pathway activation in endothelial cells that translates into increased barrier function and decreased vascular permeability (FIG. 11). In essence, the FZD4 Agonists described herein function as Norrin and Wnt7a/b mimetic molecules.

This invention also includes methods for using the FZD Agonists described herein. Described herein are methods to activate a Wnt signaling pathway, e.g., the Wnt/β-catenin signaling pathway, using the tetravalent binding antibody molecules of this invention, which are contemplated to promote the proximity of FZD receptors and Wnt co-receptors, e.g., one or more of FZD1, FZD2, FZD3, FZD4, FZD5, FZD6, FZD7, FZD8, FZD9, and FZD10 receptors and LRP5 and/or LRP6 co-receptors, on a cell wherein binding by the FZD Agonists to both FZD receptor(s) and the LRP5 and/or LPR6 co-receptor(s) activates the Wnt signaling pathway.

Blood-retina barrier (BRB) formation and retinal angiogenesis require βcatenin signaling induced by the ligand norrin (NDP [Norrie disease protein]), the receptor FZD4, co-receptor LRP5, and the TSPAN12 (tetraspanin 12). As such, an aspect of this invention is a method for promoting and/or maintaining retinal vasculature barrier function and angiogenesis by treating eye tissue, e.g., retinal tissue, with an effective amount of a tetravalent FZD4 Agonists of this invention.

Also, an aspect of this invention is a method for promoting, restoring and/or maintaining the BRB and BBB functions by treating the BRB or BBB vasculature with an effective amount of a tetravalent FZD4 Agonist described herein. A further aspect of this invention is a method for treating a subject having a disorder or condition characterized by defective retinal or brain angiogenesis characterized by reduced endothelial cell barrier function leading to vascular leakage by administering to such subject an effective amount of a FZD4 Agonists of this invention. A further aspect of this invention is a FZD4/LRP5 tetravalent binding antibody molecule or pharmaceutical composition for use in the treatment or prevention of a disorder or condition characterized by defective retinal or brain angiogenesis and/or characterized by reduced endothelial cell barrier function and/or vascular leakage. A further aspect of this invention is a method of treating or preventing a disorder or condition characterized by defective retinal or brain angiogenesis and/or reduced endothelial cell barrier function and/or vascular leakage comprising administering to a person in need thereof a therapeutically effective amount of a FZD4/LRP5 tetravalent binding antibody molecule described herein. A further aspect of the invention is the use of a FZD4/LRP5 tetravalent binding antibody molecule for the manufacture of a medicament for the treatment or prevention of a disorder or condition characterized by defective retinal or brain angiogenesis and/or reduced endothelial cell barrier function and/or vascular leakage. Such disorders or conditions include ocular disorders, including but are not limited to disorders of the retina or macula. Such disorders of the retina or macula include, but are not limited to diabetic retinopathy, retinipathy of prematurity, Coats' disease, FEVR, Norrie disease, macular degeneration, diabetic macular edema, and pediatric vitreoretinopathies. Additional disorders or conditions included in embodiments of this invention include but are not limited to Alzheimer's disease, epilepsy, multiple sclerosis, ischemia, and stroke.

An embodiment of this invention includes methods for producing vascularized cerebral organoids by promoting the barrier function of the vasculature network throughout the organoids, and thereby mimicking blood-brain-barrier function using an effective amount of a tetravalent FZD4 Agonist described herein.

Also, an embodiment of this invention is a method of treating a subject suffering from a gastrointestinal disorder, including a subject having inflammation of all or part of the intestines, also known as inflammatory bowel disease, by administering to such subject an effective amount of a pharmaceutical composition of this invention, e.g., a composition comprising a FZD5 Agonist. Examples of inflammatory bowel disease include, but are not limited to, Crohn's disease, and ulcerative colitis.

Also, an embodiment of this invention are methods for directing differentiation of iPS or other pluripotent stem cells (PSCs) towards various lineages by culturing these cells in the presence of an effective amount of a tetravalent binding antibody molecule of this invention.

Also described herein are methods for making the tetravalent binding antibody molecules of this invention.

The modular aspects of this invention allow for mixing and matching binding domains derived from FZD-binding antibodies and LRP5/6-binding antibodies on opposite termini of the Fc domain to generate a tetravalent binding antibody molecule that can engage a FZD-LRP5/6 co-receptor complexes to selectively activate Wnt signaling. The modularity and effectiveness of the tetravalent binding antibody molecules for activating Wnt signaling pathways described herein contrasts with the Wnt surrogates described in the prior art that consists of monovalent FZD and Wnt co-receptor binding ligands, or FZD and Wnt co-receptor binding ligands wherein the binding ligands are not attached to opposite ends of an Fc domain.

### BRIEF DESCRIPTION OF THE FIGURES

FIG. 1A and FIG. 1B. Single point ELISAs. FZD4-binding antibodies isolated from affinity matured libraries of the known FZD4-binding antibodies 5044 (FIG. 1A) and 5027 (FIG. 1B) bind to FZD4 sites that compete with their parental antibody. The reaction was stopped by adding 1M H₃PO₄ and the absorbance was measured spectrophotometrically at 450 nm in a microtiter plate reader; white=BSA; black and white stripe-Fc; gray=FZD4+blocking antibody; and black=FZD4.

FIG. 2. Epitope mapping of FZD4 antibodies. FZD4 and 5027 and 5044 have overlapping epitopes. The pan-FZD binder 5016 is a positive control showing that the antigens are functional, with the exception of “FZD4_Swap10”. Both FZD4-specific antibodies 5027 and 5044 are unable to bind to “FZD4 Swap 7” suggesting that these molecules bind to this region of the FZD ECD.

FIG. 3A. Size-exclusion chromatography (SEC). Analysis of FZD4 antibodies as compared to Trastuzumab. Protein elution was monitored using absorbance at 280 nM.

FIG. 3B. ELISA specificity. Measurements of the FZD4 antibodies determined against FZD4 and against FZD1 and FZD10, two members of the FZD family most-closely related to FZD4. The reaction was stopped by adding 1M H3PO4 and the absorbance was measured spectrophotometrically at 450 nm in a microtiter plate reader.

FIG. 4. Phage clonal ELISA of synthetic antibodies targeting LRP5. The results demonstrate that the synthetic antibodies bound to LRP5. The absorbance was measured spectrophotometrically at 450 nm in a microtiter plate reader; gray-BSA; light gray=his-Fc; dark gray=LRP5

FIGS. 5A and 5B. Phage clonal ELISA of synthetic antibodies targeting LRP6. The results demonstrate the synthetic antibodies bound to LRP6. The absorbance was measured spectrophotometrically at 450 nm in a microtiter plate reader; black=BSA; gray=Fc; light gray-LRP6-Fc.

FIG. 6. Modalities of tetravalent binding antibody molecules. Illustrated are: a diabody-Fc-diabody format having an FZD-binding monospecific diabody on the N-terminal of the Fc domain and a LPR5/6-binding bispecific diabody on the C-terminal of the Fc domain; a Diabody-Fc-scFv format having an N-terminal LPR5/6-binding bispecific diabody and two C-terminal FZD binding scFv; an IgG-diabody format having two FZD-binding Fabs forming an N-terminal binding domain and a bispecific LRP5/6 binding diabody forming the C-terminal binding domain; an IgG-scFv format having two FZD-binding Fabs forming an N-terminal binding domain and two LRP5/6 binding scFvs forming the C-terminal binding domain, and; a diabody-Fc-Fab format having a bispecific LRP5/6 binding diabody forming the N-terminal binding domain and two FZD-binding Fabs forming the C-terminal binding domain, wherein the Fabs are linked to the CH3 of the Fc domain via the Fab variable heavy region. It is specifically contemplated that in an alternate diabody-Fc-Fab format the Fabs are linked to the CH3 of the Fc domain via the Fab variable light region. The various domains of the tetravalent molecules, VL, VH, CH1, CH2, CH3, CL1 and Fc, are joined via linkers, e.g., peptide linkers. The Fc domain, is formed by the dimerization of the CH2 and CH3 domains of the Hole construct Fc region and Knob construct Fc region. The various domains of the tetravalent molecules, VL, VH, CH1, CH2, CH3, CL1 and Fc, are joined via linkers, e.g., peptide linkers.

FIG. 7. FZD4 Agonist having a Diabody-Fc-Fab format. The Diabody-Fc-Fab format having an LRP5-binding bispecific diabody forming a bivalent bispecific N-terminal LRP5-binding domain and two FZD4-binding Fabs forming a bivalent monospecific C-terminal FZD4-binding domain and an Fc region with attenuated effector functions due to amino acid mutations, e.g., N297G (NG) and D265A, (DANG) variants. The various domains of the tetravalent molecules, VL, VH, CH1, CH2, CH3, CL1 and Fc, are joined via linkers, e.g., peptide linkers.

FIG. 8. FZD4 Agonists having a Diabody-Fc-Fab format (ANT) bind FZD4 with high selectivity. FIG. 8A depicts the apparent selectivity of the FZD4 Agonists for the recombinant extracellular domain (ECD) of 9 of the 10 FZD as determined by biolayer interferometry (BLI). FIG. 8B demonstrates FZD agonists do not recognize common non-specific antigens. The FZD Agonists were tested at 100 nM for binding to a panel of antigens as described in Mouquet et al. Polyreactivity increases the apparent affinity of anti-HIV antibodies by heteroligation. Nature. 2010 September; 467(7315):591-595. DOI: 10.1038/nature09385, PMC3699875, and Jain T. et al. Biophysical properties of the clinical-stage antibody landscape. Proceedings of the National Academy of Sciences of the United States of America. 2017 January; 114(5):944-949. DOI: 10.1073/pnas. 1616408114, PMC5293111.

FIG. 9. FZD4 Agonists (ANT) having a Diabody-Fc-Fab format (having a LRP-binding bispecific diabody and two FZD4-binding Fabs) are stable and monomeric in solution. FIG. 9A presents the results of an analytical SEC analysis of FZD agonists as compared to trastuzumab IgG. FIG. 9B presents the results of differential scanning fluorimetry demonstrating that the FZD4 Agonists in the Diabody-Fc-Fab format have thermal denaturation profiles similar to that of trastuzumab, whereas a first generation diabody-Fc-diabody FZD4 modality (CM0199) is less optimal.

FIG. 10. FZD4-LRP5 specific FZD4 Agonists having the Diabody-Fc-Fab format (ANT). FZD4-LRP5 specific FZD4 Agonists in this format stimulate FZD4 in mouse endothelial cell line (bEND3.1) and lead to an increase in Axin2 (beta catenin target gene) gene transcription in a concentration-dependent manner.

FIG. 11A and FIG. 11B depicts a FZD4-LRP5 specific agonist having the diabody-fc-diabody format promotes endothelial cell barrier functions in a mechanism opposing VEGF-induced permeability. FIG. 11A depicts Immunofluoresence of ZO-1/CLDN3 and ZO-1/CLDN5 localization on bEnd.3 cell junctions. bEnd.3 cells were treated or not with 30 nM of F4L5.13 (aka CM0199) and Norrin (NDP) in the presence or absence of VEGF (100 ng/ml) for 1h. Starting from the top row and working downward: NT (non-treated) show no change in permeability; VEGF treatment of bEND3.1 cells leads to junction disassembly as seen by loss of plasma membrane staining of CLDN3, CLDN5 and ZO-1; Co-treatment of cells with VEGF and the FZD4 agonist CM0199 (F4L5.13) leads to a near-complete rescue of the effect of VEGF alone; the last row of FIG. 11A shows co-treatment of cells with VEGF and NDP and similarly leads to a near-complete rescue of the effect of VEGF alone, suggesting that the FZD4 Agonists described herein function as Norrin and Wnt7a/b mimetic molecules. FIG. 11B shows a transendothelial permeability assay quantifying the passage of FITC-dextran through a monolayer of bEnd.3 cells. Passage of FITC-dextran was measured after exposure of bEnd.3 cells to 100 ng/ml VEGF, 30 nM F4L5.13 or both or pretreated with VEGF for 1 h before treating with F4L5.13 for 1 h. Data are presented as mean±SD, n=5 independent experiments. Significance was calculated by one-way ANOVA with Bonferroni's multiple comparisons test (*P<0.05 as compared to VEGF treatment).

FIG. 12. Single point ELISA. FZD5 antibodies that bind the extracellular domain of FZD5 at a site overlapping with 2919 identified from affinity maturation libraries. New FZD5 antibodies bind FZD5 at a site overlapping with 2919 identified from affinity maturation libraries. Single point ELISAs were performed on 96-well Maxisorp plates coated with the ECDs of human FZD5 protein in the presence or absence of a saturating concentration of 2919 IgG. The absorbance was measured spectrophotometrically at 450 nm in a microtiter plate reader; white with black stripes=BSA; black with white stripes=Fc; gray=FZD5+blocking antibody; black=FZD5.

FIG. 13. Single point ELISA. demonstrates new FZD5 antibodies from 2928 affinity maturation library selectively bind FZD5. New FZD5 antibodies from 2928 affinity maturation library selectively bind FZD5. Single point ELISAs were performed on 96-well Maxisorp plates coated with the ECDs of human FZD2, FZD5, or FZD8 protein. The absorbance was measured spectrophotometrically at 450 nm in a microtiter plate reader; black with white stripes-Fc; white with black stripes-FZD2; gray=FZD8; black=FZD5.

FIG. 14. Luciferase assay. Pan-FZD/LRP6 ANT9 and FZD5-specific/LRP6 ANT59 activate Wnt signaling in cells. TOPFLASH cells were treated overnight with varying concentrations of FZD agonist or a non-targeting control molecule (CM0156) and TCF/LEF-driven luciferase expression was measured using a standard luciferase assay. Both molecules are able to activate FZD-mediated luciferase expression in a concentration-responsive manner. ANT9, which is able to bind to 7 of the 10 FZD receptor subtypes produces a higher maximal activation signal than the FZD5-specific ANT59.

FIG. 15. Original format ANT39 and inverted format ANT39i. The FZD4 Agonist ANT39 having a Diabody-Fc-Fab format and FZD4 Agonist ANT39i having an IgG-Diabody format (having two FZD-binding Fabs forming an N-terminal binding domain and a bispecific LRP5/6 binding diabody forming the C-terminal binding domain) and an Fc domain. The FZD binding domain of ANT39i comprises two Fab fragments attached to the N-terminus of the Fc domain and each Fab binds an FZD. The LRP5/6 co-receptor binding domain is attached to the C-terminus of the Fc domain and is composed of a diabody that binds two different sites on the co-receptor, e.g., a Wnt1 site (E1-E2) and a Wnt3 site (E3-E4) on LRP5/6. The Fabs may be specific for a particular FZD, e.g. FZD4, or may be pan-specific, binding to more than one FZD, e.g., to FZD4 and one or more other FZD. The Fc region may have attenuated effector functions due to amino acid mutations, e.g., N297G (NG) and D265A, (DANG) variants. The various domains of the tetravalent molecules, VL, VH, CH1, CH2, CH3, CL1 and Fc, are joined via linkers, e.g., peptide linkers.

FIG. 16A depicts FZD4 Agonist ANT39 having a Diabody-Fc-Fab format (having an LRP5-binding bispecific diabody forming a bivalent bispecific N-terminal LRP5-binding domain and two FZD4-binding Fabs forming a bivalent monospecific C-terminal FZD4-binding domain) with the Fc region having attenuated effector functions due amino acid mutations to N297G and D265A (DANG) variants or L234A, L235A, P331S (LALAPS) variants, and with the Fc region further comprising knob-in-hole heterodimerization variants Merrimack, Merchant or Merchant S:S (Merrimack CH3 mutations as described in WO2018/026942A1, Merchant CH3 mutations as described in Merchant A. M. et al Nature Biothechnology 1998 vol 16 p 677-681). FIG. 16A discloses SEQ ID NOS 886, 892, 891, 886, 892, 891, 886, 892, 891, 886, 892, and 891, respectively, in order of appearance. FIG. 16B depicts FZD4 Agonist ANT39i having an IgG-Fc-Diabody format (having two Fab fragments attached to the N-terminus of the Fc domain, each Fab binding to an FZD, and a LRP5/6 co-receptor binding domain attached to the C-terminus of the Fc domain that is composed of a diabody that binds two different sites on the co-receptor) and an Fc region with attenuated effector functions due to DANG or LALAPS variants, and Merrimack, Merchant or Merchant S:S heterodimerization variants. FIG. 16B discloses SEQ ID NOS 891, 886, 891, 886, 891, 886, 891, and 886, respectively, in order of appearance.

FIG. 17. Thermostability of ANT39 variants. FIG. 17 presents the results of differential scanning fluorimetry experiments demonstrating that the LALA variant of FZD4 agonist ANT39 (ANT39 LALA) has improved thermal stability relative to the parental ANT39 (containing DANG mutations in the Fc). Specifically, the LALA variant showed an improved thermal stability that is closer to the profile of a variant of Trastuzumab that contains the same Knob/Hole Fc mutations as the ANT.

FIG. 18. FZD4 Agonist ANT42 having a Diabody-Fc-Fab format. FZD4 Agonist ANT42 having an LRP5-binding bispecific diabody forming a bivalent bispecific N-terminal LRP5-binding domain and two FZD4-binding Fabs forming a bivalent monospecific C-terminal FZD4-binding domain with the Fc region having attenuated effector functions due amino acid mutations to N297G and D265A (DANG) variants or L234A, L235A, P331S (LALAPS) variants, and with the Fc region further comprising knob-in-hole heterodimerization variants Merrimack, Merchant or Merchant S:S (Merrimack CH3 mutations as described in WO2018/026942A1, Merchant CH3 mutations as described in Merchant A. M. et al Nature Biothechnology 1998 vol 16 p 677-681). And FZD4 Agonist ANT42i having an IgG-Fc-Diabody format (having two Fab fragments attached to the N-terminus of the Fc domain, each Fab binding to an FZD, and a LRP5/6 co-receptor binding domain attached to the C-terminus of the Fc domain that is composed of a diabody that binds two different sites on the co-receptor) and an Fc region with attenuated effector functions due to DANG or LALAPS variants, and Merrimack, Merchant or Merchant S:S heterodimerization variants. FIG. 18 discloses SEQ ID NOS 886, 892, 891, 891, 886, 886, 892, 891, 891, 886, 886, 892, 891, 891, 886, 886, 892, 891, 891, and 886, respectively, in order of appearance.

FIG. 19. Antibody modalities tested for FZD agonism. A) Diabody-Fc-Diabody, VH and VL were tested both FZD or LRP binding variable domains; B) Diabody-Fc-scFv; C) scFv-Fc-Diabody; D) scFv-Fc-scFv; E) IgG-Diabody; F) IgG-scFv; G) Diabody-Fc-Fab; H) Diabody-CH3-Diabody; I) Fab-Diabody. In FIG. 19, molecules B-F, H-I, comprise N-terminal variable domains that bind LRP and the C-terminal variable domains bind FZD. In FIG. 19, molecule G comprises a variable domain at the N-terminal that binds FZD and a variable domain at the C-terminal that binds LRP. These antibody formats marked with an * were tested using a Knobs-in-Holes Fc.

FIG. 20. Multiple antibody architectures are able to elicit potent FZD agonism. Paratopes targeting pan-FZD and LRP6 were configured in various arrangements as described in table 14. Canonical Wnt pathway stimulation by each antibody was determined on wild-type HEK cells expressing the TOPFLASH reporter in a blinded manner by two different scientists. Data are presented as mean±SD and are representative of 4 different experiments.

FIG. 21. Expression Titers of various FZD agonist modalities. Various FZD agonist modalities were expressed in HEK cells, purified via protein A chromatography, and expression titer was determined based on the absorbance at 280 nm. EC50 for FZD activation was determined on wild-type HEK cells expressing the TOPFLASH reporter in a blinded manner by two different scientists.

FIG. 22. Organoid viability Assay. Mouse small intestine organoids were grown in the presence of 1 μM LGK-974 to block endogenous Wnt secretion and treated with PBS, Wnt3a conditioned media or FLAg molecules as indicated. Left, representative images from n=3 independent experiments. Right, quantification of organoid viability via CellTiter-Glo luminescence assay. Bars represent mean±/−standard error from 3 independent experiments.

FIG. 23. Mouse colon histology. Histological appearance of the mouse colon following DSS treatment cycle (7 days 2% DSS, 3 days 0.5% DSS) with intraperitoneal injection of either control IgG or ANT59 (10 mg/kg) on days 4 and 7. (A) Images captured at 20× magnification showing overall architecture. (B) Images captured at 100× showing rescue of mucosal integrity with ANT59 treatment.

FIG. 24. (A) Body weight changes in mice throughout DSS treatment cycle (7 days 2% DSS, 3 days 0.5% DSS) with intraperitoneal injection of either control CM0156, PanFZD agonist or ANT59 (10 mg/kg) on days 4 and 7. (B) Left: Representative images of dissected colons from 6-8 mice per treatment group with centimeter scale for comparison. Right: colon length from each treatment group with bar representing mean colon length+/−S.D. and individual data points displayed. *** indicates p<0.0001 in one-way ANOVA, H2O indicates normal water (no DSS).

FIG. 25. Characterization of FZD5/LRP6 ANTs. ANTs were expressed in HEK cells, purified via protein A chromatography, and expression titer was determined based on the absorbance at 280 nm. Using biolayer interferometry, the apparent affinity (avidity) of each molecule for recombinant Fc-fused human FZD5 was determined and selectivity against other human FZDs was measured. Dose-response curves for the activation of a LEF/TCF reporter gene in FZD-knockout (1,2,4,5,7) HEK293 cells overexpressing FZD5. Cells were seeded in 96-well dishes for 24 hours, then treated as indicated for 17 hours. Reporter activation was assessed using the Dual-Luciferase Reporter Assay System (Promega). Data are presented as mean±SD for technical duplicates and representative of n=3 independent experiments.

### DETAILED DESCRIPTION OF THE INVENTION

Described herein are tetravalent binding antibody molecules comprising an Fc domain, with or without effector function, a bivalent FZD binding domain and a bivalent LRP-binding domain, wherein the binding domains are attached to opposite ends of the Fc domain. In an embodiment, the FZD binding domain is attached to the carboxy terminus of the Fc region and the LRP co-receptor binding domain is attached to the amino terminus of the Fc domain. Alternatively, the FZD binding domain is attached to the amino terminus of the Fc region and the co-receptor binding domain is attached to the carboxy terminus of the Fc domain. The binding domains may be attached directly to the Fc domain or attached to the Fc domain via a linker. The FZD binding domain may bind to one or to more than one FZD receptor, i.e., one or more of FZD1, FZD2, FZD3, FZD4, FZD5, FZD6, FZD7, FZD8, FZD9, and FZD10.

In an embodiment of the invention the FZD binding domain is bivalent and comprises a diabody or comprises a scfv, a V_{H}H fragment, or an Fab fragment or combinations thereof that bind FZD, and the co-receptor binding domain is bivalent and comprises a diabody or a V_{H}H fragment, an Fab, or a scFv or combinations thereof that bind the LRP5/6 co-receptor. In an embodiment of the invention the FZD binding domain is attached to the carboxy-terminus of the Fc domain and comprises two scfv, two V_{H}H fragments, two Fab fragments or a diabody that bind FZD, and the co-receptor binding domain attached to the amino terminus of the Fc domain comprises a diabody, two V_{H}H fragments or two scFvs that binds to the LRP5/6 co-receptor. When attached to the carboxy terminus of the Fc domain the FZD-binding Fabs are linked to the CH3 of the Fc domain via the Fab variable heavy region or variable light region. In other embodiments the FZD binding domain is attached to the amino terminus of the Fc domain and is comprised of two Fabs and the LRP5/6 co-receptor binding domain is attached to the carboxy terminus of the Fc domain and is comprised of a diabody or two scFvs that bind the co-receptor.

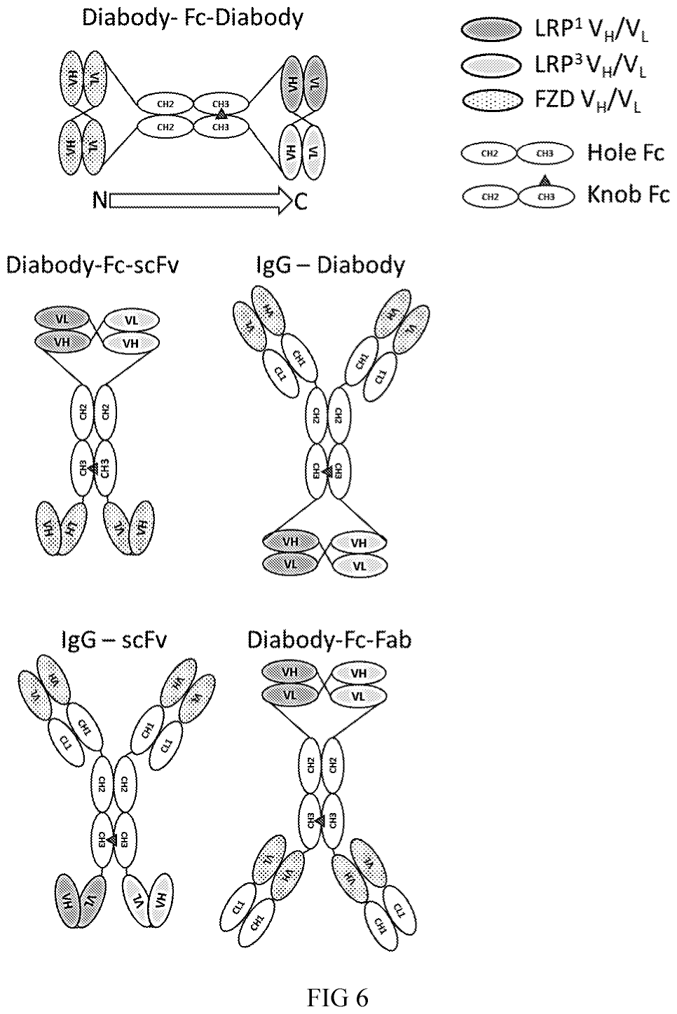

FIG. 6 illustrates a tetravalent binding antibody molecule of this invention in the Diabody-Fc-scFv format having a LRP5/6 co-receptor binding domain, an Fc domain, and a FZD binding domain. The Diabody-Fc-sFv comprises (i) an Fc domain, (ii) a bispecific diabody attached to the N-terminal of the Fc domain that binds two different sites on the co-receptor, e.g., a Wnt1 (E1-E2) site on LRP5/6, and a Wnt3 site (E3-E4) on LRP5/6, and (iii) a FZD binding domain comprising two FZD-binding scFv fragments attached to the carboxy terminus of the Fc domain. The scFv may be specific for a particular FZD, e.g. FZD4, or may be pan-specific, binding to more than one FZD, e.g. to FZD4 and one or more other FZD.

An embodiment of this invention is a tetravalent binding antibody molecule in a Diabody-Fc-scFv format having (i) an Fc domain, (ii) a LRP5/6 co-receptor binding domain that comprises a bispecific diabody that binds two different sites on the co-receptor, e.g., a Wnt1 (E1-E2) site on LRP5/6, and a Wnt3 site (E3-E4) on LRP5/6, wherein the diabody is attached to the amino terminus of the Fc domain and (iii) a FZD binding domain, attached to the carboxy terminus of the Fc domain comprising two scFv fragments each binding FZD. The scFv may be specific for the FZD, or may be pan-specific, binding to the FZD and one or more other FZD.

FIG. 6 also illustrates a tetravalent binding antibody molecule of this invention in the IgG-diabody format having (i) an Fc domain, (ii) a FZD binding domain that comprises of two Fab fragments attached to the N-terminus of the Fc domain, each Fab binding to an FZD, and (iii) a LRP5/6 co-receptor binding domain attached to the C-terminus of the Fc domain that is composed of a diabody that binds two different sites on the co-receptor, e.g., a Wnt1 site (E1-E2) and a Wnt3 site (E3-E4) on LRP5/6. The Fabs may be specific for a particular FZD, e.g. FZD4, or may be pan-specific, binding to more than one FZD, e.g., to FZD4 and one or more other FZD.

An embodiment of this invention is a tetravalent binding antibody molecule in an IgG-Diabody format comprising (i) an Fc domain, (ii) an N-terminal binding domain for a FZD, comprising two FZD-binding Fabs and (ii) a C-terminal binding domain for a LRP5 and/or LRP6 co-receptor, comprising a LRP5/6 coreceptor-binding diabody. This FZD Agonist in the IgG-Diabody format comprises,
(1) a first and second heavy chain monomer, wherein each heavy chain monomer comprises a single-chain polypeptide comprising from N-terminus to C-terminus:
   (a) a heavy chain variable domain (VH) that binds a FZD, linked to
   (b) a heavy chain constant region domain 1 (CH1 domain), linked to
   (c) an Fc region (or fragment thereof comprising a constant heavy chain domain 3 (CH3 domain)), linked to
   (d) a peptide comprising a VH that binds a LRP5/6 co-receptor, linked to a light chain variable domain (VL) that binds a LRP5/6 co-receptor, and
(2) a first and second light chain monomer, each light chain monomer comprising from N terminus to C terminus a VL that binds the FZD, linked to a constant light chain domain 1 (CL1 domain).

The first and second heavy chain monomers dimerize via their Fc regions, or fragments thereof. The linker between the VH and VL that bind the LRP5/6 is of a length that promotes the pairing of the VH and VL of the first heavy chain monomer with the VL and VH of the second heavy chain monomer thereby forming a LRP5/6 co-receptor binding diabody. The FZD-binding Fabs are formed by the pairing of each heavy chain monomer with a light chain monomer such that the VH that binds FZD4 and CH1 of each of the heavy chain monomer, pairs with the VL that binds FZD4 and CL1 of the light chain monomers. In this IgG-Diabody format, the Fabs form the FZD4-binding domain on the N-terminus of the Fc domain and the diabody forms the co-receptor-binding domain on the C-terminus of the Fc domain. The Fabs may be specific for one FZD, e.g., FZD4 or FZD5, or may be pan-specific, binding to more than one FZD, e.g., to FZD4 and/or FZD5, and in some cases more FZD. The Fc regions may dimerize via a knob-in-hole configuration. Methods for dimerizing peptides via a knob-in-hole configuration are described in WO2018/026942, inventors Van Dyk et al., Carter P. (2001) J. Immunol. Methods 248, 7-15; Ridgway et al. (1996) Protein Eng. 9, 617-621; Merchant, et al. (1998) Nat. Biotechnol. 16, 677-681, and; Atwell et al., (1997) J. Mol. Biol. 270, 26-35. The Fc regions may be Merrimack (knob chain: Q347M, Y349F, T350D, T366W and L368M; hole chain: S354I, E357L, T366S, L368A and Y407V), Merchant (knob chain: T366W; hole chain: T336S, L368A and Y407V) or Merchant S:S (Merchant mutations with additional S354C variant in the knob chain and Y349C in the hole chain). The Fc regions may also contain mutations that alter their effector function, e.g., the Fc region may have attenuated effector functions due to amino acid mutations, e.g., DANG variants and LALAPS variants.

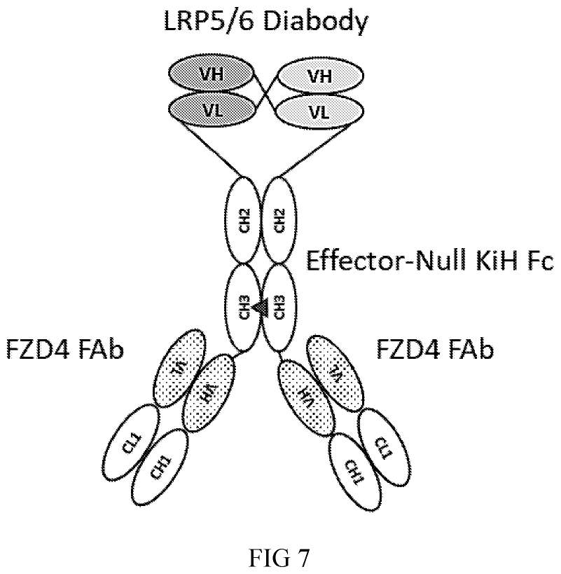

Although in FIG. 6 the peptides forming the diabody in the IgG-Diabody format are linked to the C-terminal of the Fc domain via their VH domain in a VH-VL orientation (N terminal to C terminal), in some embodiments, the peptides forming the diabody are linked to the C-terminal of the Fc domain via their VL domains in a VL-VH orientation (N-terminal to C-terminal). And, although the heavy chains are depicted as comprising a VH domain and a CH1 domain linked to the N-terminal of the Fc domain and the light chains are depicted as comprising a VL domain and CL1 domain to form the Fabs, in some embodiments (Diabody-Fc-Fab in FIG. 6 and FIG. 7A) the diabodies are fused to the N-terminus of the Fc and the Fabs are fused to the C-terminus of the Fc. In order to do this, the CH3 domain of the Fc is fused directly to the heavy chain of the Fab via its VH domain (VH-CH1) or directly to the light chain via its VL domain (VL-CL) and where the light and heavy chains still associate to form the Fabs.

FIG. 6 illustrates a tetravalent binding antibody molecule in a Diabody-Fc-Fab configuration having an LRP5/6-binding bispecific bivalent diabody forming the N-terminal binding domain, and two FZD-binding Fabs forming the C-terminal binding domain. The Fabs may be specific for a particular FZD, e.g. FZD4, or may be pan-specific, binding to more than one FZD, e.g. FZD4 and one or more other FZD. See also FIG. 7A, which illustrates a tetravalent binding antibody molecule in the Diabody-Fc-Fab format having an Fc in a knob-in-hole (KiH) configuration and an LRP5-binding bispecific bivalent diabody forming the N-terminal binding domain, and two FZD4-binding Fabs forming the C-terminal binding domain. Although FIGS. 6 and 7A illustrates the Fabs linked to the CH3 of the Fc domain (at the C-terminus) via the Fab variable heavy domain (VH), it is specifically contemplated that in an alternate diabody-Fc-Fab format the Fabs are linked to the CH3 of the Fc domain via the Fab variable light domain (VL). The various domains of the tetravalent molecules, VL, VH, CH1, CH2, CH3, CL1 and Fc, are joined via linkers, e.g., peptide linkers.

Also an embodiment of this invention is a tetravalent binding antibody molecule in the Diabody-Fc-Fab format comprising (i) an Fc domain, (ii) an N-terminal binding domain comprising a diabody that binds to the co-receptor, e.g., LRP5 and/or LRP6 co-receptor and (ii) a C-terminal binding domain comprising two Fab that bind to one or more FZD, e.g., FZD4 or FZD5. This FZD Agonist in the Diabody-Fc-Fab format comprises,
(1) a first and second heavy chain monomer, wherein each heavy chain monomer comprises a single-chain polypeptide comprising, from N-terminus to C-terminus:
   (a) a peptide comprising a heavy chain variable (VH) domain that binds a LRP5/6 co-receptor and a light chain variable (VL) domain that binds a LRP5/6 co-receptor, linked to
   (b) an Fc region (or fragment thereof comprising a constant heavy chain domain 3 (CH3 domain)), linked to
   (c) a VH domain that binds a FZD, linked to
   (d) a CH1 domain, and
(2) a first and second light chain monomer each light chain monomer comprising from N-terminus to C-terminus a VL domain that binds FZD, and a constant light chain domain 1 (CL1).

The first and second heavy chain monomers dimerize via the Fc regions or fragments thereof and a bivalent LRP5/6-binding diabody is formed by the pairing of the VH domain and VL domain that bind LRP5/6 of the first heavy chain monomer with the VL domain and VH domain that bind LRP5/6 of the second heavy chain monomer. The two FZD-binding Fabs are formed by the pairing of each heavy chain monomer with a light chain monomer such that the VL that binds the FZD and the CL1 of a light chain monomer pairs with the VH that binds the FZD and the CH1 of each of the heavy chain monomers. In this Diabody-Fc-Fab format, the diabody forms the LRP5/6 co-receptor binding domain on the amino terminus of the tetravalent molecule and the two Fabs form the FZD binding domain on the C-terminus of the tetravalent binding antibody molecule. The Fc regions may dimerize via a knob-in-hole configuration.

Methods for dimerizing peptides via a knob-in-hole configuration are described in WO2018/026942, inventors Van Dyk et al., Carter P. (2001) J. Immunol. Methods 248, 7-15; Ridgway et al. (1996) Protein Eng. 9, 617-621; Merchant, et al. (1998) Nat. Biotechnol. 16, 677-681, and; Atwell et al., (1997) J. Mol. Biol. 270, 26-35. The Fc regions may be Merrimack (knob chain: Q347M, Y349F, T350D, T366W and L368M; hole chain: S354I, E357L, T366S, L368A and Y407V), Merchant (knob chain: T366W; hole chain: T336S, L368A and Y407V) or Merchant S:S (Merchant mutations with additional S354C variant in the knob chain and Y349C in the hole chain). The Fc regions may also contain mutations that alter their effector function, e.g., the Fc region may have attenuated effector functions due to amino acid mutations, e.g., DANG variants and LALAPS variants.

Although in FIGS. 6 and 7A the peptides forming the diabody in the Diabody-Fc-Fab format are linked to the Fc domain via their VL domains, thus in a VH-VL orientation (from N-terminal to C-terminal), in some embodiments the orientation can be switched such that the peptides forming the diabody are linked to the N-terminal of the Fc domain via their VH domains, thus in a VL-VH orientation (from N-terminal to C-terminal). Also, although the heavy chains in the Diabody-Fc-Fab format are depicted as comprising a VH domain and a CH1 domain, which pair with the light chain comprising a VL and CL1 domain to form the Fabs, it is also contemplated that in some embodiments the variable and constant domains are switched such that the heavy chains comprise a VL domain and a CL1 domain and the light chains comprises the VH domain and CH1 domain and the heavy and light chains still pair to form the Fabs.

In an embodiment of this invention the binding moiety of the FZD binding domain is derived from an antibody, or an antibody fragment, that binds specifically to one FZD, e.g. FZD4 or FZD5, or is pan-specific interacting with a specific FZD, e.g. FZD4 or FZD5, and one or more additional FZD receptors (an FZD source antibody), and the co-receptor binding domain comprises a binding moiety that is derived from an antibody or antibody fragment that binds to a LPR5 and/or LRP6 (a LRP5/6 coreceptor source antibody). In an embodiment of the invention the FZD-binding antibodies bind to an extracellular cysteine rich domain (CRD) of the FZD receptor. The antibody that binds FZD may be an antibody that binds the FZD receptor and antagonizes Wnt signaling or inhibits binding of a Wnt ligand to the FZD receptor. The antibody that binds FZD may be an antibody that binds the FZD receptor without antagonizing or inhibiting binding of a Wnt ligand to the FZD receptor. The antibody that binds FZD may be an antibody that binds FZD and enhances Wnt signaling. The antibody that binds the LRP5/6 co-receptor may be an antibody that binds the LRP5/6 co-receptor and antagonizes Wnt signaling or inhibits binding of a Wnt ligand to the co-receptor, or the antibody that binds the LRP5/6 co-receptor may be an antibody that binds the co-receptor without antagonizing Wnt or Norrin signaling or inhibiting binding of a Wnt or Norrin ligand to the co-receptor.

In an embodiment of this invention the LRP5/6 co-receptor binding domain binds to a single epitope on a co-receptor, e.g., an epitope that binds to the Wnt1 (E1-E2) or Wnt3 (E3-E4) interacting domain of LRP5/6. In an embodiment of this invention the LRP5/6 co-receptor binding domain binds to two epitopes within the co-receptor, e.g., a paratope that binds to the Wnt1 (E1-E2) interacting epitope and a paratope that binds to Wnt3 (E3-E4) epitope of LRP5/6. In an embodiment of this invention the multivalent binding molecule comprises a Fc domain, wherein the Fc domain is the Fc domain of an immunoglobulin or a fragment thereof comprising the CH3 domain. In an embodiment of the invention the immunoglobulin is an IgG. In an embodiment of this invention the IgG is an IgG₁.

In an embodiment of this invention the LRP5/6 binding domain comprises a diabody comprising two peptides each comprising a heavy chain variable domain (VH) that binds to LRP5/6 linked to a light-chain variable domain (VL) that binds LRP5/6 wherein the binding domain is formed by pairing of the VH and the VL from one peptide to the VL and VH of the other peptide thereby forming the LRP5/6 binding domain.

In the tetravalent binding antibody molecules of this invention both of the binding domains are bivalent and one or both of the bivalent binding domains may be bispecific for the respective FZD receptor, e.g., FZD4 or FZD5, or LRP5/6 co-receptor. For example, the binding molecule may comprise an FZD binding domain that is bivalent and monospecific (each binding site binding to the same epitope) and the LRP 5/6 binding domain is bivalent and bispecific, binding to two different epitopes (the Wnt1 (E1-E2) and Wnt3 (E3-E4) sites on the LRP5/6 ectodomain). In an embodiment of this invention both binding domains are bivalent and bispecific, each binding domain binding to two different epitopes on their respective target FZD receptor or LRP 5/6 co-receptor.

The VH and VL domains of the FZD binding domain of the tetravalent molecules of this invention may comprise the three light chain CDRs and three heavy chain CDRs of a FZD source antibody, e.g. the FZD4 or FZD5, binding antibodies of Table 1, Table 2 or Table 6, or three light chain CDRs and three heavy chain CDRs that are at least 50%, at least 55%, at least 60%, at least 75, at least. 80%, at least 85%, at least 90%, at least at least 95%, at least 96%, at least 97%, at least 98% or at least 99% identical to the CDRs of the FZD source antibody, e.g., the FZD4 antibodies of Table 1, Table 2 or Table 6, and still retain binding to the FZD or FZD5 receptor bound by the source antibody.

The VH and VL domains of the LRP5/6 co-receptor binding domain of the tetravalent molecules of this invention may comprise the three light chain CDRs and three heavy chain CDRs of an LRP5/6 co-receptor source antibody, e.g., the LRP5/6 binding antibodies of Table 3, Table 4 or Table 6, or three light chain CDRs and three heavy chain CDRs that are at least 50%, at least 55%, at least 60%, at least 75%, at least 80%, at least 85%, at least 90%, at least 95%, at least 96%, at least 97%, at least 98% or at least 99% identical to the VH and VL of the Wnt co-receptor source antibody, e.g., the LRP5/6 binding antibodies of Table 3, Table 4 or Table 6, and still bind to the LRP5/6 co-receptor.

In an embodiment of this invention the FZD binding domain of the tetravalent binding molecule of this invention binds FZD4 (an FZD4 Agonist) or FZD5 (FZD5 Agonist) or FZD4 and/or FZD5 and one or more other FZDs (a pan-FZD Agonist) and comprises

the CDR-H1, CDR-H2 and CDR-H3 and the CDR-L1, CDR-L2 and CDR-L3 of the antibodies of Table 1, Table 2 or Table 6, or CDRs that are at least 50%, at least 55%, at least 60%, at least 75%, at least 80%, at least 85%, at least 90%, at least 95%, at least 96%, at least 97%, at least 98% or at least 99% identical to the CDR-H1, CDR-H2 and CDR-H3 and CDR-L1, CDR-L2 and CDR-L3 of the antibodies of Table 1, Table 2 or Table 6, and still bind to FZD4 or FZD5, and the LRP5/6 binding domain of the FZD4 Agonist or FZD5 Agonist or pan-FZD Agonist comprises the CDR-H1, CDR-H2 and CDR-H3 and CDR-L1, CDR-L2 and CDR-L3 of the antibodies of Table 3, Table 4 or Table 6 or the CDRs are at least 50%, at least 55%, at least 60%, at least 75%, at least 80%, at least 85%, at least 90%, at least 95%, at least 96%, at least 97%, at least 98% or at least 99% identical to the CDR-H1, CDR-H2 and CDR-H3 and CDR-L1, CDR-L2 and CDR-L3 of the antibodies in Table 3, Table 4 or Table 6, and still bind to LRP5 or LRP6.

In an embodiment, the tetravalent binding antibody molecule's FZD binding domain does not comprise a diabody, scFv, or Fab comprising the three heavy chain CDRs or three light chain CDRs of the FZD4-binding antibody 5044 in combination with a Wnt co-receptor binding domain comprising a diabody, scFv, or Fab comprising the three heavy chain CDRs and three light chain CDRs of LRP6-binding antibody 2542 and/or antibody 2539. In an embodiment, the tetravalent binding molecule does not comprise a diabody, scFv, or Fab, comprising the three heavy chain CDRs and three light chain CDRs of the FZD4-binding antibody 5027 in combination with a Wnt co-receptor binding domain comprising a diabody, scFv, or Fab comprising the three heavy chain CDRs and three light chain CDRs of LRP6-binding antibody 2542 and/or antibody 2539.

Also, an embodiment of this invention are the nucleic acid molecules encoding the tetravalent binding molecules described herein. An embodiment of this invention are the nucleic acid molecules encoding the polypeptides of the tetravalent binding molecules described herein comprising the heavy chain and light chain CDRs set forth in Tables 1, 2, 3, 4, 6. Also an embodiment of this invention are the nucleic acid molecules that encode the polypeptides of the tetravalent binding molecules, e.g., FZD5 Agonists or FZD4 Agonists, of FIGS. 7A and 7B that comprise the CDRs of Table 6. Also, an embodiment of this invention are the nucleic acid molecules that encode VH and VL domains comprising respectively the heavy chain and light chain CDRs set forth in Tables 1, 2, 3, 4, and 6. The nucleic acid molecules can be inserted into a vector and expressed in an appropriate host cell and then the tetravalent binding antibody molecules may be isolated from the cells using methods well known in the art. As such, also an aspect of this invention are expression cassettes and vectors comprising the nucleic acid molecules that encode the polypeptides of the tetravalent binding molecules, e.g., FZD4 or FZD5 Agonists, described herein, the VL and VH domains, the Fabs and the diabodies comprising the CDRs of set forth in Tables 1, 2, 3, 4, and 6, and the Fc domains described herein. An aspect of this invention are the host cells expressing these expression cassettes and vectors.

As used in this invention, the term “vector” refers to a nucleic acid delivery vehicle or plasmid that can be engineered to contain a nucleic acid molecule, e.g., a nucleic acid sequence encoding the tetravalent binding antibody molecules described herein. The vector that can express protein when inserted with a polynucleotide is called an expression vector. Vectors can be inserted into the host cell by transformation, transduction, or transfection, so that the carried genetic substances can be expressed in the host cell. Vectors are well known to the technical personnel in the field, including but not limited to: plasmid; phagemid; cosmid; artificial chromosome such as yeast artificial chromosome (YAC), bacterial artificial chromosome (BAC), or P1 derived artificial chromosome (PAC); phage such as λphage or M13 phage and animal viruses etc. Animal viruses may include but not limited to, reverse transcriptase virus (including lentivirus), adenovirus, adeno-associated virus, herpes virus (e. g. herpes simplex virus), chicken pox virus, baculovirus, papilloma virus, and papova virus (such as SV40). A vector can contain multiple components that control expression of the tetravalent binding antibody molecules described herein, including but not limited to, promoters, e.g., viral or eukaryotic promoters, e.g., a CMV promoter, signal peptides, e.g., TRYP2 signal peptide, transcription initiation factor, enhancer, selection element, and reporter gene. In addition, the vector may also contain replication initiation site(s).

As used in this invention, the term “host cell” refers to cells that can import expression cassettes and vectors, including but not limited to, prokaryotic cells such as Escherichia coli and Bacillus subtilis, fungal cells such as yeast and Aspergillus, insect cells such as S2 drosophila cells and Sf9, or animal cells, including human cells, e.g., fibroblast cells, CHO cells, COS cells, NSO cells, HeLa cells, BHK cells, or HEK293 cells.

An embodiment of this invention is a pharmaceutical composition comprising a FZD Agonist or a nucleic acid molecule, expression cassette and vector encoding a FZD Agonist described herein and a pharmaceutically acceptable carrier, diluent or excipient. The pharmaceutical composition may further comprise an additional agent, e.g., a second therapeutic antibody e.g. an anti-VEGF antibody (aflibercept, ranibizumab and bevacizumab), a growth factor, e.g., VEGF, or an agent that activates a Wnt pathway, e.g., the small molecule CHIR99021, a Norrin or R-Spondin, or a nucleic acid molecule, expression cassettes and vectors that encode the agent. The pharmaceutical composition may consist of or consist essentially of a FZD Agonist, or a nucleic acid molecule, an expression cassette or vector encoding an FZD Agonist described herein, and a pharmaceutically acceptable diluent, carrier or excipient. Suitable carriers, diluents and excipients, and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution may be e.g., from about 5 to about 8, from about 5 to 7.5 or from about 6 to 7. Further carriers include sustained release preparations such as semipermeable matrices of solid hydrophobic polymers containing the agonist, which matrices are in the form of shaped articles, e.g., films, liposomes or microparticles. It will be apparent to those persons skilled in the art that certain carriers may be more preferable depending upon, for instance, the route of administration and concentration of the FZD Agonists being administered.

This invention also includes methods for using the FZD Agonists, described herein. An embodiment of this invention is a method for activating a Wnt signaling pathway in a cell, comprising contacting a cell having an FZD receptor and a LRP5/6 co-receptor, with a tetravalent binding antibody molecule of this invention that binds the FZD, e.g., FZD4, and the LRP5/6 in an amount effective to activate Wnt signaling. It has been reported that the Norrin-FZD4 pathway plays a role in retinal angiogenesis (see Wang et al. Cell. 2012; 151(6):1332-1344.; Braunger B M, Tamm E R. Adv Exp Med Biol. 2012; 723:679-683; Ohlmann A, Tamm E R. Prog Retin Eye Res. 2012; 31(3):243-257 and; Ye et al. Trends Mol Med. 2010; 16(9):417-425). Signaling through Norrin-FZD4 pathway is necessary for development and maintenance of retinal vasculature. Mutations affecting genes of this pathway may result in several vitreoretinopathies, such as Norrie Disease, Familial Exudative Vitreoretinopathy (FEVR), and Pseudoglioma and Osteoporosis Syndrome. Additionally, Retinopathy of Prematurity (ROP) has been associated with mutations in this Norrin-FZD4 pathway, and Wnt-pathway mutations have been reported in Coats Disease and Persistent Fetal Vasculature (PFV). FZD4 signaling activated by Norrin and/or WNT7A/B pathway is also associated with CNS blood brain barrier development and homeostasis. Genetic ablation of the Norrin, FZD4, LRP5, LRP6 and the co-receptor Tetraspanin-12 (Tspan-12) result in defective angiogenesis and barrier disruption in the retinal and/or cerebellar vessels (Cho et al. (2017) Neuron 95, 1056-1073; Zhou et al., (2014) J Clin Invest 124:3825-3846). Thus, a functional Wnt signaling system plays a key fundamental role in the development of a sufficient vascular and neural network in the eye and retina to support vision and in the CNS to support BBB development and homeostasis.

An aspect of this invention is a method for promoting and/or maintaining retinal vasculature by treating eye tissue, e.g., retinal tissue, with an effective amount of a pharmaceutical compositions comprising the tetravalent antibody molecules of this invention, e.g., tetravalent antibody molecules that binds FZD4 and LRP5/6, a FZD4 Agonists, having the structures illustrated in FIG. 6 through local or systemic administration. Also, an aspect of this invention is a method for promoting and/or maintaining BBB vasculature by treating a subject in need thereof with an effective amount of a pharmaceutical compositions of this invention, e.g., a composition comprising a FZD4 Agonists having the structures depicted in FIG. 6. The BBB is initiated during development and its integrity remains vital for homeostasis and neural protection throughout life. A subject in need thereof includes a subject having a neurological condition associated with BBB dysfunction, e.g., neurodegenerative diseases such as Alzheimer's disease, as well epilepsy, multiple sclerosis, and stroke.

A further aspect of this invention is a method for treating a subject having a disorder characterized by vascular leakage, particularly retinal vascular leakage, and/or endothelial cell leakage, and disorders characterized by reduced retinal or brain endothelial cell barrier functions or a compromised BBB or BRB, e.g., diabetic retinopathy, retinipathy of prematurity, Coat's disease, FEVR, Norrie disease, macular degeneration, diabetic macular edema, and pediatric vitreoretinopathies, by administering to such subject an effective amount of a pharmaceutical compositions of this invention, e.g., a composition comprising a FZD4 Agonist having the structures depicted in FIG. 6. An effective amount of such composition is an amount sufficient, e.g., to increase or restore endothelial cell barrier functions and thereby reducing vascular leakage in such subject. The subject may be a fetus. The FZD4 Agonists of this invention particularly the FZD4 Agonist in the diabody-Fc-Fab format comprising two Fab fragments forming the FZD4 binding domain on the carboxy terminal of the Fc receptor and a binding domain for LRP5 and/or LRP6 composed of a diabody on the amino terminal of the Fc domain, e.g., as illustrated in FIG. 6, activates FZD4 and β-catenin signaling in endothelial cells, promotes barrier functions and thereby reduces endothelial cell permeability and significantly enhance angiogenesis. In particular, treatment of endothelial cells, in vivo, ex vivo or in vitro, with these FZD4 Agonists, preferably those with the diabody-Fc-Fab format, enhance the development and maintenance of retinal vasculature and/or the BRB and the BBB far more effectively than other molecules that do not have this structure.

A further aspect of the invention is a method for treating a subject having inflammation of all or part of the intestines, also known as inflammatory bowel disease, by administering to such subject an effective amount of a pharmaceutical composition of this invention, e.g., a composition comprising a FZD5 Agonist. Examples of inflammatory bowel disease include, but are not limited to, Crohn's disease, and ulcerative colitis. An effective amount of such composition is an amount sufficient to reduce, ameliorate, eliminate, or treat the inflammation. A subject in need thereof includes a subject having inflammation of the mucosal of the gastrointestinal tract. The methods disclosed herein may be practiced to reduce inflammation (e.g., inflammation associated with IBD or in a tissue affected by IBD, such as gastrointestinal tract tissue, e.g., small intestine, large intestine, or colon), activate WNT signaling, or reduce any of the histological symptoms of IBD (e.g., those disclosed herein).

The FZD Agonists of the present invention may be administered systemically or locally, e.g., by injection (e.g. subcutaneous, intravenous, intraperitoneal, intrathecal, intraocular, intravitreal, etc.), implantation, topically, or orally. Depending on the route of administration, the FZD Agonists may be coated in a material to protect the agonists from conditions that may inactivate the agonists. The tetravalent binding antibody molecules described herein may be dissolved or suspended in a pharmaceutically acceptable, preferably aqueous carrier. In addition, the composition comprising the FZD Agonists can contain excipients, such as buffers, binding agents, blasting agents, diluents, flavors, lubricants, etc. An extensive listing of excipients that can be used in such a composition, can be, for example, taken from A. Kibbe, Handbook of Pharmaceutical Excipients (Kibbe, 2000). The tetravalent binding antibody molecules can also be administered together with immune stimulating substances, such as cytokines.

An embodiment of this invention includes a method for deriving cerebral organoids with a vascular network exhibiting barrier functions by using the tetravalent antibody molecules described herein. The tetravalent binding antibody molecules described herein that activate FZD4 signaling are envisioned to promote barrier function within endothelial cells cultured with cerebral organoids and thereby promoting angiogenesis.

An embodiment of this invention includes a method for directed differentiation of multipotent or pluripotent stem cells (PSC) or induced pluripotent stem (iPS) cells comprising culturing the cells under conditions suitable for directed differentiation wherein said culturing conditions further comprise an effective amount of a tetravalent binding antibody molecule described herein. Studies in mouse and human PSCs have identified specific approaches to the addition of growth factors, including Wnt, which can induce PSC differentiation into different lineages. Methods for directed differentiation of PSCs comprising the activation of Wnt signaling are known in the art see e.g. Lam et al. (2014) Semin Nephol 34(4); 445-461; Yucer et al. (Sep. 6, 2017) Scientific Reports 7, Article number 10741. It is contemplated that the FZD Agonists, e.g. FZD4 Agonists, described herein can be used in an amount sufficient to effect activation of Wnt signaling pathways to direct differentiation of the PSCs to certain mesodermal lineages such as cardiomyocytes (cite Yoon et al. FZD4 Marks Lateral Plate Mesoderm and Signals with NORRIN to Increase Cardiomyocyte Induction from Pluripotent Stem Cell-Derived Cardiac Progenitors. Stem Cell Reports. 2018 January; 10(1):87-100. DOI: 10.1016/j.stemcr.2017.11.008.PMID: 29249665).

An embodiment of this invention is a method for enhancing tissue regeneration in a subject in need thereof by activating Wnt signaling in such subject by administering to the subject in need thereof an effective amount of a FZD Agonists described herein.

An embodiment of this invention includes a method for promoting endothelial cell barrier functions in eye tissue, e.g., retinal tissue, in a subject in need thereof, by administering an effective amount of a tetravalent binding molecule of this invention that binds FZD4 and LPR5/6, an FZD4 Agonist. In a particular embodiment the FZD4 Agonist of this invention that binds to FZD4 and a binding domain that binds to LRP5 or/and LRP6 has a diabody-Fc-Fab structure depicted in FIGS. 6 and 7. In an embodiment of this invention the FZD4 Agonists for enhancing retinal angiogenesis comprise the light chain CDRs, i.e., CDR-L1, CDR-L2, and CDR-L3 and heavy chain CDRs, i.e., CDR-H1, CDR-H2 and CDR-H3 of the FZD4-binding antibodies set forth in Tables 1, 2, and 6 and the LRP5/6-binding antibodies set forth in Tables 3, 4, and 6.

A subject as used herein may be any animal (e.g., a mammal), including, but not limited to, humans, non-human primates, horses, cows, dogs, cats, rodents, and the like. The subject may be a fetus. Typically, the subject is human.

Effective dosages and schedules for administering the FZD Agonists and nucleic acids that encode them described herein may be determined empirically, and making such determinations is within the skill in the art. Those skilled in the art will understand that the dosage of such FZD Agonists that must be administered will vary depending on, for example, the subject who will receive the antibody, the route of administration, the particular type of FZD Agonists used and other drugs being administered. Guidance in selecting appropriate doses for FZD Agonists is found in the literature on therapeutic uses of antibodies, e.g., Handbook of Monoclonal Antibodies, Ferrone, eds., Noges Publications, Park Ridge, N.J., (1985) ch. 22 and pp. 303-357; Smith, Antibodies in Human Diagnosis and Therapy, Haber, eds., Raven Press, New York (1977) pp. 365-389. The dosage ranges for the administration of the compositions are those large enough to produce the desired effect, e.g., promote endothelial cell barrier functions, vascular homeostasis, or enhance Wnt signaling. The dosage should not be so large as to cause adverse side effects, such as unwanted cross-reactions, anaphylactic reactions, and the like. Generally, the dosage will vary with the age, condition, gender and the extent of the disease or disorder, in the patient and can be determined by one of skill in the art. The dosage can be adjusted by the individual physician in the event of any contraindications. Dosage can vary, and can be administered in one or more dose administrations daily, for one or several days. While individual needs vary, determination of optimal ranges of effective amounts of the vector is within the skill of the art.

Also, an aspect of this invention is a method for making the tetravalent binding antibody molecules described herein. The amino acid sequences of FZD receptors, e.g. FZD4, and the Wnt co-receptors LRP5/6, and nucleotide sequences encoding FZD receptors and the Wnt co-receptors LRP5/6, as well as antibodies and libraries of antibodies that bind FZD, e.g., FZD4, or the Wnt co-receptors LRP5/6, are readily available or can be generated using methods well known in the art (see e.g., U.S. publication no. 2015/0232554, inventors Gurney et al. and US publication no. 2016/0194394, inventors Sidhu et al. and US20190040144, inventors Pan et al.; U.S. publication no. 2017/0166636, inventors Wu et al.; U.S. publication no. 2016/0208018, inventors Chen et al.; U.S. publication no. 2016/0053022, inventors Macheda et al.; U.S. publication no. 2015/031293, inventors Damelin et al.). And a variety of methods are known in the art for generating and screening such phage display libraries for antibodies, and antibody fragments, scFv, Fab, VL, and VH possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352:624-628(1991); Marks et al., J. Mol. Biol. 222:581-597(1992); Marks and Bradbury, in Methods in Molecular Biology 248:161-175 (Lo, ed., Human Press, Totowa, N.J., 2003); Sidhu et al., J. Mol. Biol. 338(2):299-310(2004); Lee et al., J. Mol. Biol. 340(5):1073-1093 (2004); Fellouse, Proc. Natl. Acad. Sci. USA 101(34):12467-12472(2004); and Lee et al., J. Immunol. Methods 284(1-2):119-132(2004), all incorporated herein by reference. In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12:433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12:725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227:381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360, all incorporated herein by reference. Antibodies or antibody fragments isolated from human antibody libraries are considered human antibodies or human antibody fragments herein.

In an embodiment of this invention a tetravalent binding antibody molecule in a diabody-Fc-scFv format comprising a LRP5/6 coreceptor binding domain comprising LRP5/6-binding diabody and an FZD-binding domain comprising two FZD-binding scFvs is generated by,
(a) selecting an Fc domain having a C-terminus and an N-terminus
(b) identifying an antibody that binds to an FZD receptor (the “FZD source antibody”), and
(c) identifying an antibody that binds LRP 5/6 co-receptor (“coreceptor source antibody” or “LRP 5/6 source antibody”),
(d) generating a nucleic acid molecule comprising a nucleotide sequence that encodes a polypeptide monomer comprising
   (i) a peptide comprising a VL domain linked to a VH domain, the domains comprising the heavy chain and/or light chain CDRs of the antibody of step b that bind the FZD receptor, or comprising heavy chain and/or light chain CDRs derived from the antibody of step b that still bind the FZD, linked to
   (ii) an Fc domain of step a, linked to
   (iii) a peptide comprising VL domain linked to a VH domain comprising the light chain and/or heavy chain CDRs of the antibody of step c, or comprising CDRs derived from the antibody of step c and that still bind LRP 5/6 co-receptor,
(e) expressing the nucleic acid molecule of step d to produce the polypeptide monomer and then dimerizing the polypeptide,

wherein the VH and VL that bind the FZD of each monomer form a scFv that binds FZD, and the VH and VL domains that bind the LRP 5/6 coreceptor of one monomer bind the VL and VH that binds the Wnt coreceptor of another monomer forming a LRP5/6 co-receptor-binding diabody, and

wherein the polypeptide monomer dimerizes via the Fc regions to form a tetravalent binding antibody molecule comprising an Fc domain, a FZD-binding domain comprised of two FZD-binding scFvs, and a LRP5/6 coreceptor binding domain comprised of the diabody, wherein the FZD binding domain and the LRP5/6 co-receptor binding domain are on opposite termini of Fc domain. It is contemplated that the peptides comprising the VL and VH domains that bind the FZD or the LRP may be linked to either the N or C terminus of the Fc domain via the VL domain or the VH domain provided the FZD binding domain and LRP binding domain are on opposite termini of the Fc domain. The FZD may be one or more of FZD1, FZD2, FZD3, FZD4, FZD5, FZD6, FZD7, FZD8, FZD9, and FZD10.

In an embodiment of this invention, the tetravalent binding antibody molecule has two FZD-binding Fabs, e.g., FZD4-binding Fabs, linked to one terminus of the Fc domain and two LRP5/6-binding scFvs or a LRP5/6-binding diabody linked to the other terminus of the Fc domain and is generated by,
(a) identifying the light chain complementary determining regions (CDR-L1, CDR-L2, and CDR-L3) and/or heavy chains complementary determining regions (CDR-H1, CDR-H2, and CDR-H3) of an antibody that binds to the FZD, e.g., FZD4 or FZD5, (the “FZD source antibody”) and
(b) identifying the CDR-L1, CDR-L2, and CDR-L3 and/or the CDR-H1, CDR-H2, and CDR-H3 of one or more antibodies that binds to LRP5 or LRP6, (the “LRP5/6 source antibody”),
(c) generating a nucleic acid molecule encoding a “heavy chain” polypeptide comprising
   (i) a peptide comprising an immunoglobulin constant heavy chain region 1 (CH1 domain) linked to a VH domain comprising the CDR-H1, H2 and H3 of the antibody of step a), or a CDR-H1, CDR-H2 and CDR-H3 derived from the antibody of step a) that still binds the FZD4, linked to
   (ii) an Fc region, linked to
   (iii) a peptide comprising a VL domain comprising the CDR-L1, CDR-L2 and CDR-L3 of an antibody of step b) linked to a VH domain comprising the CDR-H1, CDR-H2 and CDR-H3 of an antibody of step b), or CDR-H1, CDR-H2 and CDR-H3 derived from the antibody of step b) that binds to LRP5 or LRP6,
(d) generating a nucleic acid molecule comprising a nucleic acid sequence that encodes a “light chain” polypeptide comprising an immunoglobulin constant light region 1 (CL1) linked to a VL domain wherein the VL domain comprises the FZD light chain CDR-L1, CDR-L2 and CDR-L3 of the antibody in step a),
(e) expressing the nucleic acid molecules of (c) and (d) to produce the heavy chain polypeptide and the light chain polypeptide,

wherein two heavy chain polypeptides dimerize via their Fc regions and the VH that binds the FZD and CH1 domains of the heavy chain polypeptide pair with the VL that binds the FZD and CL1 domains of the light chain polypeptide forming two FZD Fabs and

wherein the VH and VL that binds LRP5/6 in each heavy chain polypeptide pair to form an scFv that binds LRP5/6, or the VH and VL that bind LRP5/6 of one heavy chain polypeptide in the dimer pair with the VL and VH that bind the LRP5/6 of the other heavy chain polypeptide in the dimer to form a diabody, thereby forming the tetravalent binding antibody molecule comprising an Fc domain, two FZD Fabs linked to either the N or C terminus of the Fc domain and two LRP5/6-binding scFvs or a LRP5/6-binding diabody linked to the other terminus of the Fc domain.

The FZD source antibody may be an antibody that binds specifically to one FZD, e.g., FZD4, or is a pan-specific antibody binding FZD, e.g., FZD4 or FZD5, and one or more other FZD receptors and antagonizes Wnt signaling or inhibits Wnt binding to the receptor. Alternatively, the FZD source antibody may be an antibody that binds specifically to one FZD, e.g., FZD4 or FZD5, or is a pan-specific antibody binding one FZD, e.g., FZD4 or FZD5, and one or more other FZD receptors without antagonizing Wnt signaling or inhibiting Wnt binding to the receptor. The LRP source antibody may be an antibody that binds specifically to LRP5/6, or is panspecific binding to LRP5/6 and to one or more of the Wnt co-receptors, and antagonizes Wnt signaling or inhibits Wnt binding to the co-receptor. Alternatively, the LRP5/6 source antibody may be an antibody that binds to the LRP 5/6 co-receptor, or is panspecific binding to LRP5/6 and to one or more of the Wnt co-receptors, without antagonizing Wnt signaling or inhibiting Wnt binding to the LRP5/6 co-receptor.

The FZD source antibody may be an antibody fragment that binds the FZD receptor, e.g., an Fab, a VL or VH. The light chain and heavy chain CDRs, the VH and/or VL in the FZD binding domain of the FZD Agonists may be identical to the CDRs, the VH and/or VL of the FZD source antibody or may be at least 50%, at least 55%, at least 60%, at least 75%, at least 80%, at least 85%, at least 90%, at least 95%, at least 96%, at least 97%, at least 98% or at least 99% identical to the CDRs, VH or VL of the source antibody and still retain binding to the FZD receptor. The CDRs, the VH and/or VL in the FZD binding domain of the FZD Agonists may be identical to the CDRs, the VH and/or VL of a FZD4-binding or FZD5-binding antibody of Table 1, Table 2 or Table 6, or may be at least 50%, at least 55%, at least 60%, 75%, at least 80%, at least 85%, at least 90%, at least 95%, at least 96%, at least 97%, at least 98% or at least 99% identical to the CDRs, VH or VL of a FZD4-binding or FZD5-binding antibody of Table 1 or Table 2 or Table 6 and still retain binding to the FZD receptor.

Likewise, the Wnt co-receptor source antibody may be an antibody fragment, e.g. an Fab, a VL or a VH, that binds the LRP co-receptor, e.g., LRP5/6. The light chain CDRs and heavy chain CDRs, the VH and/or VL in the Wnt co-receptor binding domain of the FZD4 Agonists may be identical to the CDRs, the VH and/or VL of the Wnt co-receptor source antibody or may be at least at least 50%, at least 55%, at least 60%, at least 75%, at least 80%, at least 85%, at least 90%, at least 95%, at least 96%, at least 97%, at least 98% or at least 99% identical to the CDRs, VHs or VLs of the source antibody and still retain binding to the LRP co-receptor. The light chain CDRs and heavy chain CDRs, the VH and/or VL in the LRP5/6 binding domain of the FZD Agonists may be identical to the light chain CDRs and heavy chain CDRs, the VH and/or VL of a LRP-binding antibody of Table 3, Table 4 or Table 6 or may be at least at least 50%, at least 55%, at least 60%, at least 75%, at least 80%, at least 85%, at least 90%, at least 95%, at least 96%, at least 97%, at least 98% or at least 99% identical to the light chain CDRs and heavy chain CDRs, VH or VL of a LRP-binding antibody of Table 3, Table 4 or Table 6 and still retain binding to the LRP co-receptor.

In an embodiment of this invention, two polypeptides of the tetravalent binding antibody molecule dimerize via knob-in-hole configuration of their Fc sequences. The tetravalent binding antibody molecules of this invention may be generated by dimerizing two polypeptides in a “knob-in-hole” configuration. The knob-in-hole configuration increases the modularity of this invention by facilitating the association of peptides that comprise binding moieties that bind different epitopes on a FZD receptor or LRP5/6 co-receptor or to epitopes on different members of the FZD receptor or co-receptor family, see e.g., FIG. 6. Methods for engineering Fc molecules via the knobs into holes design are well known in the art, see e.g., WO2018/026942, inventors Van Dyk et al., Carter P. (2001) J. Immunol. Methods 248, 7-15; Ridgway et al. (1996) Protein Eng. 9, 617-621; Merchant, et al. (1998) Nat. Biotechnol. 16, 677-681, and; Atwell et al., (1997) J. Mol. Biol. 270, 26-35.

Without wishing to be bound by theory, it is contemplated that the tetravalent binding antibody molecules of this invention facilitate the interaction of a FZD receptor and an LRP5/6 co-receptor on a cell by promoting their proximity and stabilizing conformations of the receptor proteins that are favorable for activating Wnt signaling pathways. Another embodiment of this invention is a method for facilitating the interaction of a FZD receptor and an LRP5/6 co-receptor on a cell thereby activating a Wnt signaling pathway in the cell comprising, a) selecting an Fc domain, or fragment thereof comprising a CH3 domain, having a C-terminus and an N-terminus b) linking a first bivalent binding domain, which binds the FZD receptor, on one terminus of the Fc domain and linking a second bivalent binding domain, which binds to the Wnt co-receptor, on the other terminus of the Fc domain thereby forming a tetravalent binding antibody molecule; c) contacting said tetravalent binding antibody molecule with the cell expressing said FZD receptor and Wnt co-receptor under conditions wherein the FZD receptor and co-receptor both bind to the tetravalent binding antibody molecule thereby activating the Wnt signaling pathway. The Wnt co-receptor binding domain and FZD binding domain are bivalent and each comprise a VL and/or a VH, or V_{H}H domain and one or both of the binding domains may be monospecific. In an embodiment of the invention one or both the Wnt co-receptor binding domain and FZD binding domain are bispecific. In an embodiment of the invention the Wnt co-receptor binding domain is bivalent and bispecific. The FZD binding domain may comprise a scFV that binds FZD, a V_{H}H that binds FZD, or an Fab that binds FZD, or combinations thereof, or a diabody that binds FZD. The Wnt co-receptor binding domain may comprise a scFV that binds the LRP5/6 co-receptor, a V_{H}H that binds LRP5/6, an Fab that binds the LRP5/6 co-receptor, or combinations thereof, or a diabody that binds the LRP5/6 co-receptor. In an embodiment of the invention the FZD binding domain comprises two FZD-binding Fabs and the Wnt co-receptor binding domain comprises a bispecific bivalent diabody that binds LRP5/6 on two different epitopes.

The tetravalent binding antibody molecules of this invention initiate the Wnt signaling pathway(s) that are stimulated by the FZD-co-receptor complexes, e.g., the β-catenin pathway stimulated by FZD-LRP5/6 complexes. Wnt ligands function by promoting the clustering of FZD receptors with co-receptors. Without wishing to be bound by theory, it is contemplated that the FZD Agonists described herein bind both the FZD receptor and its LRP5/6 co-receptor thereby forming a complex that mimics the binding of a Wnt molecule to the FZD receptor and LRP 5/6 co-receptor(s), which in turn activates Wnt signaling pathways, the Wnt β-catenin pathway.

An embodiment of this invention is a method for activating a Wnt signaling pathway comprising contacting a cell expressing a FZD receptor and its LRP5/6 co-receptor with an effective amount of the FZD Agonists of this invention comprising a FZD binding domain and a LRP5/6 co-receptor binding domain.

The FZD Agonists of this invention may be made recombinantly, e.g., by Gibson assembly (see Gibson et al. (2009) Nature Methods 6 (5): 343-345 and Gibson DG. (2011) Methods in Enzymology 498:349-361), or the molecules may be made synthetically e.g., using commercial synthetic apparatuses, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like. If desired, various groups may be introduced into the peptide during synthesis or during expression, which allow for linking to other molecules or to a surface.

The binding domains of the FZD Agonists may be linked to the Fc domain via a linker. In some embodiments, adjacent VH and VL domains may be attached to each other via a peptide linker. In some embodiments adjacent constant domains and variable domains are attached via a peptide linker. The linker may be, e.g. a polypeptide linker, or a non-peptidic linker. In some embodiments the constant domains and variable domains of the FZD Agonists are attached to the Fc domain via a peptide linker. Suitable linkers are well known in the art, e.g., an XTEN linker (see WO2013120683, inventors Schellenberger et al.) In some embodiments, the peptide linker comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or at least 100 amino acids. In some embodiments, the peptide linker is between 1 to 100, 5 to 75, 1 to 50, 5 to 50, 1 to 30, 1 to 25, 5 to 25, 5 to 20, 5 to 15, 5 to 10, 1-10 or 1-5 amino acids in length. The modular aspects of this invention allow for mixing and matching of binding domains derived from antibodies that bind to FZD receptor or antibodies that bind LRP5/6 co-receptor on the opposite termini of the Fc domain to generate a tetravalent binding antibody molecule that can engage FZD receptor-LRP5/6 co-receptor complexes to activate Wnt signaling.

The Fc domain of the FZD Agonists, with or without the linker, is of a length and flexibility that allows for the tetravalent binding antibody molecule of this invention to bind both the FZD receptor and its LRP5/6 co-receptor thereby stabilizing receptor conformations that are compatible with activation of downstream Wnt signaling pathways. In an embodiment of this invention the Fc domain, or fragment thereof comprising the CH3 domain, with or without the linker is greater than 100 amino acids spanning up to 300 Å, greater than 125 amino acids spanning up to 375 Å, greater than 150 amino acids spanning up to 450 Å, greater than 175 amino acids spanning up to 525 Å, or greater than 300 amino acids spanning up to 900 Å. Preferably the Fc domain is about 200 amino acids to about 300 amino acids in length.

As used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

An “affinity matured” antibody or “maturation of an antibody” refers to an antibody with one or more alterations in one or more hypervariable regions (HVRs), compared to a parent or source antibody which does not possess such alterations, such alterations resulting in an improvement in the affinity of the antibody for antigen or to other desired properties of the molecule.

By “comprising” it is meant that the recited elements are required in the composition/method/kit, but other elements may be included to form the composition/method/kit etc. within the scope of the claim. For example, a composition comprising tetravalent binding antibody molecules is a composition that may comprise other elements in addition to the tetravalent binding antibody molecules, e.g. functional moieties such as polypeptides, small molecules, or nucleic acids bound, e.g. covalently bound, to the tetravalent binding antibody molecules; agents that promote the stability of the tetravalent binding antibody molecule composition, agents that promote the solubility of the tetravalent binding antibody molecule composition, adjuvants, etc. as will be readily understood in the art, with the exception of elements that are encompassed by any negative provisos.

By “consisting essentially of”, it is meant a limitation of the scope of composition or method described to the specified materials or steps that do not materially affect the basic and novel characteristic(s) of the subject invention. For example, a tetravalent binding antibody molecule “consisting essentially of” a disclosed sequence has the amino acid sequence of the disclosed sequence plus or minus about 5 amino acid residues at the boundaries of the sequence based upon the sequence from which it was derived, e.g. about 5 residues, 4 residues, 3 residues, 2 residues or about 1 residue less than the recited bounding amino acid residue, or about 1 residue, 2 residues, 3 residues, 4 residues, or 5 residues more than the recited bounding amino acid residue.

By “consisting of”, it is meant the exclusion from the composition, method, or kit of any element, step, or ingredient not specified in the claim. For example, a tetravalent binding antibody molecule “consisting of” a disclosed sequence consists only of the disclosed amino acid sequence.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector functions, e.g., binding Fc receptors and activation of antibody-dependent cellular cytotoxicity (ADCC) and complement dependent cytotoxicity (CDC). Methods for dimerizing peptides via a knob-in-hole configuration are described in WO2018/026942, inventors Van Dyk et al., Carter P. (2001) J. Immunol. Methods 248, 7-15; Ridgway et al. (1996) Protein Eng. 9, 617-621; Merchant, et al. (1998) Nat. Biotechnol. 16, 677-681, and; Atwell et al., (1997) J. Mol. Biol. 270, 26-35. The Fc regions may be Merrimack (knob chain: Q347M, Y349F, T350D, T366W and L368M; hole chain: S354I, E357L, T366S, L368A and Y407V), Merchant (knob chain: T366W; hole chain: T336S, L368A and Y407V) or Merchant S:S (Merchant mutations with additional S354C variant in the knob chain and Y349C in the hole chain). The Fc regions may also contain mutations that alter their effector function, e.g., the Fc region may have attenuated effector functions due to amino acid mutations, e.g., DANG variants and LALAPS variants. Methods are well known in the art for mitigating antibody effector function, including for example amino acid substitutions in the Fc regions, e.g., the N297G and D265A, N297G (DANG) variants, L234A, L235A, P331S (LALAPS), LALAPS Merchant, LALAPS Merchant S-S (Merchant A. M. et al Nature Biothechnol 1998 vol 16 p 677-681) variants, or L234A, L235A, P329G (LALA-PG) substitutions, see e.g., Lo et al. “Effector Attenuating Substitutions that Maintain Antibody Stability and Reduce Toxicity in Mice. The Journal of Biological Chemistry Vol. 292, No. 9, pp. 3900-3908 Mar. 3, 2017, incorporated herein by reference. In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

Three highly divergent stretches within each of the heavy chain variable domain, VH or VH domain, and light chain variable domain, VL or VL domain, referred to as complementarity determining regions (CDRs), are interposed between more conserved flanking stretches known as “framework regions”, or “FRs”. Thus, the term “FR” refers to amino acid sequences which are naturally found between, and adjacent to, CDRs in immunoglobulins. A VH domain typically has four FRs, referred to herein as VH framework region 1 (FR1), VH framework region 2 (FR2), VH framework region 3 (FR3), and VH framework region 4 (FR4). Similarly, a VL domain typically has four FRs, referred to herein as VL framework region 1 (FR1), VL framework region 2 (FR2), VL framework region 3 (FR3), and VL framework region 4 (FR4). In an antibody molecule, the three CDRs of a VL domain (CDR-L1, CDR-L2 and CDR-L3) and the three CDRs of a VH domain (CDR-H1, CDR-H2 and CDR-H3) are disposed relative to each other in three-dimensional space to form an antigen-binding site within the antibody variable region. The surface of the antigen-binding site is complementary to a three-dimensional surface of a bound antigen. The amino acid sequences of VL and VH domains may be numbered, and CDRs and FRs therein identified/defined, according to the Kabat numbering system (Kabat et al., 1991, Sequences of Proteins of Immunological Interest, 5th Ed. Public Health Service, National Institutes of Health, Bethesda, Md.) or the INTERNATIONAL IMMUNOGENETICS INFORMATION SYSTEM (IMGT numbering system; Lefranc et al., 2003, Development and Comparative Immunology 27:55-77), both incorporated herein by reference. One of ordinary skill in the art would possess the knowledge for numbering amino acid residues of a VL domain and of a VH domain, and identifying CDRs and FRs therein, according to a routinely employed numbering system such as the IMGT numbering system, the Kabat numbering system, and the like.

The term “antibody” as referred to herein includes whole antibodies and any antigen binding fragment (i.e., “antigen-binding portion”) or single chain thereof. A “whole antibody” or full-length refers to a glycoprotein comprising at least two heavy (H) chains and two light (L) chains inter-connected by disulfide bonds, or an antigen binding portion thereof. Each heavy chain is comprised of a heavy chain variable region or domain (abbreviated herein as VH) and a heavy chain constant region. The heavy chain constant region is comprised of three domains, CH1, CH2 and CH3. Each light chain is comprised of a light chain variable region or domain (abbreviated herein as VL) and a light chain constant region. The light chain constant region is comprised of one domain, CL or CL1. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” or “antigen-binding fragment” of an antibody (or simply “antibody portion” or “antibody fragment”), as used herein, refers to one or more fragments, portions or domains of an antibody that retain the ability to specifically bind to an antigen. It has been shown that fragments of a full-length antibody can perform the antigen-binding function of an antibody. Examples of binding fragments encompassed within the term “antigen-binding portion” of an antibody include (i) an Fab fragment, a monovalent fragment consisting of the VL, VH, CL1 and CH1 domains; (ii) an F (ab′) 2 fragment, a bivalent fragment comprising two F (ab)′ fragments linked by a disulfide bridge at the hinge region; (iii) an Fd fragment consisting of the VH and CH1 domains; (iv) an Fv fragment consisting of the VL and VH domains of a single arm of an antibody; (v) a dAb fragment (Ward et al. (1989) Nature 241:544-546), which consists of a VH domain; and (vi) an isolated complementary determining region (CDR). Furthermore, although the two domains of the Fv fragment, VL and VH, are coded for by separate genes, they can be joined, using recombinant methods, by a synthetic linker that enables them to be made as a single contiguous chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies, are also encompassed (see e.g., Holliger et al. (1993) PNAS. USA 90:6444-6448).

“Diabodies,” or sometimes referred to herein as “Dia,” as used herein are dimeric antibody fragments. In each polypeptide of the diabody, a heavy-chain variable domain (VH) is linked to a light-chain variable domain (VL) but unlike single-chain Fv fragments, the linker between the VL and VH is too short for intramolecular pairing and as such each antigen-binding site is formed by pairing of the VH and VL of one polypeptide with the VH and VL of the other polypeptide. Diabodies thus have two antigen-binding sites, and can be monospecific or bispecific. (see, e.g., Holliger, P., et al. (1993) Proc. Natl. Acad. Sci. USA 90:6444-6448; Poljak, R. J., et al. (1994) Structure 2:1121-1123; Kontermann and Dubel eds., Antibody Engineering (2001) Springer-Verlag. New York. 790 pp. (ISBN 3-540-41354-5) incorporated herein by reference.

As used herein an “effective amount” of an agent, e.g., the tetravalent binding antibody molecules or a pharmaceutical composition comprising the molecules, refers to an amount effective, at dosages and for periods of time necessary, to achieve the desired result. In some embodiments, a therapeutically effective amount is one that reduces the incidence and/or severity of, stabilizes one or more characteristics of, and/or delays onset of, one or more symptoms of the disease, disorder, and/or condition. In some embodiments, the amount of a FZD Agonists administered to the subject is in the range of about 0.001 mg/kg to 10 mg/kg, 0.5 mg/kg to about 10 mg/kg, or about 0.5 mg/kg to about 1 mg/kg of the subject's body weight. For example, in some embodiments the FZD4 Agonist may be applied to the eye in an amount of, e.g., about 0.02-1.5 mg, about 0.05-1.0 mg, or about 0.1-0.5 mg per eye.

As used herein, the term “epitope” includes any protein determinant capable of specific binding to an immunoglobulin or fragment thereof, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three-dimensional structural characteristics, as well as specific charge characteristics. An antibody is said to specifically bind an antigen when the dissociation constant is ≤10 μM; e.g., ≤100 nM, preferably ≤10 nM and more preferably ≤1 nM.

The constant region of immunoglobulin molecules is also called the fragment crystallizable region, the “Fc region” or “Fc domain.” The Fc domain is composed of two identical protein fragments, derived from the second and third constant domains of the antibody's two heavy chains and the Fc domains of IgGs bear a highly conserved N-glycosylation site. Glycosylation of the Fc fragment is essential for Fc receptor-mediated activity. In an embodiment of the invention the Fc domain of the tetravalent binding antibody molecule is engineered such that it does not target the cell that binds the tetravalent binding antibody molecule for ADCC or CDC-dependent death. In an embodiment of the invention the Fc domain of the tetravalent binding antibody molecule is a peptide dimer in a knob-in-hole configuration. The peptide dimer may be a heterodimer.

The terms “individual,” “subject,” “host,” and “patient,” are used interchangeably herein and refer to any mammalian subject for whom diagnosis, treatment, or therapy is desired, particularly humans.

“LRP”, “LRP proteins” and “LRP receptors” is used herein to refer to members of the low density lipoprotein receptor-related protein family. These receptors are single-pass transmembrane proteins that bind and internalize ligands in the process of receptor-mediated endocytosis. LRP proteins LRP5 (e.g., LRP5: NP_002326.2) and LRP6 (e.g., LRP6: NP_002327.2) are included in a Wnt receptor complex required for activation on the Wnt-βcatenin signaling pathway. See also, for human/mouse LRP5 and LRP6: https://www.uniprot.org/uniprot/O75197, https://www.uniprot.org/uniprot/Q91VN0, https://www.uniprot.org/uniprot/O075581, https://www.uniprot.org/uniprot/O88572.

The term “polypeptide fragment” as used herein refers to a polypeptide that has an amino terminal and/or carboxy-terminal deletion, but where the remaining amino acid sequence is identical to the corresponding positions in the naturally-occurring sequence deduced, for example, from a full-length cDNA sequence.

As used herein the term “paratope” includes the antigen binding site in the variable region of an antibody that binds to an epitope.

“Single-chain Fv” or “scFv” antibody fragments comprise the VH and VL domains of an antibody, wherein these domains are present in a single polypeptide chain. Generally, the Fv polypeptide further comprises a polypeptide linker between the VH and VL domains which enables the scFv to form the desired structure for antigen binding. For a review of scFv and other antibody fragments, see James D. Marks, Antibody Engineering, Chapter 2, Oxford University Press (1995) (Carl K. Borrebaeck, Ed.).

“Single-domain antibody” (sdAb), or “nanobody”, is an antibody fragment consisting of a single monomeric variable antibody domain. “VHH” or “VHH fragment” as used herein refers to a human VH that has been engineered to be independent of the light chain (Nilvebrant et al. Curr Pharm Des. (2016) 22(43):6527-6537; Barthelemy et al., Journal of Biological Chemistry (2007) 283:3639-3654).

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., slowing or arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease.

The ability of the tetravalent binding antibody molecules of this invention to activate Wnt signaling can be confirmed by a number of assays. The tetravalent binding antibody molecules of this invention typically initiate a reaction or activity that is similar to or the same as that initiated by the FZD receptor's natural ligand. The tetravalent binding antibody molecules of this invention activates the Wnt signaling pathways, e.g., the canonical Wnt-βcatenin signaling pathway. As used herein, the term “activates” refers to a measurable increase in the intracellular level of a Wnt signaling pathway, e.g., the Wnt-βcatenin signaling pathway, compared with the level in the absence of a FZD Agonist of the invention.

Various methods are known in the art for measuring the level of Wnt-βcatenin activation. These include but are not limited to assays that measure: Wnt-βcatenin target gene expression; LEF/TCF reporter gene expression (such as TopFLASH, superTopFLASH, pBAR); βcatenin stabilization; LRP5/6 phosphorylation; Dishevelled phosphorylation; Axin translocation from cytoplasm to cell membrane and binding to LRP5/6. The canonical Wnt-βcatenin signaling pathway ultimately leads to changes in gene expression through the transcription factors TCF1, TCF7L1, TCF7L2 and LEF1. The transcriptional response to Wnt activation has been characterized in a number of cells and tissues. As such, global transcriptional profiling by methods well known in the art can be used to assess Wnt-βcatenin signaling activation.

Changes in Wnt-responsive gene expression are generally mediated by TCF and LEF transcription factors. A TCF reporter assay assesses changes in the transcription of TCF/LEF controlled genes to determine the level of Wnt-βcatenin signaling. A TCF reporter assay was first described by Korinek, V. et al., 1997. Also known as TOP/FOP this method involves the use of three copies of the optimal TCF motif CCTTTGATC, or three copies of the mutant motif CCTTTGGCC, upstream of a minimal c-Fos promoter driving luciferase expression (pTOPFLASH and pFOPFLASH, respectively) to determine the transactivational activity of endogenous βcatenin/TCF. A higher ratio of these two reporter activities (TOP/FOP) indicates higher βcatenin/TCF activity. A newer and more sensitive version of this reporter is called pBAR and contains 12 repeats of the TCF motifs (Biechele and Moon, Methods Mol Biol. 2008; 468:99-110, PMID: 19099249).

General methods in molecular and cellular biochemistry can be found in such standard textbooks as Molecular Cloning: A Laboratory Manual, 3rd Ed. (Sambrook et al., CSH Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998).

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

### EXAMPLES

### Example I Identification and Characterization of FZD4 Binding or FZD5 Binding Fab-Phage

a. FZD4 Antibodies from Affinity Matured Libraries of FZD4-Binding Antibody 5027 and 5044; FZD5 Antibodies from Affinity Matured Libraries of FZD5-Binding Antibody 2919 and 2928.

Affinity matured libraries of known FZD4-binding antibodies 5027 and 5044 and known FZD5-binding antibodies 2919 and 2928 were prepared using routine methods, essentially as described in US publication no. 2016/0194394, inventors Sidhu et al., see also Persson et al. J. Mol. Biol., 2013 Feb. 22; 425(4):803-11 https://pubmed.ncbi.nlm.nih.gov/23219464/, both incorporated herein in their entirety by reference.

The 6 CDRs of the heavy chain (CDR-H1, CDR-H2 and CDR-H3) and light chains (CDR-L1, CDR-L2 and CDR-L3) of antibodies 5044, 5027, 2919, and 2928 antibodies isolated from the affinity matured libraries are set forth in Table 1 and Table 2.

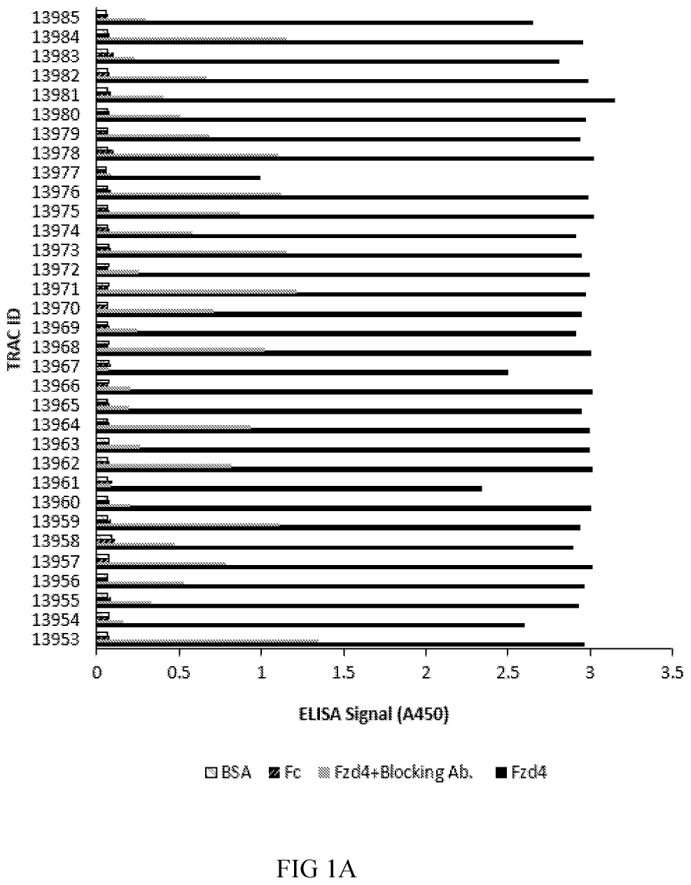

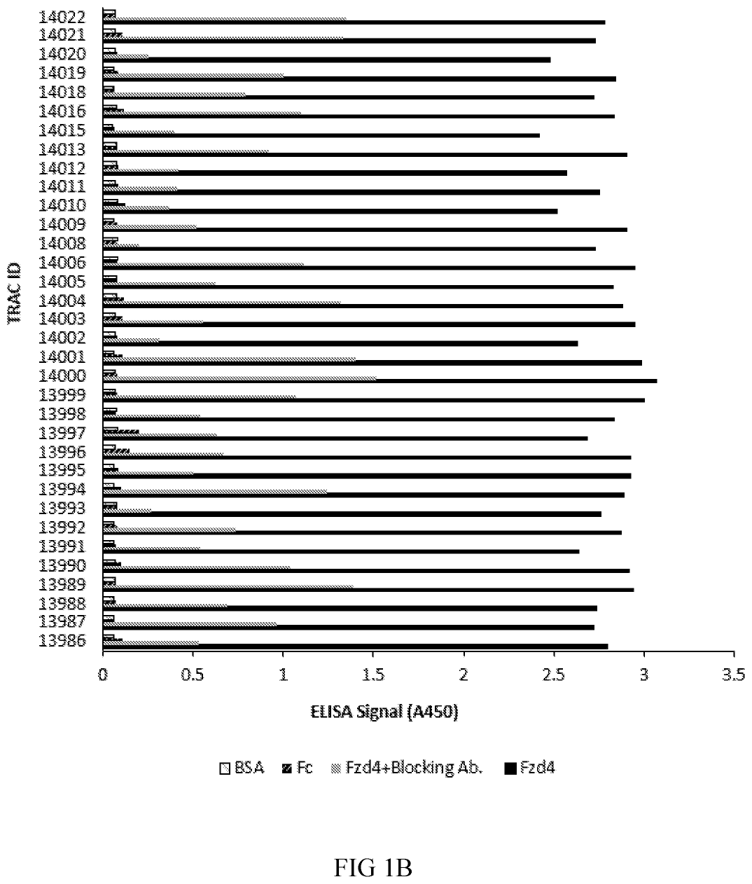

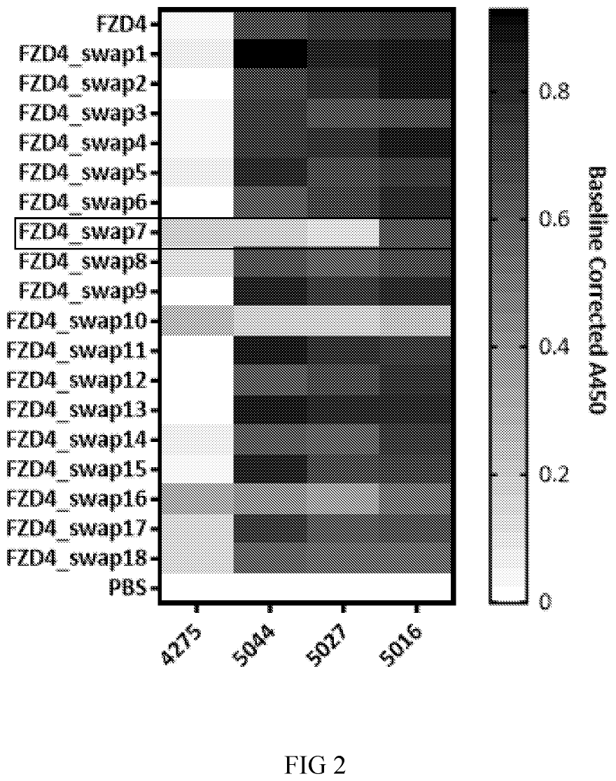

Single point ELISAs were performed on 96-well Maxisorp plates coated with the extracellular domains (ECDs) of human FZD4 protein in the presence or absence of a saturating concentration of 5027 diabody-Fc (a diabody comprising the VL and VH of 5027 linked to an Fc domain). The plates were incubated with monoclonal Fab-phage followed by incubation with horseradish peroxidase (HRP)-conjugated anti-M13 antibody. Wells were subsequently washed 8 times followed by incubations with 3,3,′5,5′-tetramethylbenidine/H₂O₂ peroxidase (TMB) substrate for 5-10 min. The reaction was stopped by adding 1M H₃PO₄ and the absorbance was measured spectrophotometrically at 450 nm in a microtiter plate reader. The results of the assay are depicted in FIG. 1 and FIG. 2 and demonstrate that the newly identified FZD4 antibodies bind FZD4 at a site overlapping with the site recognized by antibody 5027. FZD4 binding antibodies 5027 and 5044 are described in U.S. provisional application No. 62/885,781, incorporated herein by reference.

### B. Epitope Mapping of Lead FZD4 Antibodies.

ELISA assays were performed in 384-well Maxisorp plates coated with FZD4 ECD wild-type (FZD4) or mutant FZD4 proteins (FZD_swap1-18) that replace segments of the FZD4 ECD with corresponding regions from FZD5. The plates were incubated with 10 nM IgG known to bind specifically to FZD4, i.e., 5044 and 5027, or to be panspecific, i.e., 5016 (binds FZD4, FZD5, and other FZD receptors), followed by incubation with horseradish peroxidase (HRP)-conjugated anti-Kappa light chain antibody. Phosphate buffered saline (PBS) and IgG 4275 which does not bind FZD4 or FZD5 were used as controls. The wells were washed 6 times followed by incubations with 3,3,′5,5′-tetramethylbenidine/H₂O₂ peroxidase (TMB) substrate for 3-5 min. The reaction was stopped by adding 1M H₃PO₄ and the absorbance was measured spectrophotometrically at 450 nm in a microtiter plate reader, see FIG. 2. The pan-FZD binder 5016 is a positive control showing that the antigens are functional, with the exception of “FZD4_Swap10”. Both FZD4-specific antibodies 5027 and 5044 are unable to bind to “FZD4 Swap7”, suggesting that these molecules bind to this region of the FZD ECD.

### C. Characterization of FZD4 IgG.

FZD4-binding full length IgGs were expressed via transient transfection in an Expi293 cell culture system, essentially as described in Tao et al., Tailored tetravalent antibodies potently and specifically activate Wnt/Frizzled pathways in cells, organoids and mice. Elife. 2019 Aug. 27; 8:e46134. doi: 10.7554/eLife.46134; PMID: 31452509. and purified via Protein A affinity chromatography. Briefly, cells were grown to a density of approximately 2.5×10⁶ cells/ml in Expi293 Expression Media (Gibco) in baffled cell culture flasks and transfected with the appropriate vectors using FectoPRO transfection reagent (Polyplus-transfection) using standard manufacture protocols (ThermoFisher). Expression was allowed to proceed for 5 days at 37° C. and 8% CO₂ with shaking at 125 rpm. After expression, cells were removed by centrifugation and protein was purified from the conditioned media using rProtein A Sepharose (GE Healthcare). Purified protein was buffer exchanged into either PBS or a formulated stabilization buffer (36.8 mM citric acid, 63.2 mM Na₂HPO₄, 10% trehalose, 0.2 M L-arginine, 0.01% Tween-80, pH 6.0) for storage. Proteins concentrations were determined by absorbance at 280 nm and purity was confirmed by SDS-PAGE analysis.

Expression titers were determined as mg of purified protein per liter of mammalian cell culture. Size exclusion chromatography (SEC) results in Table A below are defined as “−”: evidence of multiple peaks on SEC trace, <50% monomeric species; “+”: >50% monomeric species, delayed retention time (>14 min.); “++”: >90% of major peak at/near expected retention time for a monomeric IgG. Standard retention time was determined by comparison to Trastuzumab.

TABLE A

| | Expression | SEC |
|---|---|---|
| TRAC ID | Titer (mg/l) | Result |
| 13980 | 58 | − |
| 13981 | 40 | − |
| 13982 | 43 | − |
| 13983 | 65 | ++ |
| 13984 | 52 | − |
| 13985 | 61 | ++ |
| 13956 | 47 | + |
| 13957 | 52 | + |
| 13958 | 58 | − |
| 13959 | 46 | + |
| 13962 | 38 | − |
| 13963 | 56 | − |
| 13964 | 47 | − |
| 13965 | 74 | ++ |
| 13966 | 86 | − |
| 13967 | 87 | − |
| 13968 | 89 | + |
| 13969 | 72 | − |
| 13970 | 36 | − |
| 13971 | 46 | ++ |
| 13972 | 27 | + |
| 13973 | 41 | + |
| 13974 | 41 | + |
| 13975 | 41 | ++ |
| 13979 | 49 | − |

Trac ID corresponds to the antibody number in Table 1 and Table 2.

### D. Size-Exclusion Chromatography Analysis and ELISA Specificity Measurements of the FZD4 IgGs.

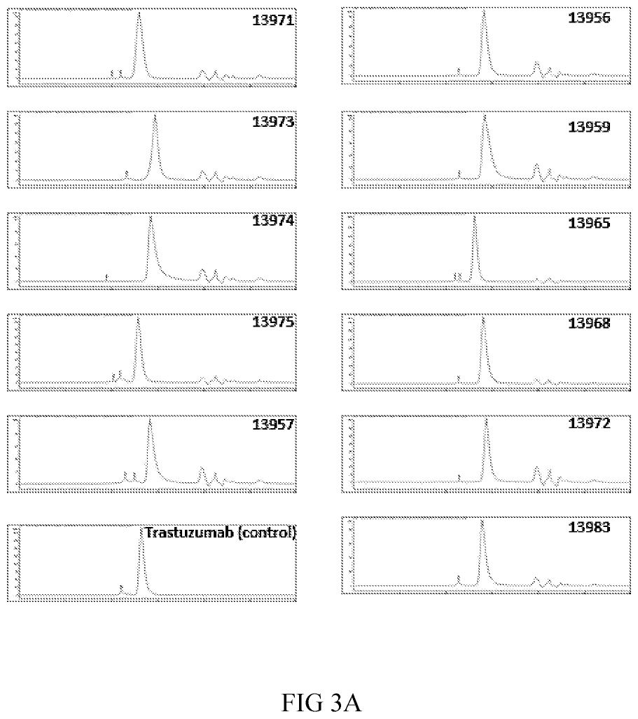

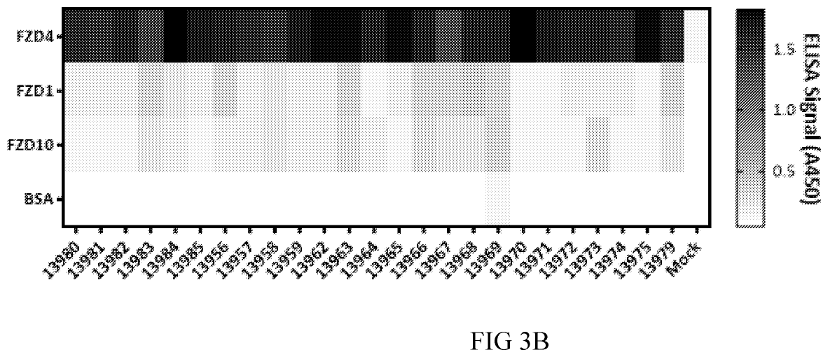

Twenty micrograms of the FZD4 binding IgGs were separated over an AdvanceBio SEC, 300 Å, 2.7 μm, 4.6×300 mm column in a mobile phase of PBS using an Agilent Bio-Inert HPLC. Protein elution was monitored using absorbance at 280 nM. The results are presented in FIG. 3A.

ELISA specificity measurements of the FZD4 antibodies were determined against FZD1 and FZD10, the two FZD family member most closely related to FZD4. ELISA assays were performed in 384-well Maxisorp plates coated with FZD ECD wild-type or mutant proteins at a concentration of 1 μg/ml and excess binding sites were blocked with 0.5% BSA. The plates were incubated with 10 nM of the FZD4 binding IgGs followed by incubation with horseradish peroxidase (HRP)-conjugated anti-Kappa light chain antibody. The wells were washed 6 times followed by incubations with 3,3,′5,5′-tetramethylbenidine/H₂O₂ peroxidase (TMB) substrate for 3-5 min. The reaction was stopped by adding 1M H₃PO₄ and the absorbance was measured spectrophotometrically at 450 nm in a microtiter plate reader. The results are presented in FIG. 3B.

### E. Identification of the CDRs of the FZD4 or FZD5 Binding Antibodies.

The amino acid sequences of the CDRs of the FZD4-binding and FZD5-binding immunoglobulins are set forth in Tables 1 and 2. The CDRs were identified according to the INTERNATIONAL IMMUNOGENETICS INFORMATION SYSTEM (IMGT numbering system; Lefranc et al., 2003, Development and Comparative Immunology 27:55-77), and annotated as described in Persson et al. J Mol Biol. 2013 Feb. 22; 425(4):803-11, both incorporated herein by reference.

TABLE 1

| | | FZD4 binding antibodies CDRs | | | | | | | | | | | |
|---|---|---|---|---|---|---|---|---|---|---|---|---|---|
| | | | SEQ | | SEQ | | SEQ | | SEQ | | SEQ | | SEQ |
| Antibody | | | ID | | ID | | ID | | ID | | ID | | ID |
| ID | Library | L1 | NO: | L2 | NO: | L3 | NO: | H1 | NO: | H2 | NO: | H3 | NO: |
| 5044 | F | SVSSA | 1 | SASSLYS | 2 | WYYAPI | 3 | LSSYSM | 24 | YISSYYGYTY | 51 | PAPGHWGF | 79 |
| 13953 | 5044AM | SVSSA | 1 | SASSLYS | 2 | GNYGPI | 4 | LAFYSI | 25 | YISPFSGITH | 52 | PAVGHLAM | 80 |
| 13954 | 5044AM | SVSSA | 1 | SASSLYS | 2 | GYYAPI | 5 | IYSYSI | 26 | YISSYYGYTY | 51 | STVGHGGM | 81 |
| 13955 | 5044AM | SVSSA | 1 | SASSLYS | 2 | WYYAPI | 3 | ISSYSI | 27 | YISPYYSYTY | 53 | PAPAHWGF | 82 |
| 13956 | 5044AM | SVSSA | 1 | SASSLYS | 2 | GYYALI | 6 | LSSYSM | 24 | YISSYAGYTS | 54 | PALGHAGM | 83 |
| 13957 | 5044AM | SVSSA | 1 | SASSLYS | 2 | WYFAPI | 7 | ISAYSI | 28 | YISPYFGLTG | 55 | PAPGHWGM | 84 |
| 13958 | 5044AM | SVSSA | 1 | SASSLYS | 2 | WYYAPI | 3 | LSSYSI | 29 | YISSYYGYTY | 51 | PVAGHGGM | 85 |
| 13959 | 5044AM | SVSSA | 1 | SASSLYS | 2 | GYNAPI | 8 | LYSYSM | 30 | FISSFYGYTD | 56 | PAVGHLAL | 86 |
| 13960 | 5044AM | SVSSA | 1 | SASSLYS | 2 | WYYAPI | 3 | LTSYSM | 31 | YISSYYGSTY | 57 | PAPGHWGM | 84 |
| 13961 | 5044AM | SVSSA | 1 | SASSLYS | 2 | WYYAPI | 3 | ISSYSM | 32 | YISSYYSYTY | 58 | PAPGYGAL | 87 |
| 13962 | 5044AM | SVSSA | 1 | SASSLYS | 2 | GYFAPI | 9 | LFSYPM | 33 | YISPYYGYTN | 59 | QTAGHAGM | 88 |
| 13963 | 5044AM | SVSSA | 1 | SASSLYS | 2 | WFNAPI | 10 | LSAYSM | 34 | YISSYYGYTY | 51 | PAPGHWGF | 79 |
| 13964 | 5044AM | SVSSA | 1 | SASSLYS | 2 | SNYAPI | 11 | ISSHSM | 35 | YISPFFSFTH | 60 | QAPGVSGI | 89 |
| 13965 | 5044AM | SVSSA | 1 | SASSLYS | 2 | WYNAPI | 12 | LSSYSM | 24 | YISSYDSITD | 61 | PAVGHMAF | 90 |
| 13966 | 5044AM | SVSSA | 1 | SASSLYS | 2 | GFYAPI | 13 | LSSYSM | 24 | YISSYYSYTA | 62 | PTPGHGGL | 91 |
| 13967 | 5044AM | SVSSA | 1 | SASSLYS | 2 | WYYAPI | 3 | ISAYAM | 36 | YISPYYGYTF | 63 | PAPGHGGM | 92 |
| 13968 | 5044AM | SVSSA | 1 | SASSLYS | 2 | GYSAPI | 14 | ISTYSM | 37 | YISPHYGFTS | 64 | PAVGHLGM | 93 |
| 13969 | 5044AM | SVSSA | 1 | SASSLYS | 2 | WYFAPI | 7 | LHAFSM | 38 | YISPYYGYTY | 65 | PAPGHWGL | 94 |
| 13970 | 5044AM | SVSSA | 1 | SASSLYS | 2 | GFYAPI | 13 | ISGYSI | 39 | YISSYYGYTF | 66 | TAPGHGAF | 95 |
| 13971 | 5044AM | SVSSA | 1 | SASSLYS | 2 | GFYAPI | 13 | ISNYSI | 40 | IISSNFGYTS | 67 | PALGHLAM | 96 |
| 13972 | 5044AM | SVSSA | 1 | SASSLYS | 2 | GYAGLI | 15 | ISAYSM | 41 | SISSYYGFTS | 68 | LAPGHPAL | 97 |
| 13973 | 5044AM | SVSSA | 1 | SASSLYS | 2 | GFSSPI | 16 | LTSYAM | 42 | YISPYYGYTY | 65 | PAAGHLAL | 98 |
| 13974 | 5044AM | SVSSA | 1 | SASSLYS | 2 | GYYAPI | 5 | LYSYSI | 43 | YISPSYGSTY | 69 | PIPGHLAF | 99 |
| 13975 | 5044AM | SVSSA | 1 | SASSLYS | 2 | GHYAPI | 17 | LSSFSM | 44 | YISSFNGSTF | 70 | PTWAHGAF | 100 |
| 13976 | 5044AM | SVSSA | 1 | SASSLYS | 2 | SFYAPI | 18 | IASYSI | 45 | YISSYYGSTY | 57 | PVLAHSAF | 101 |
| 13977 | 5044AM | SVSSA | 1 | SASSLYS | 2 | GYRAPI | 19 | IHSNSM | 46 | YISPYYSFTS | 71 | QTPGHSGM | 102 |
| 13978 | 5044AM | SVSSA | 1 | SASSLYS | 2 | WHRAPI | 20 | LSTNSM | 47 | YISPYYSFTY | 72 | QAPGPWGM | 103 |
| 13979 | 5044AM | SVSSA | 1 | SASSLYS | 2 | SFYAPI | 18 | LHSFSM | 48 | FISSYYGYTY | 73 | PAPGHGAF | 104 |
| 13980 | 5044AM | SVSSA | 1 | SASSLYS | 2 | GYYAPI | 5 | LTSYSM | 31 | SISPYYSYTN | 74 | PTTAHMAL | 105 |
| 13981 | 5044AM | SVSSA | 1 | SASSLYS | 2 | WYYAPI | 3 | ISSFSI | 49 | FINPYYSYTY | 75 | PAPGHWGM | 84 |
| 13982 | 5044AM | SVSSA | 1 | SASSLYS | 2 | GYYAPI | 5 | ISSYSM | 32 | YISSYYDYTY | 76 | PTPGHSGF | 106 |
| 13983 | 5044AM | SVSSA | 1 | SASSLYS | 2 | GDFAPF | 21 | LPYYSM | 50 | IISSYFGFTY | 77 | PAVGHGAL | 107 |
| 13984 | 5044AM | SVSSA | 1 | SASSLYS | 2 | GYSSPI | 22 | ISSHSM | 35 | YISPYYSYTY | 53 | TAPGHPAM | 108 |
| 13985 | 5044AM | SVSSA | 1 | SASSLYS | 2 | WFYAPI | 23 | ISSYSI | 27 | YISSNFGSTY | 78 | PVPAHGAF | 109 |

TABLE 2A

| | | FZD4 binding antibodies CDRs | | | | | | | | | | | |
|---|---|---|---|---|---|---|---|---|---|---|---|---|---|
| | | | SEQ | | SEQ | | SEQ | | SEQ | | SEQ | | SEQ |
| Antibody | | | ID | | ID | | ID | | ID | | ID | | ID |
| ID | Library | L1 | NO: | L2 | NO: | L3 | NO: | H1 | NO: | H2 | NO: | H3 | NO: |
| 5027 | F | SVSSA | 1 | SASSLYS | 2 | SSYSLI | 130 | SSFYFM | 148 | TVYPYLDYT | 985 | AFPGSYHPM | 199 |
| | | | | | | | | | | Y | | | |
| 13986 | 5027AM | SVSSA | 1 | SASSLYS | 2 | ASYYLI | 110 | STYFFI | 139 | TIYPYLNSTY | 166 | AYPGSYHPL | 198 |
| 13987 | 5027AM | SVSSA | 1 | SASSLYS | 2 | SNYALI | 111 | TSFYFM | 140 | SVYPYLDNTY | 167 | AFPGSYHPM | 199 |
| 13988 | 5027AM | SVSSA | 1 | SASSLYS | 2 | SSSFLI | 112 | SSFYFI | 141 | TVYSYIDITY | 168 | AFPFSYHPM | 200 |
| 13989 | 5027AM | SVSSA | 1 | SASSLYS | 2 | SSDSLI | 113 | SAYYFI | 142 | TVYPYRGYTY | 169 | GYPLAYTPL | 201 |
| 13990 | 5027AM | SVSSA | 1 | SASSLYS | 2 | STHFLI | 114 | SYFYFM | 143 | SVYPYLSYTY | 170 | AFPGSYHPM | 199 |
| 13991 | 5027AM | SVSSA | 1 | SASSLYS | 2 | ASYSLI | 115 | SSFYFI | 141 | SVYPYLDFTY | 171 | ALQGHYHPM | 202 |
| 13992 | 5027AM | SVSSA | 1 | SASSLYS | 2 | SAYTLI | 116 | TSFYYM | 144 | AIYPYLDYTY | 172 | AFPGSYLPM | 203 |
| 13993 | 5027AM | SVSSA | 1 | SASSLYS | 2 | SSVSLI | 117 | FTFYFM | 145 | SIYPYLNYTF | 173 | AFPGSYHPM | 199 |
| 13994 | 5027AM | SVSSA | 1 | SASSLYS | 2 | SYYSLI | 118 | SSYYFI | 146 | TIYPYSDNTY | 174 | GFPGRYHPL | 204 |
| 13995 | 5027AM | SVSSA | 1 | SASSLYS | 2 | SSYYLI | 119 | SSFYVM | 147 | SIYSYGNITY | 175 | AFPLSYHPM | 205 |
| 13996 | 5027AM | SVSSA | 1 | SASSLYS | 2 | ASYYLI | 110 | SSFYFM | 148 | AIYPYLSYTY | 176 | AFPGRYHGM | 206 |
| 13997 | 5027AM | SVSSA | 1 | SASSLYS | 2 | AYYFLI | 120 | SSFYYI | 149 | TVFPYLGRTY | 177 | AFPFSYTPL | 207 |
| 13998 | 5027AM | SVSSA | 1 | SASSLYS | 2 | SSFSLI | 121 | STFYFM | 150 | SVYPYLNYTY | 178 | AFPGAYSPM | 208 |
| 13999 | 5027AM | SVSSA | 1 | SASSLYS | 2 | SAYSLI | 122 | SAFYYM | 151 | TVYPYLSYTY | 179 | AFPGAYHPM | 209 |
| 14000 | 5027AM | SVSSA | 1 | SASSLYS | 2 | SSYALI | 123 | ASFYFM | 152 | TVYPYLNHTY | 180 | AFPGAYHPF | 210 |
| 14001 | 5027AM | SVSSA | 1 | SASSLYS | 2 | SYFSLI | 124 | SAFYFI | 153 | SIYPYLSYTY | 181 | AFPGAYHPM | 209 |
| 14002 | 5027AM | SVSSA | 1 | SASSLYS | 2 | SRFTLI | 125 | FPFYFM | 154 | SVYPYLNDTY | 182 | AYPGFYHPI | 211 |
| 14003 | 5027AM | SVSSA | 1 | SASSLYS | 2 | SSNTLI | 126 | SAFYFM | 155 | SVYSSLSHTY | 183 | AYPLSYHPM | 212 |
| 14004 | 5027AM | SVSSA | 1 | SASSLYS | 2 | SSFSLI | 121 | ASYYFM | 156 | SVYPYLDFTY | 171 | ALPGFYHPF | 213 |
| 14005 | 5027AM | SVSSA | 1 | SASSLYS | 2 | SSFALI | 127 | SVFYFM | 157 | SVYPYNDITY | 184 | GFPGTYHPL | 214 |
| 14006 | 5027AM | SVSSA | 1 | SASSLYS | 2 | ANYALI | 128 | SSLYYM | 158 | SVYPYLDNTH | 185 | AIPGFYHPI | 215 |
| 14008 | 5027AM | SVSSA | 1 | SASSLYS | 2 | ASYSLI | 115 | SNFYLM | 159 | SIYSYLNYTF | 186 | AFPGSYHPM | 199 |
| 14009 | 5027AM | SVSSA | 1 | SASSLYS | 2 | SSASLI | 129 | FSFYFI | 160 | SIYPYLDFTH | 187 | AFPGSYHPL | 216 |
| 14010 | 5027AM | SVSSA | 1 | SASSLYS | 2 | SSYSLI | 130 | SAFYFM | 155 | AIYPYIGYTY | 188 | PFPASYHPL | 217 |
| 14011 | 5027AM | SVSSA | 1 | SASSLYS | 2 | SSYSLI | 130 | SSLYFM | 161 | TIYPFRGTTY | 189 | AYPGRYHPL | 218 |
| 14012 | 5027AM | SVSSA | 1 | SASSLYS | 2 | SAFYLI | 131 | YYFYYM | 162 | AIYPYLGYTY | 190 | AFPGSYHPL | 216 |
| 14013 | 5027AM | SVSSA | 1 | SASSLYS | 2 | SAYFLI | 132 | SSFYFI | 141 | SVYPYLGDTY | 191 | AFPGFYHPF | 219 |
| 14015 | 5027AM | SVSSA | 1 | SASSLYS | 2 | ASSSLI | 133 | TSYYFI | 163 | SVYSYLGYTF | 192 | AFPGSYHPL | 216 |
| 14016 | 5027AM | SVSSA | 1 | SASSLYS | 2 | SIYSLI | 134 | SYFYFM | 143 | AIYPYLSYTY | 176 | AFPGSYHPM | 199 |
| 14018 | 5027AM | SVSSA | 1 | SASSLYS | 2 | ASYYLI | 110 | FSFYFI | 160 | TVYPYLSHTY | 193 | AFPGSYHPM | 199 |
| 14019 | 5027AM | SVSSA | 1 | SASSLYS | 2 | STGSLI | 135 | SDFYFI | 164 | TIYPFIGNTY | 194 | AFPGSYHPF | 220 |
| 14020 | 5027AM | SVSSA | 1 | SASSLYS | 2 | SSHSLI | 136 | SSFYFM | 148 | SVYPYVDYTY | 195 | AFPGFYHPM | 221 |
| 14021 | 5027AM | SVSSA | 1 | SASSLYS | 2 | SSYTLI | 137 | SSFYYM | 165 | TVYPYLSFTY | 196 | ALPGSYHPF | 222 |
| 14022 | 5027AM | SVSSA | 1 | SASSLYS | 2 | SYHYLI | 138 | STFYFM | 150 | SVYPYLDDTY | 197 | AYPGSYHPL | 198 |

TABLE 2B
FZD5 binding antibodies CDRs

| Anti- | Anti- | Selec- | | SEQ | | SEQ | | SEQ | | SEQ | | SEQ | | SEQ |
|---|---|---|---|---|---|---|---|---|---|---|---|---|---|---|
| body | gen | tion | | ID | | ID | | ID | | ID | | ID | | ID |
| ID | Name | ID | L1 | NO: | L2 | NO: | L3 | NO: | H1 | NO: | H2 | NO | H3 | NO: |
| 14023 | FZD5 | 2919AM | SVSSA | 1 | SASS | 2 | WYSG | 223 | LIYTYI | 308 | TIYP | 381 | GAM | |
| | | | | | LYS | | VHGL | | | | ASSS | | | |
| | | | | | | | I | | | | TS | | | |
| 14024 | FZD5 | 2919AM | SVSSA | 1 | SASS | 2 | WFSS | 224 | ITYPGM | 309 | TIFS | 382 | FGM | |
| | | | | | LYS | | AHVP | | | | SHGS | | | |
| | | | | | | | F | | | | TS | | | |
| 14025 | FZD5 | 2919AM | SVSSA | 1 | SASS | 2 | WYSY | 225 | ISYSYM | 310 | SIYS | 383 | GAL | |
| | | | | | LYS | | GHHL | | | | SSSS | | | |
| | | | | | | | I | | | | TS | | | |
| 14026 | FZD5 | 2919AM | SVSSA | 1 | SASS | 2 | WYTS | 226 | ISFFYM | 311 | TIDS | 384 | GAL | |
| | | | | | LYS | | GHVL | | | | STGS | | | |
| | | | | | | | I | | | | TT | | | |
| 14027 | FZD5 | 2919AM | SVSSA | 1 | SASS | 2 | WYAS | 227 | IPYFYM | 312 | SIYS | 385 | GAM | |
| | | | | | LYS | | DHGL | | | | SSGS | | | |
| | | | | | | | I | | | | TS | | | |
| 14028 | FZD5 | 2919AM | SVSSA | 1 | SASS | 2 | WFSP | 228 | ISYYYI | 313 | SIYP | 386 | AAF | |
| | | | | | LYS | | GNVL | | | | SSSS | | | |
| | | | | | | | I | | | | TT | | | |
| 14029 | FZD5 | 2919AM | SVSSA | 1 | SASS | 2 | WYST | 229 | ISYAYM | 314 | SIYP | 387 | YAF | |
| | | | | | LYS | | RNIL | | | | SSSS | | | |
| | | | | | | | I | | | | TA | | | |
| 14030 | FZD5 | 2919AM | SVSSA | 1 | SASS | 2 | WYFS | 230 | ITYFYM | 315 | SIYP | 388 | GGM | |
| | | | | | LYS | | DHDL | | | | SFGS | | | |
| | | | | | | | F | | | | TS | | | |
| 14031 | FZD5 | 2919AM | SVSSA | 1 | SASS | 2 | WYSS | 231 | IYYSYI | 316 | SIYS | 389 | GAM | |
| | | | | | LYS | | GDVL | | | | SNGG | | | |
| | | | | | | | I | | | | TS | | | |
| 14032 | FZD5 | 2919AM | SVSSA | 1 | SASS | 2 | WYSS | 232 | ILNTYM | 317 | SIYP | 387 | GGL | |
| | | | | | LYS | | ADVL | | | | SSSS | | | |
| | | | | | | | F | | | | TA | | | |
| 14033 | FZD5 | 2919AM | SVSSA | 1 | SASS | 2 | WYSS | 233 | ISFYYI | 318 | SIYP | 390 | GGI | |
| | | | | | LYS | | GHGL | | | | ASSS | | | |
| | | | | | | | I | | | | TS | | | |
| 14034 | FZD5 | 2919AM | SVSSA | 1 | SASS | 2 | WYSS | 234 | LAYSYM | 319 | SIYP | 391 | GAM | |
| | | | | | LYS | | GHAL | | | | SSGD | | | |
| | | | | | | | I | | | | TS | | | |
| 14035 | FZD5 | 2919AM | SVSSA | 1 | SASS | 2 | WYSS | 235 | IRYSYI | 320 | AIYS | 392 | GAM | |
| | | | | | LYS | | NHIP | | | | SSSS | | | |
| | | | | | | | I | | | | TS | | | |
| 14036 | FZD5 | 2919AM | SVSSA | 1 | SASS | 2 | WYSS | 236 | ITYSYM | 321 | TIYP | 393 | FAM | |
| | | | | | LYS | | SNVL | | | | SSGS | | | |
| | | | | | | | I | | | | TA | | | |
| 14037 | FZD5 | 2919AM | SVSSA | 1 | SASS | 2 | WYFS | 237 | IINTYM | 322 | SIYS | 394 | GAI | |
| | | | | | LYS | | DRVL | | | | APSS | | | |
| | | | | | | | I | | | | TA | | | |
| 14038 | FZD5 | 2919AM | SVSSA | 1 | SASS | 2 | WYPS | 238 | ISYSYM | 310 | TIYP | 393 | GGM | |
| | | | | | LYS | | SHVL | | | | SSGS | | | |
| | | | | | | | I | | | | TA | | | |
| 14039 | FZD5 | 2919AM | SVSSA | 1 | SASS | 2 | WYSS | 239 | LANSYM | 323 | TIYS | 395 | GAM | |
| | | | | | LYS | | RHHL | | | | SSGS | | | |
| | | | | | | | I | | | | TS | | | |
| 14040 | FZD5 | 2919AM | SVSSA | 1 | SASS | 2 | WYST | 240 | LSYTYM | 324 | SIDP | 396 | FGM | |
| | | | | | LYS | | GRVL | | | | SSGS | | | |
| | | | | | | | F | | | | TS | | | |
| 14041 | FZD5 | 2919AM | SVSSA | 1 | SASS | 2 | WFPT | 241 | ISYSYI | 325 | TIYP | 397 | FGM | |
| | | | | | LYS | | GHVL | | | | SSGS | | | |
| | | | | | | | F | | | | TG | | | |
| 14042 | FZD5 | 2919AM | SVSSA | 1 | SASS | 2 | WYAP | 242 | ISYSYI | 325 | SIYS | 383 | GAM | |
| | | | | | LYS | | RHVL | | | | SSSS | | | |
| | | | | | | | I | | | | TS | | | |
| 14043 | FZD5 | 2919AM | SVSSA | 1 | SASS | 2 | WFTS | 243 | LSYLYI | 326 | SIYP | 398 | GGM | |
| | | | | | LYS | | GLVL | | | | SSSS | | | |
| | | | | | | | F | | | | TS | | | |
| 14044 | FZD5 | 2919AM | SVSSA | 1 | SASS | 2 | WYSS | 244 | ISYAYI | 327 | SIYS | 399 | GAM | |
| | | | | | LYS | | GHVL | | | | SPSS | | | |
| | | | | | | | F | | | | TS | | | |
| 14045 | FZD5 | 2919AM | SVSSA | 1 | SASS | 2 | WYPG | 245 | IAYSYM | 328 | SIYP | 398 | GAM | |
| | | | | | LYS | | APVL | | | | SSSS | | | |
| | | | | | | | I | | | | TS | | | |
| 14046 | FZD5 | 2919AM | SVSSA | 1 | SASS | 2 | WYSS | 236 | FIWESL | 329 | TIYP | 393 | FAM | |
| | | | | | LYS | | SNVL | | LVS | | SSGS | | | |
| | | | | | | | I | | | | TA | | | |
| 14047 | FZD5 | 2919AM | SVSSA | 1 | SASS | 2 | WYSS | 246 | ISHSYM | 330 | SIDS | 400 | GAL | |
| | | | | | LYS | | ANAL | | | | SSGS | | | |
| | | | | | | | F | | | | TS | | | |
| 14048 | FZD5 | 2919AM | SVSSA | 1 | SASS | 2 | WYSS | 231 | LRYSYI | 331 | TIYS | 401 | GAL | |
| | | | | | LYS | | GDVL | | | | ASGS | | | |
| | | | | | | | I | | | | TT | | | |
| 14049 | FZD5 | 2919AM | SVSSA | 1 | SASS | 2 | WYSS | 247 | ISYAYM | 314 | SISP | 402 | GAL | |
| | | | | | LYS | | NHVL | | | | SDSS | | | |
| | | | | | | | I | | | | TS | | | |
| 14050 | FZD5 | 2919AM | SVSSA | 1 | SASS | 2 | WYSA | 248 | ISYSYM | 310 | SIDP | 403 | GAL | |
| | | | | | LYS | | AHIL | | | | SSGL | | | |
| | | | | | | | F | | | | TS | | | |
| 14051 | FZD5 | 2919AM | SVSSA | 1 | SASS | 2 | WFAS | 249 | IRYAYM | 332 | TIDS | 404 | YAM | |
| | | | | | LYS | | GHVL | | | | FSGS | | | |
| | | | | | | | I | | | | TS | | | |
| 14052 | FZD5 | 2919AM | SVSSA | 1 | SASS | 2 | WFSS | 250 | ITHLYM | 333 | SIYP | 405 | AAL | |
| | | | | | LYS | | GDSL | | | | SSGS | | | |
| | | | | | | | I | | | | TS | | | |
| 14053 | FZD5 | 2919AM | SVSSA | 1 | SASS | 2 | WYAS | 251 | LSIFYM | 334 | TIYP | 406 | GAF | |
| | | | | | LYS | | GNVL | | | | SSSS | | | |
| | | | | | | | I | | | | TS | | | |
| 14054 | FZD5 | 2919AM | SVSSA | 1 | SASS | 2 | WFSP | 252 | LSYSYI | 335 | AIYP | 407 | GAM | |
| | | | | | LYS | | GHLL | | | | SPSS | | | |
| | | | | | | | I | | | | TS | | | |
| 14055 | FZD5 | 2919AM | SVSSA | 1 | SASS | 2 | WYPS | 253 | ISYSFM | 336 | SIYS | 408 | GGL | |
| | | | | | LYS | | GHVL | | | | TSGS | | | |
| | | | | | | | F | | | | TS | | | |
| 14056 | FZD5 | 2919AM | SVSSA | 1 | SASS | 2 | WFAA | 254 | IAYVGI | 337 | AITP | 409 | FGF | |
| | | | | | LYS | | GHVL | | | | SSSN | | | |
| | | | | | | | I | | | | TS | | | |
| 14057 | FZD5 | 2919AM | SVSSA | 1 | SASS | 2 | WFRS | 255 | LSYSYM | 338 | SIDP | 410 | FGF | |
| | | | | | LYS | | AHVL | | | | SSSS | | | |
| | | | | | | | I | | | | TA | | | |
| 14058 | FZD5 | 2919AM | SVSSA | 1 | SASS | 2 | WFAS | 256 | ISYSFM | 336 | SIYS | 411 | GAM | |
| | | | | | LYS | | GHIL | | | | RSGS | | | |
| | | | | | | | I | | | | TA | | | |
| 14059 | FZD5 | 2919AM | SVSSA | 1 | SASS | 2 | WFSG | 257 | IAYFYI | 339 | TIDP | 412 | GGL | |
| | | | | | LYS | | GHAL | | | | SSGS | | | |
| | | | | | | | I | | | | TS | | | |
| 14060 | FZD5 | 2919AM | SVSSA | 1 | SASS | 2 | WFAS | 258 | LSSSYM | 340 | SIGP | 413 | FGF | |
| | | | | | LYS | | AHVL | | | | SSGS | | | |
| | | | | | | | F | | | | TS | | | |
| 14061 | FZD5 | 2919AM | SVSSA | 1 | SASS | 2 | WYSP | 259 | LSYSYM | 338 | SIYP | 398 | GGI | |
| | | | | | LYS | | GHVL | | | | SSSS | | | |
| | | | | | | | I | | | | TS | | | |
| 14062 | FZD5 | 2919AM | SVSSA | 1 | SASS | 2 | WYAS | 260 | IYYSYM | 341 | SIDP | 414 | GGM | |
| | | | | | LYS | | GLVL | | | | SSSS | | | |
| | | | | | | | I | | | | TS | | | |
| 14063 | FZD5 | 2919AM | SVSSA | 1 | SASS | 2 | WYTS | 261 | ITYSYM | 321 | SIYP | 415 | GGM | |
| | | | | | LYS | | GHVL | | | | SPSS | | | |
| | | | | | | | F | | | | TS | | | |
| 14064 | FZD5 | 2919AM | SVSSA | 1 | SASS | 2 | WFSF | 262 | LHYGGI | 342 | SISS | 416 | FGF | |
| | | | | | LYS | | PHAL | | | | SFSS | | | |
| | | | | | | | I | | | | TS | | | |
| 14066 | FZD5 | 2919AM | SVSSA | 1 | SASS | 2 | WYAS | 263 | ISYSYM | 310 | TIYS | 417 | GGM | |
| | | | | | LYS | | GTDL | | | | SSSS | | | |
| | | | | | | | I | | | | TA | | | |
| 14067 | FZD5 | 2919AM | SVSSA | 1 | SASS | 2 | WYSS | 264 | IGYAYM | 343 | SIYS | 418 | GAL | |
| | | | | | LYS | | GRLL | | | | SPGS | | | |
| | | | | | | | I | | | | TA | | | |
| 14068 | FZD5 | 2919AM | SVSSA | 1 | SASS | 2 | WFSS | 265 | LSYSSI | 344 | SICP | 419 | FGF | |
| | | | | | LYS | | PHVL | | | | FCSS | | | |
| | | | | | | | F | | | | TS | | | |
| 14069 | FZD5 | 2919AM | SVSSA | 1 | SASS | 2 | WFAS | 266 | LSYDGI | 345 | TIYS | 395 | FGL | |
| | | | | | LYS | | ASSL | | | | SSGS | | | |
| | | | | | | | F | | | | TS | | | |
| 14070 | FZD5 | 2919AM | SVSSA | 1 | SASS | 2 | WYPS | 267 | LSYAYM | 346 | SIHP | 420 | GAL | |
| | | | | | LYS | | SHVL | | | | FDGS | | | |
| | | | | | | | F | | | | TS | | | |
| 14071 | FZD5 | 2919AM | SVSSA | 1 | SASS | 2 | WFPA | 268 | ISYSGI | 347 | SISS | 421 | FGM | |
| | | | | | LYS | | HHVL | | | | SSGS | | | |
| | | | | | | | F | | | | TA | | | |
| 14072 | FZD5 | 2919AM | SVSSA | 1 | SASS | 2 | WFPS | 269 | ISSSYM | 348 | TIDP | 422 | FAM | |
| | | | | | LYS | | GNVL | | | | YAGS | | | |
| | | | | | | | F | | | | TS | | | |
| 14073 | FZD5 | 2919AM | SVSSA | 1 | SASS | 2 | WYSS | 270 | LSYSYM | 338 | SIYP | 423 | GGM | |
| | | | | | LYS | | NNVL | | | | SSGS | | | |
| | | | | | | | I | | | | TA | | | |
| 14074 | FZD5 | 2919AM | SVSSA | 1 | SASS | 2 | WFSS | 271 | LSYSGM | 349 | TIYP | 406 | FGM | |
| | | | | | LYS | | RHVL | | | | SSSS | | | |
| | | | | | | | F | | | | TS | | | |
| 14075 | FZD5 | 2919AM | SVSSA | 1 | SASS | 2 | WYFS | 272 | GFTISY | 350 | SIYP | 424 | GAM | |
| | | | | | LYS | | DHDL | | FFM | | SDSS | | | |
| | | | | | | | I | | | | TS | | | |
| 14076 | FZD5 | 2919AM | SVSSA | 1 | SASS | 2 | WYSS | 273 | ISYSFI | 351 | SIYP | 425 | GAL | |
| | | | | | LYS | | GGVL | | | | SSGH | | | |
| | | | | | | | I | | | | TS | | | |
| 14077 | FZD5 | 2919AM | SVSSA | 1 | SASS | 2 | WFPS | 274 | ISYSYM | 310 | SIDP | 396 | FGM | |
| | | | | | LYS | | GHVL | | | | SSGS | | | |
| | | | | | | | F | | | | TS | | | |
| 14078 | FZD5 | 2919AM | SVSSA | 1 | SASS | 2 | WYSS | 275 | LSYTYM | 324 | SIYS | 426 | GGM | |
| | | | | | LYS | | THVL | | | | SPGS | | | |
| | | | | | | | F | | | | TS | | | |
| 14079 | FZD5 | 2919AM | SVSSA | 1 | SASS | 2 | WYSS | 276 | ISHAYM | 352 | SIYS | 418 | FAL | |
| | | | | | LYS | | GSAL | | | | SPGS | | | |
| | | | | | | | I | | | | TA | | | |
| 14080 | FZD5 | 2919AM | SVSSA | 1 | SASS | 2 | WFSP | 277 | LLFFYM | 353 | SIDP | 427 | GAM | |
| | | | | | LYS | | GSVL | | | | SSGF | | | |
| | | | | | | | I | | | | TS | | | |
| 14081 | FZD5 | 2919AM | SVSSA | 1 | SASS | 2 | WYPS | 278 | IAYSYM | 328 | SIYP | 390 | GGM | |
| | | | | | LYS | | AHIL | | | | ASSS | | | |
| | | | | | | | F | | | | TS | | | |
| 14082 | FZD5 | 2919AM | SVSSA | 1 | SASS | 2 | WYGS | 279 | LSYFYM | 354 | SIFS | 428 | GAM | |
| | | | | | LYS | | GGIL | | | | SSSS | | | |
| | | | | | | | I | | | | TS | | | |
| 14083 | FZD5 | 2919AM | SVSSA | 1 | SASS | 2 | WFTS | 280 | GFTLIS | 355 | SIYP | 429 | GGM | |
| | | | | | LYS | | GRDL | | SSI | | APSS | | | |
| | | | | | | | F | | | | TP | | | |
| 14084 | FZD5 | 2919AM | SVSSA | 1 | SASS | 2 | WYLS | 281 | LSYSYI | 335 | SIYS | 383 | GAM | |
| | | | | | LYS | | RNIL | | | | SSSS | | | |
| | | | | | | | I | | | | TS | | | |
| 14085 | FZD5 | 2919AM | SVSSA | 1 | SASS | 2 | WFSG | 282 | ITNSYM | 356 | SIYS | 399 | GGL | |
| | | | | | LYS | | RDAL | | | | SPSS | | | |
| | | | | | | | F | | | | TS | | | |
| 14369 | FZD5 | 2928AM | SVSSA | 1 | SASS | 2 | AFFY | 283 | ISFSSI | 357 | SIYP | 430 | YYAF | 483 |
| | | | | | LYS | | PI | | | | SYGS | | | |
| | | | | | | | | | | | SF | | | |
| 14370 | FZD5 | 2928AM | SVSSA | 1 | SASS | 2 | AFYF | 284 | ISYSSM | 358 | TIYP | 431 | YYAF | 483 |
| | | | | | LYS | | PI | | | | YYSS | | | |
| | | | | | | | | | | | TL | | | |
| 14371 | FZD5 | 2928AM | SVSSA | 1 | SASS | 2 | AHYF | 285 | ISYSSM | 358 | SIYS | 432 | YYAM | 484 |
| | | | | | LYS | | PI | | | | SYSS | | | |
| | | | | | | | | | | | TY | | | |
| 14372 | FZD5 | 2928AM | SVSSA | 1 | SASS | 2 | AFYF | 284 | ISYSSM | 358 | SIYP | 433 | YYGM | 485 |
| | | | | | LYS | | PI | | | | SYSV | | | |
| | | | | | | | | | | | TY | | | |
| 14373 | FZD5 | 2928AM | SVSSA | 1 | SASS | 2 | SHYY | 286 | LSFSSM | 359 | SIYP | 434 | YYAM | 484 |
| | | | | | LYS | | PI | | | | YYGS | | | |
| | | | | | | | | | | | TF | | | |
| 14374 | FZD5 | 2928AM | SVSSA | 1 | SASS | 2 | AFYF | 284 | ISYSSM | 358 | SIYS | 432 | YYGF | 486 |
| | | | | | LYS | | PI | | | | SYSS | | | |
| | | | | | | | | | | | TY | | | |
| 14375 | FZD5 | 2928AM | SVSSA | 1 | SASS | 2 | GFYY | 287 | ISFGSI | 360 | SIYP | 435 | YYAM | 484 |
| | | | | | LYS | | PI | | | | SYSS | | | |
| | | | | | | | | | | | TF | | | |
| 14376 | FZD5 | 2928AM | SVSSA | 1 | SASS | 2 | AFYY | 288 | ITYSSI | 361 | SIYP | 436 | YYAF | 483 |
| | | | | | LYS | | PI | | | | AYSS | | | |
| | | | | | | | | | | | TY | | | |
| 14377 | FZD5 | 2928AM | SVSSA | 1 | SASS | 2 | SFYF | 289 | ISYSAI | 362 | SIYS | 437 | YYAM | 484 |
| | | | | | LYS | | PI | | | | SYSS | | | |
| | | | | | | | | | | | TF | | | |
| 14378 | FZD5 | 2928AM | SVSSA | 1 | SASS | 2 | AFYF | 284 | ISYSSM | 358 | SIYS | 438 | YYAM | 484 |
| | | | | | LYS | | PI | | | | SYGS | | | |
| | | | | | | | | | | | TY | | | |
| 14379 | FZD5 | 2928AM | SVSSA | 1 | SASS | 2 | ADYF | 290 | ISYSSM | 358 | SIYP | 439 | YYGM | 485 |
| | | | | | LYS | | PI | | | | SYSS | | | |
| | | | | | | | | | | | TY | | | |
| 14380 | FZD5 | 2928AM | SVSSA | 1 | SASS | 2 | AFYY | 288 | ISYSSI | 363 | SIYS | 440 | YYAM | 484 |
| | | | | | LYS | | PI | | | | YYGS | | | |
| | | | | | | | | | | | TY | | | |
| 14381 | FZD5 | 2928AM | SVSSA | 1 | SASS | 2 | AFYF | 284 | ISFSSI | 357 | TIYS | 441 | YYGF | 486 |
| | | | | | LYS | | PI | | | | LFGA | | | |
| | | | | | | | | | | | TF | | | |
| 14382 | FZD5 | 2928AM | SVSSA | 1 | SASS | 2 | SFYF | 289 | ISYSSM | 358 | SIYP | 439 | YYAF | 483 |
| | | | | | LYS | | PI | | | | SYSS | | | |
| | | | | | | | | | | | TY | | | |
| 14383 | FZD5 | 2928AM | SVSSA | 1 | SASS | 2 | AFHY | 291 | ISFSSI | 357 | SIYP | 439 | YYAM | 484 |
| | | | | | LYS | | PI | | | | SYSS | | | |
| | | | | | | | | | | | TY | | | |
| 14384 | FZD5 | 2928AM | SVSSA | 1 | SASS | 2 | AFYY | 288 | ISFSSI | 357 | SIYP | 442 | YYGM | 485 |
| | | | | | LYS | | PI | | | | AYGA | | | |
| | | | | | | | | | | | TF | | | |
| 14385 | FZD5 | 2928AM | SVSSA | 1 | SASS | 2 | AYYY | 292 | ISYSSI | 363 | SIYS | 437 | YYAF | 483 |
| | | | | | LYS | | PI | | | | SYSS | | | |
| | | | | | | | | | | | TF | | | |
| 14386 | FZD5 | 2928AM | SVSSA | 1 | SASS | 2 | VFYY | 293 | ISYSSM | 358 | SIYS | 432 | YYAF | 483 |
| | | | | | LYS | | PI | | | | SYSS | | | |
| | | | | | | | | | | | TY | | | |
| 14387 | FZD5 | 2928AM | SVSSA | 1 | SASS | 2 | SFYF | 289 | ISYSSM | 358 | SIYP | 443 | YYAM | 484 |
| | | | | | LYS | | PI | | | | YYSS | | | |
| | | | | | | | | | | | TY | | | |
| 14388 | FZD5 | 2928AM | SVSSA | 1 | SASS | 2 | AFYF | 284 | LSYSSM | 364 | SIYP | 436 | YYGM | 485 |
| | | | | | LYS | | PI | | | | AYSS | | | |
| | | | | | | | | | | | TY | | | |
| 14389 | FZD5 | 2928AM | SVSSA | 1 | SASS | 2 | ANYF | 294 | ISFGSI | 360 | SIYS | 432 | YYAM | 484 |
| | | | | | LYS | | PI | | | | SYSS | | | |
| | | | | | | | | | | | TY | | | |
| 14390 | FZD5 | 2928AM | SVSSA | 1 | SASS | 2 | GFYF | 295 | ISYSSI | 363 | SIYP | 444 | YYAM | 484 |
| | | | | | LYS | | PI | | | | SFGS | | | |
| | | | | | | | | | | | TY | | | |
| 14391 | FZD5 | 2928AM | SVSSA | 1 | SASS | 2 | AFYF | 284 | ISYSSM | 358 | SIYP | 445 | YYGM | 485 |
| | | | | | LYS | | PI | | | | SFSP | | | |
| | | | | | | | | | | | TY | | | |
| 14392 | FZD5 | 2928AM | SVSSA | 1 | SASS | 2 | AFYF | 284 | IAYSSM | 365 | SIYS | 446 | YYAF | 483 |
| | | | | | LYS | | PI | | | | SYGS | | | |
| | | | | | | | | | | | TF | | | |
| 14393 | FZD5 | 2928AM | SVSSA | 1 | SASS | 2 | AYYF | 296 | ISYSSI | 363 | SIYP | 447 | YYAF | 483 |
| | | | | | LYS | | PI | | | | AYSP | | | |
| | | | | | | | | | | | TY | | | |
| 14394 | FZD5 | 2928AM | SVSSA | 1 | SASS | 2 | AFIF | 297 | ISYSAM | 366 | SIYP | 439 | YYAL | 487 |
| | | | | | LYS | | PI | | | | SYSS | | | |
| | | | | | | | | | | | TY | | | |
| 14395 | FZD5 | 2928AM | SVSSA | 1 | SASS | 2 | AYYY | 292 | ISYGVI | 367 | SIYS | 448 | YYAF | 483 |
| | | | | | LYS | | PI | | | | AYSS | | | |
| | | | | | | | | | | | TF | | | |
| 14396 | FZD5 | 2928AM | SVSSA | 1 | SASS | 2 | SFYF | 289 | ISYSSI | 363 | TIYP | 449 | YYAF | 483 |
| | | | | | LYS | | PI | | | | YYDP | | | |
| | | | | | | | | | | | TY | | | |
| 14397 | FZD5 | 2928AM | SVSSA | 1 | SASS | 2 | AFYF | 284 | LSYSSM | 364 | SIYS | 438 | YYAM | 484 |
| | | | | | LYS | | PI | | | | SYGS | | | |
| | | | | | | | | | | | TY | | | |
| 14398 | FZD5 | 2928AM | SVSSA | 1 | SASS | 2 | AFYY | 288 | ISYSSI | 363 | SIYP | 450 | YYGM | 485 |
| | | | | | LYS | | PI | | | | SYGS | | | |
| | | | | | | | | | | | TY | | | |
| 14399 | FZD5 | 2928AM | SVSSA | 1 | SASS | 2 | AFYF | 284 | ISYSSM | 358 | SIYP | 451 | YYGM | 485 |
| | | | | | LYS | | PI | | | | HYGA | | | |
| | | | | | | | | | | | TF | | | |
| 14400 | FZD5 | 2928AM | SVSSA | 1 | SASS | 2 | AFYY | 288 | ISYSSI | 363 | SIYP | 452 | YYAM | 484 |
| | | | | | LYS | | PI | | | | SYGS | | | |
| | | | | | | | | | | | TF | | | |
| 14401 | FZD5 | 2928AM | SVSSA | 1 | SASS | 2 | AFFF | 298 | ISYSSI | 363 | SIYS | 453 | YYAM | 484 |
| | | | | | LYS | | PI | | | | SYSA | | | |
| | | | | | | | | | | | TY | | | |
| 14402 | FZD5 | 2928AM | SVSSA | 1 | SASS | 2 | SFYY | 299 | ISYSSM | 358 | SIYP | 439 | YYAF | 483 |
| | | | | | LYS | | PI | | | | SYSS | | | |
| | | | | | | | | | | | TY | | | |
| 14403 | FZD5 | 2928AM | SVSSA | 1 | SASS | 2 | AFYF | 284 | IPYGSM | 368 | SIYP | 452 | YYAM | 484 |
| | | | | | LYS | | PI | | | | SYGS | | | |
| | | | | | | | | | | | TF | | | |
| 14404 | FZD5 | 2928AM | SVSSA | 1 | SASS | 2 | ALYY | 300 | ISYSSM | 358 | SIYP | 435 | YYAM | 484 |
| | | | | | LYS | | PI | | | | SYSS | | | |
| | | | | | | | | | | | TF | | | |
| 14405 | FZD5 | 2928AM | SVSSA | 1 | SASS | 2 | AFYF | 284 | ISYSAM | 366 | SIYP | 454 | YYAF | 483 |
| | | | | | LYS | | PI | | | | YYGP | | | |
| | | | | | | | | | | | TY | | | |
| 14406 | FZD5 | 2928AM | SVSSA | 1 | SASS | 2 | VFYY | 293 | ISYSSI | 363 | SIYP | 455 | FYAF | 488 |
| | | | | | LYS | | PI | | | | FYGS | | | |
| | | | | | | | | | | | TF | | | |
| 14407 | FZD5 | 2928AM | SVSSA | 1 | SASS | 2 | RFYF | 301 | ISYSSI | 363 | AIYP | 456 | YYAM | 484 |
| | | | | | LYS | | PI | | | | SYSS | | | |
| | | | | | | | | | | | TN | | | |
| 14408 | FZD5 | 2928AM | SVSSA | 1 | SASS | 2 | AFYY | 288 | ISYSSI | 363 | SIYP | 457 | YYAM | 484 |
| | | | | | LYS | | PI | | | | YYSS | | | |
| | | | | | | | | | | | TH | | | |
| 14409 | FZD5 | 2928AM | SVSSA | 1 | SASS | 2 | LYYF | 302 | IAYSAM | 369 | SIYP | 458 | YYAM | 484 |
| | | | | | LYS | | PI | | | | SYST | | | |
| | | | | | | | | | | | TY | | | |
| 14410 | FZD5 | 2928AM | SVSSA | 1 | SASS | 2 | AFYF | 284 | LSFSSI | 370 | SIYP | 435 | YYAF | 483 |
| | | | | | LYS | | PI | | | | SYSS | | | |
| | | | | | | | | | | | TF | | | |
| 14411 | FZD5 | 2928AM | SVSSA | 1 | SASS | 2 | AYYF | 296 | ISYSSM | 358 | SIYS | 438 | YYGM | 485 |
| | | | | | LYS | | PI | | | | SYGS | | | |
| | | | | | | | | | | | TY | | | |
| 14412 | FZD5 | 2928AM | SVSSA | 1 | SASS | 2 | SSYF | 303 | ISYSSM | 358 | SIYS | 459 | YYAM | 484 |
| | | | | | LYS | | PI | | | | NYSS | | | |
| | | | | | | | | | | | SY | | | |
| 14413 | FZD5 | 2928AM | SVSSA | 1 | SASS | 2 | AFYF | 284 | LTYSSI | 371 | TIYP | 460 | YYAM | 484 |
| | | | | | LYS | | PI | | | | SYGS | | | |
| | | | | | | | | | | | TY | | | |
| 14414 | FZD5 | 2928AM | SVSSA | 1 | SASS | 2 | AFYF | 284 | ISYSSM | 358 | SIYP | 461 | YYAI | 489 |
| | | | | | LYS | | PI | | | | SFGS | | | |
| | | | | | | | | | | | TF | | | |
| 14415 | FZD5 | 2928AM | SVSSA | 1 | SASS | 2 | AHYF | 285 | LSYGSI | 372 | SIYP | 450 | YYGM | 485 |
| | | | | | LYS | | PI | | | | SYGS | | | |
| | | | | | | | | | | | TY | | | |
| 14416 | FZD5 | 2928AM | SVSSA | 1 | SASS | 2 | ASYF | 304 | ISYSSM | 358 | TIYP | 462 | YYAM | 484 |
| | | | | | LYS | | PI | | | | SYSS | | | |
| | | | | | | | | | | | TY | | | |
| 14417 | FZD5 | 2928AM | SVSSA | 1 | SASS | 2 | AFYF | 284 | IAYSSM | 365 | TIYS | 463 | YYAF | 483 |
| | | | | | LYS | | PI | | | | SYGA | | | |
| | | | | | | | | | | | TS | | | |
| 14418 | FZD5 | 2928AM | SVSSA | 1 | SASS | 2 | AHYY | 305 | ISYSSI | 363 | SIYP | 464 | YYAM | 484 |
| | | | | | LYS | | PI | | | | SYSS | | | |
| | | | | | | | | | | | TI | | | |
| 14419 | FZD5 | 2928AM | SVSSA | 1 | SASS | 2 | AGFF | 306 | ISYSSI | 363 | TIYP | 465 | YYGF | 486 |
| | | | | | LYS | | PI | | | | YYGA | | | |
| | | | | | | | | | | | TY | | | |
| 14420 | FZD5 | 2928AM | SVSSA | 1 | SASS | 2 | SSYY | 307 | ISYSSI | 363 | SIYS | 466 | YYAM | 484 |
| | | | | | LYS | | PI | | | | GYSA | | | |
| | | | | | | | | | | | TY | | | |
| 14421 | FZD5 | 2928AM | SVSSA | 1 | SASS | 2 | AFYF | 284 | ITYSSM | 373 | SIYP | 450 | YYGF | 486 |
| | | | | | LYS | | PI | | | | SYGS | | | |
| | | | | | | | | | | | TY | | | |
| 14422 | FZD5 | 2928AM | SVSSA | 1 | SASS | 2 | AFYF | 284 | ISYSSI | 363 | TIYP | 462 | YYAM | 484 |
| | | | | | LYS | | PI | | | | SYSS | | | |
| | | | | | | | | | | | TY | | | |
| 14423 | FZD5 | 2928AM | SVSSA | 1 | SASS | 2 | AFYF | 284 | LGYGSM | 374 | SIYP | 450 | YYAM | 484 |
| | | | | | LYS | | PI | | | | SYGS | | | |
| | | | | | | | | | | | TY | | | |
| 14424 | FZD5 | 2928AM | SVSSA | 1 | SASS | 2 | AFYF | 284 | ISYSSM | 358 | SIYP | 467 | YFAL | 490 |
| | | | | | LYS | | PI | | | | SYSA | | | |
| | | | | | | | | | | | TF | | | |
| 14425 | FZD5 | 2928AM | SVSSA | 1 | SASS | 2 | AFYY | 288 | IGYSSI | 375 | TIYP | 468 | YYAF | 483 |
| | | | | | LYS | | PI | | | | SYSS | | | |
| | | | | | | | | | | | TF | | | |
| 14426 | FZD5 | 2928AM | SVSSA | 1 | SASS | 2 | AFYY | 288 | ITYGSI | 376 | AIYS | 469 | YYAF | 483 |
| | | | | | LYS | | PI | | | | SYGS | | | |
| | | | | | | | | | | | TY | | | |
| 14427 | FZD5 | 2928AM | SVSSA | 1 | SASS | 2 | AFYY | 288 | ISYSSM | 358 | SIFP | 470 | YYGF | 486 |
| | | | | | LYS | | PI | | | | YYGS | | | |
| | | | | | | | | | | | TY | | | |
| 14428 | FZD5 | 2928AM | SVSSA | 1 | SASS | 2 | AFYF | 284 | ISYSSM | 358 | SIYP | 471 | YYAM | 484 |
| | | | | | LYS | | PI | | | | FYST | | | |
| | | | | | | | | | | | TF | | | |
| 14429 | FZD5 | 2928AM | SVSSA | 1 | SASS | 2 | SSYY | 307 | ISYGSM | 377 | SIYP | 467 | YYGM | 485 |
| | | | | | LYS | | PI | | | | SYSA | | | |
| | | | | | | | | | | | TF | | | |
| 14430 | FZD5 | 2928AM | SVSSA | 1 | SASS | 2 | AFYY | 288 | ISYSSM | 358 | SIYP | 472 | YYGM | 485 |
| | | | | | LYS | | PI | | | | SYGS | | | |
| | | | | | | | | | | | TS | | | |
| 14431 | FZD5 | 2928AM | SVSSA | 1 | SASS | 2 | AFYF | 284 | IYYSSM | 378 | SIYP | 473 | YYGM | 485 |
| | | | | | LYS | | PI | | | | TYGS | | | |
| | | | | | | | | | | | TV | | | |
| 14432 | FZD5 | 2928AM | SVSSA | 1 | SASS | 2 | AFYF | 284 | ISYSSI | 363 | TIYP | 474 | YYAM | 484 |
| | | | | | LYS | | PI | | | | NYSS | | | |
| | | | | | | | | | | | TY | | | |
| 14433 | FZD5 | 2928AM | SVSSA | 1 | SASS | 2 | AFYF | 284 | IFYSSM | 379 | SIYP | 475 | YYAM | 484 |
| | | | | | LYS | | PI | | | | SYSA | | | |
| | | | | | | | | | | | TY | | | |
| 14434 | FZD5 | 2928AM | SVSSA | 1 | SASS | 2 | AFYY | 288 | ISYSSM | 358 | SIYP | 476 | YYAM | 484 |
| | | | | | LYS | | PI | | | | GYSS | | | |
| | | | | | | | | | | | TY | | | |
| 14435 | FZD5 | 2928AM | SVSSA | 1 | SASS | 2 | AHYF | 285 | IAYSSM | 365 | TIYP | 462 | YYAM | 484 |
| | | | | | LYS | | PI | | | | SYSS | | | |
| | | | | | | | | | | | TY | | | |
| 14436 | FZD5 | 2928AM | SVSSA | 1 | SASS | 2 | AFYY | 288 | ISYSSI | 363 | SIYP | 477 | YYGM | 485 |
| | | | | | LYS | | PI | | | | SYSS | | | |
| | | | | | | | | | | | TS | | | |
| 14437 | FZD5 | 2928AM | SVSSA | 1 | SASS | 2 | AFYF | 284 | ISYSSM | 358 | SIYS | 478 | YYGM | 485 |
| | | | | | LYS | | PI | | | | GYGS | | | |
| | | | | | | | | | | | TY | | | |
| 14438 | FZD5 | 2928AM | SVSSA | 1 | SASS | 2 | AFYF | 284 | ISYSSM | 358 | SIYP | 450 | YYGM | 485 |
| | | | | | LYS | | PI | | | | SYGS | | | |
| | | | | | | | | | | | TY | | | |
| 14439 | FZD5 | 2928AM | SVSSA | 1 | SASS | 2 | AFYF | 284 | LSFSSM | 359 | SIYS | 466 | YYAL | 487 |
| | | | | | LYS | | PI | | | | GYSA | | | |
| | | | | | | | | | | | TY | | | |
| 14440 | FZD5 | 2928AM | SVSSA | 1 | SASS | 2 | AYYF | 296 | ISYSSM | 358 | SIYP | 479 | YYAF | 483 |
| | | | | | LYS | | PI | | | | SYGS | | | |
| | | | | | | | | | | | TN | | | |
| 14441 | FZD5 | 2928AM | SVSSA | 1 | SASS | 2 | AFYF | 284 | LSYSSI | 344 | SIYP | 480 | YYGM | 485 |
| | | | | | LYS | | PI | | | | TYGS | | | |
| | | | | | | | | | | | AY | | | |
| 14442 | FZD5 | 2928AM | SVSSA | 1 | SASS | 2 | AFYF | 284 | ITYSSM | 373 | TIYS | 481 | YYAM | 484 |
| | | | | | LYS | | PI | | | | SYGS | | | |
| | | | | | | | | | | | TY | | | |
| 14443 | FZD5 | 2928AM | SVSSA | 1 | SASS | 2 | AFYF | 284 | ISFSSM | 380 | AIYP | 482 | YYGF | 486 |
| | | | | | LYS | | PI | | | | YYGS | | | |
| | | | | | | | | | | | TY | | | |
| 14444 | FZD5 | 2928AM | SVSSA | 1 | SASS | 2 | AHYF | 285 | ISYSSM | 358 | SIYS | 438 | YYGM | 485 |
| | | | | | LYS | | PI | | | | SYGS | | | |
| | | | | | | | | | | | TY | | | |
| 14445 | FZD5 | 2928AM | SVSSA | 1 | SASS | 2 | ASYF | 304 | LSYSSM | 364 | SIYP | 435 | YYAM | 484 |
| | | | | | LYS | | PI | | | | SYSS | | | |
| | | | | | | | | | | | TF | | | |
| 14446 | FZD5 | 2928AM | SVSSA | 1 | SASS | 2 | AFYY | 288 | ISYGSM | 377 | SIYP | 450 | YYGM | 485 |
| | | | | | LYS | | PI | | | | SYGS | | | |
| | | | | | | | | | | | TY | | | |

### Example 2: Identification and Characterization of LRP Binding Synthetic Antibodies

### A. Phage Clonal ELISA of Synthetic Antibodies Targeting LRP5 and LRP6.

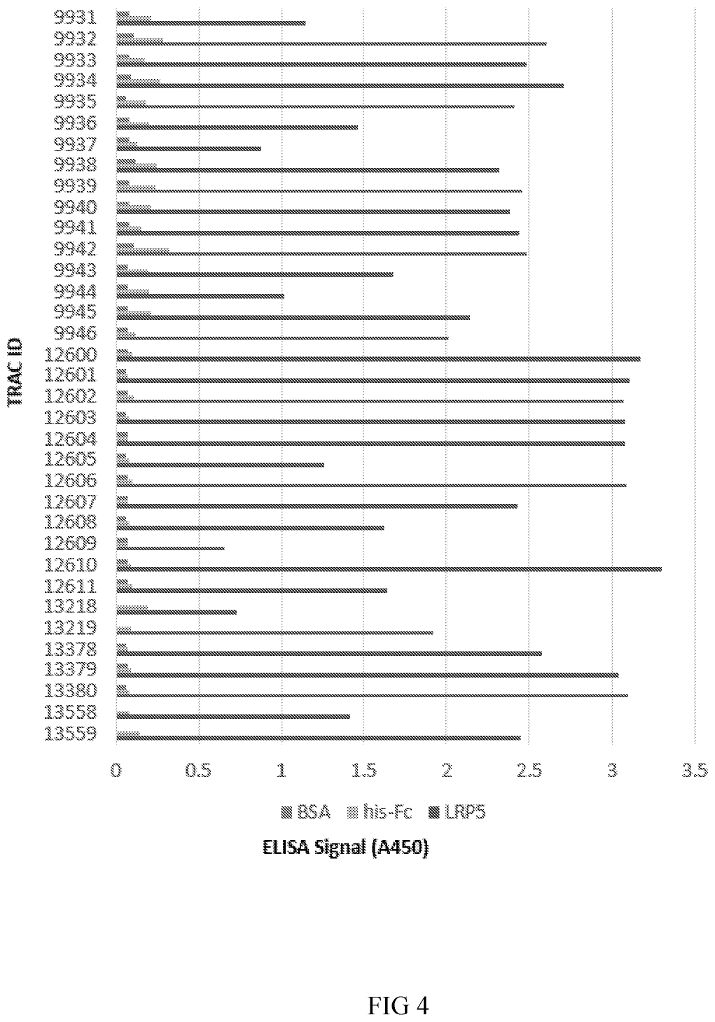

Single point ELISAs were performed on 96-well Maxisorp plates coated with the ECDs of mouse LRP5-his protein or human Fc and blocked with BSA (0.5%). The plates were incubated with monoclonal Fab-phage, or VH-phage and titers >10⁹ phage/ml followed by incubation with horseradish peroxidase (HRP)-conjugated anti-M13 antibody. The wells were washed 8 times followed by incubations with 3,3,′5,5′-tetramethylbenidine/H₂O₂ peroxidase (TMB) substrate for 5-10 min. the reaction was stopped by adding 1M H₃PO₄ and the absorbance was measured spectrophotometrically at 450 nm in a microtiter plate reader. The results are presented in FIG. 4. The results demonstrate that the synthetic antibodies bound to LRP5. LRP5 binding antibodies, 2459, 2460 and 8716, are described in U.S. provisional application No. 62/886,913, incorporated herein by reference.

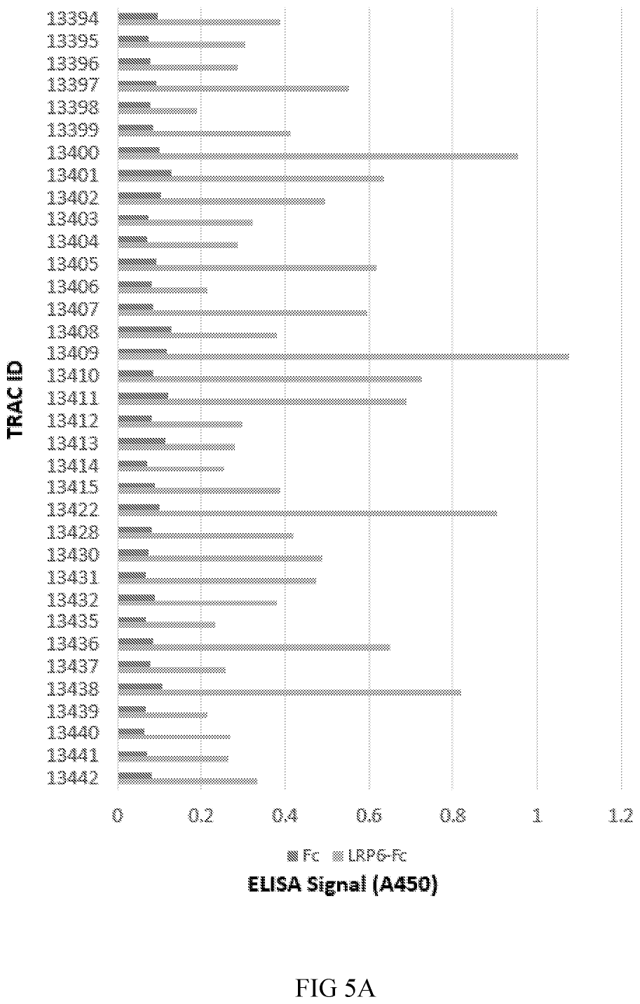

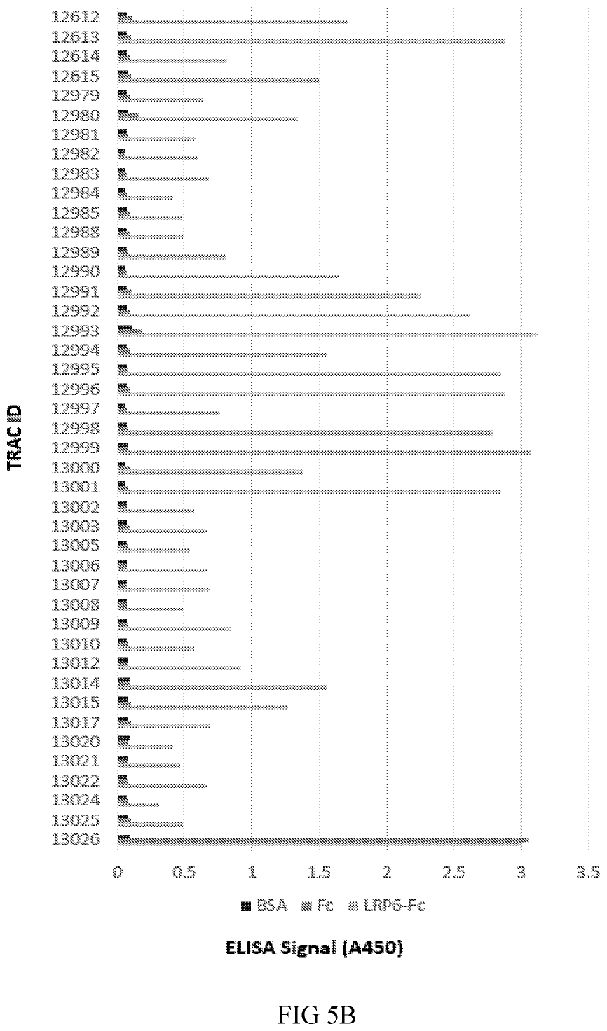

Single point ELISAs were performed on 96-well Maxisorp plates coated with the ECDs of human LRP6-Fc protein chimeras. The plates were incubated with the monoclonal Fab-phage, or VH-phage and titers >109 phage/ml followed by incubation with horseradish peroxidase (HRP)-conjugated anti-M13 antibody. The wells were washed 8 times followed by incubations with 3,3,′5,5′-tetramethylbenidine/H₂O₂ peroxidase (TMB) substrate for 5-10 min. the reaction was stopped by adding 1M H₃PO₄ and the absorbance was measured spectrophotometrically at 450 nm in a microtiter plate reader. The results are presented in FIGS. 5A and 5B. The results demonstrate the synthetic antibodies bound to LRP6. LRP6 binding antibodies, 2539, 2540, and 2542 are described in U.S. provisional application No. 62/886,918, incorporated herein by reference.

### B. Identification of the CDRs of the Synthetic Antibodies Targeting LRP5 and LRP6.

The CDRs of the LRP5-binding and LRP6-binding immunoglobulins set forth in Tables 3 and 4 were identified according to the INTERNATIONAL IMMUNOGENETICS INFORMATION SYSTEM (IMGT numbering system; Lefranc et al., 2003, Development and Comparative Immunology 27:55-77) and annotated as described in Persson et al. J Mol Biol. 2013 Feb. 22; 425(4):803-11, both incorporated herein by reference.

TABLE 3

| | | | LRP5-binding antibodies CDRs | | | | | | | | | | | |
|---|---|---|---|---|---|---|---|---|---|---|---|---|---|---|
| Anti- | | | | SEQ | | SEQ | | SEQ | | SEQ | | SEQ | | SEQ |
| body | LRP5-antibody | | | ID | | ID | | ID | | ID | | ID | | ID |
| ID | Library | Antigen | L1 | NO | L2 | NO: | L3 | NO: | H1 | NO: | H2 | NO: | H3 | NO: |
| 2459 | F | mLRP5 | SVSSA | 1 | SASSL | 2 | ASYAP | 492 | LSYYY | 527 | SIYSS | 552 | WSHVS | 584 |
| | | -his | | | YS | | I | | M | | YGYTY | | GHYSG | |
| | | | | | | | | | | | | | M | |
| 2460 | F | mLRP5 | SVSSA | 1 | SASSL | 2 | SSYSL | 130 | FSSSS | 528 | SISSS | 553 | GGSGV | 585 |
| | | -his | | | YS | | I | | I | | YGYTY | | SHYGS | |
| | | | | | | | | | | | | | VYYSW | |
| | | | | | | | | | | | | | WAL | |
| 8716 | F | mLRP5 | SVSSA | 1 | SASSL | 2 | YWAYY | 493 | FSSSS | 528 | SISSS | 553 | SWAM | 586 |
| | | -his | | | YS | | SPI | | I | | YGYTY | | | |
| 9931 | F | mLRP5 | SVSSA | 1 | SASSL | 2 | SSSGH | 494 | LYYYS | 529 | YISSY | 554 | WSHVV | 587 |
| | | -his | | | YS | | LI | | M | | YSYTS | | GAHYG | |
| | | | | | | | | | | | | | WAI | |
| 9932 | F | mLRP5 | SVSSA | 1 | SASSL | 2 | PYGYP | 495 | IYYYS | 530 | SISSY | 555 | SWWYW | 588 |
| | | -his | | | YS | | I | | M | | YGYTS | | SYHGY | |
| | | | | | | | | | | | | | AAM | |
| 9933 | F | mLRP5 | SVSSA | 1 | SASSL | 2 | FHGLI | 496 | ISSSY | 348 | SIYSY | 556 | GYSYV | 589 |
| | | -his | | | YS | | | | M | | SSYTS | | WYGVY | |
| | | | | | | | | | | | | | YHGYG | |
| | | | | | | | | | | | | | AM | |
| 9934 | F | mLRP5 | SVSSA | 1 | SASSL | 2 | YWFLI | 497 | LYYYY | 531 | SISPY | 557 | SGYGW | 590 |
| | | -his | | | YS | | | | I | | YGYTS | | YAM | |
| 9935 | F | mLRP5 | SVSSA | 1 | SASSL | 2 | HSYGY | 498 | ISYSY | 310 | SISSS | 553 | SYYWG | 591 |
| | | -his | | | YS | | PI | | M | | YGYTY | | YWAAL | |
| 9936 | F | mLRP5 | SVSSA | 1 | SASSL | 2 | YYAWY | 499 | ISYYS | 532 | SIYSS | 552 | GGVYY | 592 |
| | | -his | | | YS | | LI | | M | | YGYTY | | YPSYA | |
| | | | | | | | | | | | | | GWPYG | |
| | | | | | | | | | | | | | M | |
| 9937 | F | mLRP5 | SVSSA | 1 | SASSL | 2 | AAYSP | 500 | ISSSY | 348 | SIYPS | 558 | HYAWW | 593 |
| | | -his | | | YS | | I | | M | | YGYTY | | VGAF | |
| 9938 | F | mLRP5 | SVSSA | 1 | SASSL | 2 | YYSWY | 501 | ISYSY | 310 | SIYSY | 559 | GGGAH | 594 |
| | | -his | | | YS | | PPF | | M | | YSYTS | | GWGYS | |
| | | | | | | | | | | | | | L | |
| 9939 | F | mLRP5 | SVSSA | 1 | SASSL | 2 | SFYPI | 502 | LSSYS | 29 | SIYPY | 560 | SGPVY | 595 |
| | | -his | | | YS | | | | I | | YGYTS | | ASYSW | |
| | | | | | | | | | | | | | AWYYY | |
| | | | | | | | | | | | | | GAL | |
| 9940 | F | mLRP5 | SVSSA | 1 | SASSL | 2 | YYWYP | 503 | LSYYY | 533 | SIYSY | 561 | YSWGA | 596 |
| | | -his | | | YS | | F | | I | | YSSTY | | YGYGA | |
| | | | | | | | | | | | | | M | |
| 9941 | F | mLRP5 | SVSSA | 1 | SASSL | 2 | WGSPI | 504 | FSSSS | 528 | SIYPS | 562 | YYYHY | 597 |
| | | -his | | | YS | | | | I | | SGSTY | | SVPVY | |
| | | | | | | | | | | | | | AAL | |
| 9942 | F | mLRP5 | SVSSA | 1 | SASSL | 2 | YSSWY | 505 | LYYYS | 529 | SIYPS | 562 | SPYYG | 598 |
| | | -his | | | YS | | LI | | M | | SGSTY | | FYYSG | |
| | | | | | | | | | | | | | FYHWV | |
| | | | | | | | | | | | | | FYGF | |
| 9943 | F | mLRP5 | SVSSA | 1 | SASSL | 2 | GAYLI | 506 | LYYSS | 534 | YIYSY | 563 | AAWGW | 599 |
| | | -his | | | YS | | | | M | | YGYTY | | HPAF | |
| 9944 | F | mLRP5 | SVSSA | 1 | SASSL | 2 | HFYYY | 507 | YSSYI | 535 | YISSY | 57 | GGGFY | 500 |
| | | -his | | | YS | | PI | | | | YGSTY | | YAGGW | |
| | | | | | | | | | | | | | PYASY | |
| | | | | | | | | | | | | | AWAF | |
| 9945 | F | mLRP5 | SVSSA | 1 | SASSL | 2 | AFHSP | 508 | LYYSS | 534 | YIYPY | 564 | AWYSY | 601 |
| | | -his | | | YS | | I | | M | | YGYTS | | YVGL | |
| 9946 | F | mLRP5 | SVSSA | 1 | SASSL | 2 | AWYPI | 509 | ISYSS | 358 | SIYPY | 565 | GVYYG | 602 |
| | | -his | | | YS | | | | M | | YGSTY | | GGYWA | |
| | | | | | | | | | | | | | GGYYP | |
| | | | | | | | | | | | | | AAL | |
| 12600 | H | mLRP5 | SVSSA | 1 | SASDL | 491 | YAGAG | 510 | FTAYA | 536 | SIYPS | 566 | RSYYF | 603 |
| | | -his | | | YS | | LI | | M | | GGYTA | | AL | |
| 12601 | H | mLRP5 | SVSSA | 1 | SASDL | 491 | SYSYP | 511 | LGGYG | 537 | GIYPS | 567 | SSTYS | 604 |
| | | -his | | | YS | | I | | I | | GGYTA | | SYYYH | |
| | | | | | | | | | | | | | FYAL | |
| 12602 | H | mLRP5 | SVSSA | 1 | SASDL | 491 | YYRSR | 512 | FASAW | 538 | RISPS | 568 | STRWH | 605 |
| | | -his | | | YS | | LI | | M | | GGYTA | | SAYAY | |
| | | | | | | | | | | | | | YYSHA | |
| | | | | | | | | | | | | | M | |
| 12603 | H | mLRP5 | SVSSA | 1 | SASDL | 491 | SYFYP | 513 | IGGAA | 539 | GISAS | 569 | SRPSW | 606 |
| | | -his | | | YS | | I | | M | | GGATA | | YWSYH | |
| | | | | | | | | | | | | | YFAL | |
| 12604 | H | mLRP5 | SVSSA | 1 | SASDL | 491 | GFFPI | 514 | FSSSS | 528 | GISTS | 570 | SYYGY | 607 |
| | | -his | | | YS | | | | I | | GGATA | | SRGF | |
| 12605 | H | mLRP5 | SVSSA | 1 | SASDL | 491 | SHSYP | 515 | FASYG | 540 | SIYPS | 566 | SYSFH | 608 |
| | | -his | | | YS | | I | | M | | GGYTA | | YAWPR | |
| | | | | | | | | | | | | | YHYGA | |
| | | | | | | | | | | | | | L | |
| 12606 | H | mLRP5 | SVSSA | 1 | SASDL | 491 | SHYYP | 286 | IAGSS | 541 | GISAS | 571 | YDSGY | 609 |
| | | -his | | | YS | | I | | I | | GGYTA | | YAWYY | |
| | | | | | | | | | | | | | HDRAM | |
| 12607 | H | mLRP5 | SVSSA | 1 | SASDL | 491 | RGSGP | 516 | LTGDW | 542 | GIYPS | 572 | FGSPH | 610 |
| | | -his | | | YS | | I | | M | | GGATA | | YGM | |
| 12608 | H | mLRP5 | SVSSA | 1 | SASDL | 491 | RYSGG | 517 | FTSYS | 543 | GIYPS | 573 | PRYYA | 611 |
| | | -his | | | YS | | LI | | M | | GGSTA | | YYSGG | |
| | | | | | | | | | | | | | F | |
| 12609 | H | mLRP5 | SVSSA | 1 | SASDL | 491 | SARYY | 518 | LNAAA | 544 | GISAS | 574 | SHSSG | 612 |
| | | -his | | | YS | | YLI | | M | | RGATA | | SRSRG | |
| | | | | | | | | | | | | | L | |
| 12610 | H | mLRP5 | SVSSA | 1 | SASDL | 491 | SSYSL | 130 | LAGAG | 545 | RISTS | 575 | YSFRY | 613 |
| | | -his | | | YS | | I | | M | | GGYTA | | PSYAM | |
| 12611 | H | mLRP5 | SVSSA | 1 | SASDL | 491 | SYRGY | 519 | IGGAA | 539 | GIYAS | 576 | GSRYS | 614 |
| | | -his | | | YS | | YLI | | M | | GGATA | | GF | |
| 13218 | | 2539A | SVSSA | 1 | SASSL | 2 | FSWGL | 520 | ISYYP | 546 | YISPY | 577 | HYYFR | 615 |
| | | MmLRP | | | YS | | I | | M | | SGYTF | | WSRGM | |
| | | 5-his | | | | | | | | | | | | |
| 13219 | | 2539A | SVSSA | 1 | SASSL | 2 | YSWLP | 521 | IVFGS | 547 | HIAPY | 578 | VNFIR | 616 |
| | | MmLRP | | | YS | | I | | I | | YGFTY | | RFRGM | |
| | | 5-his | | | | | | | | | | | | |
| 13378 | | 2542A | SVSSA | 1 | SASSL | 2 | FFRPI | 522 | ISSNY | 548 | SINPS | 579 | AVRRS | 617 |
| | | MmLRP | | | YS | | | | I | | YSYTF | | QGI | |
| | | 5-his | | | | | | | | | | | | |
| 13379 | | 2539A | SVSSA | 1 | SASSL | 2 | YSRRL | 523 | LTYTS | 549 | SISPF | 580 | FPYFA | 618 |
| | | MmLRP | | | YS | | F | | M | | HGHTF | | WVGGM | |
| | | 5-his | | | | | | | | | | | | |
| 13380 | | 2539A | SVSSA | 1 | SASSL | 2 | FIRVP | 524 | ITYYS | 550 | SITSY | 581 | SHYFP | 619 |
| | | MmLRP | | | YS | | I | | M | | YGNTD | | WTVAL | |
| | | 5-his | | | | | | | | | | | | |
| 13558 | | 2540A | SVSSA | 1 | SASSL | 2 | FYVPF | 525 | N.D. | | SIYPY | 582 | ASYHA | 620 |
| | | MmLRP | | | YS | | | | | | YGFTD | | SFDGL | |
| | | 5-his | | | | | | | | | | | | |
| 13559 | | 2540A | SVSSA | 1 | SASSL | 2 | GYSFG | 526 | IADSS | 551 | SISPY | 583 | TSIFK | 621 |
| | | MmLRP | | | YS | | Q | | I | | FSYTR | | RFAGM | |
| | | 5-his | | | | | | | | | | | | |

TABLE 4

| | | | binding antibodies CDRs | | | | | | | | | | | |
|---|---|---|---|---|---|---|---|---|---|---|---|---|---|---|
| | | | | SEQ | | SEQ | | SEQ | | SEQ | | SEQ | | SEQ |
| Antibody | LRP6-antibody | | | ID | | ID | | ID | | ID | | ID | | ID |
| ID | Library | Antigen | L1 | NO: | L2 | NO: | L3 | NO: | H1 | NO: | H2 | NO: | H3 | NO: |
| 2539 | F | LRP6-Fc | SVSSA | 1 | SASSLYS | 2 | YSWGPF | 622 | ISYSSI | 363 | YISSYYG | 51 | AHYFPWA | 813 |
| | | | | | | | | | | | YTY | | GAM | |
| 2542 | F | LRP6-Fc | SVSSA | 1 | SASSLYS | 2 | YYWPI | 623 | ISSYYI | 683 | SIYSSYG | 741 | TVRGSKK | 814 |
| | | | | | | | | | | | YTS | | PYFSGWA | |
| | | | | | | | | | | | | | M | |
| 2540 | F | LRP6-Fc | SVSSA | 1 | SASSLYS | 2 | YYFLI | 624 | ISYSSI | 363 | SISPYYG | 1742 | SSYFPWF | 815 |
| | | | | | | | | | | | YTY | | SAM | |
| 12612 | H | LRP6-Fc | SVSSA | 1 | SASDLYS | 491 | YRYYWRP | 625 | FSGDAM | 684 | RISSSGG | 743 | ASYYSNY | 816 |
| | | | | | | | I | | | | YTA | | YYGPAM | |
| 12613 | H | LRP6-Fc | SVSSA | 1 | SASDLYS | 491 | GPWGLI | 626 | ISGAWM | 685 | RIYPSGG | 744 | GWFSFRS | 817 |
| | | | | | | | | | | | TTD | | YYRSYYY | |
| | | | | | | | | | | | | | YSAL | |
| 12614 | H | LRP6-Fc | SVSSA | 1 | SASDLYS | 491 | RYSSPI | 627 | FTTYSI | 686 | GISTSGG | 745 | SGHPRYY | 818 |
| | | | | | | | | | | | YTD | | SRRHSYG | |
| | | | | | | | | | | | | | M | |
| 12615 | H | LRP6-Fc | SVSSA | 1 | SASDLYS | 491 | YRYWYGP | 628 | FAASGI | 687 | YISPSGG | 746 | SYYSNYY | 819 |
| | | | | | | | I | | | | YTD | | YYYDAM | |
| 12979 | 2539AM | LRP6-Fc | SVSSA | 1 | SASSLYS | 2 | YSWALF | 629 | IFFSSM | 688 | GISSYYG | 747 | AHYFPWA | 820 |
| | | | | | | | | | | | FTY | | GAL | |
| 12980 | 2539AM | LRP6-Fc | SVSSA | 1 | SASSLYS | 2 | ISWGLI | 630 | IRYSSI | 689 | GIFSNYG | 748 | AHYFRRA | 821 |
| | | | | | | | | | | | PTT | | RGL | |
| 12981 | 2539AM | LRP6-Fc | SVSSA | 1 | SASSLYS | 2 | YFSYPI | 631 | LSYSSI | 344 | FISSYYS | 749 | SHYFPWA | 822 |
| | | | | | | | | | | | FTH | | GAM | |
| 12982 | 2539AM | LRP6-Fc | SVSSA | 1 | SASSLYS | 2 | YRWALF | 632 | LAYSSI | 690 | SISSYYG | 750 | AHYFPWA | 823 |
| | | | | | | | | | | | FTY | | GGM | |
| 12983 | 2539AM | LRP6-Fc | SVSSA | 1 | SASSLYS | 2 | YSRILF | 633 | LRYASM | 691 | YIASFYG | 751 | SYYYPRS | 824 |
| | | | | | | | | | | | DTY | | RGM | |
| 12984 | 2539AM | LRP6-Fc | SVSSA | 1 | SASSLYS | 2 | YSLRPI | 634 | LSFSSI | 370 | HISPYYG | 752 | SHYFPWA | 825 |
| | | | | | | | | | | | YTH | | VAM | |
| 12985 | 2539AM | LRP6-Fc | SVSSA | 1 | SASSLYS | 2 | YSRGLI | 635 | IFYSSI | 692 | YISSYYS | 58 | SHYFPWA | 825 |
| | | | | | | | | | | | YTY | | VAM | |
| 12986 | 2539AM | LRP6-Fc | SVSSA | 1 | SASSLYS | 2 | YFWGLI | 636 | LSYSSM | 364 | NITPYYG | 753 | SHYFPWF | 826 |
| | | | | | | | | | | | YTS | | AAM | |
| 12987 | 2539AM | LRP6-Fc | SVSSA | 1 | SASSLYS | 2 | YSWGLI | 637 | INYSSI | 693 | GISSYYS | 754 | ANYFPWA | 827 |
| | | | | | | | | | | | YTY | | YAM | |
| 12988 | 2539AM | LRP6-Fc | SVSSA | 1 | SASSLYS | 2 | DSWGLF | 638 | ISYSSM | 358 | LITSYYG | 755 | AHYFPWV | 828 |
| | | | | | | | | | | | YTT | | VGM | |
| 12989 | 2539AM | LRP6-Fc | SVSSA | 1 | SASSLYS | 2 | YYWGLI | 639 | LSYSSI | 344 | FISSYYG | 73 | SHYFPWA | 829 |
| | | | | | | | | | | | YTY | | GAL | |
| 13002 | 2540AM | LRP6-Fc | SVSSA | 1 | SASSLYS | 2 | YYYLI | 640 | IAFSSI | 694 | SISSYYG | 555 | SSYFPWF | 830 |
| | | | | | | | | | | | YTS | | SAL | |
| 13003 | 2540AM | LRP6-Fc | SVSSA | 1 | SASSLYS | 2 | YYILI | 641 | LFYSSM | 695 | FISPYYS | 756 | SSYFPWL | 831 |
| | | | | | | | | | | | FTN | | SDM | |
| 13004 | 2540AM | LRP6-Fc | SVSSA | 1 | SASSLYS | 2 | HSFPI | 642 | ISYSSM | 358 | NITSYYG | 757 | SSYFPWV | 832 |
| | | | | | | | | | | | YTT | | SGM | |
| 13005 | 2540AM | LRP6-Fc | SVSSA | 1 | SASSLYS | 2 | YHHLI | 643 | ISFASM | 696 | SISPYYS | 758 | SSYFPWF | 815 |
| | | | | | | | | | | | YTS | | SAM | |
| 13006 | 2540AM | LRP6-Fc | SVSSA | 1 | SASSLYS | 2 | YYSPI | 644 | IFYSSI | 692 | SISPYYG | 759 | SSYLPWF | 833 |
| | | | | | | | | | | | YTD | | SAL | |
| 13007 | 2540AM | LRP6-Fc | SVSSA | 1 | SASSLYS | 2 | YYYPI | 645 | ISFYSI | 697 | SISSYYS | 760 | SSYFPWF | 834 |
| | | | | | | | | | | | YTD | | TAL | |
| 13008 | 2540AM | LRP6-Fc | SVSSA | 1 | SASSLYS | 2 | YHFLI | 646 | ISYSSI | 363 | AIYSYYS | 761 | SSYFPWV | 835 |
| | | | | | | | | | | | YTI | | GGF | |
| 13009 | 2540AM | LRP6-Fc | SVSSA | 1 | SASSLYS | 2 | YYFPI | 647 | LSYSSI | 344 | SISPYYS | 758 | SSRFPWF | 836 |
| | | | | | | | | | | | YTS | | YGL | |
| 13010 | 2540AM | LRP6-Fc | SVSSA | 1 | SASSLYS | 2 | YDFLI | 648 | ISYSSM | 358 | SISSYYG | 555 | SSYFPWF | 830 |
| | | | | | | | | | | | YTS | | SAL | |
| 13394 | 2540AM | LRP6-Fc | SVSSA | 1 | SASSLYS | 2 | YYILI | 641 | LFYASM | 598 | SISSYYG | 555 | SSYFPWF | 815 |
| | | | | | | | | | | | YTS | | SAM | |
| 13395 | 2540AM | LRP6-Fc | SVSSA | 1 | SASSLYS | 2 | YYYLI | 640 | ISYSSI | 363 | SISPYYG | 762 | SSYFPWF | 815 |
| | | | | | | | | | | | FTL | | SAM | |
| 13396 | 2540AM | LRP6-Fc | SVSSA | 1 | SASSLYS | 2 | YYFPI | 647 | IAYASI | 699 | SISSYYS | 763 | SSYFPWF | 815 |
| | | | | | | | | | | | YTY | | SAM | |
| 13397 | 2540AM | LRP6-Fc | SVSSA | 1 | SASSLYS | 2 | YYFPI | 647 | ITYSSI | 361 | SISSYYG | 764 | SSYFPWS | 837 |
| | | | | | | | | | | | YTA | | SAM | |
| 13398 | 2540AM | LRP6-Fc | SVSSA | 1 | SASSLYS | 2 | NSFLI | 649 | ISRSSI | 700 | GISSYYG | 765 | SHYFPWL | 838 |
| | | | | | | | | | | | YTR | | SAL | |
| 13399 | 2540AM | LRP6-Fc | SVSSA | 1 | SASSLYS | 2 | YYNPI | 650 | ISYASI | 701 | SISPYYG | 766 | ASYYPWF | 839 |
| | | | | | | | | | | | YTR | | SAM | |
| 13400 | 2540AM | LRP6-Fc | SVSSA | 1 | SASSLYS | 2 | YGFLI | 651 | LDYSSI | 702 | SISPYYS | 767 | SSYIPWR | 840 |
| | | | | | | | | | | | YTT | | YAI | |
| 13401 | 2540AM | LRP6-Fc | SVSSA | 1 | SASSLYS | 2 | YYFLI | 624 | ISYSSI | 363 | SISPYYS | 74 | SSYFPWI | 841 |
| | | | | | | | | | | | YTN | | SAM | |
| 13402 | 2540AM | LRP6-Fc | SVSSA | 1 | SASSLYS | 2 | YYFPI | 647 | IYYYSM | 530 | GISSYYS | 768 | SSYFPWI | 842 |
| | | | | | | | | | | | YTS | | SAL | |
| 13403 | 2540AM | LRP6-Fc | SVSSA | 1 | SASSLYS | 2 | YYFPI | 647 | ISYSSM | 358 | SISPYYS | 769 | SSRFPWI | 843 |
| | | | | | | | | | | | FTS | | SGM | |
| 13404 | 2540AM | LRP6-Fc | SVSSA | 1 | SASSLYS | 2 | YNFLI | 652 | ISYSSI | 363 | SISSYYG | 68 | SSYFPWF | 830 |
| | | | | | | | | | | | FTS | | SAL | |
| 13405 | 2540AM | LRP6-Fc | SVSSA | 1 | SASSLYS | 2 | YYFPI | 647 | ISYSSI | 363 | SISSYYG | 68 | SPYFPWS | 844 |
| | | | | | | | | | | | FTS | | SAL | |
| 13406 | 2540AM | LRP6-Fc | SVSSA | 1 | SASSLYS | 2 | YYFPI | 647 | LSYSAI | 703 | YISPYYG | 65 | ASYLPWF | 845 |
| | | | | | | | | | | | YTY | | SAM | |
| 13407 | 2540AM | LRP6-Fc | SVSSA | 1 | SASSLYS | 2 | SSFLI | 653 | LSGFGI | 704 | FISSYYG | 770 | SSYLPWI | 846 |
| | | | | | | | | | | | YTA | | TAM | |
| 13408 | 2540AM | LRP6-Fc | SVSSA | 1 | SASSLYS | 2 | YVFLI | 654 | LSFASM | 705 | SISSYYG | 771 | SAYFPWF | 847 |
| | | | | | | | | | | | YTP | | AAL | |
| 13409 | 2540AM | LRP6-Fc | SVSSA | 1 | SASSLYS | 2 | YYFPI | 647 | LAYNSI | 706 | SIFSYYS | 772 | SSYFPWF | 815 |
| | | | | | | | | | | | YTN | | SAM | |
| 13410 | 2540AM | LRP6-Fc | SVSSA | 1 | SASSLYS | 2 | FYGPI | 655 | ISFSSI | 357 | SISSYYG | 773 | SSYFPWF | 848 |
| | | | | | | | | | | | YTL | | FAM | |
| 13411 | 2540AM | LRP6-Fc | SVSSA | 1 | SASSLYS | 2 | YFFLI | 656 | LYYSNI | 707 | YISSYYG | 774 | SSYFPWF | 830 |
| | | | | | | | | | | | YTS | | SAL | |
| 13412 | 2540AM | LRP6-Fc | SVSSA | 1 | SASSLYS | 2 | YYGPI | 657 | IHYYSI | 708 | SISPYYS | 775 | SSYFPWF | 815 |
| | | | | | | | | | | | FTD | | SAM | |
| 13413 | 2540AM | LRP6-Fc | SVSSA | 1 | SASSLYS | 2 | YDFLI | 648 | ISFASI | 709 | SIYSYYS | 776 | SSHFPWV | 849 |
| | | | | | | | | | | | FTN | | SAL | |
| 13414 | 2540AM | LRP6-Fc | SVSSA | 1 | SASSLYS | 2 | YYSPI | 644 | IYYSSM | 378 | SISPYYG | 557 | SSYFPWV | 850 |
| | | | | | | | | | | | YTS | | SAM | |
| 13415 | 2540AM | LRP6-Fc | SVSSA | 1 | SASSLYS | 2 | YYFLI | 624 | LSYFSM | 710 | SIYSYYG | 777 | SSHFPWF | 851 |
| | | | | | | | | | | | FTN | | SAM | |
| 13422 | 2540AM | LRP6-Fc | SVSSA | 1 | SASSLYS | 2 | FYGPI | 655 | LFYSSM | 695 | TISPYYG | 778 | SSYFPWF | 830 |
| | | | | | | | | | | | YTS | | SAL | |
| 13428 | 2540AM | LRP6-Fc | SVSSA | 1 | SASSLYS | 2 | YTLPI | 658 | ISFSSM | 380 | HISSYYG | 779 | SSYFPWF | 830 |
| | | | | | | | | | | | FTS | | SAL | |
| 13430 | 2540AM | LRP6-Fc | SVSSA | 1 | SASSLYS | 2 | YRFPI | 659 | ISYFSI | 711 | SISPYYS | 74 | SSYFPWF | 852 |
| | | | | | | | | | | | YTN | | SAI | |
| 13431 | 2540AM | LRP6-Fc | SVSSA | 1 | SASSLYS | 2 | YHLLI | 660 | LYYNSI | 712 | AIHPYYG | 780 | SSRFPWF | 853 |
| | | | | | | | | | | | YTS | | PAM | |
| 13432 | 2540AM | LRP6-Fc | SVSSA | 1 | SASSLYS | 2 | YSFPI | 661 | LSYASI | 713 | SISSYYG | 781 | SSYFPWF | 815 |
| | | | | | | | | | | | YTG | | SAM | |
| 13435 | 2540AM | LRP6-Fc | SVSSA | 1 | SASSLYS | 2 | YFLPI | 662 | LSYSSM | 364 | SISPYYG | 782 | SSYFPWF | 830 |
| | | | | | | | | | | | FTY | | SAL | |
| 13436 | 2540AM | LRP6-Fc | SVSSA | 1 | SASSLYS | 2 | YYFPI | 647 | ISYASM | 714 | SISSYYG | 783 | SSYVPWF | 854 |
| | | | | | | | | | | | YTD | | YAL | |
| 13437 | 2540AM | LRP6-Fc | SVSSA | 1 | SASSLYS | 2 | YDNLI | 663 | ITYSSI | 361 | SISSYYG | 68 | SSYFPWF | 815 |
| | | | | | | | | | | | FTS | | SAM | |
| 13438 | 2540AM | LRP6-Fc | SVSSA | 1 | SASSLYS | 2 | YLFPI | 664 | ISYYSI | 715 | SIYSYYG | 784 | ASYFPWF | 855 |
| | | | | | | | | | | | YTT | | PAM | |
| 13439 | 2540AM | LRP6-Fc | SVSSA | 1 | SASSLYS | 2 | YFFPI | 665 | LSHSSI | 716 | SISSHYG | 785 | SSYFPWF | 856 |
| | | | | | | | | | | | YTH | | FAL | |
| 13440 | 2540AM | LRP6-Fc | SVSSA | 1 | SASSLYS | 2 | YYIPI | 666 | IAYNSM | 717 | SISSYYS | 760 | SSYFPWF | 830 |
| | | | | | | | | | | | YTD | | SAL | |
| 13441 | 2540AM | LRP6-Fc | SVSSA | 1 | SASSLYS | 2 | FNFLI | 667 | LSYSSI | 344 | YISSYYG | 786 | SSYYPWF | 857 |
| | | | | | | | | | | | YTA | | SAL | |
| 13442 | 2540AM | LRP6-Fc | SVSSA | 1 | SASSLYS | 2 | YNYLI | 668 | LAYSSI | 690 | SISSYYG | 787 | SSYLPWV | 858 |
| | | | | | | | | | | | YTY | | SAL | |
| 12990 | 2542AM | LRP6-Fc | SVSSA | 1 | SASSLYS | 2 | YYWPI | 623 | ISLFFM | 718 | SIYSSYS | 788 | SFNGAKK | 859 |
| | | | | | | | | | | | YTS | | PYFSGWA | |
| | | | | | | | | | | | | | M | |
| 12991 | 2542AM | LRP6-Fc | SVSSA | 1 | SASSLYS | 2 | YYWPI | 623 | IPSYFM | 719 | SIYSSYS | 789 | NVVVSRK | 860 |
| | | | | | | | | | | | YTA | | PYFFRSA | |
| | | | | | | | | | | | | | M | |
| 12992 | 2542AM | LRP6-Fc | SVSSA | 1 | SASSLYS | 2 | YYWPI | 623 | ISSSYM | 348 | SIYSSYS | 790 | TVPGSKR | 861 |
| | | | | | | | | | | | YTG | | PYFASLA | |
| | | | | | | | | | | | | | I | |
| 12993 | 2542AM | LRP6-Fc | SVSSA | 1 | SASSLYS | 2 | YYWPI | 623 | ISSYFI | 720 | SIYPSYS | 791 | NVRGFRK | 862 |
| | | | | | | | | | | | YTS | | PYFSRWA | |
| | | | | | | | | | | | | | M | |
| 12994 | 2542AM | LRP6-Fc | SVSSA | 1 | SASSLYS | 2 | YYWPI | 623 | LSSYYM | 721 | SIYSRFS | 792 | NVPGIKR | 863 |
| | | | | | | | | | | | YTA | | PYFTAWA | |
| | | | | | | | | | | | | | M | |
| 12995 | 2542AM | LRP6-Fc | SVSSA | 1 | SASSLYS | 2 | SYWPI | 669 | LHSYYI | 722 | NIYSSYG | 793 | TIRGSKR | 864 |
| | | | | | | | | | | | YTS | | PYFAASG | |
| | | | | | | | | | | | | | L | |
| 12996 | 2542AM | LRP6-Fc | SVSSA | 1 | SASSLYS | 2 | YYWPI | 623 | LSSYYM | 721 | SIYSSYS | 788 | TARENKK | 865 |
| | | | | | | | | | | | YTS | | PYFSGWA | |
| | | | | | | | | | | | | | M | |
| 12997 | 2542AM | LRP6-Fc | SVSSA | 1 | SASSLYS | 2 | YYRPI | 670 | IGSYYM | 723 | AIYSPYG | 794 | TLPLSKK | 866 |
| | | | | | | | | | | | YTS | | PYFSLWA | |
| | | | | | | | | | | | | | F | |
| 12998 | 2542AM | LRP6-Fc | SVSSA | 1 | SASSLYS | 2 | YYWPI | 623 | LGSYYI | 724 | TIYSYYS | 795 | TFHGSKK | 867 |
| | | | | | | | | | | | YTS | | PYFSGRA | |
| | | | | | | | | | | | | | M | |
| 12999 | 2542AM | LRP6-Fc | SVSSA | 1 | SASSLYS | 2 | YYWPI | 623 | LTSYYI | 725 | SIYPSYS | 791 | SIRGSKK | 868 |
| | | | | | | | | | | | YTS | | PYFSRLA | |
| | | | | | | | | | | | | | L | |
| 13000 | 2542AM | LRP6-Fc | SVSSA | 1 | SASSLYS | 2 | YYWPI | 623 | ISTYYM | 726 | SIYPSYS | 791 | SVRGSKK | 869 |
| | | | | | | | | | | | YTS | | PYFSHWA | |
| | | | | | | | | | | | | | M | |
| 13001 | 2542AM | LRP6-Fc | SVSSA | 1 | SASSLYS | 2 | YYWPI | 623 | ISSNYI | 548 | SIYSSFG | 796 | NVRTSKK | 870 |
| | | | | | | | | | | | YTS | | PYFPLWA | |
| | | | | | | | | | | | | | M | |
| 13011 | 2460AM | LRP6-Fc | SVSSA | 1 | SASSLYS | 2 | ASNNLI | 671 | IPLYTI | 727 | SISPSYS | 797 | GVSGGSN | 871 |
| | | | | | | | | | | | YTY | | YISIFRN | |
| | | | | | | | | | | | | | SSGM | |
| 13012 | 2460AM | LRP6-Fc | SVSSA | 1 | SASSLYS | 2 | SSYSLI | 130 | IFSTSI | 728 | SITSAYS | 798 | GGLSVSH | 872 |
| | | | | | | | | | | | YTY | | YNYVHSF | |
| | | | | | | | | | | | | | KWGL | |
| 13013 | 2460AM | LRP6-Fc | SVSSA | 1 | SASSLYS | 2 | SAITPI | 672 | ISYSTM | 729 | SIVSSYS | 799 | SCSSFCN | 873 |
| | | | | | | | | | | | RTY | | YVSNYNA | |
| | | | | | | | | | | | | | RRAL | |
| 13014 | 2460AM | LRP6-Fc | SVSSA | 1 | SASSLYS | 2 | FSYSPF | 673 | LSSSGM | 730 | AIYPFDG | 800 | GNARVSN | 874 |
| | | | | | | | | | | | YTY | | YISSYYA | |
| | | | | | | | | | | | | | WRAM | |
| 13015 | 2460AM | LRP6-Fc | SVSSA | 1 | SASSLYS | 2 | SSYNLI | 674 | FSSSSI | 528 | GIFSYFS | 801 | GGYGFSY | 875 |
| | | | | | | | | | | | YTY | | YNSVTFY | |
| | | | | | | | | | | | | | RWAF | |
| 13016 | 2460AM | LRP6-Fc | SVSSA | 1 | SASSLYS | 2 | SYNFPF | 675 | IRGPSI | 731 | SISPFCG | 802 | VGSGVCN | 876 |
| | | | | | | | | | | | FTY | | SLHNYFY | |
| | | | | | | | | | | | | | KRGM | |
| 13017 | 2460AM | LRP6-Fc | SVSSA | 1 | SASSLYS | 2 | SSTVPF | 676 | FSSTYM | 732 | SITPSYG | 803 | SSSDISI | 877 |
| | | | | | | | | | | | YTN | | DNSIRYT | |
| | | | | | | | | | | | | | WGAM | |
| 13018 | 2460AM | LRP6-Fc | SVSSA | 1 | SASSLYS | 2 | SSYSLI | 130 | INSSFM | 733 | SISPSYG | 804 | GHSGVTN | 878 |
| | | | | | | | | | | | YTY | | FISFYYR | |
| | | | | | | | | | | | | | RRAM | |
| 13019 | 2460AM | LRP6-Fc | SVSSA | 1 | SASSLYS | 2 | SNYSLF | 677 | IFGYSM | 734 | SIFPCCS | 805 | GVSGLDN | 879 |
| | | | | | | | | | | | NTY | | NRTASYS | |
| | | | | | | | | | | | | | RMGM | |
| 13020 | 2460AM | LRP6-Fc | SVSSA | 1 | SASSLYS | 2 | STYSLI | 678 | FSSSTI | 735 | SIAPSFG | 806 | GAASVNH | 880 |
| | | | | | | | | | | | HTY | | IGSLYVS | |
| | | | | | | | | | | | | | WRGL | |
| 13021 | 2460AM | LRP6-Fc | SVSSA | 1 | SASSLYS | 2 | SAFSLI | 679 | LSSSTM | 736 | SISPYYS | 807 | HGSGLSH | 881 |
| | | | | | | | | | | | YTR | | NATTSYY | |
| | | | | | | | | | | | | | RSAI | |
| 13022 | 2460AM | LRP6-Fc | SVSSA | 1 | SASSLYS | 2 | SSYSLI | 130 | ISYTSI | 737 | SIFSTYS | 808 | SSSAVSR | 882 |
| | | | | | | | | | | | YTY | | NSIVYYS | |
| | | | | | | | | | | | | | RTAM | |
| 13023 | 2460AM | LRP6-Fc | SVSSA | 1 | SASSLYS | 2 | SSYTPI | 680 | FSSSVM | 738 | SIAPSYS | 809 | GVCRVSN | 883 |
| | | | | | | | | | | | FTY | | YSSYFYS | |
| | | | | | | | | | | | | | RGAM | |
| 13024 | 2460AM | LRP6-Fc | SVSSA | 1 | SASSLYS | 2 | YSNSLI | 681 | SYSTM | 729 | SITPYYS | 810 | AISHVAH | 884 |
| | | | | | | | | | | | TTS | | YDNVIYS | |
| | | | | | | | | | | | | | KWGI | |
| 13025 | 2460AM | LRP6-Fc | SVSSA | 1 | SASSLYS | 2 | SSYSLI | 130 | IASYSM | 739 | FISPYHS | 811 | GHSVLSN | 885 |
| | | | | | | | | | | | YTD | | YVSIHYY | |
| | | | | | | | | | | | | | WAGF | |
| 13026 | 8716AM | LRP6-Fc | SVSSA | 1 | SASSLYS | 2 | YWAYFSP | 682 | FSYSSM | 740 | SISSRYG | 812 | SWAM | 586 |
| | | | | | | | I | | | | YTY | | | |

### Example 3: Tetravalent Binding Antibody Molecule Formats

A. We generated various formats of tetravalent binding antibody molecules comprising pan-FZD and LRP5/6 antibody fragments, e.g., scFv, diabodies and Fab, on either end of an Fc domain, see Table 5 and FIG. 6, and assayed their Wnt agonist activity. DNA fragments encoding antibody variable domains were either amplified by PCR from phagemid DNA template or were constructed by chemical synthesis (Twist Biosciences). The DNA fragments were cloned into mammalian expression vectors (pSCSTa). Bispecific diabodies and IgGs contained an optimized version of a “knobs-in-holes” heterodimeric Fc (Ridgway et al. Protein Eng. 9, 617-621 (1996)). Diabody domains were arranged in a VH-VL orientation with the variable domains separated by a short GGGGS linker (SEQ ID NO: 886), which favors intermolecular association between VH and VL domains and thus favors diabody formation. To produce diabody fusion constructs, the diabody chains were fused to human IgG1 Fc. Diabody-fc-diabody proteins were constructed as VH-x-VL-y-[human IgG1 Fc]-z-VH-x-VL where linkers are x=GGGGS (SEQ ID NO: 886), y=LEDKTHTKVEPKSS (SEQ ID NO: 887), and Z=SGSETPGTSESATPESGGG (SEQ ID NO: 888). In this format, the human IgG1 Fc or knob-in-hole IgG1 Fc fragments spanned from position 234-478 (Kabat numbering). For scFv-Fc fusions, the variable domains were arranged in a VL-VH orientation and were connected by a long GTTAASGSSGGSSSGA linker (SEQ ID NO: 889), which favors intramolecular association between VH and VL domains and thus favors scFv formation. Variants with a Fab domain fused to the C-terminus of the Fc were generated via chemical synthesis (Twist Biosciences). For all constructs, the entire coding region was cloned into a mammalian expression vector in frame with the secretion signal peptide.

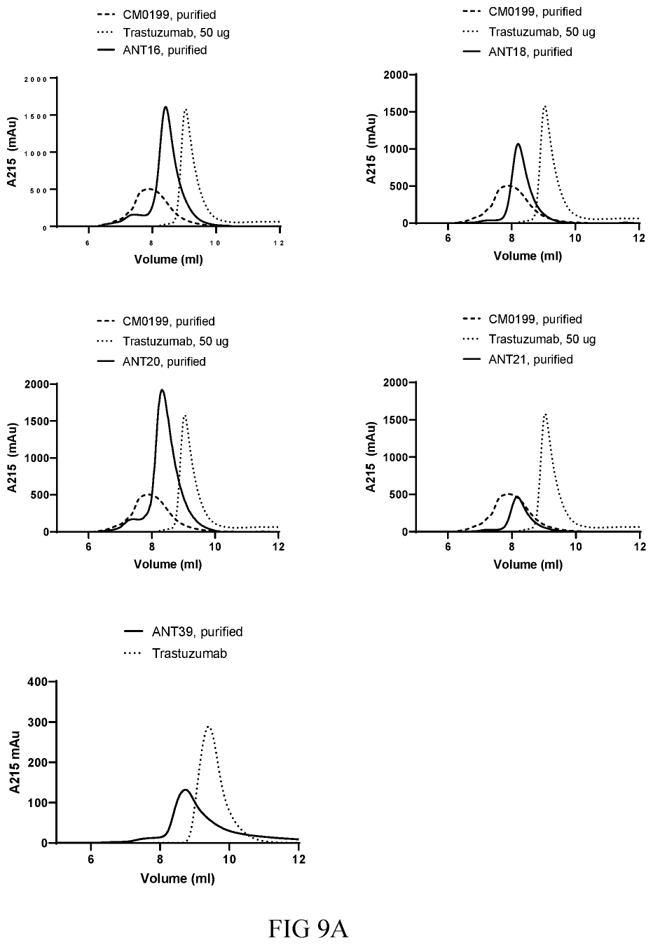

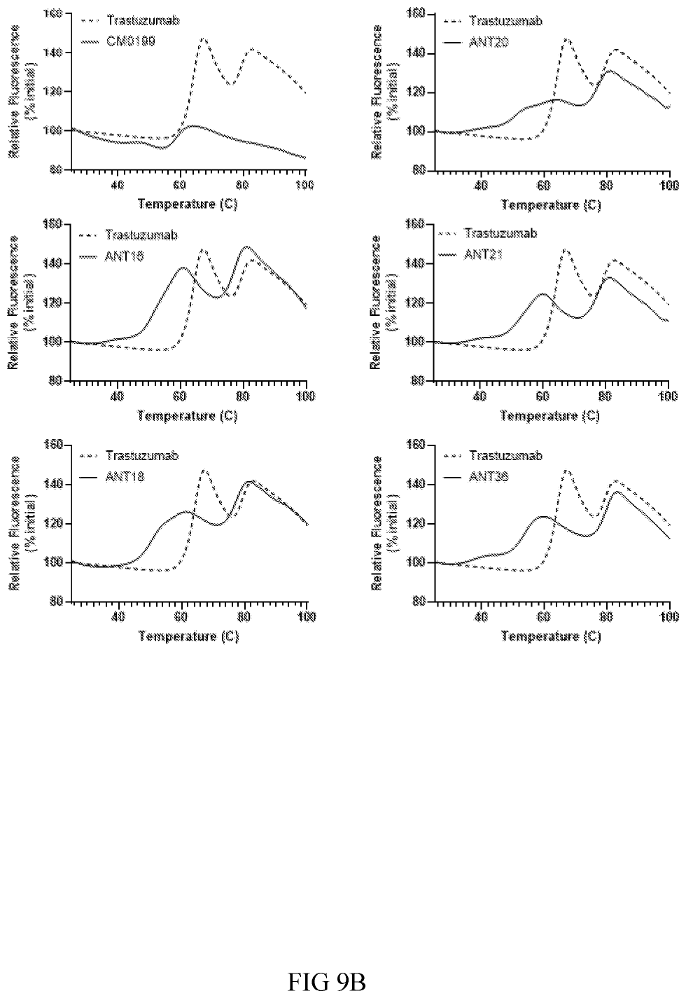

These various of tetravalent binding antibody molecules comprising pan-specific FZD and LRP5/6 antibody fragments were tested in a TOPFLASH assay to monitor beta catenin-mediated gene reporter activity. Proteins were compared against the native ligand Wnt3a. Assays were performed by plating TOPFLASH cells to ˜70% confluency in a 96-well tissue culture treated plate. Agonists were diluted in DMEM to provide a final assay concentration of 0.046 nM-100 nM and cells were treated overnight at 37° C. under 5% CO₂. Luciferase expression was quantified using the Dual-Luciferase Reporter Assay System (Promega) in 96-well black plates in accordance with the manufacturer's instructions. Briefly, HEK293T cells were transduced with lentivirus coding for the pBARIs reporter (Biechele and Moon in Wnt Signaling: Pathway Methods and Mammalian Models, E. Vincan, Ed. (Humana Press, Totowa, NJ, 2008), pp. 99-110) and with Renilla Luciferase as a control to generate a Wnt-βcatenin signaling reporter cell line. 1-2×10³ cells in 120 μl were seeded in each well of 96-well plates for 24 hours prior to transfection or stimulation. The following day, FZD Agonists or Ab protein was added, and following 15-20 hours of stimulation, cells were lysed and luminescence was measured in accordance with the dual luciferase protocol (Promega) using an Envision plate reader (PerkinElmer). For the FZD4 Agonist assay, FZD4 cDNA was transfected for 6 hours prior to adding the FZD Agonist. For the Wnt inhibition assays, Wnt1 was introduced by cDNA transfection or WNT3A protein was applied for 6 hours prior to the addition of Ab protein. All assays were repeated at least three times. The results are presented in Table 5. As shown in Table 5, each of the tetravalent formats activate FZD signaling to differing degrees when clustering FZD4 and LRP5. These formats were also evaluated for stability, homogeneity and yield production from Expi293 (FIGS. 3 and 9). From these analyses, the Diabody-Fc-Fab format provides the best balance of activity, expression, stability. Finally, we applied the same modality arrangement for FZD5 and LRP6 and we observed potent agonist activity. The results in Table 5 show that the various tetravalent modalities elicit WNT agonism and that engagement of 2 LRP5/6 epitopes produces WNT signaling activity (maxima) higher than with 1 LRP5/6 epitope.

TABLE 5

| | | | Binding | | | | |
|---|---|---|---|---|---|---|---|
| | | | specificity | Topflash | | % Wnt | |
| | Modality | | (Paratope | EC50 (nM) | | Maxima | |
| Molecule | Recombinantly | Paratopes | Stoichiometry) | Avg Stdev | | Avg Stdev | |
| Wnt3a | produced ligand | NA | FZD/LRP | 6 | 3 | 100% | ND |
| CM0011 | FZD Diabody- | 5019 (FZD Diabody); | FZD1/2/4/5/7/8, | 0.3 | 0.1 | 63% | 10% |
| | Fc-LRP Diabody | 2539, 2542 (LRP diabody) | LRP6¹, LRP6³ (2:1:1) | | | | |
| ANT-4 | FZD Diabody- | 5016(FZD Diabody); | FZD1/2/4/5/7/8, | 0.5 | 0.1 | 63% | 6% |
| | Fc-LRP Fab | 2540, 2542 (LRP Fab) | LRP6¹, LRP6³ (2:1:1) | | | | |
| ANT-5 | FZD Diabody- | 5016(FZD Diabody); | FZD1/2/4/5/7/8, | 0.5 | 0.1 | 59% | 8% |
| | Fc-LRP Fab | 2540, 2542 (LRP Fab) | LRP6¹, LRP6³ (2:1:1) | | | | |
| CT0001 | FZD IgG-LRP | 5016(FZD IgG); 2539, | FZD1/2/4/5/7/8, | 0.3 | 0.1 | 57% | 17% |
| | Diabody | 2542 (LRP Diabody) | LRP6¹, LRP6³ (2:1:1) | | | | |
| CT0002 | LRP Diabody-Fc- | 2539, 2542 (LRP Diabody); | FZD1/2/4/5/7/8, | 0.5 | 0.1 | 55% | 22% |
| | FZD scFv (VL-VH) | 5016 (FZD scFv) | LRP6¹, LRP6³ (2:1:1) | | | | |
| CM0126 | FZD Diabody- | 5016(FZD Diabody); | FZD1/2/4/5/7/8, | 0.3 | 0.2 | 52% | 1% |
| | Fc-LRP Diabody | 2539, 2542 (LRP Diabody) | LRP6¹, LRP6³ (2:1:1) | | | | |
| ANT-1 | LRP Diabody- | 2540, 2542 (LRP Diabody); | FZD1/2/4/5/7/8, | 0.7 | 0.5 | 49% | 22% |
| | Fc-FZD Fab | 5016 (FZD Fab) | LRP6¹, LRP6³ (2:1:1) | | | | |
| CT0003 | FZD Diabody- | 5016 (FZD Diabody); | FZD1/2/4/5/7/8, | 0.4 | 0.1 | 42% | 16% |
| | Fc-LRP scFv | 2539, 2542 (LRP scFv) | LRP6¹, LRP6¹ (2:1:1) | | | | |
| ANT-2 | LRP Diabody- | 2540, 2542(LRP Diabody); | FZD1/2/4/5/7/8, | 0.3 | 0.1 | 38% | 25% |
| | Fc-FZD Fab | 5016(FZD Fab) | LRP6¹, LRP6³ (2:1:1) | | | | |
| CM0042 | FZD Diabody- | 5019(FZD Diabody); | FZD1/2/4/5/7/8, | 0.9 | 0.8 | 33% | 22% |
| | Fc-LRP Diabody | 2539 (LRP Diabody) | LRP6³ (2:2) | | | | |
| CM0302 | FZD IgG-LRP | 5056(FZD IgG)- | FZD1/2/4/5/7/8/10, | 0.3 | 0.2 | 18% | 8% |
| | Diabody | 2539(LRP Diabody) | LRP6³ (2:2) | | | | |
| CM0303 | FZD IgG-LRP | 5056(FZD IgG); | FZD1/2/4/5/7/8/10, | 3.7 | 2.1 | 17% | 13% |
| | scFv | 2539(LRP scFv) | LRP6³ (2:2) | | | | |
| CM0299 | FZD IgG-LRP | 5016(FZD IgG)-, | FZD1/2/4/5/7/8, | 0.1 | 0.1 | 14% | 5% |
| | Diabody | 2539(LRP Diabody) | LRP6³ (2:2) | | | | |

| | | | Binding | | | | |
|---|---|---|---|---|---|---|---|
| | | | specificity | Topflash | | % Wnt | |
| | Modality | | (Paratope | EC₅₀ (nM) | | Maxima | |
| Molecule | Recombinantly | Paratopes | Stoichiometry) | Avg Stdev | | Avg Stdev | |
| Wnt3a | produced ligand | NA | FZD/LRP | 6 | 3 | 100% | ND |
| CM0300 | FZD IgG-LRP scFv | 5016(FZD IgG)-, | FZD1/2/4/5/7/8, | 0.3 | 0.2 | 5% | 2% |
| | | 2539(LRP scFv) | LRP6³ (2:2) | | | | |
| CM0301 | FZD IgG-LRP scFv | 5016(FZD IgG)-, | FZD1/2/4/5/7/8, | 0.2 | 0.2 | 1% | 1% |
| | | 2542(LRP scFv) | LRP6¹ (2:2) | | | | |
| CM0156 | Diabody-Fc-Diabody | 4275 | no FZD/LRP binding | NA | NA | NA | NA |
| 4275 | IgG | 4275 | no FZD/LRP binding | NA | NA | NA | NA |

### B. Diabody-Fc-Fab Format FZD4 Agonists.

FZD Agonists having a bispecific LRP5-binding diabody and a FZD4 binding domain comprising FZD4-binding Fabs (FZD4 Agonists), a FZD5 binding domain comprising FZD5-binding Fabs (FZD5 Agonists), or a FZD binding domain that binds multiple FZD (pan-FZD Agonist) were generated using a knob-in-holes system. Briefly, the constructs were generated by chemical synthesis (Twist Biosciences) or by standard molecular biology techniques in a mammalian expression vector (pSCSTa). Diabody constructs were arranged in a VH-VL manner with a short (GGGGS (SEQ ID NO: 886)) linker linking the VH and VL to favor intermolecular pairing. For bispecific diabody arrangements, the variable domains for paratopes A and B, respectively, were arranged as VH(A)-VL(B) on the Hole Fc chain and VH(B)-VL(A) on the Knob Fc chain to facilitate proper paratope formation. Diabodies were fused to the N-terminus of an optimized knob-in-holes heterodimeric Fc (Ridgway et al. Protein Eng. 9, 617-621 (1996) via a GGGGSGGGGSEPKSS linker (SEQ ID NO: 890). The Fc region also contains the effector-null mutations D278A and N314G (Kabat numbering), corresponding to D655A/N297G (EU numbering). Fab domains were fused to the C-terminus of the heterodimeric Fc via a GGGSGGGSGGGSGGGSGSTG linker (SEQ ID NO: 891). Directly to this linker was fused the N-terminus of the Fab VH domain followed by CH1, terminating at T238 (Kabat numbering). This Fab pairs with a standard kappa light chain which was cloned as described above. For all constructs, the entire coding region was cloned into a mammalian expression vector in frame with the secretion signal peptide.

In addition, Diabody-fc-Fab formats were constructed as VH-x-VL-y-[human IgG1 Fc]-z-VH where linkers are x=GGGGS (SEQ ID NO: 886), y=GGGGSGGGGSEPKSSDKTHT (SEQ ID NO: 892), and z=GGGSGGGSGGGSGGGSGSTG (SEQ ID NO: 891). Diabody domains were arranged in a VH-VL orientation with the variable domains separated by a short GGGGS linker (SEQ ID NO: 886), which favors intermolecular association between VH and VL domains and thus favors diabody formation. Further, the Fc region may exhibit attenuated effector functions due amino acid mutations to N297G and D265A (DANG) variants or L234A, L235A, P331S (LALAPS) variants, and with the Fc region further comprising knob-in-hole heterodimerization variants Merrimack, Merchant or Merchant S:S.

FIG. 7 is an illustration of the Diabody-Fc-Fab format FZD4 Agonists having a LRP5 binding domain comprised of a diabody that is bivalent and bispecific for LRP5 and a FZD4 binding domain comprised of two FZD4 binding Fab fragments formed by a VL and CL1 of the light chain construct pairing with the VH and CH1 of each of the heavy chain hole and heavy chain knob constructs. Table 12 presents the amino acid sequences of heavy chains and light chains of FZD4 Agonists ANT's (Diabody-Fc-Fab format): the heavy chain knob construct (ANT16 knob), the heavy chain hole construct (ANT hole) and the light chain construct. The light chain and heavy chain variable CDRs are in bold underlined italics.

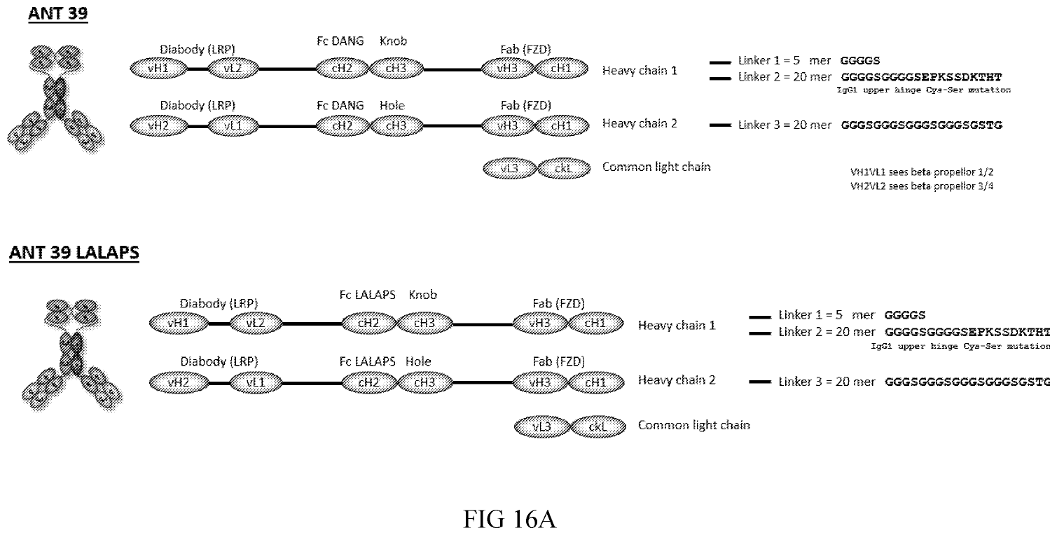

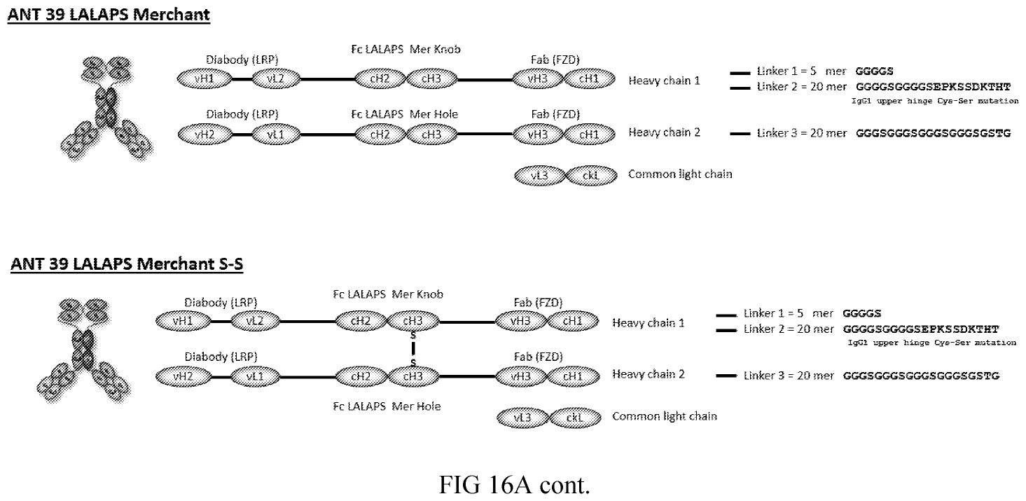

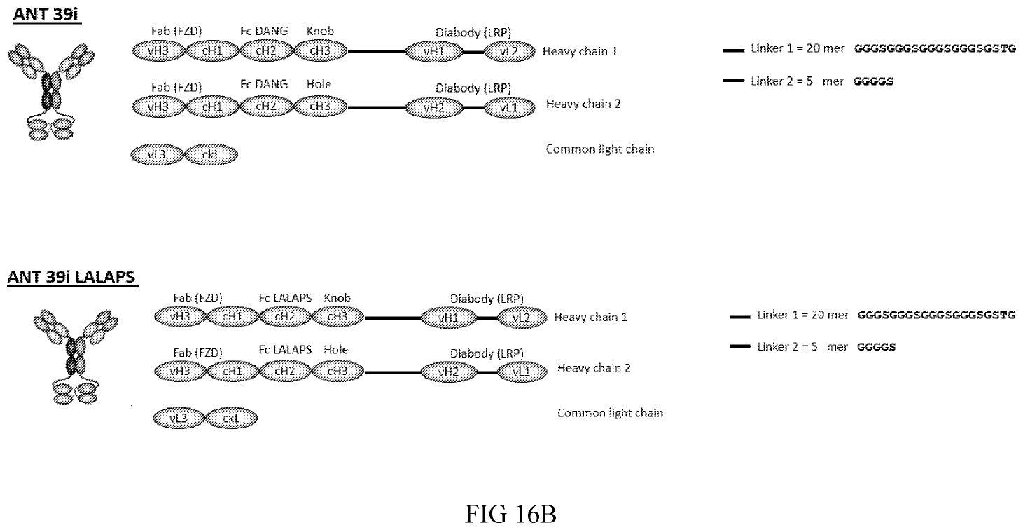

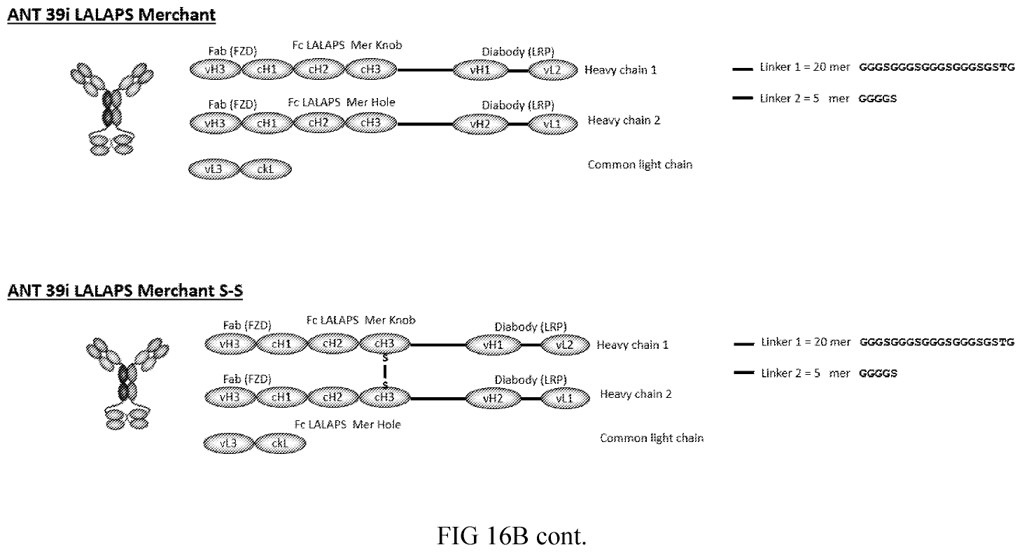

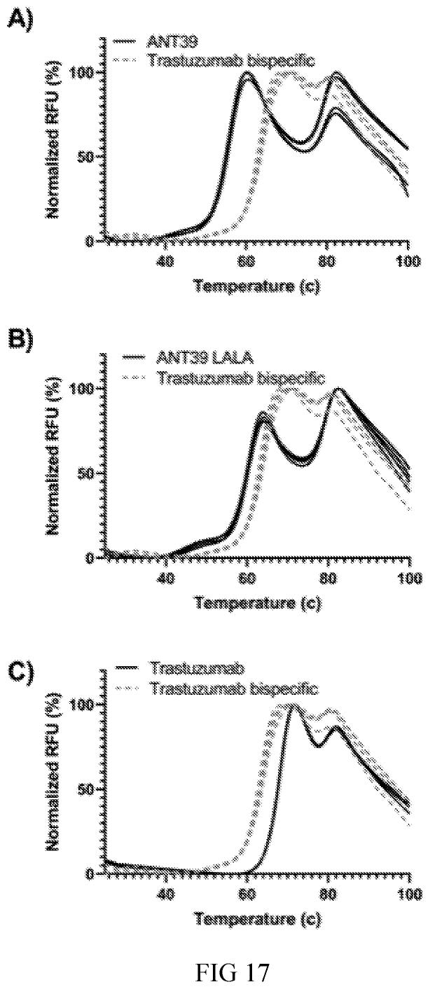

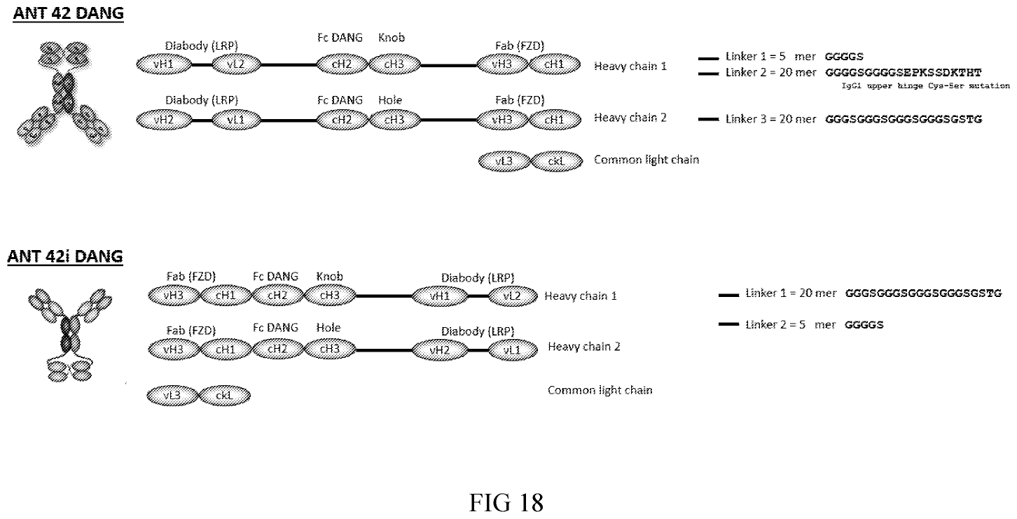

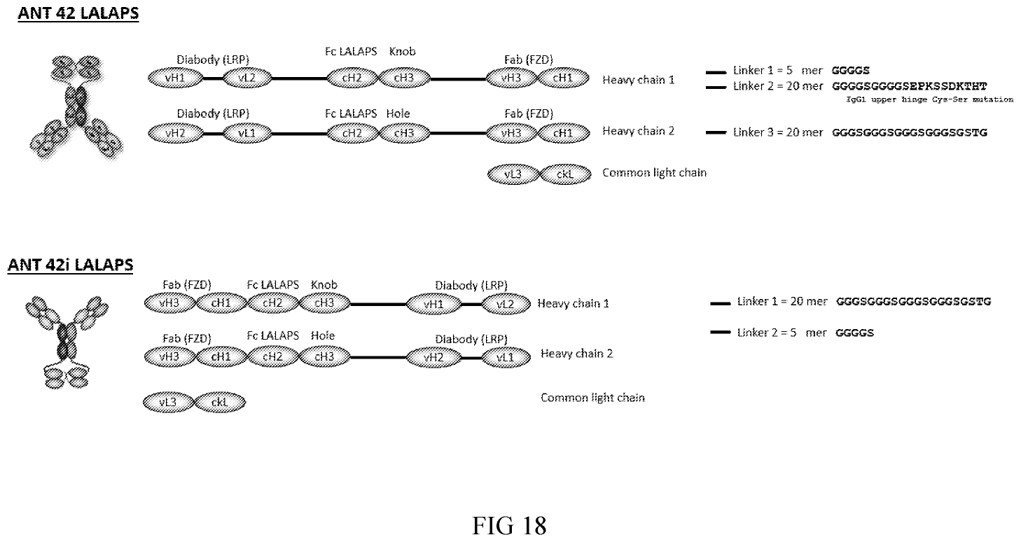

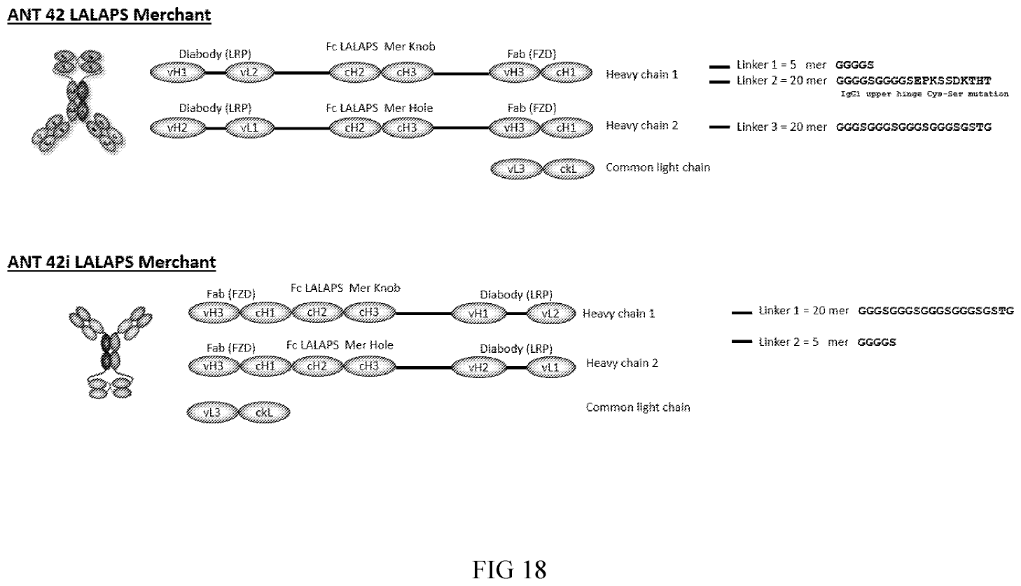

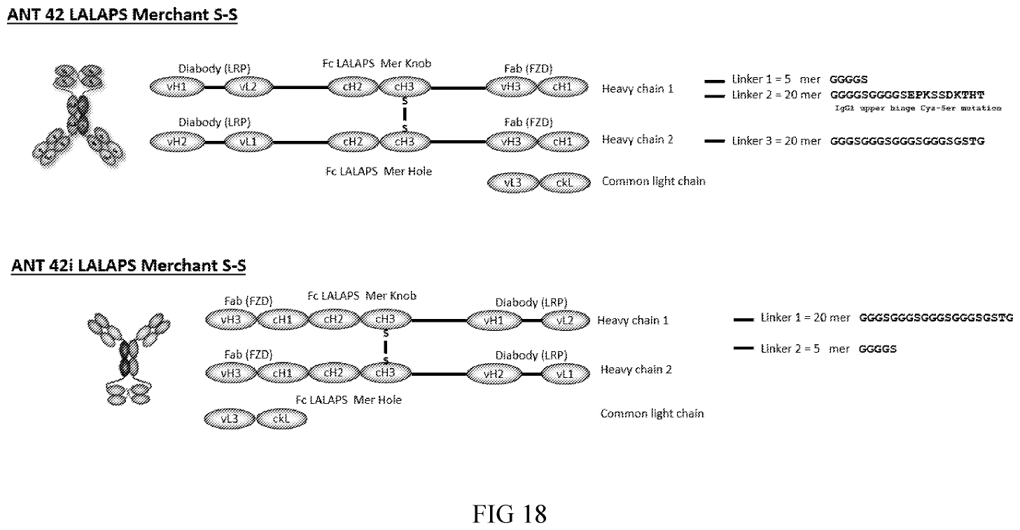

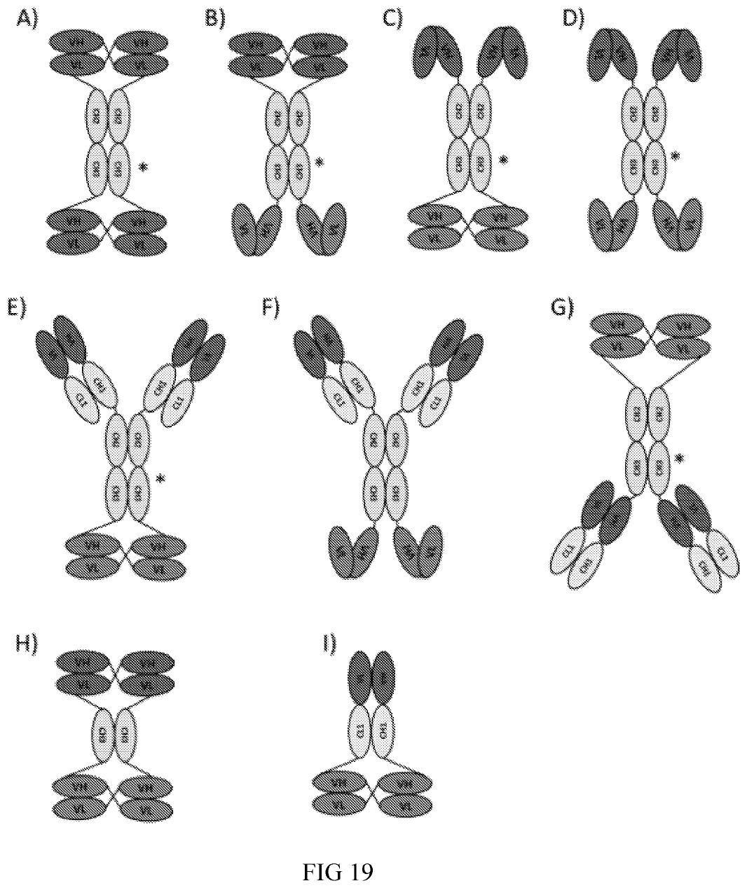

FIG. 16A depicts Diabody-Fc-Fab format FZD4 Agonists having Fc regions with attenuated effector functions due to amino acid mutations, e.g., N297G (NG) and D265A, (DANG) variants, and/or LALAPS variants, and with the Fc region further comprising knob-in-hole heterodimerization variants Merrimack, Merchant or Merchant S:S

### B. IgG-Diabody Format FZD4 Agonists.

FZD Agonists having two FZD-binding Fabs forming an N-terminal binding domain and a bispecific LRP5/6 binding diabody forming the C-terminal binding domain and an Fc domain were generated using a knob-in-holes system. IgG-Diabody proteins were constructed as VH-[human IgG1 Fc]-y-VH-x-VL where linkers are x=GGGGS (SEQ ID NO: 886) and y=GGGSGGGSGGGSGGGSGSTG (SEQ ID NO: 891).

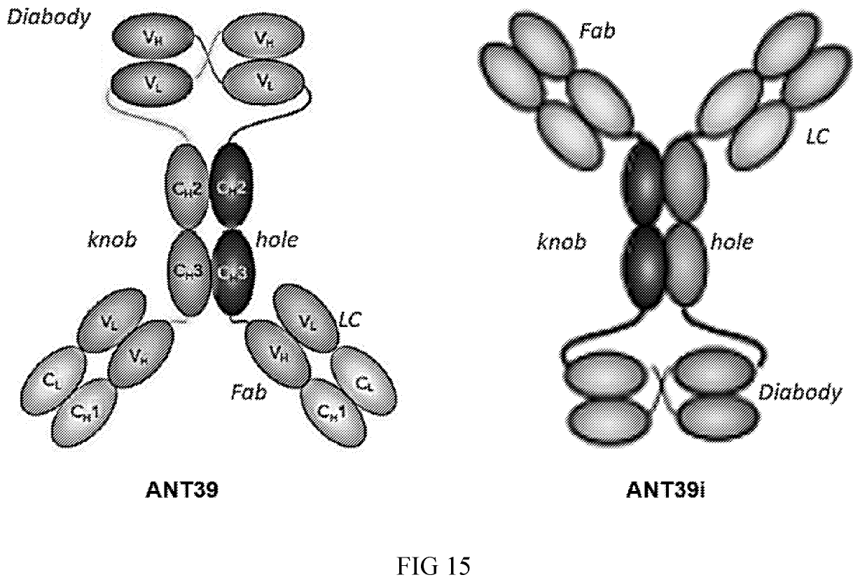

FIG. 15 presents an illustration of the IgG-Diabody format FZD4 Agonists having an FZD binding domain comprising two Fab fragments attached to the N-terminus of the Fc domain with each Fab binding an FZD. The LRP5/6 co-receptor binding domain is attached to the C-terminus of the Fc domain and is composed of a diabody that binds two different sites on the co-receptor, e.g., a Wnt1 site (E1-E2) and a Wnt3 site (E3-E4) on LRP5/6. The Fabs may be specific for a particular FZD, e.g. FZD4, or may be pan-specific, binding to more than one FZD, e.g., to FZD4 and one or more other FZD.

FIG. 16B depicts IgG-Diabody FZD4 Agonists having Fc regions with attenuated effector functions due to amino acid mutations, e.g., N297G (NG) and D265A, (DANG) variants, and/or LALAPS variants, and with the Fc region further comprising knob-in-hole heterodimerization variants Merrimack, Merchant or Merchant S:S. Table 13 presents the amino acid sequences of heavy chains and light chains of FZD4 Agonist, ANT39 (Diabody-Fc-Fab format) and ANT39wi (IgG-Diabody format): the heavy chain knob construct (ANT39 and ANT39i knob), the heavy chain hole construct (ANT39 and ANT39i hole) and the light chain construct. Also included in Table 13 are amino acid sequences of heavy chains and light chains of FZD4 Agonist, ANT39 and ANT39i variants DANG, LALAPS, LALAPS Merchant and LALAPS Merchant S-S. The light chain and heavy chain variable CDRs are in bold underlined italics.

TABLE 6A

| | LRP5 Diabody Site 1 | | | | | | LRP5 Diabody Site 2 | | | | |
|---|---|---|---|---|---|---|---|---|---|---|---|
| | | | SEQ | | SEQ | | SEQ | | SEQ | | SEQ |
| | | | ID | [3] | ID | [4] | ID | [5] | ID | [6] | ID |
| Molecule | [1] | [2] | NO | CDR-H2 | NO | CDR-H3 | NO | CDR-L2 | NO | CDR-L3 | NO |
| ANT16-Hole | D | FSSSSI | 528 | SISSSYGYTY | 553 | SWAM | 586 | SASDLYS | 491 | YAGAGLI | 510 |
| ANT16-Knob | D | FTAYAM | 536 | SIYPSGGYTA | 566 | RSYYFAL | 603 | SASSLYS | 2 | YWAYYSPI | 493 |
| ANT18-Hole | N | LSYYYM | 527 | SIYSSYGYTY | 552 | WSHVSGHYSGM | 584 | SASSLYS | 2 | SSYSLI | 130 |
| ANT18-Knob | N | FSSSSI | 528 | SISSSYGYTY | 553 | GGSGVSHYGSV | 585 | SASSLYS | 2 | ASYAPI | 492 |
| | | | | | | YYSWWAL | | | | | |
| ANT20-Hole | N | LSYYYM | 527 | SIYSSYGYTY | 552 | WSHVSGHYSGM | 584 | SASDLYS | 491 | YAGAGLI | 510 |
| ANT20-Knob | D | FTAYAM | 536 | SIYPSGGYTA | 566 | RSYYFAL | 603 | SASSLYS | 2 | ASYAPI | 492 |
| ANT21-Hole | N | FSSSSI | 528 | SISSSYGYTY | 553 | SWAM | 586 | SASSLYS | 2 | SSYSLI | 130 |
| ANT21-Knob | N | FSSSSI | 528 | SISSSYGYTY | 553 | GGSGVSHYGSV | 585 | SASSLYS | 2 | YWAYYS | 493 |
| | | | | | | YYSWWAL | | | | PI | |
| ANT36-Hole | N | LSYYYM | 527 | SIYSSYGYTY | 552 | WSHVSGHYSGM | 584 | SASSLYS | 2 | SSYSLI | 130 |
| ANT36-Knob | N | FSSSSI | 528 | SISSSYGYTY | 553 | GGSGVSHYGSV | 585 | SASSLYS | 2 | ASYAPI | 492 |
| | | | | | | YYSWWAL | | | | | |
| ANT39-Hole | D | FSSSSI | 528 | SISSSYGYTY | 553 | SWAM | 586 | SASDLYS | 491 | YAGAGLI | 510 |
| ANT39-Knob | D | FTAYAM | 536 | SIYPSGGYTA | 566 | RSYYFAL | 603 | SASSLYS | 2 | YWAYYS | 493 |
| | | | | | | | | | | PI | |
| ANT42-Hole | D | FSSSSI | 528 | SISSSYGYTY | 553 | SWAM | 586 | SASDLYS | 491 | YAGAGLI | 510 |
| ANT42-Knob | D | FTAYAM | 536 | SIYPSGGYTA | 566 | RSYYFAL | 603 | SASSLYS | 2 | YWAYYSPI | 493 |

In all the molecules the LRP5 Diabody site 2 CDR-LI is SVSSA (SEQ ID NO: 1)

TABLE 6B

| | | FZD4 FAb | | | | | | | |
|---|---|---|---|---|---|---|---|---|---|
| | | | SEQ | | SEQ | | SEQ | | SEQ |
| | | [8] | ID | [9] | ID | [10] | ID | [11] | ID |
| Molecule | [7] | CDR-H1 | NO: | CDR-H2 | NO: | CDR-H3 | NO: | CDR-L3 | NO: |
| ANT16-Hole | T | LSSYSM | 24 | YISSYYGYTY | 51 | PAPGHWGF | 79 | WYYAPI | 3 |
| ANT16-Knob | T | LSSYSM | 24 | YISSYYGYTY | 51 | PAPGHWGF | 79 | WYYAPI | 3 |
| ANT18-Hole | T | LSSYSM | 24 | YISSYYGYTY | 51 | PAPGHWGF | 79 | WYYAPI | 3 |
| ANT18-Knob | T | LSSYSM | 24 | YISSYYGYTY | 51 | PAPGHWGF | 79 | WYYAPI | 3 |
| ANT20-Hole | T | LSSYSM | 24 | YISSYYGYTY | 51 | PAPGHWGF | 79 | WYYAPI | 3 |
| ANT20-Knob | T | LSSYSM | 24 | YISSYYGYTY | 51 | PAPGHWGF | 79 | WYYAPI | 3 |
| ANT21-Hole | T | LSSYSM | 24 | YISSYYGYTY | 51 | PAPGHWGF | 79 | WYYAPI | 3 |
| ANT21-Knob | T | LSSYSM | 24 | YISSYYGYTY | 51 | PAPGHWGF | 79 | WYYAPI | 3 |
| ANT36-Hole | T | LSSYSM | 24 | YISSYDSITD | 61 | PAVGHMAF | 90 | WYNAPI | 12 |
| ANT36-Knob | T | LSSYSM | 24 | YISSYDSITD | 61 | PAVGHMAF | 90 | WYNAPI | 12 |
| ANT39-Hole | T | LSSYSM | 24 | YISSYDSITD | 61 | PAVGHMAF | 90 | WYNAPI | 12 |
| ANT39-Knob | T | LSSYSM | 24 | YISSYDSITD | 61 | PAVGHMAF | 90 | WYNAPI | 12 |
| ANT42-Hole | T | SDFYFI | 164 | TIYPFIGNTY | 194 | AFPGSYHPF | 220 | STGSLI | 135 |
| ANT42-Knob | T | SDFYFI | 164 | TIYPFIGNTY | 194 | AFPGSYHPF | 220 | STGSLI | 135 |

In all the molecules the FZD FAB CDR-LI and CDR-L2 are respectively SVSSA (SEQ ID NO: 1) and SASSLYS (SEQ ID NO: 2)

TABLE 6C

| | LRP6 Diabody Site 1 | | | | | | LRP6 Diabody Site 2 | | | | |
|---|---|---|---|---|---|---|---|---|---|---|---|
| | | | SEQ | | SEQ | | SEQ | | SEQ | | SEQ |
| | | [2] | ID | [3] | ID | [4] | ID | [5] | ID | [6] | ID |
| Molecule | [1] | CDR-H1 | NO | CDR-H2 | NO | CDR-H3 | NO | CDR-L2 | NO | CDR-L3 | NO |
| ANT59- | D | ISSYFI | 720 | SIYPSY | 791 | NVRGFRKP | 862 | SASSLYS | 2 | YFFPI | 665 |
| Hole | | | | SYTS | | YFSRWAM | | | | | |
| ANT59- | D | LSHSSI | 716 | SISSHY | 785 | SSYFPWFF | 856 | SASSLYS | 2 | YYWPI | 623 |
| Knob | | | | GYTH | | AL | | | | | |
| ANT9- | D | ISSYFI | 720 | SIYPSY | 791 | NVRGFRKP | 862 | SASSLYS | 2 | YFFPI | 665 |
| Hole | | | | SYTS | | YFSRWAM | | | | | |
| ANT9- | D | LSHSSI | 716 | SISSHY | 785 | SSYFPWFF | 856 | SASSLYS | 2 | YYWPI | 623 |
| Knob | | | | GYTH | | AL | | | | | |

In all the molecules the CDR-LI of all LRP abs are SVSSA (SEQ ID NO: 1)

TABLE 6D

| | FZD Paratope | | | | | | | | | |
|---|---|---|---|---|---|---|---|---|---|---|
| | | | | SEQ | | SEQ | | SEQ | | SEQ |
| | FZD | | H1 (8) | ID | H2 (9) | ID | H3 (10) | ID | L3 (11) | ID |
| Molecule | Specificity | 7 | CDR-H1 | NO | CDR-H2 | NO | CDR-H3 | NO | CDR-L3 | NO |
| ANT59-Hole | 5 | N | IAYSSM | 365 | TIYPSYSSTY | 462 | YYAM | 484 | AHYFPI | 285 |
| ANT59-Knob | 5 | N | IAYSSM | 365 | TIYPSYSSTY | 462 | YYAM | 484 | AHYFPI | 285 |
| ANT9-Hole | 1, 2, 4, | N | IHSSSI | 893 | ATYSSFGSIT | 894 | YHHPFGYAL | 895 | GVYLF | 896 |
| | 5, 7, 8 | | | | | | | | | |
| ANT9-Knob | 1, 2, 4, | N | IHSSSI | 893 | ATYSSFGSIT | 894 | YHHPFGYAL | 895 | GVYLF | 896 |
| | 5, 7, 8 | | | | | | | | | |

In all molecules the CDR-L1 for FZD FAB are SVSSA (SEQ ID NO: 1) and CDR-L2 are SASSLYS (SEQ ID NO: 2)

C. FZD Agonists are Highly Specific for FZD4, Bind with High Specificity and are Stable in Solution.

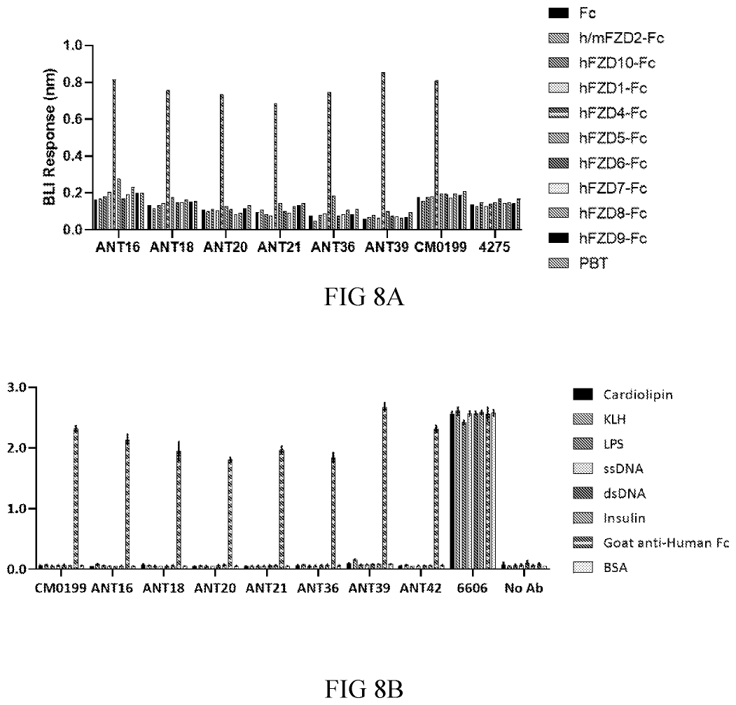

Using biolayer interferometry (BLI) we have found that FZD4 Agonists described herein are highly specific for FZD4 over other FZD receptors. Recombinant FZD ECD proteins were immobilized on BLI sensors. The FZD4 Agonists in the Diabody-Fc-Fab format, having a LRP5 binding domain comprised of a diabody that is bivalent and bispecific for LRP5 and a FZD4 binding domain comprised of two FZD4 binding Fab fragments, were tested at a concentration of 100 nM in a buffer of PBS+0.05% Tween-20 and 1% BSA for binding to the ECD proteins. The results are presented in FIG. 8A. Controls in the assay included CM0199, a diabody-Fc-diabody format FZD agonist that recognizes FZD4 and LRP5 and Immunoglobulin 4275, which is an IgG that does not bind FZD or LRP.

The FZD4 Agonists also did not recognize common non-specific antigens. The FZD4 Agonists were tested at 100 nM for binding to a panel of antigens essentially as described in Monquet et al. “Polyreactivity increases the apparent affinity of anti-HIV antibodies by heteroligation” Nature 2010 Sep. 30; 467(7315):591-5(PMC3699875), and Jain et al., “Biophysical properties of the clinical-stage antibody landscape” Proc Natl Acad Sci 2017 Jan. 31; 114(5):944-949. (PMC5293111). Controls in the assay included CM0199, a diabody-Fc-diabody format FZD agonist that recognizes FZD4 and LRP5 and immunoglobulin 6606, which is an IgG that is particularly prone to non-specific binding in this assay. The results are presented in FIG. 8B.

The FZD4 Agonists comprising binding domains for FZD4 and LRP5 bind both FZD4 and LRP5 with high affinity. The apparent affinity of the FZD4 Agonists for recombinant ECD of FZD4 were determined by biolayer interferometry essentially as described in Elife. 2019 Aug. 27; 8: e46134. Briefly, BLI assays were performed using an Octet HTX instrument (ForteBio). For measuring binding to antigen, FZD-Fc proteins were captured on AHQ BLI sensors (18-5001, ForteBio) to achieve a BLI response of 0.6-1 nm and remaining Fc-binding sites were saturated with human Fc (009-000-008, Jackson ImmunoResearch). FZD-coated or control (Fc-coated) sensors were transferred into 100-0.1 nM tetravalent FZD agonist in assay buffer (PBS, 1% BSA, 0.05% Tween20) and association was monitored for 300 s. Sensors were then transferred into assay buffer and dissociation was monitored for an additional 300 s. Shake speed was 1000 rpm and temperature was 25° C. The results are presented in Table 7.

TABLE 7

| | FZD4 | LRP5 |
|---|---|---|
| Molecule | KD (nM) | EC50 (nM) |
| CM0199 | 0.7 | 7.5 |
| ANT16 | 0.6 | 1.4 |
| ANT18 | 2.6 | ND |
| ANT20 | 0.7 | ND |
| ANT21 | 2.2 | ND |
| ANT36 | <0.1* | ND |
| ANT39 | 0.3 | ND |

The FZD4 Agonists were also analyzed by SEC as compared to trastuzumab IgG. The results are presented in FIG. 9A and demonstrate that the diabody-Fc-Fab format Agonists are stable and homogenous in solution.

The FZD4 Agonists are also stable in solution. Purified FZD4 Agonists, ANT16, ANT18, ANT20, ANT21 and ANT 36 were resuspended to 1 mg/ml (except for ANT18, which was resuspended at 0.34 mg/ml) in 10 mM Histidine, 140 mM NaCl, 0.9% sucrose, pH 6 and stored either at 4° C. or 40° C. for a period of 6 days. Samples were removed at various time points, centrifuged to remove precipitated protein and residual protein concentration was measured. The results are presented in Tables 8 and 9.

TABLE 8

| | |
|---|---|
| Time at 40 | Soluble Protein Remaining (%) |

| degrees (days) | 0 | 1 | 2 | 6 |
|---|---|---|---|---|
| CM0199 | 100% | 60% | 64% | 40% |
| ANT-16 | 100% | 102% | 104% | 104% |
| ANT-18 | 100% | 92% | 83% | 105% |
| ANT-20 | 100% | 97% | 67% | 104% |
| ANT-21 | 100% | 102% | 75% | 102% |
| ANT-36 | 100% | 100% | 149% | 106% |

TABLE 9

| | |
|---|---|
| | Active Protein |
| Time at 40 | Remaining (%) |

| degrees (days) | 0 | 6 |
|---|---|---|
| CM0199 | 100% | 42% |
| ANT-16 | 100% | 34% |
| ANT-18 | 100% | 42% |
| ANT-20 | 100% | 30% |
| ANT-21 | 100% | 25% |

On Day 6, the amount of FZD4-specific binding sites remaining in the samples were quantified using BLI. Analysis by differential scanning fluorimetry showed that the FZD4 Agonists having a Diabody-Fc-Fab formats with an LRP-binding diabody on the N-terminal of the Fc domain and two FZD4-binding Fabs on the C-terminal of the Fc domain, have thermal denaturation profiles similar to that of Trastuzumab. IgGs generally display two peaks in a thermal stability assay, the first corresponding to CH2, the later to the Fab domain and CH3, see FIG. 9B.

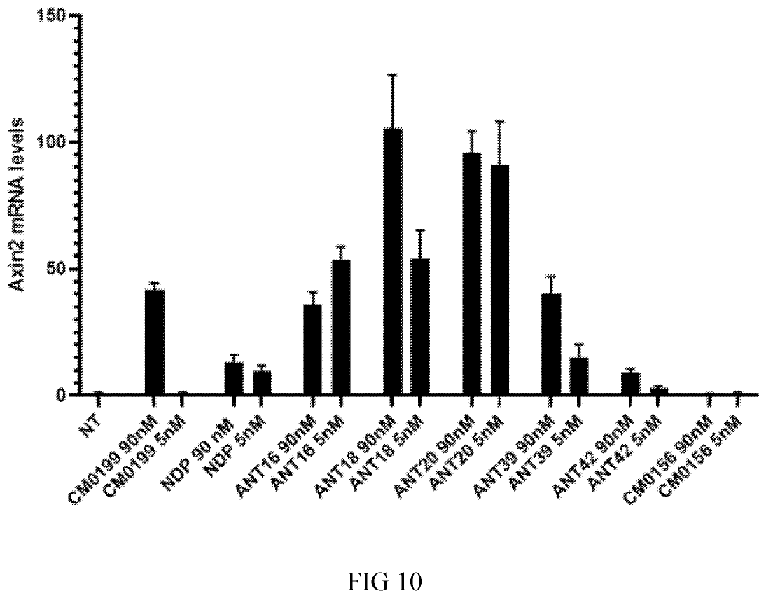

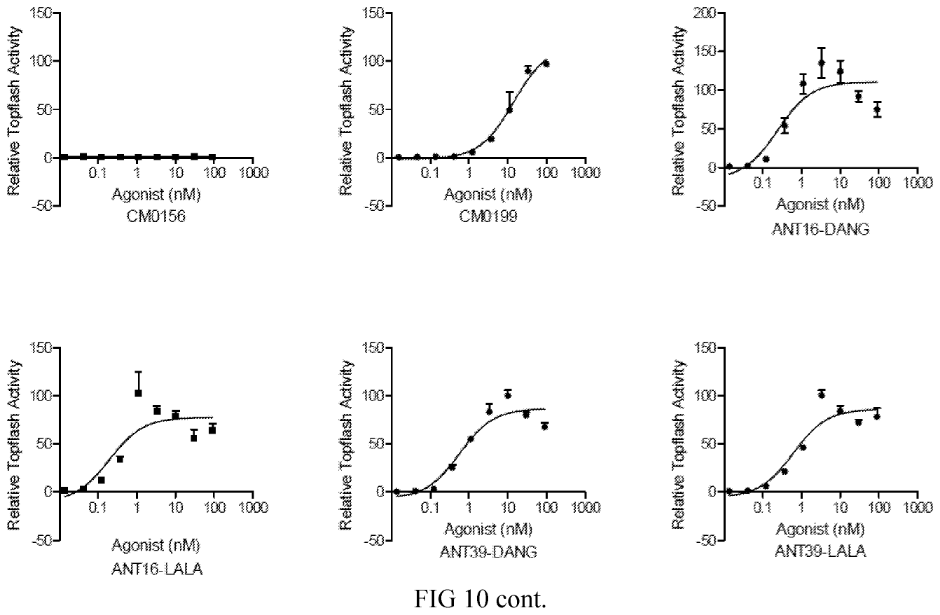

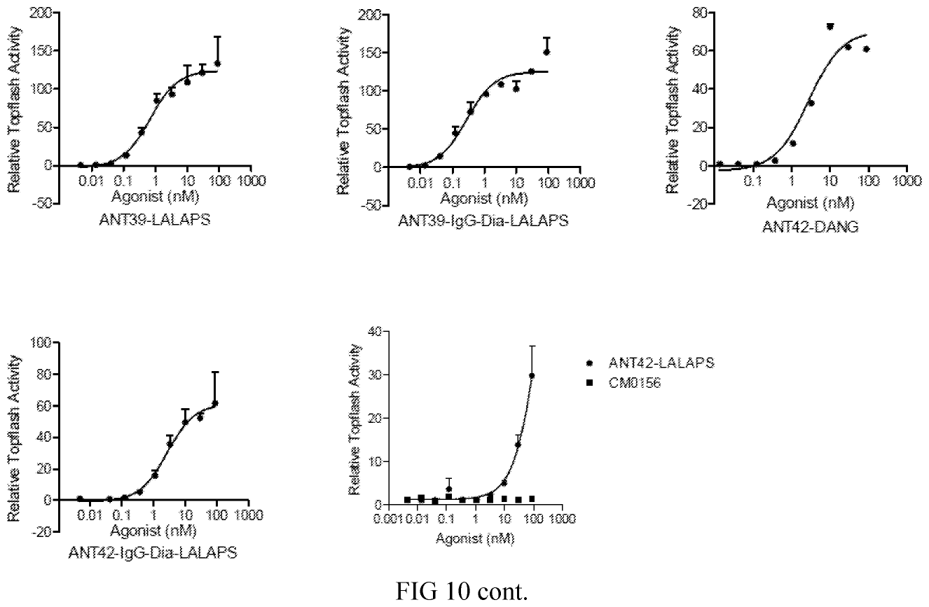

The FZD4 Agonists were also assayed for induction of the beta-catenin target gene AXIN2 in a mouse endothelial cell line (bEND3.1) and were shown to induce transcription in a concentration dependent manner. These results are presented in FIG. 10.

TABLE 10

| Antibody | | Co-Receptor |
|---|---|---|
| No. | FZD recognized | recognized |
| 5019 | FZD1, FZD2, FZD4, FZD5, FZD7, FZD8 | |
| 5027 | FZD4 | |
| 5044 | FZD4 | |
| 5016 | FZD1, FZD2, FZD4, FZD5, FZD7, FZD8 | |
| 5056 | FZD1, FZD 2, FZD 4, FZD5, FZD7, FZD8, | |
| | FZD10 | |
| 2459 | | LRP5-W3 |
| 2460 | | LRP5-W1 |
| 8716 | | LRP5-W3 |
| 12600 | | LRP5-W1 |
| 12608 | | LPR5-W1 |
| 2539 | | LRP6-W3 |
| 2540 | | LRP6-W3 |
| 2542 | | LRP6-W1 |
| CM0199 | FZD4 | LRP5 |
| 6066 | N/A Positive control for non-specificity assay | |
| 4275 | no FZD/LRP binding | |

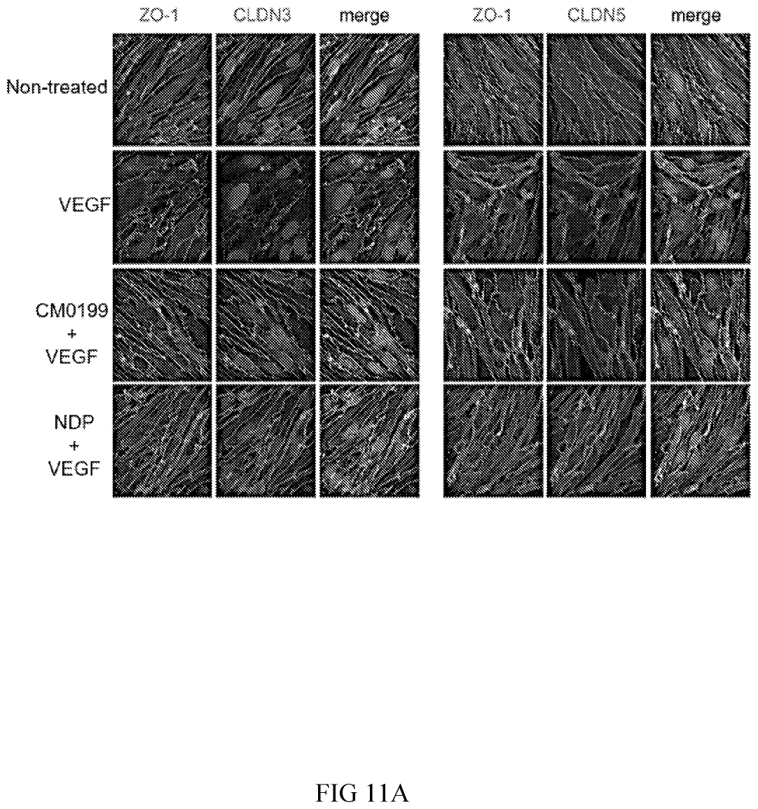

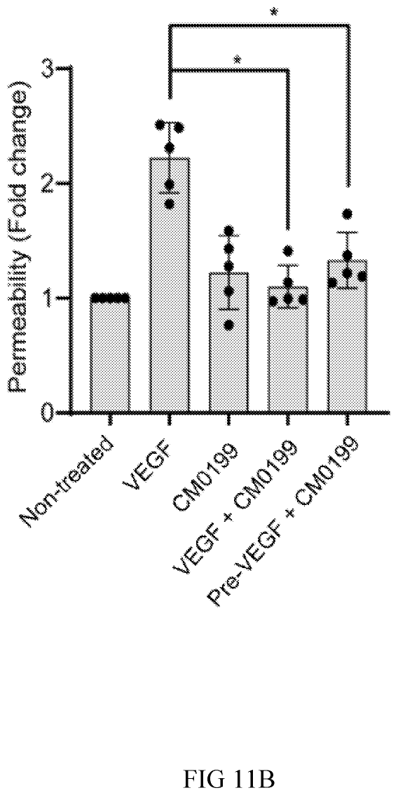

EXAMPLE 4. The FZD4 agonist was assayed for its ability to oppose the effect on cell junction disassembly and increased permeability mediated by VEGF, a cytokine released during tissue hypoxia. VEGF treatment of bEND3.1 cells leads to junction disassembly as seen by loss of plasma membrane staining of CLDN3, CLDN5 and ZO-1. Co-treatment of cells with VEGF and the FZD4 agonist leads to a near-complete rescue of this effect (FIG. 11). This decrease cell-cell junction stability mediated by VEGF treatment translates into increase endothelial cell permeability as monitor in a transendothelial permeability assay measuring the passage of 40-kDa FITC-dextran across a confluent endothelial monolayer of bEnd.3 grown on transwell filters. Co-treatment of cells with VEGF and the FZD4 agonist completely rescues that VEGF-mediated increase in cell permeability. These results indicate that the FZD4 agonist promotes endothelial cell barrier functions in a mechanism independent of VEGF.

A) Immunofluoresence of ZO-1 (green)/CLDN3 (red) and ZO-1 (green)/CLDN5 (red) localization on bEnd.3 cell junctions. bEnd.3 cells were treated or not with 30 nM of F4L5.13 (aka CM0199) and Norrin in the presence or absence of VEGF (100 ng/ml) for 1h. DAPI (blue) stain the nucleus. B) Transendothelial permeability was determined by measuring the passage of FITC-dextran through the bEnd.3 monolayer. Passage of FITC-dextran was measured after bEnd.3 treatment with VEGF (100 ng/ml) and F4L5.13 (30 nM) alone or simultaneously or upon pre-treatment with VEGF for 1h followed by F4L5.13 treatment for 1h. Error bar indicate SEM, n=5. The results are presented in FIG. 11.

### Example 5. New FZD5 Antibodies Bind FZD5 at a Site Overlapping with 2919 Identified from Affinity Maturation Libraries

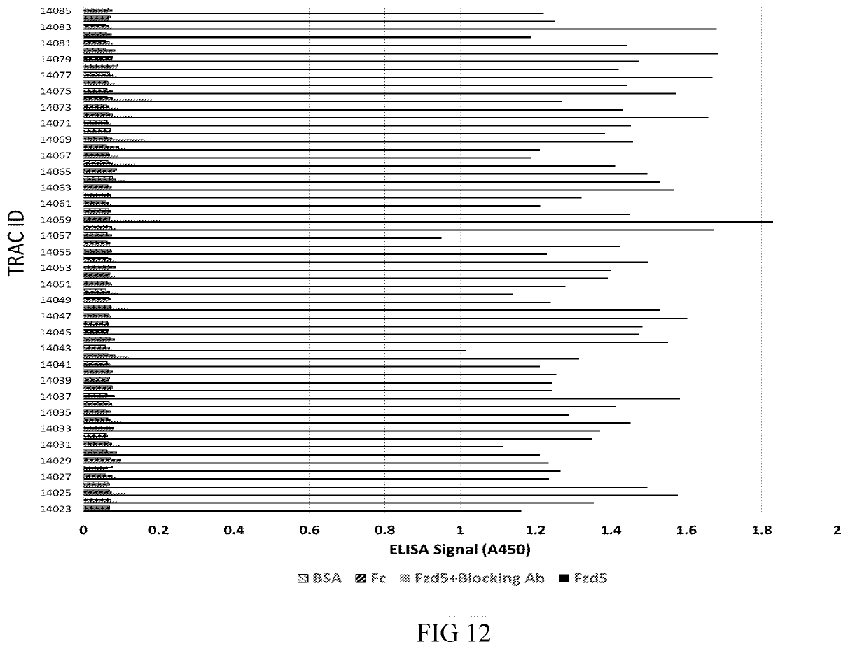

Single point ELISAs were performed on 96-well Maxisorp plates coated with the ECDs of human FZD5 protein in the presence or absence of a saturating concentration of 2919 IgG. The plates were incubated with the monoclonal Fab-phage followed by incubation with horseradish peroxidase (HRP)-conjugated anti-M13 antibody. Wells were subsequently washed 8 times followed by incubations with 3,3,′5,5′-tetramethylbenidine/H₂O₂ peroxidase (TMB) substrate for 5-10 min. the reaction was stopped by adding 1M H₃PO₄ and the absorbance was measured spectrophotometrically at 450 nm in a microtiter plate reader. The results are presented in FIG. 12.

### Example 6. New FZD5 Antibodies from 2928 Affinity Maturation Library Selectively Bind FZD5

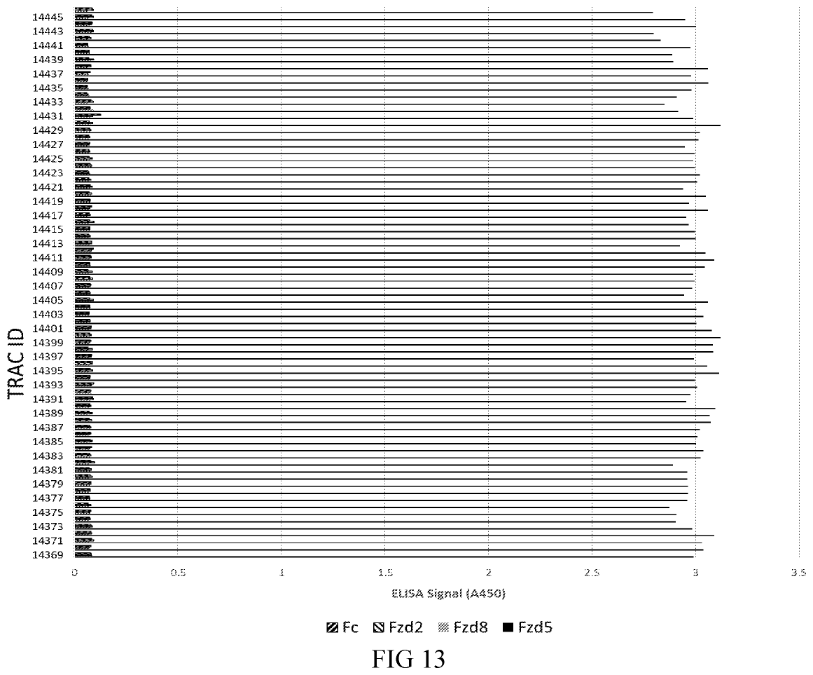

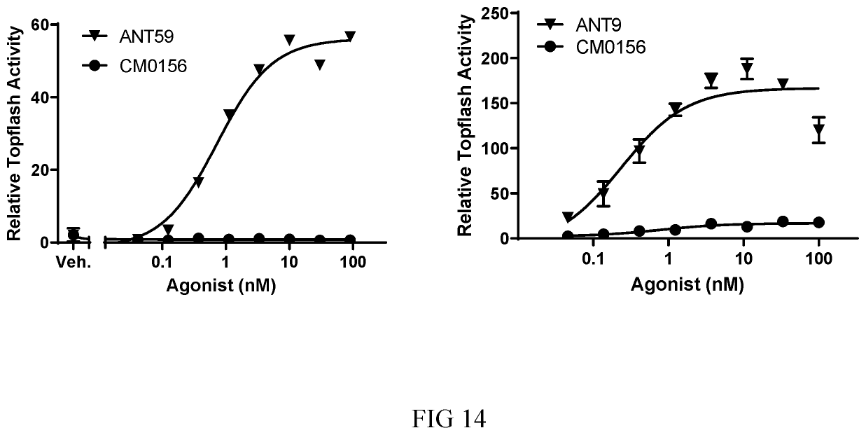

Single point ELISAs were performed on 96-well Maxisorp plates coated with the ECDs of human FZD2, FZD5, or FZD8 protein. The plates were incubated with the monoclonal Fab-phage followed by incubation with horseradish peroxidase (HRP)-conjugated anti-M13 antibody. Wells were subsequently washed 8 times followed by incubations with 3,3,′5,5′-tetramethylbenidine/H₂O₂ peroxidase (TMB) substrate for 5-10 min. the reaction was stopped by adding 1M H₃PO₄ and the absorbance was measured spectrophotometrically at 450 nm in a microtiter plate reader. The results are presented in FIG. 13.

### Example 7. Pan-FZD/LRP6 ANT9 and FZD5-Specific/LRP6 ANT9 Activate Wnt Signaling in Cells

TOPFLASH HEK293 cells were treated overnight with varying concentrations of FZD agonist or a non-targeting control molecule (CM0156) and TCF/LEF-driven luciferase expression was measured using a standard luciferase assay. Both molecules are able to activate FZD-mediated luciferase expression in a concentration-responsive manner. ANT9, which is able to bind to 7 of the 10 FZD receptor subtypes produces a higher maximal activation signal than the FZD5-specific ANT59.

### In Vivo Experiments

### DSS Induced Colitis Model

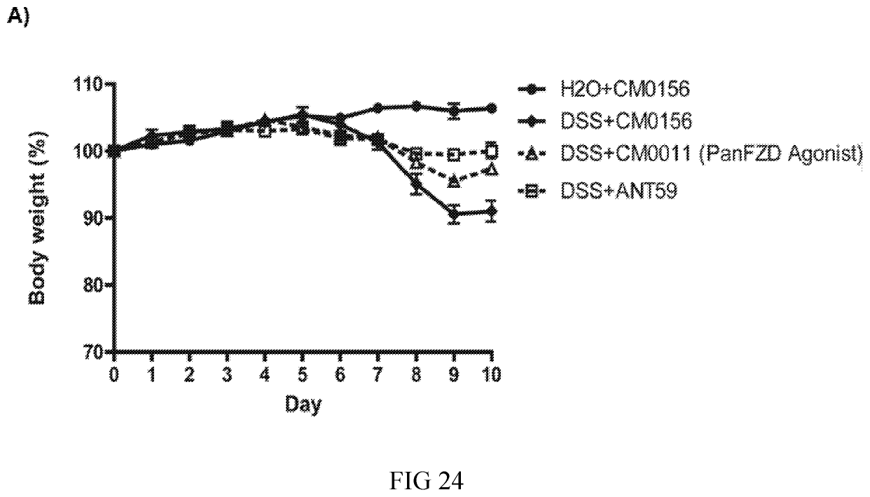

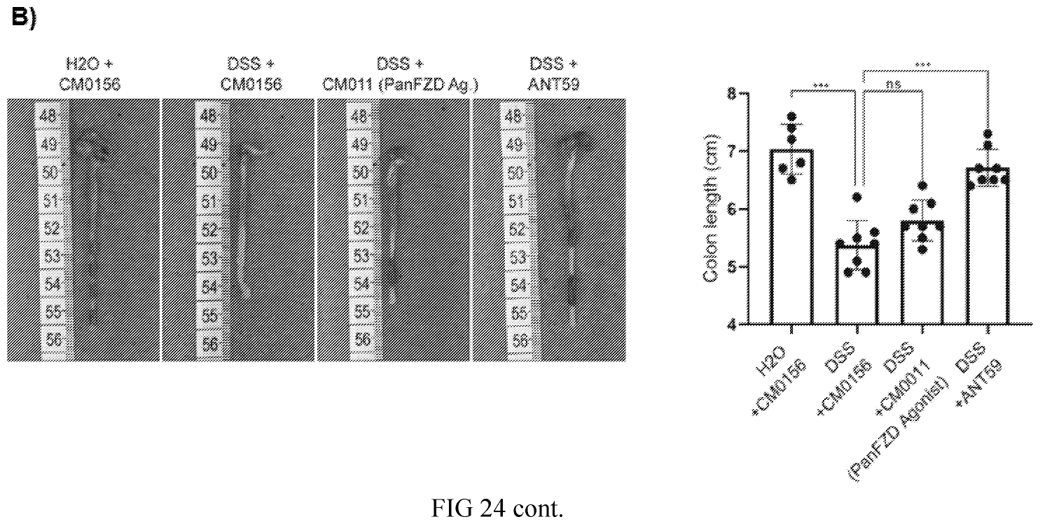

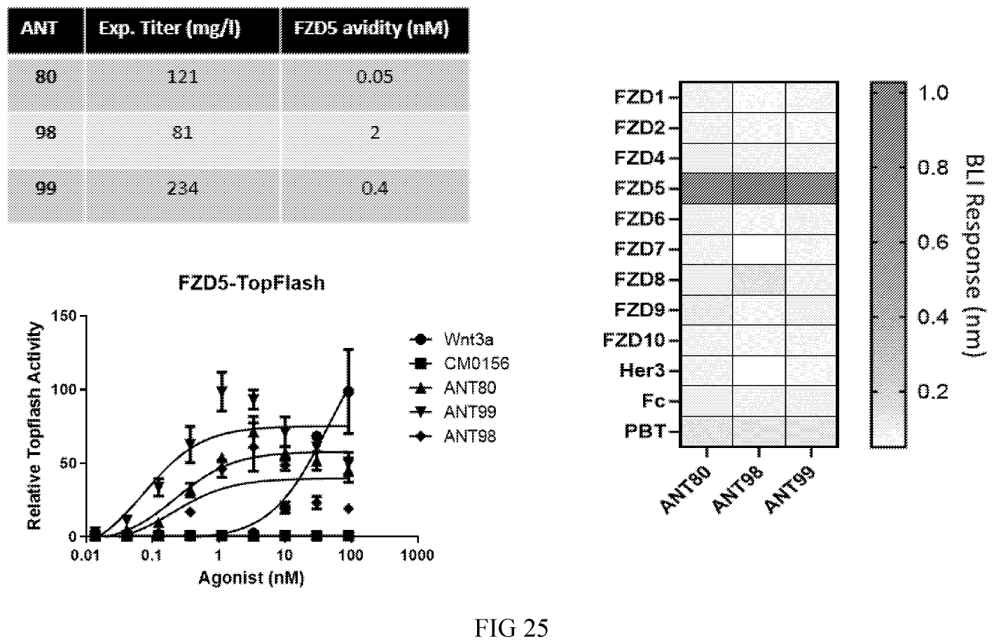

In FIG. 24, C57/BL6 mice were given 2% DSS in the drinking water for 7 days and 0.5% DSS for an additional 3 days to induce colitis. Control-FLAg, Pan-FLAg and ANT59 were administered via intraperitoneal injection on days 4 and 7 at a dosage of 10 mg/kg. Mice were weighed daily. On day 10 mice were euthanized and tissues were harvested for measurement of colon length and histology.

### Histology

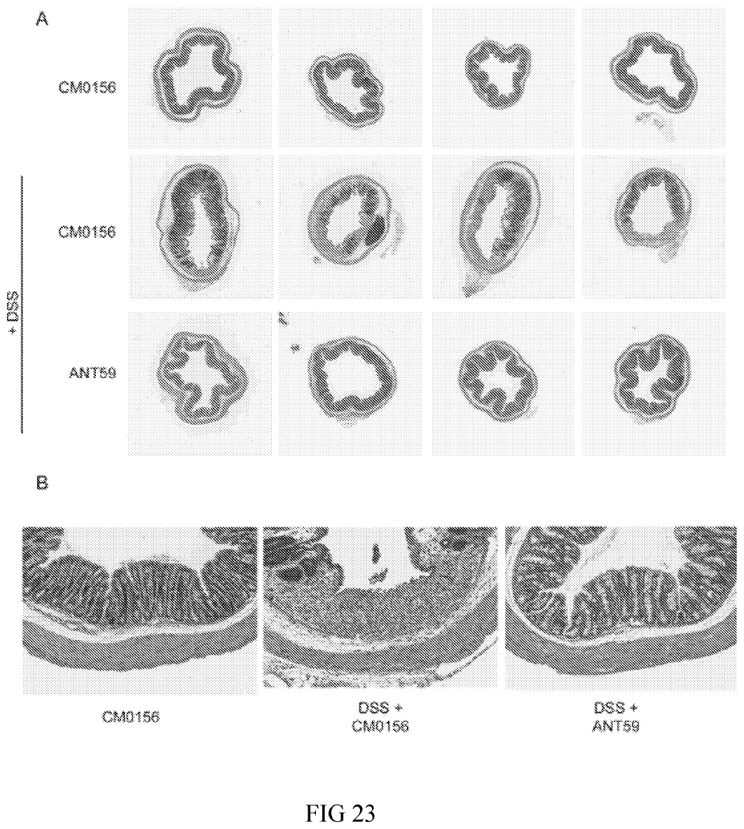

For histological analysis, harvested tissues were fixed in 4% paraformaldehyde and embedded in paraffin. Sections of 5 μm were stained with haematoxylin and eosin (H&E). Images were captured using a Nikon Eclipse microscope (FIG. 23).

### Organoid Culture and Viability Measurement

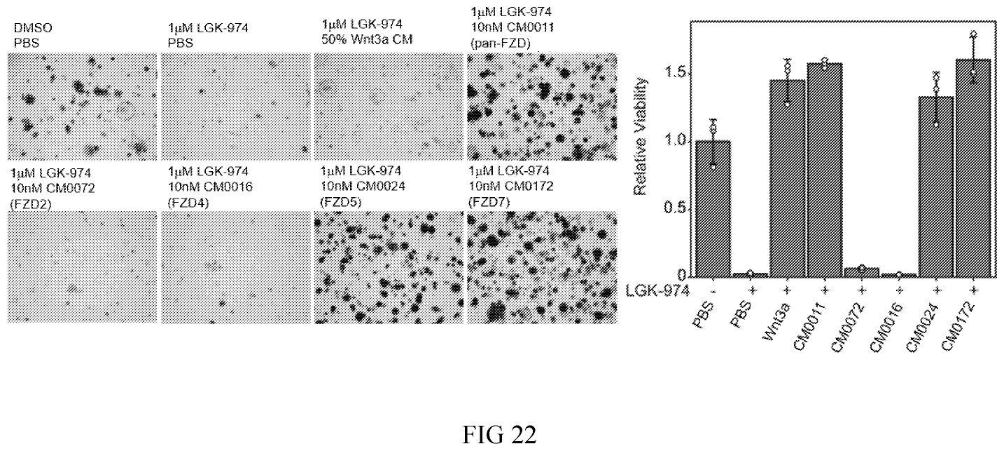

Small intestine crypts were harvested from 8-week-old, female, C57BL/6 mice and cultured as previously described (O'Rourke et al., 2016). Organoid cultures were passaged and embedded in 25 μl Growth Factor Reduced Matrigel (Corning, 356231) and plated in triplicates in a 48-well plate. Organoid cultures were treated with DMSO, 1 μM LGK974, 1 μM LGK974+50% WNT3A conditioned media, 1 μM LGK974+30 nM Pan-FLAg, 1 μM LGK974+30 nM FZD2-FLAg, 1 μM LGK974+30 nM FZD4-FLAg, 1 μM LGK974+30 nM FZD5-FLAg, 1 μM LGK974+30 nM FZD7-FLAg. Treatments were prepared in 250 μl of complete media, added to each well on day of passaging and changed every 2-3 days. At the endpoint (7 days), 150 μl Cell Titer-Glo3D (Promega) was added to 150 μl media in each well. Organoids were lysed on a rocking platform for 30 min at room temperature. The luminescence reading was measured in duplicates for 20 μl lysate from each well on the Envision multilabel plate reader. The average luminescence reading for each condition was normalized to the control condition to calculate relative viability (FIG. 22).

EXAMPLE 8. Transient expression of 8 ANT39 variants. A series of eight ANT39 variants (FIGS. 16A and 16B) were transiently expressed in CHO cells using standard manufacture lipid based protocols (ThermoFisher). Briefly cells were grown to a density of approximately 2.0×106 cells/ml in growth media and relevant DNAs were transfected with appropriate transfection reagent. For each variant two alternate input plasmid ratios were tested, either 1:1:2 or 2:1:3 (knob heavy chain: hole heavy chain: light chain). Conditioned media was harvested 7 days later, purified by Protein A Sepharose and the titre measured.

TABLE 11

| | DNA Ratio | Titer |
|---|---|---|
| Molecule | (Knob:Hole:LC) | (mg/L) |
| ANT39 | 1:1:2 | 390 |
| | 2:1:3 | 290 |
| ANT39i | 1:1:2 | 260 |
| | 2:1:3 | 250 |
| ANT39 LALAPS | 1:1:2 | 330 |
| | 2:1:3 | 250 |
| ANT39i LALAPS | 1:1:2 | 300 |
| | 2:1:3 | 270 |
| ANT39 LALAPS | 1:1:2 | 340 |
| Merchant | 2:1:3 | 280 |
| ANT39i LALAPS | 1:1:2 | 240 |
| Merchant | 2:1:3 | 280 |
| ANT39 LALAPS | 1:1:2 | 320 |
| Merchant S:S | 2:1:3 | 190 |
| ANT39i LALAPS | 1:1:2 | 300 |
| Merchant S:S | 2:1:3 | 250 |
| ANT42* | 1:1:2 | 107 |
| ANT42 LALA* | 1:1:2 | 287 |
| ANT42 LALAPS* | 1:1:2 | 95 |
| ANT42i LALAPS* | 1:1:2 | 49 |

*Variants were transiently expressed in HEK293 cells

Those skilled in the art will recognize, or be able to ascertain, using no more than routine experimentation, numerous equivalents to the specific procedures described herein. Such equivalents are considered to be within the scope of the inventions. Various substitutions, alterations and modifications may be made to the invention without departing from the spirit and scope of the invention. Other aspects, advantages, and modifications are within the scope of the invention.

The contents of all references, issued patents, and published patent applications cited through this application are hereby incorporated by reference. The appropriate component, process and methods of those patents, applications and other documents may be selected for the invention and embodiments thereof.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

To clarify the use of and to hereby provide notice to the public, the phrases “at least one of <A>, <B>, . . . and <N>” or “at least one of <A>, <B>, . . . or <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed. Unless otherwise indicated or the context suggests otherwise, as used herein, “a” or “an” means “at least one” or “one or more.”

TABLE 12
Diabody-Fc-Fab amino acid sequences of the “heavy chain” hole construct,
“heavy chain” knob construct and the “light chain” construct of the
FZD4 Agonists, ANT16, ANT18, ANT20, ANT21, ANT39, and ANT42.
The CDRs of the chains are underlined, italicized. The CDRs may be
replaced with the CDRs of another antibody to alter the binding specificity,
e.g., the specificity could be altered to bind another site on the
FZD or LPR5/6, or to another FZD or LPR.
FZD4 Agonist ANT16

| | |
|---|---|
| Knob | EVQLVESGGGLVQPGGSLRLSCAASGFDFTAYAMHWVRQAPGKGL |
| Hc | EWVASIYPSGGYTAYADSVKGRFTISADTSKNTAYLQMNSLRAED |
| construct | TAVYYCARRSYYFALDYWGQGTLVTVSSGGGGSDIQMTQSPSSLS |
| | ASVGDRVTITCRASQSVSSAVAWYQQKPGKAPKLLIYSASSLYSG |
| | VPSRFSGSRSGTDFTLTISSLQPEDFATYYCQQYWAYYSPITFGQ |
| | GTKVEIKGGGGSGGGGSEPKTSDKTHTCPPCPAPELLGGPSVFLF |
| | PPKPKDTLMISRTPEVTCVVVAVSHEDPEVKFNWYVDGVEVHNAK |
| | TKPREEQYGSTYRVVSVLTVLHQDWLNGKEYKCKVSNKALPAPIE |
| | KTISKAKGQPREPMVFDLPPSREEMTKNQVSLWCMVKGFYPSDIA |
| | VEWESNGQPENNYKTTPPVLDSDGSFFLYSKLTVDKSRWQQGNVF |
| | SCSVMHEALHNHYTQKSLSLSPGKGGGSGGGSGGGSGGGSGSTGE |
| | VQLVESGGGLVQPGGSLRLSCAASGFTLSSYSMHWVRQAPGKGLE |
| | WVAYISSYYGYTYYADSVKGRFTISADTSKNTAYLQMNSLRAEDT |
| | AVYYCARPAPGHWGFDYWGQGTLVTVSSASTKGPSVFPLAPSSKS |
| | TSGGTAALGCLVKDYFPEPVTVSWNSGALTSGVHTFPAVLQSSGL |
| | YSLSSVVTVPSSSLGTQTYICNVNHKPSNTKVDKKVEPKSCDKTH |
| | T |
| | (SEQ ID NO: 897) |
| Hole | EVQLVESGGGLVQPGGSLRLSCAASGFDFSSSSIHWVRQAPGKGL |
| Hc | EWVASISSSYGYTYYADSVKGRFTISADTSKNTAYLQMNSLRAED |
| construct | TAVYYCARSWAMDYWGQGTLVTVSSGGGGSDIQMTQSPSSLSASV |
| | GDRVTITCRASQSVSSAVAWYQQKPGKAPKLLIYSASDLYSGVPS |
| | RFSGSRSGTDFTLTISSLQPEDFATYYCQQYAGAGLITFGQGTKV |
| | EIKGGGGSGGGGSEPKSSDKTHTCPPCPAPELLGGPSVFLFPPKP |
| | KDTLMISRTPEVTCVVVAVSHEDPEVKFNWYVDGVEVHNAKTKPR |
| | EEQYGSTYRVVSVLTVLHQDWLNGKEYKCKVSNKALPAPIEKTIS |
| | KAKGQPREPQVYTLPPIRELMTSNQVSLSCAVKGFYPSDIAVEWE |
| | SNGQPENNYKTTPPVLDSDGSFFLVSKLTVDKSRWQQGNVFSCSV |
| | MHEALHNHYTQKSLSLSPGKGGGSGGGSGGGSGGGSGSTGEVQLV |
| | ESGGGLVQPGGSLRLSCAASGFTLSSYSMHWVRQAPGKGLEWVAY |
| | ISSYYGYTYYADSVKGRFTISADTSKNTAYLQMNSLRAEDTAVYY |
| | CARPAPGHWGFDYWGQGTLVTVSSASTKGPSVFPLAPSSKSTSGG |
| | TAALGCLVKDYFPEPVTVSWNSGALTSGVHTFPAVLQSSGLYSLS |
| | SVVTVPSSSLGTQTYICNVNHKPSNTKVDKKVEPKSCDKTHT |
| | (SEQ ID NO: 898) |
| Light | DIQMTQSPSSLSASVGDRVTITCRASQSVSSAVAWYQQKPGKAPK |
| Chain | LLIYSASSLYSGVPSRFSGSRSGTDFTLTISSLQPEDFATYYCQQ |
| | WYYAPITFGQGTKVEIKRTVAAPSVFIFPPSDEQLKSGTASVVCL |
| | LNNFYPREAKVQWKVDNALQSGNSQESVTEQDSKDSTYSLSSTLT |
| | LSKADYEKHKVYACEVTHQGLSSPVTKSFNRGEC |
| | (SEQ ID NO: 899) |

FZD4 Agonist ANT18

| | |
|---|---|
| Knob | MNLLLILTFVAAAVAEVQLVESGGGMVQPGGSLRLSCAASGFNFS |
| Hc | SSSIHWVRQAPGKGLEWVASISSSYGYTYYADSVKGRFTISADTS |
| construct | KNTAYLQMNSLRAEDTAVYYCARGGSGVSHYGSVYYSWWALDYWG |
| | QGTLVTVSSGGGGSDIQMTQSPSSLSASVGDRVTITCRASQSVSS |
| | AVAWYQQKPGKAPKLLIYSASSLYSGVPSRFSGSRSGTDFTLTIS |
| | SLQPEDFATYYCQQASYAPITFGQGTKVEIKGGGGSGGGGSEPKT |
| | SDKTHTCPPCPAPELLGGPSVFLFPPKPKDTLMISRTPEVTCVVV |
| | AVSHEDPEVKFNWYVDGVEVHNAKTKPREEQYGSTYRVVSVLTVL |
| | HQDWLNGKEYKCKVSNKALPAPIEKTISKAKGQPREPMVEDLPPS |
| | REEMTKNQVSLWCMVKGFYPSDIAVEWESNGQPENNYKTTPPVLD |
| | SDGSFFLYSKLTVDKSRWQQGNVFSCSVMHEALHNHYTQKSLSLS |
| | PGKGGGSGGGSGGGSGGGSGSTGEVQLVESGGGLVQPGGSLRLSC |
| | AASGFTLSSYSMHWVRQAPGKGLEWVAYISSYYGYTYYADSVKGR |
| | FTISADTSKNTAYLQMNSLRAEDTAVYYCARPAPGHWGFDYWGQG |
| | TLVTVSSASTKGPSVFPLAPSSKSTSGGTAALGCLVKDYFPEPVT |
| | VSWNSGALTSGVHTFPAVLQSSGLYSLSSVVTVPSSSLGTQTYIC |
| | NVNHKPSNTKVDKKVEPKSCDKTHT |
| | (SEQ ID NO: 900) |
| Hole | MNLLLILTFVAAAVAEVQLVESGGGLVQPGGSLRLSCAASGFNLS |
| Hc | YYYMHWVRQAPGKGLEWVASIYSSYGYTYYADSVKGRFTISADTS |
| construct | KNTAYLQMNSLRAEDTAVYYCARWSHVSGHYSGMDYWGQGTLVTV |
| | SSGGGGSDIQMTQSPSSLSASVGDRVTITCRASQSVSSAVAWYQQ |
| | KPGKAPKLLIYSASSLYSGVPSRFSGSRSGTDFTLTISSLQPEDF |
| | ATYYCQQSSYSLITFGQGTKVEIKGGGGSGGGGSEPKSSDKTHTC |
| | PPCPAPELLGGPSVFLFPPKPKDTLMISRTPEVTCVVVAVSHEDP |
| | EVKFNWYVDGVEVHNAKTKPREEQYGSTYRVVSVLTVLHQDWLNG |
| | KEYKCKVSNKALPAPIEKTISKAKGQPREPQVYTLPPIRELMTSN |
| | QVSLSCAVKGFYPSDIAVEWESNGQPENNYKTTPPVLDSDGSFFL |
| | VSKLTVDKSRWQQGNVFSCSVMHEALHNHYTQKSLSLSPGKGGGS |
| | GGGSGGGSGGGSGSTGEVQLVESGGGLVQPGGSLRLSCAASGFTL |
| | SSYSMHWVRQAPGKGLEWVAYISSYYGYTYYADSVKGRFTISADT |
| | SKNTAYLQMNSLRAEDTAVYYCARPAPGHWGFDYWGQGTLVTVSS |
| | ASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPS |
| | NTKVDKKVEPKSCDKTHT |
| | (SEQ ID NO: 901) |
| Light Chain | MNLLLILTFVAAAVADIQMTQSPSSLSASVGDRVTITCRASQSVS |
| | SAVAWYQQKPGKAPKLLIYSASSLYSGVPSRFSGSRSGT |
| | DFTLTISSLQPEDFATYYCQQWYYAPITFGQGTKVEIKRTVAAPS |
| | VFIFPPSDEQLKSGTASVVCLLNNFYPREAKVQWKVDNALQSGNS |
| | QESVTEQDSKDSTYSLSSTLTLSKADYEKHKVYACEVTHQGLSSP |
| | VTKSFNRGEC |
| | (SEQ ID NO: 902) |

FZD4 Agonist ANT20

| | |
|---|---|
| Knob | MNLLLILTFVAAAVAEVQLVESGGGLVQPGGSLRLSCAASGFDF |
| Hc | AYAMHWVRQAPGKGLEWVASIYPSGGYTAYADSVKGRFTISADTS |
| construct | KNTAYLQMNSLRAEDTAVYYCARRSYYFALDYWGQGTLVTVSSGG |
| | GGSDIQMTQSPSSLSASVGDRVTITCRASQSVSSAVAWYQQKPGK |
| | APKLLIYSASSLYSGVPSRFSGSRSGTDFTLTISSLQPEDFATYY |
| | CQQASYAPITFGQGTKVEIKGGGGSGGGGSEPKTSDKTHTCPPCP |
| | APELLGGPSVFLFPPKPKDTLMISRTPEVTCVVVAVSHEDPEVKF |
| | NWYVDGVEVHNAKTKPREEQYGSTYRVVSVLTVLHQDWLNGKEYK |
| | CKVSNKALPAPIEKTISKAKGQPREPMVFDLPPSREEMTKNQVSL |
| | WCMVKGFYPSDIAVEWESNGQPENNYKTTPPVLDSDGSFFLYSKL |
| | TVDKSRWQQGNVFSCSVMHEALHNHYTQKSLSLSPGKGGGSGGGS |
| | GGGSGGGSGSTGEVQLVESGGGLVQPGGSLRLSCAASGFTLSSYS |
| | MHWVRQAPGKGLEWVAYISSYYGYTYYADSVKGRFTISADTSKNT |
| | AYLQMNSLRAEDTAVYYCARPAPGHWGFDYWGQGTLVTVSSASTK |
| | GPSVFPLAPSSKSTSGGTAALGCLVKDYFPEPVTVSWNSGALTSG |
| | VHTFPAVLQSSGLYSLSSVVTVPSSSLGTQTYICNVNHKPSNTKV |
| | DKKVEPKSCDKTHT |
| | (SEQ ID NO: 903) |
| Hole | MNLLLILTFVAAAVAEVQLVESGGGLVQPGGSLRLSCAASGFNLS |
| Hc | YYYMHWVRQAPGKGLEWVASIYSSYGYTYYADSVKGRFTISADTS |
| construct | KNTAYLQMNSLRAEDTAVYYCARWSHVSGHYSGMDYWGQGTLVTV |
| | SSGGGGSDIQMTQSPSSLSASVGDRVTITCRASQSVSSAVAWYQQ |
| | KPGKAPKLLIYSASDLYSGVPSRFSGSRSGTDFTLTISSLQPEDF |
| | ATYYCQQYAGAGLITFGQGTKVEIKGGGGSGGGGSEPKSSDKTHT |
| | CPPCPAPELLGGPSVFLFPPKPKDTLMISRTPEVTCVVVAVSHED |
| | PEVKFNWYVDGVEVHNAKTKPREEQYGSTYRVVSVLTVLHQDWLN |
| | GKEYKCKVSNKALPAPIEKTISKAKGQPREPQVYTLPPIRELMTS |
| | NQVSLSCAVKGFYPSDIAVEWESNGQPENNYKTTPPVLDSDGSFF |
| | LVSKLTVDKSRWQQGNVFSCSVMHEALHNHYTQKSLSLSPGKGGG |
| | SGGGSGGGSGGGSGSTGEVQLVESGGGLVQPGGSLRLSCAASGFT |
| | LSSYSMHWVRQAPGKGLEWVAYISSYYGYTYYADSVKGRFTISAD |
| | TSKNTAYLQMNSLRAEDTAVYYCARPAPGHWGFDYWGQGTLVTVS |
| | SASTKGPSVFPLAPSSKSTSGGTAALGCLVKDYFPEPVTVSWNSG |
| | ALTSGVHTFPAVLQSSGLYSLSSVVTVPSSSLGTQTYICNVNHKP |
| | SNTKVDKKVEPKSCDKTHT |
| | (SEQ ID NO: 904) |
| Light | MNLLLILTFVAAAVADIQMTQSPSSLSASVGDRVTITCRASQSVS |
| Chain | SAVAWYQQKPGKAPKLLIYSASSLYSGVPSRFSGSRSGTDFTLTI |
| | SSLOPEDFATYYCQQWYYAPITFGQGTKVEIKRTVAAPSVFIFPP |
| | SDEQLKSGTASVVCLLNNFYPREAKVQWKVDNALQSGNSQESVTE |
| | QDSKDSTYSLSSTLTLSKADYEKHKVYACEVTHQGLSSPVTKSFN |
| | RGEC |
| | (SEQ ID NO: 902) |

FZD4 Agonist ANT21

| | |
|---|---|
| Knob | MNLLLILTFVAAAVAEVQLVESGGGLVQPGGSLRLSCAASGENFS |
| Hc | SSSIHWVRQAPGKGLEWVASISSSYGYTYYADSVKGRFTISADTS |
| construct | KNTAYLQMNSLRAEDTAVYYCARGGSGVSHYGSVYYSWWALDYWG |
| | QGTLVTVSSGGGGSDIQMTQSPSSLSASVGDRVTITCRASQSVSS |
| | AVAWYQQKPGKAPKLLIYSASSLYSGVPSRFSGSRSGTDFTLTIS |
| | SLQPEDFATYYCQQYWAYYSPITFGQGTKVEIKGGGGSGGGGSEP |
| | KTSDKTHTCPPCPAPELLGGPSVFLFPPKDTLMISRTPEVTCVVV |
| | AVSHEDPEVKFNWYDGVEVHNAKTKPREEQYGSTRYRVVSVLTVL |
| | HQDQLNGKEYKCKVSNKALPAPEIKTISKAKGQPREPMVEDLPP |
| | SREEMTKNQVSLWCMVKGFYPSDIAVEWESNGQPENNYKTTPPVL |
| | DSDGSFFLYSKLTVDKSRWQQGNVFSCSVMHEALHNHYTQKSLSL |
| | SPGKGGGSGGGSGGGSGGGSGSTGEVQLVESGGGLVQPGGSLRLS |
| | CAASGFTLSSYSMHWVRQAPGKGLEWVAYISSYYGYTYYADSVKG |
| | RFTISADTSKNTAYLQMNSLRAEDTAVYYCARPAPGHWGFDYWGQ |
| | GTLVTVSSASTKGPSVFPLAPSSKSTSGGTAALGCLVKDYFPEPV |
| | TVSWNSGALTSGVHTFPAVLQSSGLYSLSSVVTVPSSSLGTQTYI |
| | CNVNHKPSNTKVDKKVEPKSCDKTHT |
| | (SEQ ID NO: 905) |
| Hole | MNLLLILTFVAAAVAEVQLVESGGGLVQPGGSLRLSCAASGENFS |
| Hc | SSSIHWVRQAPGKGLEWVASISSSYGYTYYADSVKGRFTISADTS |
| construct | KNTAYLQMNSLRAEDTAVYYCARSWAMDYWGQGTLVTVSSGGGGS |
| | DIQMTQSPSSLSASVGDRVTITCRASQSVSSAVAWYQQKPGKAPK |
| | LLIYSASSLYSGVPSRFSGSRSGTDFTLTISSLQPEDFATYYCQQ |
| | SSYSLITFGQGTKVEIKGGGGSGGGGSEPKSSDKTHTCPPCPAPE |
| | LLGGPSVFLFPPKPKDTLMISRTPEVTCVVVAVSHEDPEVKFNWY |
| | VDGVEVHNAKTKPREEQYGSTYRVVSVLTVLHQDWLNGKEYKCKV |
| | SNKALPAPIEKTISKAKGQPREPQVYTLPPIRELMTSNQVSLSCA |
| | VKGFYPSDIAVEWESNGQPENNYKTTPPVLDSDGSFFLVSKLTVD |
| | KSRWQQGNVFSCSVMHEALHNHYTQKSLSLSPGKGGGSGGGSGGG |
| | SGGGSGSTGEVQLVESGGGLVQPGGSLRLSCAASGFTLSSYSMHW |
| | VRQAPGKGLEWVAYISSYYGYTYYADSVKGRFTISADTSKNTAYL |
| | QMNSLRAEDTAVYYCARPAPGHWGFDYWGQGTLVTVSSASTKGPS |
| | VFPLAPSSKSTSGGTAALGCLVKDYFPEPVTVSWNSGALTSGVHT |
| | FPAVLQSSGLYSLSSVVTVPSSSLGTQTYICNVNHKPSNTKVDKK |
| | VEPKSCDKTHT |
| | (SEQ ID NO: 906) |
| Light Chain | MNLLLILTFVAAAVADIQMTQSPSSLSASVGDRVTITCRASQSVS |
| | SAVAWYQQKPGKAPKLLIYSASSLYSGVPSRFSGSRSGTDFTLTI |
| | SSLQPEDFATYYCQQWYYAPITFGQGTKVEIKRTVAAPSVFIFPP |
| | SDEQLKSGTASVVCLLNNFYPREAKVQWKVDNALQSGNSQESVTE |
| | QDSKDSTYSLSSTLTLSKADYEKHKVYACEVTHQGLSSPVTKSFN |
| | RGEC |
| | (SEQ ID NO: 902) |

FZD4 Agonist ANT39

| | |
|---|---|
| Knob Hc | MNLLLILTFVAAAVAEVQLVESGGGLVQPGGSLRLSCAASGEDFT |
| construct | AYAMHWVRQAPGKGLEWVASIYPSGGYTAYADSVKGRFTISADTS |
| | KNTAYLQMNSLRAEDTAVYYCARRSYYFALDYWGQGTLVTVSSGG |
| | GGSDIQMTQSPSSLSASVGDRVTITCRASQSVSSAVAWYQQKPGK |
| | APKLLIYSASSLYSGVPSRFSGSRSGTDFTLTISSLQPEDFATYY |
| | CQQYWAYYSPITFGQGTKVEIKGGGGSGGGGSEPKTSDKTHTCPP |
| | CPAPELLGGPSVFLFPPKPKDTLMISRTPEVTCVVVAVSHEDPEV |
| | KFNWYVDGVEVHNAKTKPREEQYGSTYRVVSVLTVLHQDWLNGKE |
| | YKCKVSNKALPAPIEKTISKAKGQPREPMVFDLPPSREEMTKNQV |
| | SLWCMVKGFYPSDIAVEWESNGQPENNYKTTPPVLDSDGSFFLYS |
| | KLTVDKSRWQQGNVFSCSVMHEALHNHYTQKSLSLSPGKGGGSGG |
| | GSGGGSGGGSGSTGEVQLVESGGGLVQPGGSLRLSCAASGFTLSS |
| | YSMHWVRQAPGKGLEWVAYISSYDSITDYADSVKGRFTISADTSK |
| | NTAYLQMNSLRAEDTAVYYCARPAVGHMAFDYWGQGTLVTVSSAS |
| | TKGPSVFPLAPSSKSTSGGTAALGCLVKDYFPEPVTVSWNSGALT |
| | SGVHTFPAVLQSSGLYSLSSVVTVPSSSLGTQTYICNVNHKPSNT |
| | KVDKKVEPKSCDKTHT |
| | (SEQ ID NO: 907) |
| Hole Hc | MNLLLILTFVAAAVAEVQLVESGGGLVQPGGSLRLSCAASGFDES |
| construct | SSSIHWVRQAPGKGLEWVASISSSYGYTYYADSVKGRFTISADTS |
| | KNTAYLQMNSLRAEDTAVYYCARSWAMDYWGQGTLVTVSSGGGGS |
| | DIQMTQSPSSLSASVGDRVTITCRASQSVSSAVAWYQQKPGKAPK |
| | LLIYSASDLYSGVPSRFSGSRSGTDFTLTISSLQPEDFATYYCQQ |
| | YAGAGLITFGQGTKVEIKGGGGSGGGGSEPKSSDKTHTCPPCPAP |
| | ELLGGPSVFLFPPKPKDTLMISRTPEVTCVVVAVSHEDPEVKFNW |
| | YVDGVEVHNAKTKPREEQYGSTYRVVSVLTVLHQDWLNGKEYKCK |
| | VSNKALPAPIEKTISKAKGQPREPQVYTLPPIRELMTSNQVSLSC |
| | AVKGFYPSDIAVEWESNGQPENNYKTTPPVLDSDGSFFLVSKLTV |
| | DKSRWQQGNVFSCSVMHEALHNHYTQKSLSLSPGKGGGSGGGSGG |
| | GSGGGSGSTGEVQLVESGGGLVQPGGSLRLSCAASGFTLSSYSMH |
| | WVRQAPGKGLEWVAYISSYDSITDYADSVKGRFTISADTSKNTAY |
| | LQMNSLRAEDTAVYYCARPAVGHMAFDYWGQGTLVTVSSASTKGP |
| | SVFPLAPSSKSTSGGTAALGCLVKDYFPEPVTVSWNSGALTSGVH |
| | TFPAVLQSSGLYSLSSVVTVPSSSLGTQTYICNVNHKPSNTKVDK |
| | KVEPKSCDKTHT |
| | (SEQ ID NO: 908) |
| Light Chain | MNLLLILTFVAAAVADIQMTQSPSSLSASVGDRVTITCRASQSVS |
| | SAVAWYQQKPGKAPKLLIYSASSLYSGVPSRFSGSRSGTDFTLTI |
| | SSLQPEDFATYYCQQWYNAPITFGQGTKVEIKRTVAAPSVFIFPP |
| | SDEQLKSGTASVVCLLNNFYPREAKVQWKVDNALQSGNSQESVTE |
| | QDSKDSTYSLSSTLTLSKADYEKHKVYACEVTHQGLSSPVTKSFN |
| | RGEC |
| | (SEQ ID NO: 909) |

FZD4 Agonist ANT42

| | |
|---|---|
| Knob | MNLLLILTFVAAAVAEVQLVESGGGLVQPGGSLRLSCAASGFDFT |
| Hc | AYAMHWVRQAPGKGLEWVASIYPSGGYTAYADSVKGRFTISADTS |
| construct | KNTAYLQMNSLRAEDTAVYYCARRSYYFALDYWGQGTLVTVSSGG |
| | GGSDIQMTQSPSSLSASVGDRVTITCRASQSVSSAVAWYQQKPGK |
| | APKLLIYSASSLYSGVPSRFSGSRSGTDFTLTISSLQPEDFATYY |
| | CQQYWAYYSPITFGQGTKVEIKGGGGSGGGGSEPKTSDKTHTCPP |
| | CPAPELLGGPSVFLFPPKPKDTLMISRTPEVTCVVVAVSHEDPEV |
| | KFNWYVDGVEVHNAKTKPREEQYGSTYRVVSVLTVLHQDWLNGKE |
| | YKCKVSNKALPAPIEKTISKAKGQPREPMVFDLPPSREEMTKNQV |
| | SLWCMVKGFYPSDIAVEWESNGQPENNYKTTPPVLDSDGSFFLYS |
| | KLTVDKSRWQQGNVFSCSVMHEALHNHYTQKSLSLSPGKGGGSGG |
| | GSGGGSGGGSGSTGEVQLVESGGGLVQPGGSLRLSCAASGFTSDF |
| | YFIHWVRQAPGKGLEWVATIYPFIGNTYYADSVKGRFTISADTSK |
| | NTAYLQMNSLRAEDTAVYYCARAFPGSYHPFDYWGQGTLVTVSSA |
| | STKGPSVFPLAPSSKSTSGGTAALGCLVKDYFPEPVTVSWNSGAL |
| | TSGVHTFPAVLQSSGLYSLSSVVTVPSSSLGTQTYICNVNHKPSN |
| | TKVDKKVEPKSCDKTHT |
| | (SEQ ID NO: 910) |
| Hole | MNLLLILTFVAAAVAEVQLVESGGGLVQPGGSLRLSCAASGFDES |
| Hc | SSSIHWVRQAPGKGLEWVASISSSYGYTYYADSVKGRFTISADTS |
| construct | KNTAYLQMNSLRAEDTAVYYCARSWAMDYWGQGTLVTVSSGGGGS |
| | DIQMTQSPSSLSASVGDRVTITCRASQSVSSAVAWYQQKPGKAPK |
| | LLIYSASDLYSGVPSRFSGSRSGTDFTLTISSLQPEDFATYYCQQ |
| | YAGAGLITFGQGTKVEIKGGGGSGGGGSEPKSSDKTHTCPPCPAP |
| | ELLGGPSVFLFPPKPKDTLMISRTPEVTCVVVAVSHEDPEVKFNW |
| | YVDGVEVHNAKTKPREEQYGSTYRVVSVLTVLHQDWLNGKEYKCK |
| | VSNKALPAPIEKTISKAKGQPREPQVYTLPPIRELMTSNQVSLSC |
| | AVKGFYPSDIAVEWESNGQPENNYKTTPPVLDSDGSFFLVSKLTV |
| | DKSRWQQGNVFSCSVMHEALHNHYTQKSLSLSPGKGGGSGGGSGG |
| | GSGGGSGSTGEVQLVESGGGLVQPGGSLRLSCAASGFTSDFYFIH |
| | WVRQAPGKGLEWVATIYPFIGNTYYADSVKGRFTISADTSKNTAY |
| | LQMNSLRAEDTAVYYCARAFPGSYHPFDYWGQGTLVTVSSASTKG |
| | PSVFPLAPSSKSTSGGTAALGCLVKDYFPEPVTVSWNSGALTSGV |
| | HTFPAVLQSSGLYSLSSVVTVPSSSLGTQTYICNVNHKPSNTKVD |
| | KKVEPKSCDKTHT |
| | (SEQ ID NO: 911) |
| Light Chain | MNLLLILTFVAAAVADIQMTQSPSSLSASVGDRVTITCRASQSVS |
| | SAVAWYQQKPGKAPKLLIYSASSLYSGVPSRFSGSRSGTDFTLTI |
| | SSLQPEDFATYYCQQSTGSLITFGQGTKVEIKRTVAAPSVFIFPP |
| | SDEQLKSGTASVVCLLNNFYPREAKVQWKVDNALQSGNSQESVTE |
| | QDSKDSTYSLSSTLTLSKADYEKHKVYACEVTHQGLSSPVTKSFN |
| | RGEC |
| | (SEQ ID NO: 912) |

FZD5-LRP6 ANT 59

| | |
|---|---|
| Knob | EVQLVESGGGLVQPGGSLRLSCAASGFDLSHSSIHWVRQAPGKGL |
| Hc | EWVASISSHYGYTHYADSVKGRFTISADTSKNTAYLQMNSLRAED |
| construct | TAVYYCARSSYFPWFFALDYWGQGTLVTVSSGGGGSDIQMTQSPS |
| | SLSASVGDRVTITCRASQSVSSAVAWYQQKPGKAPKLLIYSASSL |
| | YSGVPSRFSGSRSGTDFTLTISSLQPEDFATYYCQQYYWPITFGQ |
| | GTKVEIKGGGGSGGGGSEPKTSDKTHTCPPCPAPELLGGPSVFLF |
| | PPKPKDTLMISRTPEVTCVVVAVSHEDPEVKFNWYVDGVEVHNAK |
| | TKPREEQYGSTYRVVSVLTVLHQDWLNGKEYKCKVSNKALPAPIE |
| | KTISKAKGQPREPMVFDLPPSREEMTKNQVSLWCMVKGFYPSDIA |
| | VEWESNGQPENNYKTTPPVLDSDGSFFLYSKLTVDKSRWQQGNVF |
| | SCSVMHEALHNHYTQKSLSLSPGKGGGSGGGSGGGSGGGSGSTGE |
| | VQLVESGGGLVQPGGSLRLSCAASGENIAYSSMHWVRQAPGKGLE |
| | WVATIYPSYSSTYYADSVKGRFTISADTSKNTAYLQMNSLRAEDT |
| | AVYYCARYYAMDYWGQGTLVTVSSASTKGPSVFPLAPSSKSTSGG |
| | TAALGCLVKDYFPEPVTVSWNSGALTSGVHTFPAVLQSSGLYSLS |
| | SVVTVPSSSLGTQTYICNVNHKPSNTKVDKKVEPKSCDKTHT |
| | (SEQ ID NO: 913) |
| Hole | EVQLVESGGGLVQPGGSLRLSCAASGFDISSYFIHWVRQAPGKGL |
| Hc | EWVASIYPSYSYTSYADSVKGRFTISADTSKNTAYLQMNSLRAED |
| construct | TAVYYCARNVRGFRKPYFSRWAMDYWGQGTLVTVSSGGGGSDIQM |
| | TQSPSSLSASVGDRVTITCRASQSVSSAVAWYQQKPGKAPKLLIY |
| | SASSLYSGVPSRFSGSRSGTDFTLTISSLQPEDFATYYCQQYFFP |
| | ITFGQGTKVEIKGGGGSGGGGSEPKSSDKTHTCPPCPAPELLGGP |
| | SVFLFPPKPKDTLMISRTPEVTCVVVAVSHEDPEVKFNWYVDGVE |
| | VHNAKTKPREEQYGSTYRVVSVLTVLHQDWLNGKEYKCKVSNKAL |
| | PAPIEKTISKAKGQPREPQVYTLPPIRELMTSNQVSLSCAVKGFY |
| | PSDIAVEWESNGQPENNYKTTPPVLDSDGSFFLVSKLTVDKSRWQ |
| | QGNVFSCSVMHEALHNHYTQKSLSLSPGKGGGSGGGSGGGSGGGS |
| | GSTGEVQLVESGGGLVQPGGSLRLSCAASGENIAYSSMHWVRQAP |
| | GKGLEWVATIYPSYSSTYYADSVKGRFTISADTSKNTAYLQMNSL |
| | RAEDTAVYYCARYYAMDYWGQGTLVTVSSASTKGPSVFPLAPSSK |
| | STSGGTAALGCLVKDYFPEPVTVSWNSGALTSGVHTFPAVLQSSG |
| | LYSLSSVVTVPSSSLGTQTYICNVNHKPSNTKVDKKVEPKSCDKT |
| | HT |
| | (SEQ ID NO: 914) |
| Light | DIQMTQSPSSLSASVGDRVTITCRASQSVSSAVAWYQQKPGKAPK |
| Chain | LLIYSASSLYSGVPSRFSGSRSGTDFTLTISSLQPEDFATYYCQQ |
| | AHYFPITFGQGTKVEIKRTVAAPSVFIFPPSDEQLKSGTASVVCL |
| | LNNFYPREAKVQWKVDNALQSGNSQESVTEQDSKDSTYSLSSTLT |
| | LSKADYEKHKVYACEVTHQGLSSPVTKSFNRGEC |
| | (SEQ ID NO: 915) |

pFZD-LRP6 ANT9

| | |
|---|---|
| Knob | EVQLVESGGGLVQPGGSLRLSCAASGFDLSHSSIHWVRQAPGKGL |
| Hc | EWVASISSHYGYTHYADSVKGRFTISADTSKNTAYLQMNSLRAED |
| construct | TAVYYCARSSYFPWFFALDYWGQGTLVTVSSGGGGSDIQMTQSPS |
| | SLSASVGDRVTITCRASQSVSSAVAWYQQKPGKAPKLLIYSASSL |
| | YSGVPSRFSGSRSGTDFTLTISSLQPEDFATYYCQQYYWPITFGQ |
| | GTKVEIKGGGGSGGGGSEPKTSDKTHTCPPCPAPELLGGPSVFLF |
| | PPKPKDTLMISRTPEVTCVVVAVSHEDPEVKFNWYVDGVEVHNAK |
| | TKPREEQYGSTYRVVSVLTVLHQDWLNGKEYKCKVSNKALPAPIE |
| | KTISKAKGQPREPMVFDLPPSREEMTKNQVSLWCMVKGFYPSDIA |
| | VEWESNGQPENNYKTTPPVLDSDGSFFLYSKLTVDKSRWQQGNVF |
| | SCSVMHEALHNHYTQKSLSLSPGKGGGSGGGSGGGSGGGSGSTGE |
| | VQLVESGGGLVQPGGSLRLSCAASGFNIHSSSIHWVRQAPGKGLE |
| | WVAATYSSFGSITYADSVKGRFTISADTSKNTAYLQMNSLRAEDT |
| | AVYYCARYHHPFGYALDYWGQGTLVTVSSASTKGPSVFPLAPSSK |
| | STSGGTAALGCLVKDYFPEPVTVSWNSGALTSGVHTFPAVLQSSG |
| | LYSLSSVVTVPSSSLGTQTYICNVNHKPSNTKVDKKVEPKSCDKT |
| | HT |
| | (SEQ ID NO: 916) |
| Hole | EVQLVESGGGLVQPGGSLRLSCAASGFDISSYFIHWVRQAPGKGL |
| Hc | EWVASIYPSYSYTSYADSVKGRFTISADTSKNTAYLQMNSLRAED |
| construct | TAVYYCARNVRGFRKPYFSRWAMDYWGQGTLVTVSSGGGGSDIQM |
| | TQSPSSLSASVGDRVTITCRASQSVSSAVAWYQQKPGKAPKLLIY |
| | SASSLYSGVPSRFSGSRSGTDFTLTISSLQPEDFATYYCQQYFFP |
| | ITFGQGTKVEIKGGGGGGGGSEPKSSDKTHTCPPCPAPELLGGPS |
| | VFLFPPKPKDTLMISRTPEVTCVVVAVSHEDPEVKFNWYVDGVEV |
| | HNAKTKPREEQYGSTYRVVSVLTVLHQDWLNGKEYKCKVSNKALP |
| | APIEKTISKAKGQPREPQVYTLPPIRELMTSNQVSLSCAVKGFYP |
| | SDIAVEWESNGQPENNYKTTPPVLDSDGSFFLVSKLTVDKSRWQQ |
| | GNVFSCSVMHEALHNHYTQKSLSLSPGKGGGSGGGSGGGSGGGSG |
| | STGEVQLVESGGGLVQPGGSLRLSCAASGFNIHSSSIHWVRQAPG |
| | KGLEWVAATYSSFGSITYADSVKGRFTISADTSKNTAYLQMNSLR |
| | AEDTAVYYCARYHHPFGYALDYWGQGTLVTVSSASTKGPSVFPLA |
| | PSSKSTSGGTAALGCLVKDYFPEPVTVSWNSGALTSGVHTFPAVL |
| | QSSGLYSLSSVVTVPSSSLGTQTYICNVNHKPSNTKVDKKVEPKS |
| | CDKTHT |
| | (SEQ ID NO: 917) |
| Light | DIQMTQSPSSLSASVGDRVTITCRASQSVSSAVAWYQQKPGKAPK |
| Chain | LLIYSASSLYSGVPSRFSGSRSGTDFTLTISSLQPEDFATYYCQQ |
| | GVYLFTFGQGTKVEIKRTVAAPSVFIFPPSDEQLKSGTASVVCLL |
| | NNFYPREAKVQWKVDNALQSGNSQESVTEQDSKDSTYSLSSTLTL |
| | SKADYEKHKVYACEVTHQGLSSPVTKSFNRGEC |
| | (SEQ ID NO: 918) |

diabody-Fc-diabody FZD4 CM0199

| | |
|---|---|
| Knob | EFEVQLVESGGGLVQPGGSLRLSCAASGENSSFYFMHWVRQAPGK |
| Hc | GLEWVATVYPYLDYTYYADSVKGRFTISADTSKNTAYLQMNSLRA |
| construct | EDTAVYYCARAFPGSYHPMDYWGQGTLVTVSSGGGGSDIQMTQSP |
| | SSLSASVGDRVTITCRASQSVSSAVAWYQQKPGKAPKLLIYSASS |
| | LYSGVPSRFSGSRSGTDFTLTISSLQPEDFATYYCQQSSYSLITF |
| | GQGTKVEIKLEDKTHTKVEPKTSDKTHTCPPCPAPELLGGPSVFL |
| | FPPKPKDTLMISRTPEVTCVVVDVSHEDPEVKFNWYVDGVEVHNA |
| | KTKPREEQYNSTYRVVSVLTVLHQDWLNGKEYKCKVSNKALPAPI |
| | EKTISKAKGQPREPMVFDLPPSREEMTKNQVSLWCMVKGFYPSDI |
| | AVEWESNGQPENNYKTTPPVLDSDGSFFLYSKLTVDKSRWQQGNV |
| | FSCSVMHEALHNHYTQKSLSLSPGKSGSETPGTSESATPESGGGE |
| | VQLVESGGGLVQPGGSLRLSCAASGENFSSSSIHWVRQAPGKGLE |
| | WVASISSSYGYTYYADSVKGRFTISADTSKNTAYLQMNSLRAEDT |
| | AVYYCARGGSGVSHYGSVYYSWWALDYWGQGTLVTVSSGGGGSDI |
| | QMTQSPSSLSASVGDRVTITCRASQSVSSAVAWYQQKPGKAPKLL |
| | IYSASSLYSGVPSRFSGSRSGTDFTLTISSLQPEDFATYYCQQAS |
| | YAPITFGQGTKVEIK |
| | (SEQ ID NO: 919) |
| Hole | EFEVQLVESGGGLVQPGGSLRLSCAASGENSSFYFMHWVRQAPGK |
| Hc | GLEWVATVYPYLDYTYYADSVKGRFTISADTSKNTAYLQMNSLRA |
| construct | EDTAVYYCARAFPGSYHPMDYWGQGTLVTVSSGGGGSDIQMTQSP |
| | SSLSASVGDRVTITCRASQSVSSAVAWYQQKPGKAPKLLIYSASS |
| | LYSGVPSRFSGSRSGTDFTLTISSLQPEDFATYYCQQSSYSLITF |
| | GQGTKVEIKLEDKTHTKVEPKSSDKTHNCPPCPAPELLGGPSVFL |
| | FPPKPKDTLMISRTPEVTCVVVDVSHEDPEVKFNWYVDGVEVHNA |
| | KTKPREEQYNSTYRVVSVLTVLHQDWLNGKEYKCKVSNKALPAPI |
| | EKTISKAKGQPREPQVYTLPPIRELMTSNQVSLSCAVKGFYPSDI |
| | AVEWESNGQPENNYKTTPPVLDSDGSFFLVSKLTVDKSRWQQGNV |
| | FSCSVMHEALHNHYTQKSLSLSPGKSGSETPGTSESATPESGGGE |
| | VQLVESGGGLVQPGGSLRLSCAASGFNLSYYYMHWVRQAPGKGLE |
| | WVASIYSSYGYTYYADSVKGRFTISADTSKNTAYLQMNSLRAEDT |
| | AVYYCARWSHVSGHYSGMDYWGQGTLVTVSSGGGGSDIQMTQSPS |
| | SLSASVGDRVTITCRASQSVSSAVAWYQQKPGKAPKLLIYSASSL |
| | YSGVPSRFSGSRSGTDFTLTISSLQPEDFATYYCQQSSYSLITFG |
| | QGTKVEIK |
| | (SEQ ID NO: 920) |

TABLE 13
Diabody-Fc-Fab and IgG-Diabody amino acid sequences of the “heavy chain”
hole construct, “heavy chain” knob construct and the “light chain” construct
of the FZD4 Agonists, ANT39 and ANT42, ANT39 and ANT42, and ANT39i and ANT42i
having Fc domain amino acid mutations DANG, LALAPS, LALAPS and Merchant or
LALAPS and Merchant S-S. Mature sequences do not include the 5′ leader peptide.
The V-region CDRs of the chains are underlined, italicized and bolded, Fc null
mutations are italicized, CH3 heterodimerisation mutations are underlined and
italicized, CH3 cys disulphide bridges are bolded, and linkers are underlined.

ANT39i HC hole DANG
MNLLLILTFVAAAVAEVQLVESGGGLVQPGGSLRLSCAASGFTLSSYSMHWVRQAPGKGLEWVAYISSYDSITDYADSVKGRFTISAD
TSKNTAYLQMNSLRAEDTAVYYCARPAVGHMAFDYWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKKVEPKSCDKTHTCPPCPAPELLGGPSVFLFPPKP
KDTLMISRTPEVTCVVVAVSHEDPEVKFNWYVDGVEVHNAKTKPREEQYGSTYRVVSVLTVLHQDWLNGKEYKCKVSNKALPAPIEKT
ISKAKGQPREPQVYTLPPIRELMTSNQVSLSCAVKGFYPSDIAVEWESNGQPENNYKTTPPVLDSDGSFFLVSKLTVDKSRWQQGNVF
SCSVMHEALHNHYTQKSLSLSPGKGGGSGGGSGGGSGGGSGSTGEVQLVESGGGLVQPGGSLRLSCAASGFDFSSSSIHWVRQAPGKG
LEWVASISSSYGYTYYADSVKGRFTISADTSKNTAYLQMNSLRAEDTAVYYCARSWAMDYWGQGTLVTVSSGGGGSDIQMTQSPSSLS
ASVGDRVTITCRASQSVSSAVAWYQQKPGKAPKLLIYSASDLYSGVPSRFSGSRSGTDFTLTISSLQPEDFATYYCQQYAGAGLITFG
QGTKVEIK* (SEQ ID NO: 921)
ANT39i HC hole LALAPS
MNLLLILTFVAAAVAEVQLVESGGGLVQPGGSLRLSCAASGFTLSSYSMHWVRQAPGKGLEWVAYISSYDSITDYADSVKGRFTISAD
TSKNTAYLQMNSLRAEDTAVYYCARPAVGHMAFDYWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYNSTYRVVSVLTVLHQDWLNGKEYKCKVSNKALPASIEKT
ISKAKGQPREPQVYTLPPIRELMTSNQVSLSCAVKGFYPSDIAVEWESNGQPENNYKTTPPVLDSDGSFFLVSKLTVDKSRWQQGNVF
SCSVMHEALHNHYTQKSLSLSPGKGGGSGGGSGGGSGGGSGSTGEVQLVESGGGLVQPGGSLRLSCAASGFDFSSSSIHWVRQAPGKG
LEWVASISSSYGYTYYADSVKGRFTISADTSKNTAYLQMNSLRAEDTAVYYCARSWAMDYWGQGTLVTVSSGGGGSDIQMTQSPSSLS
ASVGDRVTITCRASQSVSSAVAWYQQKPGKAPKLLIYSASDLYSGVPSRFSGSRSGTDFTLTISSLQPEDFATYYCQQYAGAGLITFG
QGTKVEIK* (SEQ ID NO: 922)
ANT39i HC hole LALAPS Merchant
MNLLLILTFVAAAVAEVQLVESGGGLVQPGGSLRLSCAASGFTLSSYSMHWVRQAPGKGLEWVAYISSYDSITDYADSVKGRFTISAD
TSKNTAYLQMNSLRAEDTAVYYCARPAVGHMAFDYWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYNSTYRVVSVLTVLHQDWLNGKEYKCKVSNKALPASIEKT
ISKAKGQPREPQVYTLPPSREEMTKNQVSLSCAVKGFYPSDIAVEWESNGQPENNYKTTPPVLDSDGSFFLVSKLTVDKSRWQQGNVF
SCSVMHEALHNHYTQKSLSLSPGKGGGSGGGSGGGSGGGSGSTGEVQLVESGGGLVQPGGSLRLSCAASGFDFSSSSIHWVRQAPGKG
LEWVASISSSYGYTYYADSVKGRFTISADTSKNTAYLQMNSLRAEDTAVYYCARSWAMDYWGQGTLVTVSSGGGGSDIQMTQSPSSLS
ASVGDRVTITCRASQSVSSAVAWYQQKPGKAPKLLIYSASDLYSGVPSRFSGSRSGTDFTLTISSLQPEDFATYYCQQYAGAGLITFG
QGTKVEIK* (SEQ ID NO: 923)
ANT39i HC hole LALAPS Merchant S-S
MNLLLILTFVAAAVAEVQLVESGGGLVQPGGSLRLSCAASGFTLSSYSMHWVRQAPGKGLEWVAYISSYDSITDYADSVKGRFTISAD
TSKNTAYLQMNSLRAEDTAVYYCARPAVGHMAFDYWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYNSTYRVVSVLTVLHQDWLNGKEYKCKVSNKALPASIEKT
ISKAKGQPREPQVCTLPPSREEMTKNQVSLSCAVKGFYPSDIAVEWESNGQPENNYKTTPPVLDSDGSFFLVSKLTVDKSRWQQGNVF
SCSVMHEALHNHYTQKSLSLSPGKGGGSGGGSGGGSGGGSGSTGEVQLVESGGGLVQPGGSLRLSCAASGFDFSSSSIHWVRQAPGKG
LEWVASISSSYGYTYYADSVKGRFTISADTSKNTAYLQMNSLRAEDTAVYYCARSWAMDYWGQGTLVTVSSGGGGSDIQMTQSPSSLS
ASVGDRVTITCRASQSVSSAVAWYQQKPGKAPKLLIYSASDLYSGVPSRFSGSRSGTDFTLTISSLQPEDFATYYCQQYAGAGLITFG
QGTKVEIK* (SEQ ID NO: 924)
Mature ANT39i HC hole DANG
EVQLVESGGGLVQPGGSLRLSCAASGFTLSSYSMHWVRQAPGKGLEWVAYISSYDSITDYADSVKGRFTISADTSKNTAYLQMNSLRA
EDTAVYYCARPAVGHMAFDYWGQGTLVTVSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAPELLGGPSVFLFPPKPKDTLMISRTPEVTCV
VVAVSHEDPEVKFNWYVDGVEVHNAKTKPREEQYGSTYRVVSVLTVLHQDWLNGKEYKCKVSNKALPAPIEKTISKAKGQPREPQVYT
LPPIRELMTSNQVSLSCAVKGFYPSDIAVEWESNGQPENNYKTTPPVLDSDGSFFLVSKLTVDKSRWQQGNVFSCSVMHEALHNHYTQ
KSLSLSPGKGGGSGGGSGGGSGGGSGSTGEVQLVESGGGLVQPGGSLRLSCAASGFDFSSSSIHWVRQAPGKGLEWVASISSSYGYTY
YADSVKGRFTISADTSKNTAYLQMNSLRAEDTAVYYCARSWAMDYWGQGTLVTVSSGGGGSDIQMTQSPSSLSASVGDRVTITCRASQ
SVSSAVAWYQQKPGKAPKLLIYSASDLYSGVPSRFSGSRSGTDFTLTISSLQPEDFATYYCQQYAGAGLITFGQGTKVEIK* (SEQ
ID NO: 925)
Mature ANT39i HC hole LALAPS
EVQLVESGGGLVQPGGSLRLSCAASGFTLSSYSMHWVRQAPGKGLEWVAYISSYDSITDYADSVKGRFTISADTSKNTAYLQMNSLRA
EDTAVYYCARPAVGHMAFDYWGQGTLVTVSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAPEAAGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKALPASIEKTISKAKGQPREPQVYT
LPPIRELMTSNQVSLSCAVKGFYPSDIAVEWESNGQPENNYKTTPPVLDSDGSFFLVSKLTVDKSRWQQGNVFSCSVMHEALHNHYTQ
KSLSLSPGKGGGSGGGSGGGSGGGSGSTGEVQLVESGGGLVQPGGSLRLSCAASGFDFSSSSIHWVRQAPGKGLEWVASISSSYGYTY
YADSVKGRFTISADTSKNTAYLQMNSLRAEDTAVYYCARSWAMDYWGQGTLVTVSSGGGGSDIQMTQSPSSLSASVGDRVTITCRASQ
SVSSAVAWYQQKPGKAPKLLIYSASDLYSGVPSRFSGSRSGTDFTLTISSLQPEDFATYYCQQYAGAGLITFGQGTKVEIK* (SEQ
ID NO: 926)
Mature ANT39i HC hole LALAPS Merchant
EVQLVESGGGLVQPGGSLRLSCAASGFTLSSYSMHWVRQAPGKGLEWVAYISSYDSITDYADSVKGRFTISADTSKNTAYLQMNSLRA
EDTAVYYCARPAVGHMAFDYWGQGTLVTVSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAPEAAGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKALPASIEKTISKAKGQPREPQVYT
LPPSREEMTKNQVSLSCAVKGFYPSDIAVEWESNGQPENNYKTTPPVLDSDGSFFLVSKLTVDKSRWQQGNVFSCSVMHEALHNHYTQ
KSLSLSPGKGGGSGGGSGGGSGGGSGSTGEVQLVESGGGLVQPGGSLRLSCAASGFDFSSSSIHWVRQAPGKGLEWVASISSSYGYTY
YADSVKGRFTISADTSKNTAYLQMNSLRAEDTAVYYCARSWAMDYWGQGTLVTVSSGGGGSDIQMTQSPSSLSASVGDRVTITCRASQ
SVSSAVAWYQQKPGKAPKLLIYSASDLYSGVPSRFSGSRSGTDFTLTISSLQPEDFATYYCQQYAGAGLITFGQGTKVEIK* (SEQ
ID NO: 927)
Mature ANT39i HC hole LALAPS Merchant S-S
EVQLVESGGGLVQPGGSLRLSCAASGFTLSSYSMHWVRQAPGKGLEWVAYISSYDSITDYADSVKGRFTISADTSKNTAYLQMNSLRA
EDTAVYYCARPAVGHMAFDYWGQGTLVTVSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAPEAAGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKALPASIEKTISKAKGQPREPQVCT
LPPSREEMTKNQVSLSCAVKGFYPSDIAVEWESNGQPENNYKTTPPVLDSDGSFFLVSKLTVDKSRWQQGNVFSCSVMHEALHNHYTQ
KSLSLSPGKGGGSGGGSGGGSGGGSGSTGEVQLVESGGGLVQPGGSLRLSCAASGFDFSSSSIHWVRQAPGKGLEWVASISSSYGYTY
YADSVKGRFTISADTSKNTAYLQMNSLRAEDTAVYYCARSWAMDYWGQGTLVTVSSGGGGSDIQMTQSPSSLSASVGDRVTITCRASQ
SVSSAVAWYQQKPGKAPKLLIYSASDLYSGVPSRFSGSRSGTDFTLTISSLQPEDFATYYCQQYAGAGLITFGQGTKVEIK* (SEQ
ID NO: 928)
ANT39i HC knob DANG
MNLLLILTFVAAAVAEVQLVESGGGLVQPGGSLRLSCAASGFTLSSYSMHWVRQAPGKGLEWVAYISSYDSITDYADSVKGRFTISAD
TSKNTAYLQMNSLRAEDTAVYYCARPAVGHMAFDYWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKKVEPKSCDKTHTCPPCPAPELLGGPSVFLFPPKP
KDTLMISRTPEVTCVVVAVSHEDPEVKFNWYVDGVEVHNAKTKPREEQYGSTYRVVSVLTVLHQDWLNGKEYKCKVSNKALPAPIEKT
ISKAKGQPREPMVFDLPPSREEMTKNQVSLWCMVKGFYPSDIAVEWESNGQPENNYKTTPPVLDSDGSFFLYSKLTVDKSRWQQGNVF
SCSVMHEALHNHYTQKSLSLSPGKGGGSGGGSGGGSGGGSGSTGEVQLVESGGGLVQPGGSLRLSCAASGFDFTAYAMHWVRQAPGKG
LEWVASIYPSGGYTAYADSVKGRFTISADTSKNTAYLQMNSLRAEDTAVYYCARRSYYFALDYWGQGTLVTVSSGGGGSDIQMTQSPS
SLSASVGDRVTITCRASQSVSSAVAWYQQKPGKAPKLLIYSASSLYSGVPSRFSGSRSGTDFTLTISSLQPEDFATYYCQQYWAYYSP
ITFGQGTKVEIK* (SEQ ID NO: 929)
ANT39i HC Knob LALAPS
MNLLLILTFVAAAVAEVQLVESGGGLVQPGGSLRLSCAASGFTLSSYSMHWVRQAPGKGLEWVAYISSYDSITDYADSVKGRFTISAD
TSKNTAYLQMNSLRAEDTAVYYCARPAVGHMAFDYWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYNSTYRVVSVLTVLHQDWLNGKEYKCKVSNKALPASIEKT
ISKAKGQPREPMVFDLPPSREEMTKNQVSLWCMVKGFYPSDIAVEWESNGQPENNYKTTPPVLDSDGSFFLYSKLTVDKSRWQQGNVF
SCSVMHEALHNHYTQKSLSLSPGKGGGSGGGSGGGSGGGSGSTGEVQLVESGGGLVQPGGSLRLSCAASGEDFTAYAMHWVRQAPGKG
LEWVASIYPSGGYTAYADSVKGRFTISADTSKNTAYLQMNSLRAEDTAVYYCARRSYYFALDYWGQGTLVTVSSGGGGSDIQMTQSPS
SLSASVGDRVTITCRASQSVSSAVAWYQQKPGKAPKLLIYSASSLYSGVPSRFSGSRSGTDFTLTISSLQPEDFATYYCQQYWAYYSP
ITFGQGTKVEIK* (SEQ ID NO: 930)
ANT39i HC Knob LALAPS Merchant
MNLLLILTFVAAAVAEVQLVESGGGLVQPGGSLRLSCAASGFTLSSYSMHWVRQAPGKGLEWVAYISSYDSITDYADSVKGRFTISAD
TSKNTAYLQMNSLRAEDTAVYYCARPAVGHMAFDYWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYNSTYRVVSVLTVLHQDWLNGKEYKCKVSNKALPASIEKT
ISKAKGQPREPQVYTLPPSREEMTKNQVSLWCLVKGFYPSDIAVEWESNGQPENNYKTTPPVLDSDGSFFLYSKLTVDKSRWQQGNVF
SCSVMHEALHNHYTQKSLSLSPGKGGGSGGGSGGGSGGGSGSTGEVQLVESGGGLVQPGGSLRLSCAASGFDFTAYAMHWVRQAPGKG
LEWVASIYPSGGYTAYADSVKGRFTISADTSKNTAYLQMNSLRAEDTAVYYCARRSYYFALDYWGQGTLVTVSSGGGGSDIQMTQSPS
SLSASVGDRVTITCRASQSVSSAVAWYQQKPGKAPKLLIYSASSLYSGVPSRFSGSRSGTDFTLTISSLQPEDFATYYCQQYWAYYSP
ITFGQGTKVEIK* (SEQ ID NO: 931)
ANT39i HC Knob LALAPS Merchant S-S
MNLLLILTFVAAAVAEVQLVESGGGLVQPGGSLRLSCAASGFTLSSYSMHWVRQAPGKGLEWVAYISSYDSITDYADSVKGRFTISAD
TSKNTAYLQMNSLRAEDTAVYYCARPAVGHMAFDYWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYNSTYRVVSVLTVLHQDWLNGKEYKCKVSNKALPASIEKT
ISKAKGQPREPQVYTLPPCREEMTKNQVSLWCLVKGFYPSDIAVEWESNGQPENNYKTTPPVLDSDGSFFLYSKLTVDKSRWQQGNVF
SCSVMHEALHNHYTQKSLSLSPGKGGGSGGGSGGGSGGGSGSTGEVQLVESGGGLVQPGGSLRLSCAASGFDFTAYAMHWVRQAPGKG
LEWVASIYPSGGYTAYADSVKGRFTISADTSKNTAYLQMNSLRAEDTAVYYCARRSYYFALDYWGQGTLVTVSSGGGGSDIQMTQSPS
SLSASVGDRVTITCRASQSVSSAVAWYQQKPGKAPKLLIYSASSLYSGVPSRFSGSRSGTDFTLTISSLQPEDFATYYCQQYWAYYSP
ITFGQGTKVEIK* (SEQ ID NO: 932)
Mature ANT39i HC knob DANG
EVQLVESGGGLVQPGGSLRLSCAASGFTLSSYSMHWVRQAPGKGLEWVAYISSYDSITDYADSVKGRFTISADTSKNTAYLQMNSLRA
EDTAVYYCARPAVGHMAFDYWGQGTLVTVSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAPELLGGPSVFLFPPKPKDTLMISRTPEVTCV
VVAVSHEDPEVKFNWYVDGVEVHNAKTKPREEQYGSTYRVVSVLTVLHQDWLNGKEYKCKVSNKALPAPIEKTISKAKGQPREPMVFD
LPPSREEMTKNQVSLWCMVKGFYPSDIAVEWESNGQPENNYKTTPPVLDSDGSFFLYSKLTVDKSRWQQGNVFSCSVMHEALHNHYTQ
KSLSLSPGKGGGSGGGSGGGSGGGSGSTGEVQLVESGGGLVQPGGSLRLSCAASGFDFTAYAMHWVRQAPGKGLEWVASIYPSGGYTA
YADSVKGRFTISADTSKNTAYLQMNSLRAEDTAVYYCARRSYYFALDYWGQGTLVTVSSGGGGSDIQMTQSPSSLSASVGDRVTITCR
ASQSVSSAVAWYQQKPGKAPKLLIYSASSLYSGVPSRFSGSRSGTDFTLTISSLQPEDFATYYCQQYWAYYSPITFGQGTKVEIK*
(SEQ ID NO: 933)
Mature ANT39i HC Knob LALAPS
EVQLVESGGGLVQPGGSLRLSCAASGFTLSSYSMHWVRQAPGKGLEWVAYISSYDSITDYADSVKGRFTISADTSKNTAYLQMNSLRA
EDTAVYYCARPAVGHMAFDYWGQGTLVTVSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAPEAAGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKALPASIEKTISKAKGQPREPMVFD
LPPSREEMTKNQVSLWCMVKGFYPSDIAVEWESNGQPENNYKTTPPVLDSDGSFFLYSKLTVDKSRWQQGNVFSCSVMHEALHNHYTQ
KSLSLSPGKGGGSGGGSGGGSGGGSGSTGEVQLVESGGGLVQPGGSLRLSCAASGFDFTAYAMHWVRQAPGKGLEWVASIYPSGGYTA
YADSVKGRFTISADTSKNTAYLQMNSLRAEDTAVYYCARRSYYFALDYWGQGTLVTVSSGGGGSDIQMTQSPSSLSASVGDRVTITCR
ASQSVSSAVAWYQQKPGKAPKLLIYSASSLYSGVPSRFSGSRSGTDFTLTISSLQPEDFATYYCQQYWAYYSPITFGQGTKVEIK*
(SEQ ID NO: 934)
Mature ANT39i HC Knob LALAPS Merchant
EVQLVESGGGLVQPGGSLRLSCAASGFTLSSYSMHWVRQAPGKGLEWVAYISSYDSITDYADSVKGRFTISADTSKNTAYLQMNSLRA
EDTAVYYCARPAVGHMAFDYWGQGTLVTVSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAPEAAGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKALPASIEKTISKAKGQPREPQVYT
LPPSREEMTKNQVSLWCLVKGFYPSDIAVEWESNGQPENNYKTTPPVLDSDGSFFLYSKLTVDKSRWQQGNVFSCSVMHEALHNHYTQ
KSLSLSPGKGGGSGGGSGGGSGGGSGSTGEVQLVESGGGLVQPGGSLRLSCAASGFDFTAYAMHWVRQAPGKGLEWVASIYPSGGYTA
YADSVKGRFTISADTSKNTAYLQMNSLRAEDTAVYYCARRSYYFALDYWGQGTLVTVSSGGGGSDIQMTQSPSSLSASVGDRVTITCR
ASQSVSSAVAWYQQKPGKAPKLLIYSASSLYSGVPSRFSGSRSGTDFTLTISSLQPEDFATYYCQQYWAYYSPITFGQGTKVEIK*
(SEQ ID NO: 935)
Mature ANT39i HC Knob LALAPS Merchant S-S
EVQLVESGGGLVQPGGSLRLSCAASGFTLSSYSMHWVRQAPGKGLEWVAYISSYDSITDYADSVKGRFTISADTSKNTAYLQMNSLRA
EDTAVYYCARPAVGHMAFDYWGQGTLVTVSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAPEAAGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKALPASIEKTISKAKGQPREPQVYT
LPPCREEMTKNQVSLWCLVKGFYPSDIAVEWESNGQPENNYKTTPPVLDSDGSFFLYSKLTVDKSRWQQGNVFSCSVMHEALHNHYTQ
KSLSLSPGKGGGSGGGSGGGSGGGSGSTGEVQLVESGGGLVQPGGSLRLSCAASGFDFTAYAMHWVRQAPGKGLEWVASIYPSGGYTA
YADSVKGRFTISADTSKNTAYLQMNSLRAEDTAVYYCARRSYYFALDYWGQGTLVTVSSGGGGSDIQMTQSPSSLSASVGDRVTITCR
ASQSVSSAVAWYQQKPGKAPKLLIYSASSLYSGVPSRFSGSRSGTDFTLTISSLQPEDFATYYCQQYWAYYSPITFGQGTKVEIK*
(SEQ ID NO: 936)
ANT39 Hc Hole DANG
MNLLLILTFVAAAVAEVQLVESGGGLVQPGGSLRLSCAASGFDFSSSSIHWVRQAPGKGLEWVASISSSYGYTYYADSVKGRFTISAD
TSKNTAYLQMNSLRAEDTAVYYCARSWAMDYWGQGTLVTVSSGGGGSDIQMTQSPSSLSASVGDRVTITCRASQSVSSAVAWYQQKPG
KAPKLLIYSASDLYSGVPSRFSGSRSGTDFTLTISSLQPEDFATYYCQQYAGAGLITFGQGTKVEIKGGGGSGGGGSEPKSSDKTHTC
PPCPAPELLGGPSVFLFPPKPKDTLMISRTPEVTCVVVAVSHEDPEVKFNWYVDGVEVHNAKTKPREEQYGSTYRVVSVLTVLHQDWL
NGKEYKCKVSNKALPAPIEKTISKAKGQPREPQVYTLPPIRELMTSNQVSLSCAVKGFYPSDIAVEWESNGQPENNYKTTPPVLDSDG
SFFLVSKLTVDKSRWQQGNVFSCSVMHEALHNHYTQKSLSLSPGKGGGSGGGSGGGSGGGSGSTGEVQLVESGGGLVQPGGSLRLSCA
ASGFTLSSYSMHWVRQAPGKGLEWVAYISSYDSITDYADSVKGRFTISADTSKNTAYLQMNSLRAEDTAVYYCARPAVGHMAFDYWGQ
GTLVTVSSASTKGPSVFPLAPSSKSTSGGTAALGCLVKDYFPEPVTVSWNSGALTSGVHTFPAVLQSSGLYSLSSVVTVPSSSLGTQT
YICNVNHKPSNTKVDKKVEPKSCDKTHT* (SEQ ID NO: 908)
ANT39 Hc Hole LALAPS
MNLLLILTFVAAAVAEVQLVESGGGLVQPGGSLRLSCAASGFDFSSSSIHWVRQAPGKGLEWVASISSSYGYTYYADSVKGRFTISAD
TSKNTAYLQMNSLRAEDTAVYYCARSWAMDYWGQGTLVTVSSGGGGSDIQMTQSPSSLSASVGDRVTITCRASQSVSSAVAWYQQKPG
KAPKLLIYSASDLYSGVPSRFSGSRSGTDFTLTISSLQPEDFATYYCQQYAGAGLITFGQGTKVEIKGGGGSGGGGSEPKSSDKTHTC
PPCPAPEAAGGPSVFLFPPKPKDTLMISRTPEVTCVVVDVSHEDPEVKFNWYVDGVEVHNAKTKPREEQYNSTYRVVSVLTVLHQDWL
NGKEYKCKVSNKALPASIEKTISKAKGQPREPQVYTLPPIRELMTSNQVSLSCAVKGFYPSDIAVEWESNGQPENNYKTTPPVLDSDG
SFFLVSKLTVDKSRWQQGNVFSCSVMHEALHNHYTQKSLSLSPGKGGGSGGGSGGGSGGGSGSTGEVQLVESGGGLVQPGGSLRLSCA
ASGFTLSSYSMHWVRQAPGKGLEWVAYISSYDSITDYADSVKGRFTISADTSKNTAYLQMNSLRAEDTAVYYCARPAVGHMAFDYWGQ
GTLVTVSSASTKGPSVFPLAPSSKSTSGGTAALGCLVKDYFPEPVTVSWNSGALTSGVHTFPAVLQSSGLYSLSSVVTVPSSSLGTQT
YICNVNHKPSNTKVDKKVEPKSCDKTHT* (SEQ ID NO: 937)
ANT39 Hc Hole LALAPS Merchant
MNLLLILTFVAAAVAEVQLVESGGGLVQPGGSLRLSCAASGFDFSSSSIHWVRQAPGKGLEWVASISSSYGYTYYADSVKGRFTISAD
TSKNTAYLQMNSLRAEDTAVYYCARSWAMDYWGQGTLVTVSSGGGGSDIQMTQSPSSLSASVGDRVTITCRASQSVSSAVAWYQQKPG
KAPKLLIYSASDLYSGVPSRFSGSRSGTDFTLTISSLQPEDFATYYCQQYAGAGLITFGQGTKVEIKGGGGSGGGGSEPKSSDKTHTC
PPCPAPEAAGGPSVFLFPPKPKDTLMISRTPEVTCVVVDVSHEDPEVKFNWYVDGVEVHNAKTKPREEQYNSTYRVVSVLTVLHQDWL
NGKEYKCKVSNKALPASIEKTISKAKGQPREPQVYTLPPSREEMTKNQVSLSCAVKGFYPSDIAVEWESNGQPENNYKTTPPVLDSDG
SFFLVSKLTVDKSRWQQGNVFSCSVMHEALHNHYTQKSLSLSPGKGGGSGGGSGGGSGGGSGSTGEVQLVESGGGLVQPGGSLRLSCA
ASGFTLSSYSMHWVRQAPGKGLEWVAYISSYDSITDYADSVKGRFTISADTSKNTAYLQMNSLRAEDTAVYYCARPAVGHMAFDYWGQ
GTLVTVSSASTKGPSVFPLAPSSKSTSGGTAALGCLVKDYFPEPVTVSWNSGALTSGVHTFPAVLQSSGLYSLSSVVTVPSSSLGTQT
YICNVNHKPSNTKVDKKVEPKSCDKTHT* (SEQ ID NO: 938)
ANT39 Hc Hole LALAPS Merchant S-S
MNLLLILTFVAAAVAEVQLVESGGGLVQPGGSLRLSCAASGFDFSSSSIHWVRQAPGKGLEWVASISSSYGYTYYADSVKGRFTISAD
TSKNTAYLQMNSLRAEDTAVYYCARSWAMDYWGQGTLVTVSSGGGGSDIQMTQSPSSLSASVGDRVTITCRASQSVSSAVAWYQQKPG
KAPKLLIYSASDLYSGVPSRFSGSRSGTDFTLTISSLQPEDFATYYCQQYAGAGLITFGQGTKVEIKGGGGSGGGGSEPKSSDKTHTC
PPCPAPEAAGGPSVFLFPPKPKDTLMISRTPEVTCVVVDVSHEDPEVKFNWYVDGVEVHNAKTKPREEQYNSTYRVVSVLTVLHQDWL
NGKEYKCKVSNKALPASIEKTISKAKGQPREPQVCTLPPSREEMTKNQVSLSCAVKGFYPSDIAVEWESNGQPENNYKTTPPVLDSDG
SFFLVSKLTVDKSRWQQGNVFSCSVMHEALHNHYTQKSLSLSPGKGGGSGGGSGGGSGGGSGSTGEVQLVESGGGLVQPGGSLRLSCA
ASGFTLSSYSMHWVRQAPGKGLEWVAYISSYDSITDYADSVKGRFTISADTSKNTAYLQMNSLRAEDTAVYYCARPAVGHMAFDYWGQ
GTLVTVSSASTKGPSVFPLAPSSKSTSGGTAALGCLVKDYFPEPVTVSWNSGALTSGVHTFPAVLQSSGLYSLSSVVTVPSSSLGTQT
YICNVNHKPSNTKVDKKVEPKSCDKTHT* (SEQ ID NO: 939)
Mature ANT39 Hc Hole DANG
EVQLVESGGGLVQPGGSLRLSCAASGFDFSSSSIHWVRQAPGKGLEWVASISSSYGYTYYADSVKGRFTISADTSKNTAYLQMNSLRA
EDTAVYYCARSWAMDYWGQGTLVTVSSGGGGSDIQMTQSPSSLSASVGDRVTITCRASQSVSSAVAWYQQKPGKAPKLLIYSASDLYS
GVPSRFSGSRSGTDFTLTISSLQPEDFATYYCQQYAGAGLITFGQGTKVEIKGGGGSGGGGSEPKSSDKTHTCPPCPAPELLGGPSVF
LFPPKPKDTLMISRTPEVTCVVVAVSHEDPEVKFNWYVDGVEVHNAKTKPREEQYGSTYRVVSVLTVLHQDWLNGKEYKCKVSNKALP
APIEKTISKAKGQPREPQVYTLPPIRELMTSNQVSLSCAVKGFYPSDIAVEWESNGQPENNYKTTPPVLDSDGSFFLVSKLTVDKSRW
QQGNVFSCSVMHEALHNHYTQKSLSLSPGKGGGSGGGSGGGSGGGSGSTGEVQLVESGGGLVQPGGSLRLSCAASGFTLSSYSMHWVR
QAPGKGLEWVAYISSYDSITDYADSVKGRFTISADTSKNTAYLQMNSLRAEDTAVYYCARPAVGHMAFDYWGQGTLVTVSSASTKGPS
VFPLAPSSKSTSGGTAALGCLVKDYFPEPVTVSWNSGALTSGVHTFPAVLQSSGLYSLSSVVTVPSSSLGTQTYICNVNHKPSNTKVD
KKVEPKSCDKTHT* (SEQ ID NO: 940)
Mature ANT39 Hc Hole LALAPS
EVQLVESGGGLVQPGGSLRLSCAASGFDFSSSSIHWVRQAPGKGLEWVASISSSYGYTYYADSVKGRFTISADTSKNTAYLQMNSLRA
EDTAVYYCARSWAMDYWGQGTLVTVSSGGGGSDIQMTQSPSSLSASVGDRVTITCRASQSVSSAVAWYQQKPGKAPKLLIYSASDLYS
GVPSRFSGSRSGTDFTLTISSLQPEDFATYYCQQYAGAGLITFGQGTKVEIKGGGGSGGGGSEPKSSDKTHTCPPCPAPEAAGGPSVF
LFPPKPKDTLMISRTPEVTCVVVDVSHEDPEVKFNWYVDGVEVHNAKTKPREEQYNSTYRVVSVLTVLHQDWLNGKEYKCKVSNKALP
ASIEKTISKAKGQPREPQVYTLPPIRELMTSNQVSLSCAVKGFYPSDIAVEWESNGQPENNYKTTPPVLDSDGSFFLVSKLTVDKSRW
QQGNVFSCSVMHEALHNHYTQKSLSLSPGKGGGSGGGSGGGSGGGSGSTGEVQLVESGGGLVQPGGSLRLSCAASGFTLSSYSMHWVR
QAPGKGLEWVAYISSYDSITDYADSVKGRFTISADTSKNTAYLQMNSLRAEDTAVYYCARPAVGHMAFDYWGQGTLVTVSSASTKGPS
VFPLAPSSKSTSGGTAALGCLVKDYFPEPVTVSWNSGALTSGVHTFPAVLQSSGLYSLSSVVTVPSSSLGTQTYICNVNHKPSNTKVD
KKVEPKSCDKTHT* (SEQ ID NO: 941)
Mature ANT39 Hc Hole LALAPS Merchant
EVQLVESGGGLVQPGGSLRLSCAASGFDFSSSSIHWVRQAPGKGLEWVASISSSYGYTYYADSVKGRFTISADTSKNTAYLQMNSLRA
EDTAVYYCARSWAMDYWGQGTLVTVSSGGGGSDIQMTQSPSSLSASVGDRVTITCRASQSVSSAVAWYQQKPGKAPKLLIYSASDLYS
GVPSRFSGSRSGTDFTLTISSLQPEDFATYYCQQYAGAGLITFGQGTKVEIKGGGGSGGGGSEPKSSDKTHTCPPCPAPEAAGGPSVF
LFPPKPKDTLMISRTPEVTCVVVDVSHEDPEVKFNWYVDGVEVHNAKTKPREEQYNSTYRVVSVLTVLHQDWLNGKEYKCKVSNKALP
ASIEKTISKAKGQPREPQVYTLPPSREEMTKNQVSLSCAVKGFYPSDIAVEWESNGQPENNYKTTPPVLDSDGSFFLVSKLTVDKSRW
QQGNVFSCSVMHEALHNHYTQKSLSLSPGKGGGSGGGSGGGSGGGSGSTGEVQLVESGGGLVQPGGSLRLSCAASGFTLSSYSMHWVR
QAPGKGLEWVAYISSYDSITDYADSVKGRFTISADTSKNTAYLQMNSLRAEDTAVYYCARPAVGHMAFDYWGQGTLVTVSSASTKGPS
VFPLAPSSKSTSGGTAALGCLVKDYFPEPVTVSWNSGALTSGVHTFPAVLQSSGLYSLSSVVTVPSSSLGTQTYICNVNHKPSNTKVD
KKVEPKSCDKTHT* (SEQ ID NO: 942)
Mature ANT39 Hc Hole LALAPS Merchant S-S
EVQLVESGGGLVQPGGSLRLSCAASGFDFSSSSIHWVRQAPGKGLEWVASISSSYGYTYYADSVKGRFTISADTSKNTAYLQMNSLRA
EDTAVYYCARSWAMDYWGQGTLVTVSSGGGGSDIQMTQSPSSLSASVGDRVTITCRASQSVSSAVAWYQQKPGKAPKLLIYSASDLYS
GVPSRFSGSRSGTDFTLTISSLQPEDFATYYCQQYAGAGLITFGQGTKVEIKGGGGGGGGSEPKSSDKTHTCPPCPAPEAAGGPSVFL
FPPKPKDTLMISRTPEVTCVVVDVSHEDPEVKFNWYVDGVEVHNAKTKPREEQYNSTYRVVSVLTVLHQDWLNGKEYKCKVSNKALPA
SIEKTISKAKGQPREPQVCTLPPSREEMTKNQVSLSCAVKGFYPSDIAVEWESNGQPENNYKTTPPVLDSDGSFFLESKLTVDKSRWQ
QGNVFSCSVMHEALHNHYTQKSLSLSPGKGGGSGGGSGGGSGGGSGSTGEVQLVESGGGLVQPGGSLRLSCAASGFTLSSYSMHWVRQ
APGKGLEWVAYISSYDSITDYADSVKGRFTISADTSKNTAYLQMNSLRAEDTAVYYCARPAVGHMAFDYWGQGTLVTVSSASTKGPSV
FPLAPSSKSTSGGTAALGCLVKDYFPEPVTVSWNSGALTSGVHTFPAVLQSSGLYSLSSVVTVPSSSLGTQTYICNVNHKPSNTKVDK
KVEPKSCDKTHT* (SEQ ID NO: 943)
ANT39 Hc knob DANG
MNLLLILTFVAAAVAEVQLVESGGGLVQPGGSLRLSCAASGEDFTAYAMHWVRQAPGKGLEWVASIYPSGGYTAYADSVKGRFTISAD
TSKNTAYLQMNSLRAEDTAVYYCARRSYYFALDYWGQGTLVTVSSGGGGSDIQMTQSPSSLSASVGDRVTITCRASQSVSSAVAWYQQ
KPGKAPKLLIYSASSLYSGVPSRFSGSRSGTDFTLTISSLQPEDFATYYCQQYWAYYSPITFGQGTKVEIKGGGGSGGGGSEPKSSDK
THTCPPCPAPELLGGPSVFLFPPKPKDTLMISRTPEVTCVVVAVSHEDPEVKFNWYVDGVEVHNAKTKPREEQYGSTYRVVSVLTVLH
QDWLNGKEYKCKVSNKALPAPIEKTISKAKGQPREPMVFDLPPSREEMTKNQVSLWCMVKGFYPSDIAVEWESNGQPENNYKTTPPVL
DSDGSFFLYSKLTVDKSRWQQGNVFSCSVMHEALHNHYTQKSLSLSPGKGGGSGGGSGGGSGGGSGSTGEVQLVESGGGLVQPGGSLR
LSCAASGFTLSSYSMHWVRQAPGKGLEWVAYISSYDSITDYADSVKGRFTISADTSKNTAYLQMNSLRAEDTAVYYCARPAVGHMAFD
YWGQGTLVTVSSASTKGPSVFPLAPSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHT* (SEQ ID NO: 944)
ANT39 Hc knob LALAPS
MNLLLILTFVAAAVAEVQLVESGGGLVQPGGSLRLSCAASGFDFTAYAMHWVRQAPGKGLEWVASIYPSGGYTAYADSVKGRFTISAD
TSKNTAYLQMNSLRAEDTAVYYCARRSYYFALDYWGQGTLVTVSSGGGGSDIQMTQSPSSLSASVGDRVTITCRASQSVSSAVAWYQQ
KPGKAPKLLIYSASSLYSGVPSRFSGSRSGTDFTLTISSLQPEDFATYYCQQYWAYYSPITFGQGTKVEIKGGGGSGGGGSEPKSSDK
THTCPPCPAPEAAGGPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPASIEKTISKAKGQPREPMVFDLPPSREEMTKNQVSLWCMVKGFYPSDIAVEWESNGQPENNYKTTPPVL
DSDGSFFLYSKLTVDKSRWQQGNVFSCSVMHEALHNHYTQKSLSLSPGKGGGSGGGSGGGSGGGSGSTGEVQLVESGGGLVQPGGSLR
LSCAASGFTLSSYSMHWVRQAPGKGLEWVAYISSYDSITDYADSVKGRFTISADTSKNTAYLQMNSLRAEDTAVYYCARPAVGHMAFD
YWGQGTLVTVSSASTKGPSVFPLAPSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHT* (SEQ ID NO: 945)
ANT39 Hc knob LALAPS Merchant
MNLLLILTFVAAAVAEVQLVESGGGLVQPGGSLRLSCAASGEDFTAYAMHWVRQAPGKGLEWVASIYPSGGYTAYADSVKGRFTISAD
TSKNTAYLQMNSLRAEDTAVYYCARRSYYFALDYWGQGTLVTVSSGGGGSDIQMTQSPSSLSASVGDRVTITCRASQSVSSAVAWYQQ
KPGKAPKLLIYSASSLYSGVPSRFSGSRSGTDFTLTISSLQPEDFATYYCQQYWAYYSPITFGQGTKVEIKGGGGSGGGGSEPKSSDK
THTCPPCPAPEAAGGPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPASIEKTISKAKGQPREPQVYTLPPSREEMTKNQVSLWCLVKGFYPSDIAVEWESNGQPENNYKTTPPVL
DSDGSFFLYSKLTVDKSRWQQGNVFSCSVMHEALHNHYTQKSLSLSPGKGGGSGGGSGGGSGGGSGSTGEVQLVESGGGLVQPGGSLR
LSCAASGFTLSSYSMHWVRQAPGKGLEWVAYISSYDSITDYADSVKGRFTISADTSKNTAYLQMNSLRAEDTAVYYCARPAVGHMAFD
YWGQGTLVTVSSASTKGPSVFPLAPSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHT* (SEQ ID NO: 946)
ANT39 Hc knob LALAPS Merchant S-S
MNLLLILTFVAAAVAEVQLVESGGGLVQPGGSLRLSCAASGFDFTAYAMHWVRQAPGKGLEWVASIYPSGGYTAYADSVKGRFTISAD
TSKNTAYLQMNSLRAEDTAVYYCARRSYYFALDYWGQGTLVTVSSGGGGSDIQMTQSPSSLSASVGDRVTITCRASQSVSSAVAWYQQ
KPGKAPKLLIYSASSLYSGVPSRFSGSRSGTDFTLTISSLQPEDFATYYCQQYWAYYSPITFGQGTKVEIKGGGGSGGGGSEPKSSDK
THTCPPCPAPEAAGGPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPASIEKTISKAKGQPREPQVYTLPPCREEMTKNQVSLWCLVKGFYPSDIAVEWESNGQPENNYKTTPPVL
DSDGSFFLYSKLTVDKSRWQQGNVFSCSVMHEALHNHYTQKSLSLSPGKGGGSGGGSGGGSGGGSGSTGEVQLVESGGGLVQPGGSLR
LSCAASGFTLSSYSMHWVRQAPGKGLEWVAYISSYDSITDYADSVKGRFTISADTSKNTAYLQMNSLRAEDTAVYYCARPAVGHMAFD
YWGQGTLVTVSSASTKGPSVFPLAPSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHT* (SEQ ID NO: 947)
Mature ANT39 Hc knob DANG
EVQLVESGGGLVQPGGSLRLSCAASGFDFTAYAMHWVRQAPGKGLEWVASIYPSGGYTAYADSVKGRFTISADTSKNTAYLQMNSLRA
EDTAVYYCARRSYYFALDYWGQGTLVTVSSGGGGSDIQMTQSPSSLSASVGDRVTITCRASQSVSSAVAWYQQKPGKAPKLLIYSASS
LYSGVPSRFSGSRSGTDFTLTISSLQPEDFATYYCQQYWAYYSPITFGQGTKVEIKGGGGSGGGGSEPKSSDKTHTCPPCPAPELLGG
PSVFLFPPKPKDTLMISRTPEVTCVVVAVSHEDPEVKFNWYVDGVEVHNAKTKPREEQYGSTYRVVSVLTVLHQDWLNGKEYKCKVSN
KALPAPIEKTISKAKGQPREPMVFDLPPSREEMTKNQVSLWCMVKGFYPSDIAVEWESNGQPENNYKTTPPVLDSDGSFFLYSKLTVD
KSRWQQGNVFSCSVMHEALHNHYTQKSLSLSPGKGGGSGGGSGGGSGGGSGSTGEVQLVESGGGLVQPGGSLRLSCAASGFTLSSYSM
HWVRQAPGKGLEWVAYISSYDSITDYADSVKGRFTISADTSKNTAYLQMNSLRAEDTAVYYCARPAVGHMAFDYWGQGTLVTVSSAST
KGPSVFPLAPSSKSTSGGTAALGCLVKDYFPEPVTVSWNSGALTSGVHTFPAVLQSSGLYSLSSVVTVPSSSLGTQTYICNVNHKPSN
TKVDKKVEPKSCDKTHT* (SEQ ID NO: 948)
Mature ANT39 Hc knob LALAPS
EVQLVESGGGLVQPGGSLRLSCAASGEDFTAYAMHWVRQAPGKGLEWVASIYPSGGYTAYADSVKGRFTISADTSKNTAYLQMNSLRA
EDTAVYYCARRSYYFALDYWGQGTLVTVSSGGGGSDIQMTQSPSSLSASVGDRVTITCRASQSVSSAVAWYQQKPGKAPKLLIYSASS
LYSGVPSRFSGSRSGTDFTLTISSLQPEDFATYYCQQYWAYYSPITFGQGTKVEIKGGGGSGGGGSEPKSSDKTHTCPPCPAPEAAGG
PSVFLFPPKPKDTLMISRTPEVTCVVVDVSHEDPEVKFNWYVDGVEVHNAKTKPREEQYNSTYRVVSVLTVLHQDWLNGKEYKCKVSN
KALPASIEKTISKAKGQPREPMVFDLPPSREEMTKNQVSLWCMVKGFYPSDIAVEWESNGQPENNYKTTPPVLDSDGSFFLYSKLTVD
KSRWQQGNVFSCSVMHEALHNHYTQKSLSLSPGKGGGSGGGSGGGSGGGSGSTGEVQLVESGGGLVQPGGSLRLSCAASGFTLSSYSM
HWVRQAPGKGLEWVAYISSYDSITDYADSVKGRFTISADTSKNTAYLQMNSLRAEDTAVYYCARPAVGHMAFDYWGQGTLVTVSSAST
KGPSVFPLAPSSKSTSGGTAALGCLVKDYFPEPVTVSWNSGALTSGVHTFPAVLQSSGLYSLSSVVTVPSSSLGTQTYICNVNHKPSN
TKVDKKVEPKSCDKTHT* (SEQ ID NO: 949)
Mature ANT39 Hc knob LALAPS Merchant
EVQLVESGGGLVQPGGSLRLSCAASGFDFTAYAMHWVRQAPGKGLEWVASIYPSGGYTAYADSVKGRFTISADTSKNTAYLQMNSLRA
EDTAVYYCARRSYYFALDYWGQGTLVTVSSGGGGSDIQMTQSPSSLSASVGDRVTITCRASQSVSSAVAWYQQKPGKAPKLLIYSASS
LYSGVPSRFSGSRSGTDFTLTISSLQPEDFATYYCQQYWAYYSPITFGQGTKVEIKGGGGSGGGGSEPKSSDKTHTCPPCPAPEAAGG
PSVFLFPPKPKDTLMISRTPEVTCVVVDVSHEDPEVKFNWYVDGVEVHNAKTKPREEQYNSTYRVVSVLTVLHQDWLNGKEYKCKVSN
KALPASIEKTISKAKGQPREPQVYTLPPSREEMTKNQVSLWCLVKGFYPSDIAVEWESNGQPENNYKTTPPVLDSDGSFFLYSKLTVD
KSRWQQGNVFSCSVMHEALHNHYTQKSLSLSPGKGGGSGGGSGGGSGGGSGSTGEVQLVESGGGLVQPGGSLRLSCAASGFTLSSYSM
HWVRQAPGKGLEWVAYISSYDSITDYADSVKGRFTISADTSKNTAYLQMNSLRAEDTAVYYCARPAVGHMAFDYWGQGTLVTVSSAST
KGPSVFPLAPSSKSTSGGTAALGCLVKDYFPEPVTVSWNSGALTSGVHTFPAVLQSSGLYSLSSVVTVPSSSLGTQTYICNVNHKPSN
TKVDKKVEPKSCDKTHT* (SEQ ID NO: 950)
Mature ANT39 Hc knob LALAPS Merchant
EVQLVESGGGLVQPGGSLRLSCAASGFDFTAYAMHWVRQAPGKGLEWVASIYPSGGYTAYADSVKGRFTISADTSKNTAYLQMNSLRA
EDTAVYYCARRSYYFALDYWGQGTLVTVSSGGGGSDIQMTQSPSSLSASVGDRVTITCRASQSVSSAVAWYQQKPGKAPKLLIYSASS
LYSGVPSRFSGSRSGTDFTLTISSLQPEDFATYYCQQYWAYYSPITFGQGTKVEIKGGGGSGGGGSEPKSSDKTHTCPPCPAPEAAGG
PSVFLFPPKPKDTLMISRTPEVTCVVVDVSHEDPEVKFNWYVDGVEVHNAKTKPREEQYNSTYRVVSVLTVLHQDWLNGKEYKCKVSN
KALPASIEKTISKAKGQPREPQVYTLPPCREEMTKNQVSLWCLVKGFYPSDIAVEWESNGQPENNYKTTPPVLDSDGSFFLYSKLTVD
KSRWQQGNVFSCSVMHEALHNHYTQKSLSLSPGKGGGSGGGSGGGSGGGSGSTGEVQLVESGGGLVQPGGSLRLSCAASGFTLSSYSM
HWVRQAPGKGLEWVAYISSYDSITDYADSVKGRFTISADTSKNTAYLQMNSLRAEDTAVYYCARPAVGHMAFDYWGQGTLVTVSSAST
KGPSVFPLAPSSKSTSGGTAALGCLVKDYFPEPVTVSWNSGALTSGVHTFPAVLQSSGLYSLSSVVTVPSSSLGTQTYICNVNHKPSN
TKVDKKVEPKSCDKTHT* (SEQ ID NO: 951)
ANT39 LC
MNLLLILTFVAAAVADIQMTQSPSSLSASVGDRVTITCRASQSVSSAVAWYQQKPGKAPKLLIYSASSLYSGVPSRFSGSRSGTDFTL
TISSLQPEDFATYYCQQWYNAPITFGQGTKVEIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC* (SEQ ID NO: 909)
Mature ANT39 LC
DIQMTQSPSSLSASVGDRVTITCRASQSVSSAVAWYQQKPGKAPKLLIYSASSLYSGVPSRFSGSRSGTDFTLTISSLQPEDFATYYC
QQWYNAPITFGQGTKVEIKRTVAAPSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC (SEQ ID NO: 952)
ANT42i HC hole DANG
MNLLLILTFVAAAVAEVQLVESGGGLVQPGGSLRLSCAASGFTSDFYFIHWVRQAPGKGLEWVATIYPFIGNTYYADSVKGRFTISAD
TSKNTAYLQMNSLRAEDTAVYYCARAFPGSYHPFDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVEPKSCDKTHTCPPCPAPELLGGPSVFLFPPK
PKDTLMISRTPEVTCVVVAVSHEDPEVKFNWYVDGVEVHNAKTKPREEQYGSTYRVVSVLTVLHQDWLNGKEYKCKVSNKALPAPIEK
TISKAKGQPREPQVYTLPPIRELMTSNQVSLSCAVKGFYPSDIAVEWESNGQPENNYKTTPPVLDSDGSFFLVSKLTVDKSRWQQGNV
FSCSVMHEALHNHYTQKSLSLSPGKGGGSGGGSGGGSGGGSGSTGEVQLVESGGGLVQPGGSLRLSCAASGFDFSSSSIHWVRQAPGK
GLEWVASISSSYGYTYYADSVKGRFTISADTSKNTAYLQMNSLRAEDTAVYYCARSWAMDYWGQGTLVTVSSGGGGSDIQMTQSPSSL
SASVGDRVTITCRASQSVSSAVAWYQQKPGKAPKLLIYSASDLYSGVPSRFSGSRSGTDFTLTISSLQPEDFATYYCQQYAGAGLITF
GQGTKVEIK* (SEQ ID NO: 953)
ANT42i HC hole LALAPS
MNLLLILTFVAAAVAEVQLVESGGGLVQPGGSLRLSCAASGFTSDFYFIHWVRQAPGKGLEWVATIYPFIGNTYYADSVKGRFTISAD
TSKNTAYLQMNSLRAEDTAVYYCARAFPGSYHPFDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVEPKSCDKTHTCPPCPAPEAAGGPSVFLFPPK
PKDTLMISRTPEVTCVVVDVSHEDPEVKFNWYVDGVEVHNAKTKPREEQYNSTYRVVSVLTVLHQDWLNGKEYKCKVSNKALPASIEK
TISKAKGQPREPQVYTLPPIRELMTSNQVSLSCAVKGFYPSDIAVEWESNGQPENNYKTTPPVLDSDGSFFLVSKLTVDKSRWQQGNV
FSCSVMHEALHNHYTQKSLSLSPGKGGGSGGGSGGGSGGGSGSTGEVQLVESGGGLVQPGGSLRLSCAASGFDFSSSSIHWVRQAPGK
GLEWVASISSSYGYTYYADSVKGRFTISADTSKNTAYLQMNSLRAEDTAVYYCARSWAMDYWGQGTLVTVSSGGGGSDIQMTQSPSSL
SASVGDRVTITCRASQSVSSAVAWYQQKPGKAPKLLIYSASDLYSGVPSRFSGSRSGTDFTLTISSLQPEDFATYYCQQYAGAGLITF
GQGTKVEIK* (SEQ ID NO: 954)
ANT42i HC hole LALAPS Merchant
MNLLLILTFVAAAVAEVQLVESGGGLVQPGGSLRLSCAASGFTSDFYFIHWVRQAPGKGLEWVATIYPFIGNTYYADSVKGRFTISAD
TSKNTAYLQMNSLRAEDTAVYYCARAFPGSYHPFDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVEPKSCDKTHTCPPCPAPEAAGGPSVFLFPPK
PKDTLMISRTPEVTCVVVDVSHEDPEVKFNWYVDGVEVHNAKTKPREEQYNSTYRVVSVLTVLHQDWLNGKEYKCKVSNKALPASIEK
TISKAKGQPREPQVYTLPPSREEMTKNQVSLSCAVKGFYPSDIAVEWESNGQPENNYKTTPPVLDSDGSFFLVSKLTVDKSRWQQGNV
FSCSVMHEALHNHYTQKSLSLSPGKGGGSGGGSGGGSGGGSGSTGEVQLVESGGGLVQPGGSLRLSCAASGFDFSSSSIHWVRQAPGK
GLEWVASISSSYGYTYYADSVKGRFTISADTSKNTAYLQMNSLRAEDTAVYYCARSWAMDYWGQGTLVTVSSGGGGSDIQMTQSPSSL
SASVGDRVTITCRASQSVSSAVAWYQQKPGKAPKLLIYSASDLYSGVPSRFSGSRSGTDFTLTISSLQPEDFATYYCQQYAGAGLITF
GQGTKVEIK* (SEQ ID NO: 955)
ANT42i HC hole LALAPS Merchant S-S
MNLLLILTFVAAAVAEVQLVESGGGLVQPGGSLRLSCAASGFTSDFYFIHWVRQAPGKGLEWVATIYPFIGNTYYADSVKGRFTISAD
TSKNTAYLQMNSLRAEDTAVYYCARAFPGSYHPFDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVEPKSCDKTHTCPPCPAPEAAGGPSVFLFPPK
PKDTLMISRTPEVTCVVVDVSHEDPEVKFNWYVDGVEVHNAKTKPREEQYNSTYRVVSVLTVLHQDWLNGKEYKCKVSNKALPASIEK
TISKAKGQPREPQVCTLPPSREEMTKNQVSLSCAVKGFYPSDIAVEWESNGQPENNYKTTPPVLDSDGSFFLVSKLTVDKSRWQQGNV
FSCSVMHEALHNHYTQKSLSLSPGKGGGSGGGSGGGSGGGSGSTGEVQLVESGGGLVQPGGSLRLSCAASGFDFSSSSIHWVRQAPGK
GLEWVASISSSYGYTYYADSVKGRFTISADTSKNTAYLQMNSLRAEDTAVYYCARSWAMDYWGQGTLVTVSSGGGGSDIQMTQSPSSL
SASVGDRVTITCRASQSVSSAVAWYQQKPGKAPKLLIYSASDLYSGVPSRFSGSRSGTDFTLTISSLQPEDFATYYCQQYAGAGLITF
GQGTKVEIK* (SEQ ID NO: 956)
Mature ANT42i HC hole DANG
EVQLVESGGGLVQPGGSLRLSCAASGFTSDFYFIHWVRQAPGKGLEWVATIYPFIGNTYYADSVKGRFTISADTSKNTAYLQMNSLRA
EDTAVYYCARAFPGSYHPFDYWGQGTLVTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPELLGGPSVFLFPPKPKDTLMISRTPEVTC
VVVAVSHEDPEVKFNWYVDGVEVHNAKTKPREEQYGSTYRVVSVLTVLHQDWLNGKEYKCKVSNKALPAPIEKTISKAKGQPREPQVY
TLPPIRELMTSNQVSLSCAVKGFYPSDIAVEWESNGQPENNYKTTPPVLDSDGSFFLVSKLTVDKSRWQQGNVFSCSVMHEALHNHYT
QKSLSLSPGKGGGSGGGSGGGSGGGSGSTGEVQLVESGGGLVQPGGSLRLSCAASGFDFSSSSIHWVRQAPGKGLEWVASISSSYGYT
YYADSVKGRFTISADTSKNTAYLQMNSLRAEDTAVYYCARSWAMDYWGQGTLVTVSSGGGGSDIQMTQSPSSLSASVGDRVTITCRAS
QSVSSAVAWYQQKPGKAPKLLIYSASDLYSGVPSRFSGSRSGTDFTLTISSLQPEDFATYYCQQYAGAGLITFGQGTKVEIK* (SEQ
ID NO: 957)
Mature ANT42i HC hole LALAPS
EVQLVESGGGLVQPGGSLRLSCAASGFTSDFYFIHWVRQAPGKGLEWVATIYPFIGNTYYADSVKGRFTISADTSKNTAYLQMNSLRA
EDTAVYYCARAFPGSYHPFDYWGQGTLVTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPEAAGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPASIEKTISKAKGQPREPQVY
TLPPIRELMTSNQVSLSCAVKGFYPSDIAVEWESNGQPENNYKTTPPVLDSDGSFFLVSKLTVDKSRWQQGNVFSCSVMHEALHNHYT
QKSLSLSPGKGGGSGGGSGGGSGGGSGSTGEVQLVESGGGLVQPGGSLRLSCAASGFDFSSSSIHWVRQAPGKGLEWVASISSSYGYT
YYADSVKGRFTISADTSKNTAYLQMNSLRAEDTAVYYCARSWAMDYWGQGTLVTVSSGGGGSDIQMTQSPSSLSASVGDRVTITCRAS
QSVSSAVAWYQQKPGKAPKLLIYSASDLYSGVPSRFSGSRSGTDFTLTISSLQPEDFATYYCQQYAGAGLITFGQGTKVEIK* (SEQ
ID NO: 958)
Mature ANT42i HC hole LALAPS Merchant
EVQLVESGGGLVQPGGSLRLSCAASGFTSDFYFIHWVRQAPGKGLEWVATIYPFIGNTYYADSVKGRFTISADTSKNTAYLQMNSLRA
EDTAVYYCARAFPGSYHPFDYWGQGTLVTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPEAAGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPASIEKTISKAKGQPREPQVY
TLPPSREEMTKNQVSLSCAVKGFYPSDIAVEWESNGQPENNYKTTPPVLDSDGSFFLVSKLTVDKSRWQQGNVFSCSVMHEALHNHYT
QKSLSLSPGKGGGSGGGSGGGSGGGSGSTGEVQLVESGGGLVQPGGSLRLSCAASGFDFSSSSIHWVRQAPGKGLEWVASISSSYGYT
YYADSVKGRFTISADTSKNTAYLQMNSLRAEDTAVYYCARSWAMDYWGQGTLVTVSSGGGGSDIQMTQSPSSLSASVGDRVTITCRAS
QSVSSAVAWYQQKPGKAPKLLIYSASDLYSGVPSRFSGSRSGTDFTLTISSLQPEDFATYYCQQYAGAGLITFGQGTKVEIK* (SEQ
ID NO: 959)
Mature ANT42i HC hole LALAPS Merchant S-S
EVQLVESGGGLVQPGGSLRLSCAASGFTSDFYFIHWVRQAPGKGLEWVATIYPFIGNTYYADSVKGRFTISADTSKNTAYLQMNSLRA
EDTAVYYCARAFPGSYHPFDYWGQGTLVTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPEAAGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPASIEKTISKAKGQPREPQVC
TLPPSREEMTKNQVSLSCAVKGFYPSDIAVEWESNGQPENNYKTTPPVLDSDGSFFLVSKLTVDKSRWQQGNVFSCSVMHEALHNHYT
QKSLSLSPGKGGGSGGGSGGGSGGGSGSTGEVQLVESGGGLVQPGGSLRLSCAASGFDFSSSSIHWVRQAPGKGLEWVASISSSYGYT
YYADSVKGRFTISADTSKNTAYLQMNSLRAEDTAVYYCARSWAMDYWGQGTLVTVSSGGGGSDIQMTQSPSSLSASVGDRVTITCRAS
QSVSSAVAWYQQKPGKAPKLLIYSASDLYSGVPSRFSGSRSGTDFTLTISSLQPEDFATYYCQQYAGAGLITFGQGTKVEIK* (SEQ
ID NO: 960)
ANT42i HC knob DANG
MNLLLILTFVAAAVAEVQLVESGGGLVQPGGSLRLSCAASGFTSDFYFIHWVRQAPGKGLEWVATIYPFIGNTYYADSVKGRFTISAD
TSKNTAYLQMNSLRAEDTAVYYCARAFPGSYHPFDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVEPKSCDKTHTCPPCPAPELLGGPSVFLFPPK
PKDTLMISRTPEVTCVVVAVSHEDPEVKFNWYVDGVEVHNAKTKPREEQYGSTYRVVSVLTVLHQDWLNGKEYKCKVSNKALPAPIEK
TISKAKGQPREPMVFDLPPSREEMTKNQVSLWCMVKGFYPSDIAVEWESNGQPENNYKTTPPVLDSDGSFFLYSKLTVDKSRWQQGNV
FSCSVMHEALHNHYTQKSLSLSPGKGGGSGGGSGGGSGGGSGSTGEVQLVESGGGLVQPGGSLRLSCAASGEDFTAYAMHWVRQAPGK
GLEWVASIYPSGGYTAYADSVKGRFTISADTSKNTAYLQMNSLRAEDTAVYYCARRSYYFALDYWGQGTLVTVSSGGGGSDIQMTQSP
SSLSASVGDRVTITCRASQSVSSAVAWYQQKPGKAPKLLIYSASSLYSGVPSRFSGSRSGTDFTLTISSLQPEDFATYYCQQYWAYYS
PITFGQGTKVEIK* (SEQ ID NO: 961)
ANT42i HC Knob LALAPS
MNLLLILTFVAAAVAEVQLVESGGGLVQPGGSLRLSCAASGFTSDFYFIHWVRQAPGKGLEWVATIYPFIGNTYYADSVKGRFTISAD
TSKNTAYLQMNSLRAEDTAVYYCARAFPGSYHPFDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVEPKSCDKTHTCPPCPAPEAAGGPSVFLFPPK
PKDTLMISRTPEVTCVVVDVSHEDPEVKFNWYVDGVEVHNAKTKPREEQYNSTYRVVSVLTVLHQDWLNGKEYKCKVSNKALPASIEK
TISKAKGQPREPMVFDLPPSREEMTKNQVSLWCMVKGFYPSDIAVEWESNGQPENNYKTTPPVLDSDGSFFLYSKLTVDKSRWQQGNV
FSCSVMHEALHNHYTQKSLSLSPGKGGGSGGGSGGGSGGGSGSTGEVQLVESGGGLVQPGGSLRLSCAASGFDFTAYAMHWVRQAPGK
GLEWVASIYPSGGYTAYADSVKGRFTISADTSKNTAYLQMNSLRAEDTAVYYCARRSYYFALDYWGQGTLVTVSSGGGGSDIQMTQSP
SSLSASVGDRVTITCRASQSVSSAVAWYQQKPGKAPKLLIYSASSLYSGVPSRFSGSRSGTDFTLTISSLQPEDFATYYCQQYWAYYS
PITFGQGTKVEIK* (SEQ ID NO: 962)
ANT42i HC Knob LALAPS Merchant
MNLLLILTFVAAAVAEVQLVESGGGLVQPGGSLRLSCAASGFTSDFYFIHWVRQAPGKGLEWVATIYPFIGNTYYADSVKGRFTISAD
TSKNTAYLQMNSLRAEDTAVYYCARAFPGSYHPFDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVEPKSCDKTHTCPPCPAPEAAGGPSVFLFPPK
PKDTLMISRTPEVTCVVVDVSHEDPEVKFNWYVDGVEVHNAKTKPREEQYNSTYRVVSVLTVLHQDWLNGKEYKCKVSNKALPASIEK
TISKAKGQPREPQVYTLPPSREEMTKNQVSLWCLVKGFYPSDIAVEWESNGQPENNYKTTPPVLDSDGSFFLYSKLTVDKSRWQQGNV
FSCSVMHEALHNHYTQKSLSLSPGKGGGSGGGSGGGSGGGSGSTGEVQLVESGGGLVQPGGSLRLSCAASGFDFTAYAMHWVRQAPGK
GLEWVASIYPSGGYTAYADSVKGRFTISADTSKNTAYLQMNSLRAEDTAVYYCARRSYYFALDYWGQGTLVTVSSGGGGSDIQMTQSP
SSLSASVGDRVTITCRASQSVSSAVAWYQQKPGKAPKLLIYSASSLYSGVPSRFSGSRSGTDFTLTISSLQPEDFATYYCQQYWAYYS
PITFGQGTKVEIK* (SEQ ID NO: 963)
ANT42i HC Knob LALAPS Merchant S-S
MNLLLILTFVAAAVAEVQLVESGGGLVQPGGSLRLSCAASGFTSDFYFIHWVRQAPGKGLEWVATIYPFIGNTYYADSVKGRFTISAD
TSKNTAYLQMNSLRAEDTAVYYCARAFPGSYHPFDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVEPKSCDKTHTCPPCPAPEAAGGPSVFLFPPK
PKDTLMISRTPEVTCVVVDVSHEDPEVKFNWYVDGVEVHNAKTKPREEQYNSTYRVVSVLTVLHQDWLNGKEYKCKVSNKALPASIEK
TISKAKGQPREPQVYTLPPCREEMTKNQVSLWCLVKGFYPSDIAVEWESNGQPENNYKTTPPVLDSDGSFFLYSKLTVDKSRWQQGNV
FSCSVMHEALHNHYTQKSLSLSPGKGGGSGGGSGGGSGGGSGSTGEVQLVESGGGLVQPGGSLRLSCAASGFDFTAYAMHWVRQAPGK
GLEWVASIYPSGGYTAYADSVKGRFTISADTSKNTAYLQMNSLRAEDTAVYYCARRSYYFALDYWGQGTLVTVSSGGGGSDIQMTQSP
SSLSASVGDRVTITCRASQSVSSAVAWYQQKPGKAPKLLIYSASSLYSGVPSRFSGSRSGTDFTLTISSLQPEDFATYYCQQYWAYYS
PITFGQGTKVEIK* (SEQ ID NO: 964)
Mature ANT42i HC knob DANG
EVQLVESGGGLVQPGGSLRLSCAASGFTSDFYFIHWVRQAPGKGLEWVATIYPFIGNTYYADSVKGRFTISADTSKNTAYLQMNSLRA
EDTAVYYCARAFPGSYHPFDYWGQGTLVTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPELLGGPSVFLFPPKPKDTLMISRTPEVTC
VVVAVSHEDPEVKFNWYVDGVEVHNAKTKPREEQYGSTYRVVSVLTVLHQDWLNGKEYKCKVSNKALPAPIEKTISKAKGQPREPMVF
DLPPSREEMTKNQVSLWCMVKGFYPSDIAVEWESNGQPENNYKTTPPVLDSDGSFFLYSKLTVDKSRWQQGNVFSCSVMHEALHNHYT
QKSLSLSPGKGGGSGGGSGGGSGGGSGSTGEVQLVESGGGLVQPGGSLRLSCAASGFDFTAYAMHWVRQAPGKGLEWVASIYPSGGYT
AYADSVKGRFTISADTSKNTAYLQMNSLRAEDTAVYYCARRSYYFALDYWGQGTLVTVSSGGGGSDIQMTQSPSSLSASVGDRVTITC
RASQSVSSAVAWYQQKPGKAPKLLIYSASSLYSGVPSRFSGSRSGTDFTLTISSLQPEDFATYYCQQYWAYYSPITFGQGTKVEIK*
(SEQ ID NO: 965)
Mature ANT42i HC Knob LALAPS
EVQLVESGGGLVQPGGSLRLSCAASGFTSDFYFIHWVRQAPGKGLEWVATIYPFIGNTYYADSVKGRFTISADTSKNTAYLQMNSLRA
EDTAVYYCARAFPGSYHPFDYWGQGTLVTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPEAAGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPASIEKTISKAKGQPREPMVF
DLPPSREEMTKNQVSLWCMVKGFYPSDIAVEWESNGQPENNYKTTPPVLDSDGSFFLYSKLTVDKSRWQQGNVFSCSVMHEALHNHYT
QKSLSLSPGKGGGSGGGSGGGSGGGSGSTGEVQLVESGGGLVQPGGSLRLSCAASGEDFTAYAMHWVRQAPGKGLEWVASIYPSGGYT
AYADSVKGRFTISADTSKNTAYLQMNSLRAEDTAVYYCARRSYYFALDYWGQGTLVTVSSGGGGSDIQMTQSPSSLSASVGDRVTITC
RASQSVSSAVAWYQQKPGKAPKLLIYSASSLYSGVPSRFSGSRSGTDFTLTISSLQPEDFATYYCQQYWAYYSPITFGQGTKVEIK*
(SEQ ID NO: 966)
Mature ANT42i HC Knob LALAPS Merchant
EVQLVESGGGLVQPGGSLRLSCAASGFTSDFYFIHWVRQAPGKGLEWVATIYPFIGNTYYADSVKGRFTISADTSKNTAYLQMNSLRA
EDTAVYYCARAFPGSYHPFDYWGQGTLVTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPEAAGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPASIEKTISKAKGQPREPQVY
TLPPSREEMTKNQVSLWCLVKGFYPSDIAVEWESNGQPENNYKTTPPVLDSDGSFFLYSKLTVDKSRWQQGNVFSCSVMHEALHNHYT
QKSLSLSPGKGGGSGGGSGGGSGGGSGSTGEVQLVESGGGLVQPGGSLRLSCAASGFDFTAYAMHWVRQAPGKGLEWVASIYPSGGYT
AYADSVKGRFTISADTSKNTAYLQMNSLRAEDTAVYYCARRSYYFALDYWGQGTLVTVSSGGGGSDIQMTQSPSSLSASVGDRVTITC
RASQSVSSAVAWYQQKPGKAPKLLIYSASSLYSGVPSRFSGSRSGTDFTLTISSLQPEDFATYYCQQYWAYYSPITFGQGTKVEIK*
(SEQ ID NO: 967)
Mature ANT42i HC Knob LALAPS Merchant S-S
EVQLVESGGGLVQPGGSLRLSCAASGFTSDFYFIHWVRQAPGKGLEWVATIYPFIGNTYYADSVKGRFTISADTSKNTAYLQMNSLRA
EDTAVYYCARAFPGSYHPFDYWGQGTLVTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPEAAGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPASIEKTISKAKGQPREPQVY
TLPPCREEMTKNQVSLWCLVKGFYPSDIAVEWESNGQPENNYKTTPPVLDSDGSFFLYSKLTVDKSRWQQGNVFSCSVMHEALHNHYT
QKSLSLSPGKGGGSGGGSGGGSGGGSGSTGEVQLVESGGGLVQPGGSLRLSCAASGEDFTAYAMHWVRQAPGKGLEWVASIYPSGGYT
AYADSVKGRFTISADTSKNTAYLQMNSLRAEDTAVYYCARRSYYFALDYWGQGTLVTVSSGGGGSDIQMTQSPSSLSASVGDRVTITC
RASQSVSSAVAWYQQKPGKAPKLLIYSASSLYSGVPSRFSGSRSGTDFTLTISSLQPEDFATYYCQQYWAYYSPITFGQGTKVEIK*
(SEQ ID NO: 968)
ANT42 Hc Hole DANG
MNLLLILTFVAAAVAEVQLVESGGGLVQPGGSLRLSCAASGFDFSSSSIHWVRQAPGKGLEWVASISSSYGYTYYADSVKGRFTISAD
TSKNTAYLQMNSLRAEDTAVYYCARSWAMDYWGQGTLVTVSSGGGGSDIQMTQSPSSLSASVGDRVTITCRASQSVSSAVAWYQQKPG
KAPKLLIYSASDLYSGVPSRFSGSRSGTDFTLTISSLQPEDFATYYCQQYAGAGLITFGQGTKVEIKGGGGSGGGGSEPKSSDKTHTC
PPCPAPELLGGPSVFLFPPKPKDTLMISRTPEVTCVVVAVSHEDPEVKFNWYVDGVEVHNAKTKPREEQYGSTYRVVSVLTVLHQDWL
SFFNGKEYKCKVSNKALPAPIEKTISKAKGQPREPQVYTLPPIRELMTSNQVSLSCAVKGFYPSDIAVEWESNGQPENNYKTTPPVLD
SDGLVSKLTVDKSRWQQGNVFSCSVMHEALHNHYTQKSLSLSPGKGGGSGGGSGGGSGGGSGSTGEVQLVESGGGLVQPGGSLRLSCA
ASGFTSDFYFIHWVRQAPGKGLEWVATIYPFIGNTYYADSVKGRFTISADTSKNTAYLQMNSLRAEDTAVYYCARAFPGSYHPFDYWG
QGTLVTVSSASTKGPSVFPLAPSSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHT* (SEQ ID NO: 911)
ANT42 Hc Hole LALAPS
MNLLLILTFVAAAVAEVQLVESGGGLVQPGGSLRLSCAASGFDFSSSSIHWVRQAPGKGLEWVASISSSYGYTYYADSVKGRFTISAD
TSKNTAYLQMNSLRAEDTAVYYCARSWAMDYWGQGTLVTVSSGGGGSDIQMTQSPSSLSASVGDRVTITCRASQSVSSAVAWYQQKPG
KAPKLLIYSASDLYSGVPSRFSGSRSGTDFTLTISSLQPEDFATYYCQQYAGAGLITFGQGTKVEIKGGGGSGGGGSEPKSSDKTHTC
PPCPAPEAAGGPSVFLFPPKPKDTLMISRTPEVTCVVVDVSHEDPEVKFNWYVDGVEVHNAKTKPREEQYNSTYRVVSVLTVLHQDWL
NGKEYKCKVSNKALPASIEKTISKAKGQPREPQVYTLPPIRELMTSNQVSLSCAVKGFYPSDIAVEWESNGQPENNYKTTPPVLDSDG
SFFLVSKLTVDKSRWQQGNVFSCSVMHEALHNHYTQKSLSLSPGKGGGSGGGSGGGSGGGSGSTGEVQLVESGGGLVQPGGSLRLSCA
ASGFTSDFYFIHWVRQAPGKGLEWVATIYPFIGNTYYADSVKGRFTISADTSKNTAYLQMNSLRAEDTAVYYCARAFPGSYHPFDYWG
QGTLVTVSSASTKGPSVFPLAPSSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHT* (SEQ ID NO: 969)
ANT42 Hc Hole LALAPS Merchant
MNLLLILTFVAAAVAEVQLVESGGGLVQPGGSLRLSCAASGFDFSSSSIHWVRQAPGKGLEWVASISSSYGYTYYADSVKGRFTISAD
TSKNTAYLQMNSLRAEDTAVYYCARSWAMDYWGQGTLVTVSSGGGGSDIQMTQSPSSLSASVGDRVTITCRASQSVSSAVAWYQQKPG
KAPKLLIYSASDLYSGVPSRFSGSRSGTDFTLTISSLQPEDFATYYCQQYAGAGLITFGQGTKVEIKGGGGSGGGGSEPKSSDKTHTC
PPCPAPEAAGGPSVFLFPPKPKDTLMISRTPEVTCVVVDVSHEDPEVKFNWYVDGVEVHNAKTKPREEQYNSTYRVVSVLTVLHQDWL
NGKEYKCKVSNKALPASIEKTISKAKGQPREPQVYTLPPSREEMTKNQVSLSCAVKGFYPSDIAVEWESNGQPENNYKTTPPVLDSDG
SFFLVSKLTVDKSRWQQGNVFSCSVMHEALHNHYTQKSLSLSPGKGGGSGGGSGGGSGGGSGSTGEVQLVESGGGLVQPGGSLRLSCA
ASGFTSDFYFIHWVRQAPGKGLEWVATIYPFIGNTYYADSVKGRFTISADTSKNTAYLQMNSLRAEDTAVYYCARAFPGSYHPFDYWG
QGTLVTVSSASTKGPSVFPLAPSSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHT* (SEQ ID NO: 970)
ANT42 Hc Hole LALAPS Merchant S-S
MNLLLILTFVAAAVAEVQLVESGGGLVQPGGSLRLSCAASGFDFSSSSIHWVRQAPGKGLEWVASISSSYGYTYYADSVKGRFTISAD
TSKNTAYLQMNSLRAEDTAVYYCARSWAMDYWGQGTLVTVSSGGGGSDIQMTQSPSSLSASVGDRVTITCRASQSVSSAVAWYQQKPG
KAPKLLIYSASDLYSGVPSRFSGSRSGTDFTLTISSLQPEDFATYYCQQYAGAGLITFGQGTKVEIKGGGGSGGGGSEPKSSDKTHTC
PPCPAPEAAGGPSVFLFPPKPKDTLMISRTPEVTCVVVDVSHEDPEVKFNWYVDGVEVHNAKTKPREEQYNSTYRVVSVLTVLHQDWL
NGKEYKCKVSNKALPASIEKTISKAKGQPREPQVCTLPPSREEMTKNQVSLSCAVKGFYPSDIAVEWESNGQPENNYKTTPPVLDSDG
SFFLVSKLTVDKSRWQQGNVFSCSVMHEALHNHYTQKSLSLSPGKGGGSGGGSGGGSGGGSGSTGEVQLVESGGGLVQPGGSLRLSCA
ASGFTSDFYFIHWVRQAPGKGLEWVATIYPFIGNTYYADSVKGRFTISADTSKNTAYLQMNSLRAEDTAVYYCARAFPGSYHPFDYWG
QGTLVTVSSASTKGPSVFPLAPSSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHT* (SEQ ID NO: 971)
Mature ANT42 Hc Hole DANG
EVQLVESGGGLVQPGGSLRLSCAASGFDFSSSSIHWVRQAPGKGLEWVASISSSYGYTYYADSVKGRFTISADTSKNTAYLQMNSLRA
EDTAVYYCARSWAMDYWGQGTLVTVSSGGGGSDIQMTQSPSSLSASVGDRVTITCRASQSVSSAVAWYQQKPGKAPKLLIYSASDLYS
GVPSRFSGSRSGTDFTLTISSLQPEDFATYYCQQYAGAGLITFGQGTKVEIKGGGGSGGGGSEPKSSDKTHTCPPCPAPELLGGPSVF
LFPPKPKDTLMISRTPEVTCVVVAVSHEDPEVKFNWYVDGVEVHNAKTKPREEQYGSTYRVVSVLTVLHQDWLNGKEYKCKVSNKALP
APIEKTISKAKGQPREPQVYTLPPIRELMTSNQVSLSCAVKGFYPSDIAVEWESNGQPENNYKTTPPVLDSDGSFFLVSKLTVDKSRW
QQGNVFSCSVMHEALHNHYTQKSLSLSPGKGGGSGGGSGGGSGGGSGSTGEVQLVESGGGLVQPGGSLRLSCAASGFTSDFYFIHWVR
QAPGKGLEWVATIYPFIGNTYYADSVKGRFTISADTSKNTAYLQMNSLRAEDTAVYYCARAFPGSYHPFDYWGQGTLVTVSSASTKGP
SVFPLAPSSKSTSGGTAALGCLVKDYFPEPVTVSWNSGALTSGVHTFPAVLQSSGLYSLSSVVTVPSSSLGTQTYICNVNHKPSNTKV
DKKVEPKSCDKTHT* (SEQ ID NO: 972)
Mature ANT42 Hc Hole LALAPS
EVQLVESGGGLVQPGGSLRLSCAASGFDFSSSSIHWVRQAPGKGLEWVASISSSYGYTYYADSVKGRFTISADTSKNTAYLQMNSLRA
EDTAVYYCARSWAMDYWGQGTLVTVSSGGGGSDIQMTQSPSSLSASVGDRVTITCRASQSVSSAVAWYQQKPGKAPKLLIYSASDLYS
GVPSRFSGSRSGTDFTLTISSLQPEDFATYYCQQYAGAGLITFGQGTKVEIKGGGGSGGGGSEPKSSDKTHTCPPCPAPEAAGGPSVF
LFPPKPKDTLMISRTPEVTCVVVDVSHEDPEVKFNWYVDGVEVHNAKTKPREEQYNSTYRVVSVLTVLHQDWLNGKEYKCKVSNKALP
ASIEKTISKAKGQPREPQVYTLPPIRELMTSNQVSLSCAVKGFYPSDIAVEWESNGQPENNYKTTPPVLDSDGSFFLVSKLTVDKSRW
QQGNVFSCSVMHEALHNHYTQKSLSLSPGKGGGSGGGSGGGSGGGSGSTGEVQLVESGGGLVQPGGSLRLSCAASGFTSDFYFIHWVR
QAPGKGLEWVATIYPFIGNTYYADSVKGRFTISADTSKNTAYLQMNSLRAEDTAVYYCARAFPGSYHPFDYWGQGTLVTVSSASTKGP
SVFPLAPSSKSTSGGTAALGCLVKDYFPEPVTVSWNSGALTSGVHTFPAVLQSSGLYSLSSVVTVPSSSLGTQTYICNVNHKPSNTKV
DKKVEPKSCDKTHT* (SEQ ID NO: 973)
Mature ANT42 Hc Hole LALAPS Merchant
EVQLVESGGGLVQPGGSLRLSCAASGFDFSSSSIHWVRQAPGKGLEWVASISSSYGYTYYADSVKGRFTISADTSKNTAYLQMNSLRA
EDTAVYYCARSWAMDYWGQGTLVTVSSGGGGSDIQMTQSPSSLSASVGDRVTITCRASQSVSSAVAWYQQKPGKAPKLLIYSASDLYS
GVPSRFSGSRSGTDFTLTISSLQPEDFATYYCQQYAGAGLITFGQGTKVEIKGGGGSGGGGSEPKSSDKTHTCPPCPAPEAAGGPSVF
LFPPKPKDTLMISRTPEVTCVVVDVSHEDPEVKFNWYVDGVEVHNAKTKPREEQYNSTYRVVSVLTVLHQDWLNGKEYKCKVSNKALP
ASIEKTISKAKGQPREPQVYTLPPSREEMTKNQVSLSCAVKGFYPSDIAVEWESNGQPENNYKTTPPVLDSDGSFFLVSKLTVDKSRW
QQGNVFSCSVMHEALHNHYTQKSLSLSPGKGGGSGGGSGGGSGGGSGSTGEVQLVESGGGLVQPGGSLRLSCAASGFTSDFYFIHWVR
QAPGKGLEWVATIYPFIGNTYYADSVKGRFTISADTSKNTAYLQMNSLRAEDTAVYYCARAFPGSYHPFDYWGQGTLVTVSSASTKGP
SVFPLAPSSKSTSGGTAALGCLVKDYFPEPVTVSWNSGALTSGVHTFPAVLQSSGLYSLSSVVTVPSSSLGTQTYICNVNHKPSNTKV
DKKVEPKSCDKTHT* (SEQ ID NO: 974)
Mature ANT42 Hc Hole LALAPS Merchant S-S
EVQLVESGGGLVQPGGSLRLSCAASGFDFSSSSIHWVRQAPGKGLEWVASISSSYGYTYYADSVKGRFTISADTSKNTAYLQMNSLRA
EDTAVYYCARSWAMDYWGQGTLVTVSSGGGGSDIQMTQSPSSLSASVGDRVTITCRASQSVSSAVAWYQQKPGKAPKLLIYSASDLYS
GVPSRFSGSRSGTDFTLTISSLQPEDFATYYCQQYAGAGLITFGQGTKVEIKGGGGSGGGGSEPKSSDKTHTCPPCPAPEAAGGPSVF
LFPPKPKDTLMISRTPEVTCVVVDVSHEDPEVKFNWYVDGVEVHNAKTKPREEQYNSTYRVVSVLTVLHQDWLNGKEYKCKVSNKALP
ASIEKTISKAKGQPREPQVCTLPPSREEMTKNQVSLSCAVKGFYPSDIAVEWESNGQPENNYKTTPPVLDSDGSFFLVSKLTVDKSRW
QQGNVFSCSVMHEALHNHYTQKSLSLSPGKGGGSGGGSGGGSGGGSGSTGEVQLVESGGGLVQPGGSLRLSCAASGFTSDFYFIHWVR
QAPGKGLEWVATIYPFIGNTYYADSVKGRFTISADTSKNTAYLQMNSLRAEDTAVYYCARAFPGSYHPFDYWGQGTLVTVSSASTKGP
SVFPLAPSSKSTSGGTAALGCLVKDYFPEPVTVSWNSGALTSGVHTFPAVLQSSGLYSLSSVVTVPSSSLGTQTYICNVNHKPSNTKV
DKKVEPKSCDKTHT* (SEQ ID NO: 975)
ANT42 Hc knob DANG
MNLLLILTFVAAAVAEVQLVESGGGLVQPGGSLRLSCAASGFDFTAYAMHWVRQAPGKGLEWVASIYPSGGYTAYADSVKGRFTISAD
TSKNTAYLQMNSLRAEDTAVYYCARRSYYFALDYWGQGTLVTVSSGGGGSDIQMTQSPSSLSASVGDRVTITCRASQSVSSAVAWYQQ
KPGKAPKLLIYSASSLYSGVPSRFSGSRSGTDFTLTISSLQPEDFATYYCQQYWAYYSPITFGQGTKVEIKGGGGSGGGGSEPKSSDK
THTCPPCPAPELLGGPSVFLFPPKPKDTLMISRTPEVTCVVVAVSHEDPEVKFNWYVDGVEVHNAKTKPREEQYGSTYRVVSVLTVLH
QDWLNGKEYKCKVSNKALPAPIEKTISKAKGQPREPMVFDLPPSREEMTKNQVSLWCMVKGFYPSDIAVEWESNGQPENNYKTTPPVL
DSDGSFFLYSKLTVDKSRWQQGNVFSCSVMHEALHNHYTQKSLSLSPGKGGGSGGGSGGGSGGGSGSTGEVQLVESGGGLVQPGGSLR
LSCAASGFTSDFYFIHWVRQAPGKGLEWVATIYPFIGNTYYADSVKGRFTISADTSKNTAYLQMNSLRAEDTAVYYCARAFPGSYHPF
DYWGQGTLVTVSSASTKGPSVFPLAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHT* (SEQ ID NO: 976)
ANT42 Hc knob LALAPS
MNLLLILTFVAAAVAEVQLVESGGGLVQPGGSLRLSCAASGEDFTAYAMHWVRQAPGKGLEWVASIYPSGGYTAYADSVKGRFTISAD
TSKNTAYLQMNSLRAEDTAVYYCARRSYYFALDYWGQGTLVTVSSGGGGSDIQMTQSPSSLSASVGDRVTITCRASQSVSSAVAWYQQ
KPGKAPKLLIYSASSLYSGVPSRFSGSRSGTDFTLTISSLQPEDFATYYCQQYWAYYSPITFGQGTKVEIKGGGGSGGGGSEPKSSDK
THTCPPCPAPEAAGGPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPASIEKTISKAKGQPREPMVFDLPPSREEMTKNQVSLWCMVKGFYPSDIAVEWESNGQPENNYKTTPPVL
DSDGSFFLYSKLTVDKSRWQQGNVFSCSVMHEALHNHYTQKSLSLSPGKGGGSGGGSGGGSGGGSGSTGEVQLVESGGGLVQPGGSLR
LSCAASGFTSDFYFIHWVRQAPGKGLEWVATIYPFIGNTYYADSVKGRFTISADTSKNTAYLQMNSLRAEDTAVYYCARAFPGSYHPF
DYWGQGTLVTVSSASTKGPSVFPLAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHT* (SEQ ID NO: 977)
ANT42 Hc knob LALAPS Merchant
MNLLLILTFVAAAVAEVQLVESGGGLVQPGGSLRLSCAASGFDFTAYAMHWVRQAPGKGLEWVASIYPSGGYTAYADSVKGRFTISAD
TSKNTAYLQMNSLRAEDTAVYYCARRSYYFALDYWGQGTLVTVSSGGGGSDIQMTQSPSSLSASVGDRVTITCRASQSVSSAVAWYQQ
KPGKAPKLLIYSASSLYSGVPSRFSGSRSGTDFTLTISSLQPEDFATYYCQQYWAYYSPITFGQGTKVEIKGGGGSGGGGSEPKSSDK
THTCPPCPAPEAAGGPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPASIEKTISKAKGQPREPQVYTLPPSREEMTKNQVSLWCLVKGFYPSDIAVEWESNGQPENNYKTTPPVL
DSDGSFFLYSKLTVDKSRWQQGNVFSCSVMHEALHNHYTQKSLSLSPGKGGGSGGGSGGGSGGGSGSTGEVQLVESGGGLVQPGGSLR
LSCAASGFTSDFYFIHWVRQAPGKGLEWVATIYPFIGNTYYADSVKGRFTISADTSKNTAYLQMNSLRAEDTAVYYCARAFPGSYHPF
DYWGQGTLVTVSSASTKGPSVFPLAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHT* (SEQ ID NO: 978)
ANT42 Hc knob LALAPS Merchant S-S
MNLLLILTFVAAAVAEVQLVESGGGLVQPGGSLRLSCAASGFDFTAYAMHWVRQAPGKGLEWVASIYPSGGYTAYADSVKGRFTISAD
TSKNTAYLQMNSLRAEDTAVYYCARRSYYFALDYWGQGTLVTVSSGGGGSDIQMTQSPSSLSASVGDRVTITCRASQSVSSAVAWYQQ
KPGKAPKLLIYSASSLYSGVPSRFSGSRSGTDFTLTISSLQPEDFATYYCQQYWAYYSPITFGQGTKVEIKGGGGSGGGGSEPKSSDK
THTCPPCPAPEAAGGPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPASIEKTISKAKGQPREPQVYTLPPCREEMTKNQVSLWCLVKGFYPSDIAVEWESNGQPENNYKTTPPVL
DSDGSFFLYSKLTVDKSRWQQGNVFSCSVMHEALHNHYTQKSLSLSPGKGGGSGGGSGGGSGGGSGSTGEVQLVESGGGLVQPGGSLR
LSCAASGFTSDFYFIHWVRQAPGKGLEWVATIYPFIGNTYYADSVKGRFTISADTSKNTAYLQMNSLRAEDTAVYYCARAFPGSYHPF
DYWGQGTLVTVSSASTKGPSVFPLAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHT* (SEQ ID NO: 979)
Mature ANT42 Hc knob DANG
EVQLVESGGGLVQPGGSLRLSCAASGEDFTAYAMHWVRQAPGKGLEWVASIYPSGGYTAYADSVKGRFTISADTSKNTAYLQMNSLRA
EDTAVYYCARRSYYFALDYWGQGTLVTVSSGGGGSDIQMTQSPSSLSASVGDRVTITCRASQSVSSAVAWYQQKPGKAPKLLIYSASS
LYSGVPSRFSGSRSGTDFTLTISSLQPEDFATYYCQQYWAYYSPITFGQGTKVEIKGGGGSGGGGSEPKSSDKTHTCPPCPAPELLGG
PSVFLFPPKPKDTLMISRTPEVTCVVVAVSHEDPEVKFNWYVDGVEVHNAKTKPREEQYGSTYRVVSVLTVLHQDWLNGKEYKCKVSN
KALPAPIEKTISKAKGQPREPMVFDLPPSREEMTKNQVSLWCMVKGFYPSDIAVEWESNGQPENNYKTTPPVLDSDGSFFLYSKLTVD
KSRWQQGNVFSCSVMHEALHNHYTQKSLSLSPGKGGGSGGGSGGGSGGGSGSTGEVQLVESGGGLVQPGGSLRLSCAASGFTSDFYFI
HWVRQAPGKGLEWVATIYPFIGNTYYADSVKGRFTISADTSKNTAYLQMNSLRAEDTAVYYCARAFPGSYHPFDYWGQGTLVTVSSAS
TKGPSVFPLAPSSKSTSGGTAALGCLVKDYFPEPVTVSWNSGALTSGVHTFPAVLQSSGLYSLSSVVTVPSSSLGTQTYICNVNHKPS
NTKVDKKVEPKSCDKTHT* (SEQ ID NO: 980)
Mature ANT42 Hc knob LALAPS
EVQLVESGGGLVQPGGSLRLSCAASGFDFTAYAMHWVRQAPGKGLEWVASIYPSGGYTAYADSVKGRFTISADTSKNTAYLQMNSLRA
EDTAVYYCARRSYYFALDYWGQGTLVTVSSGGGGSDIQMTQSPSSLSASVGDRVTITCRASQSVSSAVAWYQQKPGKAPKLLIYSASS
LYSGVPSRFSGSRSGTDFTLTISSLQPEDFATYYCQQYWAYYSPITFGQGTKVEIKGGGGGGGGSEPKSSDKTHTCPPCPAPEAAGGP
SVFLFPPKPKDTLMISRTPEVTCVVVDVSHEDPEVKFNWYVDGVEVHNAKTKPREEQYNSTYRVVSVLTVLHQDWLNGKEYKCKVSNK
ALPASIEKTISKAKGQPREPMVFDLPPSREEMTKNQVSLWCMVKGFYPSDIAVEWESNGQPENNYKTTPPVLDSDGSFFLYSKLTVDK
SRWQQGNVFSCSVMHEALHNHYTQKSLSLSPGKGGGSGGGSGGGSGGGSGSTGEVQLVESGGGLVQPGGSLRLSCAASGFTSDFYFIH
WVRQAPGKGLEWVATIYPFIGNTYYADSVKGRFTISADTSKNTAYLQMNSLRAEDTAVYYCARAFPGSYHPFDYWGQGTLVTVSSAST
KGPSVFPLAPSSKSTSGGTAALGCLVKDYFPEPVTVSWNSGALTSGVHTFPAVLQSSGLYSLSSVVTVPSSSLGTQTYICNVNHKPSN
TKVDKKVEPKSCDKTHT* (SEQ ID NO: 981)
Mature ANT42 Hc knob LALAPS Merchant
EVQLVESGGGLVQPGGSLRLSCAASGFDFTAYAMHWVRQAPGKGLEWVASIYPSGGYTAYADSVKGRFTISADTSKNTAYLQMNSLRA
EDTAVYYCARRSYYFALDYWGQGTLVTVSSGGGGSDIQMTQSPSSLSASVGDRVTITCRASQSVSSAVAWYQQKPGKAPKLLIYSASS
LYSGVPSRFSGSRSGTDFTLTISSLQPEDFATYYCQQYWAYYSPITFGQGTKVEIKGGGGGGGGSEPKSSDKTHTCPPCPAPEAAGGP
SVFLFPPKPKDTLMISRTPEVTCVVVDVSHEDPEVKFNWYVDGVEVHNAKTKPREEQYNSTYRVVSVLTVLHQDWLNGKEYKCKVSNK
ALPASIEKTISKAKGQPREPQVYTLPPSREEMTKNQVSLWCLVKGFYPSDIAVEWESNGQPENNYKTTPPVLDSDGSFFLYSKLTVDK
SRWQQGNVFSCSVMHEALHNHYTQKSLSLSPGKGGGSGGGSGGGSGGGSGSTGEVQLVESGGGLVQPGGSLRLSCAASGFTSDFYFIH
WVRQAPGKGLEWVATIYPFIGNTYYADSVKGRFTISADTSKNTAYLQMNSLRAEDTAVYYCARAFPGSYHPFDYWGQGTLVTVSSAST
KGPSVFPLAPSSKSTSGGTAALGCLVKDYFPEPVTVSWNSGALTSGVHTFPAVLQSSGLYSLSSVVTVPSSSLGTQTYICNVNHKPSN
TKVDKKVEPKSCDKTHT* (SEQ ID NO: 982)
Mature ANT42 Hc knob LALAPS Merchant S-S
EVQLVESGGGLVQPGGSLRLSCAASGEDFTAYAMHWVRQAPGKGLEWVASIYPSGGYTAYADSVKGRFTISADTSKNTAYLQMNSLRA
EDTAVYYCARRSYYFALDYWGQGTLVTVSSGGGGSDIQMTQSPSSLSASVGDRVTITCRASQSVSSAVAWYQQKPGKAPKLLIYSASS
LYSGVPSRFSGSRSGTDFTLTISSLQPEDFATYYCQQYWAYYSPITFGQGTKVEIKGGGGSGGGGSEPKSSDKTHTCPPCPAPEAAGG
PSVFLFPPKPKDTLMISRTPEVTCVVVDVSHEDPEVKFNWYVDGVEVHNAKTKPREEQYNSTYRVVSVLTVLHQDWLNGKEYKCKVSN
KALPASIEKTISKAKGQPREPQVYTLPPCREEMTKNQVSLWCLVKGFYPSDIAVEWESNGQPENNYKTTPPVLDSDGSFFLYSKLTVD
KSRWQQGNVFSCSVMHEALHNHYTQKSLSLSPGKGGGSGGGSGGGSGGGSGSTGEVQLVESGGGLVQPGGSLRLSCAASGFTSDFYFI
HWVRQAPGKGLEWVATIYPFIGNTYYADSVKGRFTISADTSKNTAYLQMNSLRAEDTAVYYCARAFPGSYHPFDYWGQGTLVTVSSAS
TKGPSVFPLAPSSKSTSGGTAALGCLVKDYFPEPVTVSWNSGALTSGVHTFPAVLQSSGLYSLSSVVTVPSSSLGTQTYICNVNHKPS
NTKVDKKVEPKSCDKTHT* (SEQ ID NO: 983)
ANT42 LC
MNLLLILTFVAAAVADIQMTQSPSSLSASVGDRVTITCRASQSVSSAVAWYQQKPGKAPKLLIYSASSLYSGVPSRFSGSRSGTDFTL
TISSLQPEDFATYYCQQSTGSLITFGQGTKVEIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC* (SEQ ID NO: 912)
Mature ANT42 LC
DIQMTQSPSSLSASVGDRVTITCRASQSVSSAVAWYQQKPGKAPKLLIYSASSLYSGVPSRFSGSRSGTDFTLTISSLQPEDFATYYC
QQSTGSLITFGQGTKVEIKRTVAAPSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC (SEQ ID NO: 984)
ANT80-1 Hole
MNLLLILTFVAAAVAEVQLVESGGGLVQPGGSLRLSCAASGFTLSYSYIHWVRQAPGKGLEWVASIYSYTGATDYADSVKGRFTISAD
TSKNTAYLQMNSLRAEDTAVYYCARYWSTNRILSYGGMDYWGQGTLVTVSSGGGGSDIQMTQSPSSLSASVGDRVTITCRASQSVSSA
VAWYQQKPGKAPKLLIYSASSLYSGVPSRFSGSRSGTDFTLTISSLQPEDFATYYCQQYFDPITFGQGTKVEIKGGGGSGGGGSEPKS
SDKTHTCPPCPAPELLGGPSVFLFPPKPKDTLMISRTPEVTCVVVAVSHEDPEVKFNWYVDGVEVHNAKTKPREEQYGSTYRVVSVLT
VLHQDWLNGKEYKCKVSNKALPAPIEKTISKAKGQPREPQVYTLPPIRELMTSNQVSLSCAVKGFYPSDIAVEWESNGQPENNYKTTP
PVLDSDGSFFLVSKLTVDKSRWQQGNVFSCSVMHEALHNHYTQKSLSLSPGKGGGSGGGSGGGSGGGSGSTGEVQLVESGGGLVQPGG
SLRLSCAASGENIAYSSMHWVRQAPGKGLEWVATIYPSYSSTYYADSVKGRFTISADTSKNTAYLQMNSLRAEDTAVYYCARYYAMDY
WGQGTLVTVSSASTKGPSVFPLAPSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHT (SEQ ID NO: 986)
ANT80-2 Knob
MNLLLILTFVAAAVAEVQLVESGGGLVQPGGSLRLSCAASGFDLSHSSIHWVRQAPGKGLEWVASISSHYGYTHYADSVKGRFTISAD
TSKNTAYLQMNSLRAEDTAVYYCARSSYFPWFFALDYWGQGTLVTVSSGGGGSDIQMTQSPSSLSASVGDRVTITCRASQDVSTAVAW
YQQKPGKAPKLLIYSASFLYSGVPSRFSGSRSGTDFTLTISSLQPEDFATYYCQQHYTTPPTFGQGTKVEIKGGGGSGGGGSEPKTSD
KTHTCPPCPAPELLGGPSVFLFPPKPKDTLMISRTPEVTCVVVAVSHEDPEVKFNWYVDGVEVHNAKTKPREEQYGSTYRVVSVLTVL
HQDWLNGKEYKCKVSNKALPAPIEKTISKAKGQPREPMVFDLPPSREEMTKNQVSLWCMVKGFYPSDIAVEWESNGQPENNYKTTPPV
LDSDGSFFLYSKLTVDKSRWQQGNVFSCSVMHEALHNHYTQKSLSLSPGKGGGSGGGSGGGSGGGSGSTGEVQLVESGGGLVQPGGSL
RLSCAASGENIAYSSMHWVRQAPGKGLEWVATIYPSYSSTYYADSVKGRFTISADTSKNTAYLQMNSLRAEDTAVYYCARYYAMDYWG
QGTLVTVSSASTKGPSVFPLAPSSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHT (SEQ ID NO: 987)
ANT98-1 Hole
MNLLLILTFVAAAVAEVQLVESGGGLVQPGGSLRLSCAASGFTLSYSYIHWVRQAPGKGLEWVASIYSYTGATDYADSVKGRFTISAD
TSKNTAYLQMNSLRAEDTAVYYCARYWSTNRILSYGGMDYWGQGTLVTVSSGGGGSDIQMTQSPSSLSASVGDRVTITCRASQSVSSA
VAWYQQKPGKAPKLLIYSASSLYSGVPSRFSGSRSGTDFTLTISSLQPEDFATYYCQQYFDPITFGQGTKVEIKGGGGSGGGGSEPKS
SDKTHTCPPCPAPELLGGPSVFLFPPKPKDTLMISRTPEVTCVVVAVSHEDPEVKFNWYVDGVEVHNAKTKPREEQYGSTYRVVSVLT
VLHQDWLNGKEYKCKVSNKALPAPIEKTISKAKGQPREPQVYTLPPIRELMTSNQVSLSCAVKGFYPSDIAVEWESNGQPENNYKTTP
PVLDSDGSFFLVSKLTVDKSRWQQGNVFSCSVMHEALHNHYTQKSLSLSPGKGGGSGGGSGGGSGGGSGSTGEVQLVESGGGLVQPGG
SLRLSCAASGENIAYSSMHWVRQAPGKGLEWVATIYPSYSSTYYADSVKGRFTISADTSKNTAYLQMNSLRAEDTAVYYCARDYAMDY
WGQGTLVTVSSASTKGPSVFPLAPSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHT (SEQ ID NO: 988)
ANT98-2 Knob
MNLLLILTFVAAAVAEVQLVESGGGLVQPGGSLRLSCAASGFDLSHSSIHWVRQAPGKGLEWVASISSHYGYTHYADSVKGRFTISAD
TSKNTAYLQMNSLRAEDTAVYYCARSSYFPWFFALDYWGQGTLVTVSSGGGGSDIQMTQSPSSLSASVGDRVTITCRASQDVSTAVAW
YQQKPGKAPKLLIYSASFLYSGVPSRFSGSRSGTDFTLTISSLQPEDFATYYCQQHYTTPPTFGQGTKVEIKGGGGSGGGGSEPKTSD
KTHTCPPCPAPELLGGPSVFLFPPKPKDTLMISRTPEVTCVVVAVSHEDPEVKFNWYVDGVEVHNAKTKPREEQYGSTYRVVSVLTVL
HQDWLNGKEYKCKVSNKALPAPIEKTISKAKGQPREPMVFDLPPSREEMTKNQVSLWCMVKGFYPSDIAVEWESNGQPENNYKTTPPV
LDSDGSFFLYSKLTVDKSRWQQGNVFSCSVMHEALHNHYTQKSLSLSPGKGGGSGGGSGGGSGGGSGSTGEVQLVESGGGLVQPGGSL
RLSCAASGENIAYSSMHWVRQAPGKGLEWVATIYPSYSSTYYADSVKGRFTISADTSKNTAYLQMNSLRAEDTAVYYCARDYAMDYWG
QGTLVTVSSASTKGPSVFPLAPSSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHT (SEQ ID NO: 989)
ANT99-1 Hole
MNLLLILTFVAAAVAEVQLVESGGGLVQPGGSLRLSCAASGFTLSYSYIHWVRQAPGKGLEWVASIYSYTGATDYADSVKGRFTISAD
TSKNTAYLQMNSLRAEDTAVYYCARYWSTNRILSYGGMDYWGQGTLVTVSSGGGGSDIQMTQSPSSLSASVGDRVTITCRASQSVSSA
VAWYQQKPGKAPKLLIYSASSLYSGVPSRFSGSRSGTDFTLTISSLQPEDFATYYCQQYFDPITFGQGTKVEIKGGGGSGGGGSEPKS
SDKTHTCPPCPAPELLGGPSVFLFPPKPKDTLMISRTPEVTCVVVAVSHEDPEVKFNWYVDGVEVHNAKTKPREEQYGSTYRVVSVLT
VLHQDWLNGKEYKCKVSNKALPAPIEKTISKAKGQPREPQVYTLPPIRELMTSNQVSLSCAVKGFYPSDIAVEWESNGQPENNYKTTP
PVLDSDGSFFLVSKLTVDKSRWQQGNVFSCSVMHEALHNHYTQKSLSLSPGKGGGSGGGSGGGSGGGSGSTGEVQLVESGGGLVQPGG
SLRLSCAASGENIAYSSMHWVRQAPGKGLEWVATIYPSYSSTYYADSVKGRFTISADTSKNTAYLQMNSLRAEDTAVYYCARYYDMDY
WGQGTLVTVSSASTKGPSVFPLAPSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHT (SEQ ID NO: 990)
ANT99-2 Knob
MNLLLILTFVAAAVAEVQLVESGGGLVQPGGSLRLSCAASGFDLSHSSIHWVRQAPGKGLEWVASISSHYGYTHYADSVKGRFTISAD
TSKNTAYLQMNSLRAEDTAVYYCARSSYFPWFFALDYWGQGTLVTVSSGGGGSDIQMTQSPSSLSASVGDRVTITCRASQDVSTAVAW
YQQKPGKAPKLLIYSASFLYSGVPSRFSGSRSGTDFTLTISSLQPEDFATYYCQQHYTTPPTFGQGTKVEIKGGGGSGGGGSEPKTSD
KTHTCPPCPAPELLGGPSVFLFPPKPKDTLMISRTPEVTCVVVAVSHEDPEVKFNWYVDGVEVHNAKTKPREEQYGSTYRVVSVLTVL
HQDWLNGKEYKCKVSNKALPAPIEKTISKAKGQPREPMVFDLPPSREEMTKNQVSLWCMVKGFYPSDIAVEWESNGQPENNYKTTPPV
LDSDGSFFLYSKLTVDKSRWQQGNVFSCSVMHEALHNHYTQKSLSLSPGKGGGSGGGSGGGSGGGSGSTGEVQLVESGGGLVQPGGSL
RLSCAASGFNIAYSSMHWVRQAPGKGLEWVATIYPSYSSTYYADSVKGRFTISADTSKNTAYLQMNSLRAEDTAVYYCARYYDMDYWG
QGTLVTVSSASTKGPSVFPLAPSSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHT (SEQ ID NO: 991)
ANT80 ANT98 ANT99 LC
MNLLLILTFVAAAVADIQMTQSPSSLSASVGDRVTITCRASQSVSSAVAWYQQKPGKAPKLLIYSASSLYSGVPSRFSGSRSGTDFTL
TISSLQPEDFATYYCQQAHYFPITFGQGTKVEIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC (SEQ ID NO: 992)
CM0042 (Dia-Fc-Dia)
MNLLLILTFVAAAVAEFEVQLVESGGGLVQPGGSLRLSCAASGFNIGSSSIHWVRQAPGKGLEWVASIYSAFASTSYADSVKGRFTIS
ADTSKNTAYLQMNSLRAEDTAVYYCARYHFPFGFALDYWGQGTLVTVSSGGGGSDIQMTQSPSSLSASVGDRVTITCRASQSVSSAVA
WYQQKPGKAPKLLIYSASSLYSGVPSRFSGSRSGTDFTLTISSLQPEDFATYYCQQGVYLFTFGQGTKVEIKLEDKTHTKVEPKSSDK
THTCPPCPAPELLGGPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGKSGSETPGTSESATPESGGGEVQLVESGGGLVQPGGSLRL
SCAASGFNISYSSIHWVRQAPGKGLEWVAYISSYYGYTYYADSVKGRFTISADTSKNTAYLQMNSLRAEDTAVYYCARAHYFPWAGAM
DYWGQGTLVTVSSGGGGSDIQMTQSPSSLSASVGDRVTITCRASQSVSSAVAWYQQKPGKAPKLLIYSASSLYSGVPSRFSGSRSGTD
FTLTISSLQPEDFATYYCQQYSWGPFTFGQGTKVEIK (SEQ ID NO: 993)
CM0011 Hole (Dia-Fc-Dia)
MNLLLILTFVAAAVAEFEVQLVESGGGLVQPGGSLRLSCAASGFNIGSSSIHWVRQAPGKGLEWVASIYSAFASTSYADSVKGRFTIS
ADTSKNTAYLQMNSLRAEDTAVYYCARYHFPFGFALDYWGQGTLVTVSSGGGGSDIQMTQSPSSLSASVGDRVTITCRASQSVSSAVA
WYQQKPGKAPKLLIYSASSLYSGVPSRFSGSRSGTDFTLTISSLQPEDFATYYCQQGVYLFTFGQGTKVEIKLEDKTHTKVEPKSSDK
THNCPPCPAPELLGGPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPIRELMTSNQVSLSCAVKGFYPSDIAVEWESNGQPENNYKTTPPVL
DSDGSFFLVSKLTVDKSRWQQGNVFSCSVMHEALHNHYTQKSLSLSPGKSGSETPGTSESATPESGGGEVQLVESGGGLVQPGGSLRL
SCAASGFNISSYYIHWVRQAPGKGLEWVASIYSSYGYTSYADSVKGRFTISADTSKNTAYLQMNSLRAEDTAVYYCARTVRGSKKPYE
SGWAMDYWGQGTLVTVSSGGGGSDIQMTQSPSSLSASVGDRVTITCRASQSVSSAVAWYQQKPGKAPKLLIYSASSLYSGVPSRFSGS
RSGTDFTLTISSLQPEDFATYYCQQYSWGPFTFGQGTKVEIK (SEQ ID NO: 994)
CM0011 Knob (Dia-Fc-Dia)
MNLLLILTFVAAAVAEFEVQLVESGGGLVQPGGSLRLSCAASGFNIGSSSIHWVRQAPGKGLEWVASIYSAFASTSYADSVKGRFTIS
ADTSKNTAYLQMNSLRAEDTAVYYCARYHFPFGFALDYWGQGTLVTVSSGGGGSDIQMTQSPSSLSASVGDRVTITCRASQSVSSAVA
WYQQKPGKAPKLLIYSASSLYSGVPSRFSGSRSGTDFTLTISSLQPEDFATYYCQQGVYLFTFGQGTKVEIKLEDKTHTKVEPKTSDK
THTCPPCPAPELLGGPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPMVFDLPPSREEMTKNQVSLWCMVKGFYPSDIAVEWESNGQPENNYKTTPPVL
DSDGSFFLYSKLTVDKSRWQQGNVFSCSVMHEALHNHYTQKSLSLSPGKSGSETPGTSESATPESGGGEVQLVESGGGLVQPGGSLRL
SCAASGFNISYSSIHWVRQAPGKGLEWVAYISSYYGYTYYADSVKGRFTISADTSKNTAYLQMNSLRAEDTAVYYCARAHYFPWAGAM
DYWGQGTLVTVSSGGGGSDIQMTQSPSSLSASVGDRVTITCRASQSVSSAVAWYQQKPGKAPKLLIYSASSLYSGVPSRFSGSRSGTD
FTLTISSLQPEDFATYYCQQYYWPITFGQGTKVEIK (SEQ ID NO: 995)
CM0126 Hole (Dia-Fc-Dia)
MNLLLILTFVAAAVAEFEVQLVESGGGLVQPGGSLRLSCAASGFNIHSSSIHWVRQAPGKGLEWVAATYSSFGSITYADSVKGRFTIS
ADTSKNTAYLQMNSLRAEDTAVYYCARYHHPFGYALDYWGQGTLVTVSSGGGGSDIQMTQSPSSLSASVGDRVTITCRASQSVSSAVA
WYQQKPGKAPKLLIYSASSLYSGVPSRFSGSRSGTDFTLTISSLQPEDFATYYCQQGVYLFTFGQGTKVEIKLEDKTHTKVEPKSSDK
THNCPPCPAPELLGGPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPIRELMTSNQVSLSCAVKGFYPSDIAVEWESNGQPENNYKTTPPVL
DSDGSFFLVSKLTVDKSRWQQGNVFSCSVMHEALHNHYTQKSLSLSPGKSGSETPGTSESATPESGGGEVQLVESGGGLVQPGGSLRL
SCAASGFNISSYYIHWVRQAPGKGLEWVASIYSSYGYTSYADSVKGRFTISADTSKNTAYLQMNSLRAEDTAVYYCARTVRGSKKPYF
SGWAMDYWGQGTLVTVSSGGGGSDIQMTQSPSSLSASVGDRVTITCRASQSVSSAVAWYQQKPGKAPKLLIYSASSLYSGVPSRFSGS
RSGTDFTLTISSLQPEDFATYYCQQYSWGPFTFGQGTKVEIK (SEQ ID NO: 996)
CM0126 Knob (Dia-Fc-Dia)
MNLLLILTFVAAAVAEFEVQLVESGGGLVQPGGSLRLSCAASGFNIHSSSIHWVRQAPGKGLEWVAATYSSFGSITYADSVKGRFTIS
ADTSKNTAYLQMNSLRAEDTAVYYCARYHHPFGYALDYWGQGTLVTVSSGGGGSDIQMTQSPSSLSASVGDRVTITCRASQSVSSAVA
WYQQKPGKAPKLLIYSASSLYSGVPSRFSGSRSGTDFTLTISSLQPEDFATYYCQQGVYLFTFGQGTKVEIKLEDKTHTKVEPKTSDK
THTCPPCPAPELLGGPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPMVFDLPPSREEMTKNQVSLWCMVKGFYPSDIAVEWESNGQPENNYKTTPPVL
DSDGSFFLYSKLTVDKSRWQQGNVFSCSVMHEALHNHYTQKSLSLSPGKSGSETPGTSESATPESGGGEVQLVESGGGLVQPGGSLRL
SCAASGFNISYSSIHWVRQAPGKGLEWVAYISSYYGYTYYADSVKGRFTISADTSKNTAYLQMNSLRAEDTAVYYCARAHYFPWAGAM
DYWGQGTLVTVSSGGGGSDIQMTQSPSSLSASVGDRVTITCRASQSVSSAVAWYQQKPGKAPKLLIYSASSLYSGVPSRFSGSRSGTD
FTLTISSLQPEDFATYYCQQYYWPITFGQGTKVEIK (SEQ ID NO: 997)
CM0107 Hole (Dia-Fc-scFv)
MNLLLILTFVAAAVAEFEVQLVESGGGLVQPGGSLRLSCAASGFNIGSSSIHWVRQAPGKGLEWVASIYSAFASTSYADSVKGRFTIS
ADTSKNTAYLQMNSLRAEDTAVYYCARYHFPFGFALDYWGQGTLVTVSSGGGGSDIQMTQSPSSLSASVGDRVTITCRASQSVSSAVA
WYQQKPGKAPKLLIYSASSLYSGVPSRFSGSRSGTDFTLTISSLQPEDFATYYCQQGVYLFTFGQGTKVEIKLEDKTHTKVEPKSSDK
THNCPPCPAPELLGGPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPIRELMTSNQVSLSCAVKGFYPSDIAVEWESNGQPENNYKTTPPVL
DSDGSFFLVSKLTVDKSRWQQGNVFSCSVMHEALHNHYTQKSLSLSPGKSGSETPGTSESATPESGGGDIQMTQSPSSLSASVGDRVT
ITCRASQSVSSAVAWYQQKPGKAPKLLIYSASSLYSGVPSRFSGSRSGTDFTLTISSLQPEDFATYYCQQYSWGPFTFGQGTKVEIKG
TTAASGSSGGSSSGAEVQLVESGGGLVQPGGSLRLSCAASGFNISYSSIHWVRQAPGKGLEWVAYISSYYGYTYYADSVKGRFTISAD
TSKNTAYLQMNSLRAEDTAVYYCARAHYFPWAGAMDYWGQGTLVTVSS (SEQ ID NO: 998)
CM0107 Knob (Dia-Fc-scFv)
MNLLLILTFVAAAVAEFEVQLVESGGGLVQPGGSLRLSCAASGFNIGSSSIHWVRQAPGKGLEWVASIYSAFASTSYADSVKGRFTIS
ADTSKNTAYLQMNSLRAEDTAVYYCARYHFPFGFALDYWGQGTLVTVSSGGGGSDIQMTQSPSSLSASVGDRVTITCRASQSVSSAVA
WYQQKPGKAPKLLIYSASSLYSGVPSRFSGSRSGTDFTLTISSLQPEDFATYYCQQGVYLFTFGQGTKVEIKLEDKTHTKVEPKTSDK
THTCPPCPAPELLGGPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPMVFDLPPSREEMTKNQVSLWCMVKGFYPSDIAVEWESNGQPENNYKTTPPVL
DSDGSFFLYSKLTVDKSRWQQGNVFSCSVMHEALHNHYTQKSLSLSPGKSGSETPGTSESATPESGGGDIQMTQSPSSLSASVGDRVT
ITCRASQSVSSAVAWYQQKPGKAPKLLIYSASSLYSGVPSRFSGSRSGTDFTLTISSLQPEDFATYYCQQYSWGPFTFGQGTKVEIKG
TTAASGSSGGSSSGAEVQLVESGGGLVQPGGSLRLSCAASGFNISYSSIHWVRQAPGKGLEWVAYISSYYGYTYYADSVKGRFTISAD
TSKNTAYLQMNSLRAEDTAVYYCARAHYFPWAGAMDYWGQGTLVTVSS (SEQ ID NO: 999)
CM0108 Hole (Dia-Fc-scFv)
MNLLLILTFVAAAVAEFEVQLVESGGGLVQPGGSLRLSCAASGFNIGSSSIHWVRQAPGKGLEWVASIYSAFASTSYADSVKGRFTIS
ADTSKNTAYLQMNSLRAEDTAVYYCARYHFPFGFALDYWGQGTLVTVSSGGGGSDIQMTQSPSSLSASVGDRVTITCRASQSVSSAVA
WYQQKPGKAPKLLIYSASSLYSGVPSRFSGSRSGTDFTLTISSLQPEDFATYYCQQGVYLFTFGQGTKVEIKLEDKTHTKVEPKSSDK
THNCPPCPAPELLGGPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPIRELMTSNQVSLSCAVKGFYPSDIAVEWESNGQPENNYKTTPPVL
DSDGSFFLVSKLTVDKSRWQQGNVFSCSVMHEALHNHYTQKSLSLSPGKSGSETPGTSESATPESGGGDIQMTQSPSSLSASVGDRVT
ITCRASQSVSSAVAWYQQKPGKAPKLLIYSASSLYSGVPSRFSGSRSGTDFTLTISSLQPEDFATYYCQQYYWPITFGQGTKVEIKGT
TAASGSSGGSSSGAEVQLVESGGGLVQPGGSLRLSCAASGFNISSYYIHWVRQAPGKGLEWVASIYSSYGYTSYADSVKGRFTISADT
SKNTAYLQMNSLRAEDTAVYYCARTVRGSKKPYFSGWAMDYWGQGTLVTVSS (SEQ ID NO: 1000)
CM0108 Knob (Dia-Fc-scFv)
MNLLLILTFVAAAVAEFEVQLVESGGGLVQPGGSLRLSCAASGFNIGSSSIHWVRQAPGKGLEWVASIYSAFASTSYADSVKGRFTIS
ADTSKNTAYLQMNSLRAEDTAVYYCARYHFPFGFALDYWGQGTLVTVSSGGGGSDIQMTQSPSSLSASVGDRVTITCRASQSVSSAVA
WYQQKPGKAPKLLIYSASSLYSGVPSRFSGSRSGTDFTLTISSLQPEDFATYYCQQGVYLFTFGQGTKVEIKLEDKTHTKVEPKTSDK
THTCPPCPAPELLGGPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPMVFDLPPSREEMTKNQVSLWCMVKGFYPSDIAVEWESNGQPENNYKTTPPVL
DSDGSFFLYSKLTVDKSRWQQGNVFSCSVMHEALHNHYTQKSLSLSPGKSGSETPGTSESATPESGGGDIQMTQSPSSLSASVGDRVT
ITCRASQSVSSAVAWYQQKPGKAPKLLIYSASSLYSGVPSRFSGSRSGTDFTLTISSLQPEDFATYYCQQYYWPITFGQGTKVEIKGT
TAASGSSGGSSSGAEVQLVESGGGLVQPGGSLRLSCAASGFNISSYYIHWVRQAPGKGLEWVASIYSSYGYTSYADSVKGRFTISADT
SKNTAYLQMNSLRAEDTAVYYCARTVRGSKKPYFSGWAMDYWGQGTLVTVSS (SEQ ID NO: 1001)
CM0108 Hole (Dia-Fc-scFv)
MNLLLILTFVAAAVAEFEVQLVESGGGLVQPGGSLRLSCAASGFNIGSSSIHWVRQAPGKGLEWVASIYSAFASTSYADSVKGRFTIS
ADTSKNTAYLQMNSLRAEDTAVYYCARYHFPFGFALDYWGQGTLVTVSSGGGGSDIQMTQSPSSLSASVGDRVTITCRASQSVSSAVA
WYQQKPGKAPKLLIYSASSLYSGVPSRFSGSRSGTDFTLTISSLQPEDFATYYCQQGVYLFTFGQGTKVEIKLEDKTHTKVEPKSSDK
THNCPPCPAPELLGGPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPIRELMTSNQVSLSCAVKGFYPSDIAVEWESNGQPENNYKTTPPVL
DSDGSFFLVSKLTVDKSRWQQGNVFSCSVMHEALHNHYTQKSLSLSPGKSGSETPGTSESATPESGGGDIQMTQSPSSLSASVGDRVT
ITCRASQSVSSAVAWYQQKPGKAPKLLIYSASSLYSGVPSRFSGSRSGTDFTLTISSLQPEDFATYYCQQYYWPITFGQGTKVEIKGT
TAASGSSGGSSSGAEVQLVESGGGLVQPGGSLRLSCAASGFNISSYYIHWVRQAPGKGLEWVASIYSSYGYTSYADSVKGRFTISADT
SKNTAYLQMNSLRAEDTAVYYCARTVRGSKKPYFSGWAMDYWGQGTLVTVSS (SEQ ID NO: 1000)
CM0108 Knob (Dia-Fc-scFv)
MNLLLILTFVAAAVAEFEVQLVESGGGLVQPGGSLRLSCAASGFNIGSSSIHWVRQAPGKGLEWVASIYSAFASTSYADSVKGRFTIS
ADTSKNTAYLQMNSLRAEDTAVYYCARYHFPFGFALDYWGQGTLVTVSSGGGGSDIQMTQSPSSLSASVGDRVTITCRASQSVSSAVA
WYQQKPGKAPKLLIYSASSLYSGVPSRFSGSRSGTDFTLTISSLQPEDFATYYCQQGVYLFTFGQGTKVEIKLEDKTHTKVEPKTSDK
THTCPPCPAPELLGGPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPMVFDLPPSREEMTKNQVSLWCMVKGFYPSDIAVEWESNGQPENNYKTTPPVL
DSDGSFFLYSKLTVDKSRWQQGNVFSCSVMHEALHNHYTQKSLSLSPGKSGSETPGTSESATPESGGGDIQMTQSPSSLSASVGDRVT
ITCRASQSVSSAVAWYQQKPGKAPKLLIYSASSLYSGVPSRFSGSRSGTDFTLTISSLQPEDFATYYCQQYYWPITFGQGTKVEIKGT
TAASGSSGGSSSGAEVQLVESGGGLVQPGGSLRLSCAASGFNISSYYIHWVRQAPGKGLEWVASIYSSYGYTSYADSVKGRFTISADT
SKNTAYLQMNSLRAEDTAVYYCARTVRGSKKPYFSGWAMDYWGQGTLVTVSS (SEQ ID NO: 1001)
CM0109 Hole (Dia-Fc-scFv)
MNLLLILTFVAAAVAEFEVQLVESGGGLVQPGGSLRLSCAASGFNIGSSSIHWVRQAPGKGLEWVASIYSAFASTSYADSVKGRFTIS
ADTSKNTAYLQMNSLRAEDTAVYYCARYHFPFGFALDYWGQGTLVTVSSGGGGSDIQMTQSPSSLSASVGDRVTITCRASQSVSSAVA
WYQQKPGKAPKLLIYSASSLYSGVPSRFSGSRSGTDFTLTISSLQPEDFATYYCQQGVYLFTFGQGTKVEIKLEDKTHTKVEPKSSDK
THNCPPCPAPELLGGPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPIRELMTSNQVSLSCAVKGFYPSDIAVEWESNGQPENNYKTTPPVL
DSDGSFFLVSKLTVDKSRWQQGNVFSCSVMHEALHNHYTQKSLSLSPGKSGSETPGTSESATPESGGGDIQMTQSPSSLSASVGDRVT
ITCRASQSVSSAVAWYQQKPGKAPKLLIYSASSLYSGVPSRFSGSRSGTDFTLTISSLQPEDFATYYCQQYYWPITFGQGTKVEIKGT
TAASGSSGGSSSGAEVQLVESGGGLVQPGGSLRLSCAASGFNISSYYIHWVRQAPGKGLEWVASIYSSYGYTSYADSVKGRFTISADT
SKNTAYLQMNSLRAEDTAVYYCARTVRGSKKPYFSGWAMDYWGQGTLVTVSS (SEQ ID NO: 1000)
CM0109 Knob (Dia-Fc-scFv)
MNLLLILTFVAAAVAEFEVQLVESGGGLVQPGGSLRLSCAASGFNIGSSSIHWVRQAPGKGLEWVASIYSAFASTSYADSVKGRFTIS
ADTSKNTAYLQMNSLRAEDTAVYYCARYHFPFGFALDYWGQGTLVTVSSGGGGSDIQMTQSPSSLSASVGDRVTITCRASQSVSSAVA
WYQQKPGKAPKLLIYSASSLYSGVPSRFSGSRSGTDFTLTISSLQPEDFATYYCQQGVYLFTFGQGTKVEIKLEDKTHTKVEPKTSDK
THTCPPCPAPELLGGPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPMVFDLPPSREEMTKNQVSLWCMVKGFYPSDIAVEWESNGQPENNYKTTPPVL
DSDGSFFLYSKLTVDKSRWQQGNVFSCSVMHEALHNHYTQKSLSLSPGKSGSETPGTSESATPESGGGDIQMTQSPSSLSASVGDRVT
ITCRASQSVSSAVAWYQQKPGKAPKLLIYSASSLYSGVPSRFSGSRSGTDFTLTISSLQPEDFATYYCQQYSWGPFTFGQGTKVEIKG
TTAASGSSGGSSSGAEVQLVESGGGLVQPGGSLRLSCAASGFNISYSSIHWVRQAPGKGLEWVAYISSYYGYTYYADSVKGRFTISAD
TSKNTAYLQMNSLRAEDTAVYYCARAHYFPWAGAMDYWGQGTLVTVSS (SEQ ID NO: 999)
CT003 Hole (Dia-Fc-scFv)
MNLLLILTFVAAAVAEFEVQLVESGGGLVQPGGSLRLSCAASGFNISSYYIHWVRQAPGKGLEWVASIYSSYGYTSYADSVKGRFTIS
ADTSKNTAYLQMNSLRAEDTAVYYCARTVRGSKKPYFSGWAMDYWGQGTLVTVSSGGGGSDIQMTQSPSSLSASVGDRVTITCRASQS
VSSAVAWYQQKPGKAPKLLIYSASSLYSGVPSRFSGSRSGTDFTLTISSLQPEDFATYYCQQYSWGPFTFGQGTKVEIKLEDKTHTKV
EPKSSDKTHNCPPCPAPELLGGPSVFLFPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPIRELMTSNQVSLSCAVKGFYPSDIAVEWESNGQPENNY
KTTPPVLDSDGSFFLVSKLTVDKSRWQQGNVFSCSVMHEALHNHYTQKSLSLSPGKSGSETPGTSESATPESGGGEVQLVESGGGLVQ
PGGSLRLSCAASGFNIHSSSIHWVRQAPGKGLEWVAATYSSFGSITYADSVKGRFTISADTSKNTAYLQMNSLRAEDTAVYYCARYHH
PFGYALDYWGQGTLVTVSSGTTAASGSSGGSSSGADIQMTQSPSSLSASVGDRVTITCRASQSVSSAVAWYQQKPGKAPKLLIYSASS
LYSGVPSRFSGSRSGTDFTLTISSLQPEDFATYYCQQGVYLFTFGQGTKVEIK (SEQ ID NO: 1002)
CT003 Knob (Dia-Fc-scFv)
MNLLLILTFVAAAVAEFEVQLVESGGGLVQPGGSLRLSCAASGFNISYSSIHWVRQAPGKGLEWVAYISSYYGYTYYADSVKGRFTIS
ADTSKNTAYLQMNSLRAEDTAVYYCARAHYFPWAGAMDYWGQGTLVTVSSGGGGDIQMTQSPSSLSASVGDRVTITCRASQSVSSAVA
WYQQKPGKAPKLLIYSASSLYSGVPSRFSGSRSGTDFTLTISSLQPEDFATYYCQQYYWPITFGQGTKVEIKLEDKTHTKVEPKTSDK
THTCPPCPAPELLGGPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPMVFDLPPSREEMTKNQVSLWCMVKGFYPSDIAVEWESNGQPENNYKTTPPVL
DSDGSFFLYSKLTVDKSRWQQGNVFSCSVMHEALHNHYTQKSLSLSPGKSGSETPGTSESATPESGGGEVQLVESGGGLVQPGGSLRL
SCAASGFNIHSSSIHWVRQAPGKGLEWVAATYSSFGSITYADSVKGRFTISADTSKNTAYLQMNSLRAEDTAVYYCARYHHPFGYALD
YWGQGTLVTVSSGTTAASGSSGGSSSGADIQMTQSPSSLSASVGDRVTITCRASQSVSSAVAWYQQKPGKAPKLLIYSASSLYSGVPS
RFSGSRSGTDFTLTISSLQPEDFATYYCQQGVYLFTFGQGTKVEIK (SEQ ID NO: 1003)
CM0112 Hole (scFv-Fc-Dia)
MNLLLILTFVAAAVAEFDIQMTQSPSSLSASVGDRVTITCRASQSVSSAVAWYQQKPGKAPKLLIYSASSLYSGVPSRFSGSRSGTDF
TLTISSLQPEDFATYYCQQGVYLFTFGQGTKVEIKGTTAASGSSGGSSSGAEVQLVESGGGLVQPGGSLRLSCAASGFNIGSSSIHWV
RQAPGKGLEWVASIYSAFASTSYADSVKGRFTISADTSKNTAYLQMNSLRAEDTAVYYCARYHFPFGFALDYWGQGTLVTVSSLEDKT
HTKVEPKSSDKTHNCPPCPAPELLGGPSVFLFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPPIRELMTSNQVSLSCAVKGFYPSDIAVEWESNGQP
ENNYKTTPPVLDSDGSFFLVSKLTVDKSRWQQGNVFSCSVMHEALHNHYTQKSLSLSPGKSGSETPGTSESATPESGGGEVQLVESGG
GLVQPGGSLRLSCAASGFNISSYYIHWVRQAPGKGLEWVASIYSSYGYTSYADSVKGRFTISADTSKNTAYLQMNSLRAEDTAVYYCA
RTVRGSKKPYFSGWAMDYWGQGTLVTVSSGGGGSDIQMTQSPSSLSASVGDRVTITCRASQSVSSAVAWYQQKPGKAPKLLIYSASSL
YSGVPSRFSGSRSGTDFTLTISSLQPEDFATYYCQQYSWGPFTFGQGTKVEIK (SEQ ID NO: 1004)
CM0112 Knob (scFv-Fc-Dia)
MNLLLILTFVAAAVAEFDIQMTQSPSSLSASVGDRVTITCRASQSVSSAVAWYQQKPGKAPKLLIYSASSLYSGVPSRFSGSRSGTDF
TLTISSLQPEDFATYYCQQGVYLFTFGQGTKVEIKGTTAASGSSGGSSSGAEVQLVESGGGLVQPGGSLRLSCAASGFNIGSSSIHWV
RQAPGKGLEWVASIYSAFASTSYADSVKGRFTISADTSKNTAYLQMNSLRAEDTAVYYCARYHFPFGFALDYWGQGTLVTVSSLEDKT
HTKVEPKTSDKTHTCPPCPAPELLGGPSVFLFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPMVFDLPPSREEMTKNQVSLWCMVKGFYPSDIAVEWESNGQP
ENNYKTTPPVLDSDGSFFLYSKLTVDKSRWQQGNVFSCSVMHEALHNHYTQKSLSLSPGKSGSETPGTSESATPESGGGEVQLVESGG
GLVQPGGSLRLSCAASGFNISYSSIHWVRQAPGKGLEWVAYISSYYGYTYYADSVKGRFTISADTSKNTAYLQMNSLRAEDTAVYYCA
RAHYFPWAGAMDYWGQGTLVTVSSGGGGSDIQMTQSPSSLSASVGDRVTITCRASQSVSSAVAWYQQKPGKAPKLLIYSASSLYSGVP
SRFSGSRSGTDFTLTISSLQPEDFATYYCQQYYWPITFGQGTKVEIK (SEQ ID NO: 1005)
CM0110 Hole (scFv-Fc-scFv)
MNLLLILTFVAAAVAEFDIQMTQSPSSLSASVGDRVTITCRASQSVSSAVAWYQQKPGKAPKLLIYSASSLYSGVPSRFSGSRSGTDF
TLTISSLQPEDFATYYCQQGVYLFTFGQGTKVEIKGTTAASGSSGGSSSGAEVQLVESGGGLVQPGGSLRLSCAASGFNIGSSSIHWV
RQAPGKGLEWVASIYSAFASTSYADSVKGRFTISADTSKNTAYLQMNSLRAEDTAVYYCARYHFPFGFALDYWGQGTLVTVSSLEDKT
HTKVEPKSSDKTHNCPPCPAPELLGGPSVFLFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPPIRELMTSNQVSLSCAVKGFYPSDIAVEWESNGQP
ENNYKTTPPVLDSDGSFFLVSKLTVDKSRWQQGNVFSCSVMHEALHNHYTQKSLSLSPGKSGSETPGTSESATPESGGGDIQMTQSPS
SLSASVGDRVTITCRASQSVSSAVAWYQQKPGKAPKLLIYSASSLYSGVPSRFSGSRSGTDFTLTISSLQPEDFATYYCQQYYWPITF
GQGTKVEIKGTTAASGSSGGSSSGAEVQLVESGGGLVQPGGSLRLSCAASGFNISSYYIHWVRQAPGKGLEWVASIYSSYGYTSYADS
VKGRFTISADTSKNTAYLQMNSLRAEDTAVYYCARTVRGSKKPYFSGWAMDYWGQGTLVTVSS (SEQ ID NO: 1006)
CM0110 Knob (scFv-Fc-scFv)
MNLLLILTFVAAAVAEFDIQMTQSPSSLSASVGDRVTITCRASQSVSSAVAWYQQKPGKAPKLLIYSASSLYSGVPSRFSGSRSGTDF
TLTISSLQPEDFATYYCQQGVYLFTFGQGTKVEIKGTTAASGSSGGSSSGAEVQLVESGGGLVQPGGSLRLSCAASGFNIGSSSIHWV
RQAPGKGLEWVASIYSAFASTSYADSVKGRFTISADTSKNTAYLQMNSLRAEDTAVYYCARYHFPFGFALDYWGQGTLVTVSSLEDKT
HTKVEPKTSDKTHTCPPCPAPELLGGPSVFLFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPMVFDLPPSREEMTKNQVSLWCMVKGFYPSDIAVEWESNGQP
ENNYKTTPPVLDSDGSFFLYSKLTVDKSRWQQGNVFSCSVMHEALHNHYTQKSLSLSPGKSGSETPGTSESATPESGGGDIQMTQSPS
SLSASVGDRVTITCRASQSVSSAVAWYQQKPGKAPKLLIYSASSLYSGVPSRFSGSRSGTDFTLTISSLQPEDFATYYCQQYSWGPFT
FGQGTKVEIKGTTAASGSSGGSSSGAEVQLVESGGGLVQPGGSLRLSCAASGFNISYSSIHWVRQAPGKGLEWVAYISSYYGYTYYAD
SVKGRFTISADTSKNTAYLQMNSLRAEDTAVYYCARAHYFPWAGAMDYWGQGTLVTVSS (SEQ ID NO: 1007)
CT001 CM0299 CM0300 CM0301 ANT1 LC
MNLLLILTFVAAAVADIQMTQSPSSLSASVGDRVTITCRASQSVSSAVAWYQQKPGKAPKLLIYSASSLYSGVPSRFSGSRSGTDFTL
TISSLQPEDFATYYCQQGVYLFTFGQGTKVEIKRTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC (SEQ ID NO: 1008)
CT001 Hole (bsIgG-dia)
MNLLLILTFVAAAVAEVQLVESGGGLVQPGGSLRLSCAASGFNIHSSSIHWVRQAPGKGLEWVAATYSSFGSITYADSVKGRFTISAD
TSKNTAYLQMNSLRAEDTAVYYCARYHHPFGYALDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTVLHQDWLNGKEYKCKVSNKALPAPIEK
TISKAKGQPREPQVYTLPPIRELMTSNQVSLSCAVKGFYPSDIAVEWESNGQPENNYKTTPPVLDSDGSFFLVSKLTVDKSRWQQGNV
FSCSVMHEALHNHYTQKSLSLSPGKSGSETPGTSESATPESGGGEVQLVESGGGLVQPGGSLRLSCAASGFNISSYYIHWVRQAPGKG
LEWVASIYSSYGYTSYADSVKGRFTISADTSKNTAYLQMNSLRAEDTAVYYCARTVRGSKKPYFSGWAMDYWGQGTLVTVSSGGGGSD
IQMTQSPSSLSASVGDRVTITCRASQSVSSAVAWYQQKPGKAPKLLIYSASSLYSGVPSRFSGSRSGTDFTLTISSLQPEDFATYYCQ
QYSWGPFTFGQGTKVEIK (SEQ ID NO: 1009)
CT001 Knob (bsIgG-dia)
MNLLLILTFVAAAVAEVQLVESGGGLVQPGGSLRLSCAASGFNIHSSSIHWVRQAPGKGLEWVAATYSSFGSITYADSVKGRFTISAD
TSKNTAYLQMNSLRAEDTAVYYCARYHHPFGYALDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTVLHQDWLNGKEYKCKVSNKALPAPIEK
TISKAKGQPREPMVFDLPPSREEMTKNQVSLWCMVKGFYPSDIAVEWESNGQPENNYKTTPPVLDSDGSFFLYSKLTVDKSRWQQGNV
FSCSVMHEALHNHYTQKSLSLSPGKSGSETPGTSESATPESGGGEVQLVESGGGLVQPGGSLRLSCAASGFNISYSSIHWVRQAPGKG
LEWVAYISSYYGYTYYADSVKGRFTISADTSKNTAYLQMNSLRAEDTAVYYCARAHYFPWAGAMDYWGQGTLVTVSSGGGGSDIQMTQ
SPSSLSASVGDRVTITCRASQSVSSAVAWYQQKPGKAPKLLIYSASSLYSGVPSRFSGSRSGTDFTLTISSLQPEDFATYYCQQYYWP
ITFGQGTKVEIK (SEQ ID NO: 1010)
CM0299 Hc (IgG-Dia)
MNLLLILTFVAAAVAEVQLVESGGGLVQPGGSLRLSCAASGFNIHSSSIHWVRQAPGKGLEWVAATYSSFGSITYADSVKGRFTISAD
TSKNTAYLQMNSLRAEDTAVYYCARYHHPFGYALDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGSFFLYSKLTVDKSRWQQGNV
FSCSVMHEALHNHYTQKSLSLSPGKSGSETPGTSESATPESGGGEVQLVESGGGLVQPGGSLRLSCAASGFNISYSSIHWVRQAPGKG
LEWVAYISSYYGYTYYADSVKGRFTISADTSKNTAYLQMNSLRAEDTAVYYCARAHYFPWAGAMDYWGQGTLVTVSSGGGGSDIQMTQ
SPSSLSASVGDRVTITCRASQSVSSAVAWYQQKPGKAPKLLIYSASSLYSGVPSRFSGSRSGTDFTLTISSLQPEDFATYYCQQYSWG
PFTFGQGTKVEIK (SEQ ID NO: 1011)
CM0300 Hc (IgG-scFv)
MNLLLILTFVAAAVAEVQLVESGGGLVQPGGSLRLSCAASGFNIHSSSIHWVRQAPGKGLEWVAATYSSFGSITYADSVKGRFTISAD
TSKNTAYLQMNSLRAEDTAVYYCARYHHPFGYALDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGSFFLYSKLTVDKSRWQQGNV
FSCSVMHEALHNHYTQKSLSLSPGKSGSETPGTSESATPESGGGDIQMTQSPSSLSASVGDRVTITCRASQSVSSAVAWYQQKPGKAP
KLLIYSASSLYSGVPSRFSGSRSGTDFTLTISSLQPEDFATYYCQQYSWGPFTFGQGTKVEIKGTTAASGSSGGSSSGAEVQLVESGG
GLVQPGGSLRLSCAASGFNISYSSIHWVRQAPGKGLEWVAYISSYYGYTYYADSVKGRFTISADTSKNTAYLQMNSLRAEDTAVYYCA
RAHYFPWAGAMDYWGQGTLVTVSS (SEQ ID NO: 1012)
CM0301 Hc (IgG-scFv)
MNLLLILTFVAAAVAEVQLVESGGGLVQPGGSLRLSCAASGFNIHSSSIHWVRQAPGKGLEWVAATYSSFGSITYADSVKGRFTISAD
TSKNTAYLQMNSLRAEDTAVYYCARYHHPFGYALDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGSFFLYSKLTVDKSRWQQGNV
FSCSVMHEALHNHYTQKSLSLSPGKSGSETPGTSESATPESGGGDIQMTQSPSSLSASVGDRVTITCRASQSVSSAVAWYQQKPGKAP
KLLIYSASSLYSGVPSRFSGSRSGTDFTLTISSLQPEDFATYYCQQYYWPITFGQGTKVEIKGTTAASGSSGGSSSGAEVQLVESGGG
LVQPGGSLRLSCAASGFNISSYYIHWVRQAPGKGLEWVASIYSSYGYTSYADSVKGRFTISADTSKNTAYLQMNSLRAEDTAVYYCAR
TVRGSKKPYFSGWAMDYWGQGTLVTVSS (SEQ ID NO: 1013)
ANT1 Hole (Dia-Fc-Fab)
MNLLLILTFVAAAVAEFEVQLVESGGGLVQPGGSLRLSCAASGFNISSYYIHWVRQAPGKGLEWVASIYSSYGYTSYADSVKGRFTIS
ADTSKNTAYLQMNSLRAEDTAVYYCARTVRGSKKPYFSGWAMDYWGQGTLVTVSSGGGGSDIQMTQSPSSLSASVGDRVTITCRASQS
VSSAVAWYQQKPGKAPKLLIYSASSLYSGVPSRFSGSRSGTDFTLTISSLQPEDFATYYCQQYYFLITFGQGTKVEIKLEDKTHTKVE
PKSSDKTHNCPPCPAPELLGGPSVFLFPPKPKDTLMISRTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPIRELMTSNQVSLSCAVKGFYPSDIAVEWESNGQPENNYK
TTPPVLDSDGSFFLVSKLTVDKSRWQQGNVFSCSVMHEALHNHYTQKSLSLSPGKSGSETPGTSESATPESGGGEVQLVESGGGLVQP
GGSLRLSCAASGFNIHSSSIHWVRQAPGKGLEWVAATYSSFGSITYADSVKGRFTISADTSKNTAYLQMNSLRAEDTAVYYCARYHHP
FGYALDYWGQGTLVTVSSASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHT (SEQ ID NO: 1014)
ANT1 Knob (Dia-Fc-Fab)
MNLLLILTFVAAAVAEFEVQLVESGGGLVQPGGSLRLSCAASGFNISYSSIHWVRQAPGKGLEWVASISPYYGYTYYADSVKGRFTIS
ADTSKNTAYLQMNSLRAEDTAVYYCARSSYFPWESAMDYWGQGTLVTVSSGGGGSDIQMTQSPSSLSASVGDRVTITCRASQSVSSAV
AWYQQKPGKAPKLLIYSASSLYSGVPSRFSGSRSGTDFTLTISSLQPEDFATYYCQQYYWPITFGQGTKVEIKLEDKTHTKVEPKTSD
KTHTCPPCPAPELLGGPSVFLFPPKPKDTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPMVFDLPPSREEMTKNQVSLWCMVKGFYPSDIAVEWESNGQPENNYKTTPPV
LDSDGSFFLYSKLTVDKSRWQQGNVFSCSVMHEALHNHYTQKSLSLSPGKSGSETPGTSESATPESGGGEVQLVESGGGLVQPGGSLR
LSCAASGFNIHSSSIHWVRQAPGKGLEWVAATYSSFGSITYADSVKGRFTISADTSKNTAYLQMNSLRAEDTAVYYCARYHHPFGYAL
DYWGQGTLVTVSSASTKGPSVFPLAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHT (SEQ ID NO: 1015)
Fab-Dia Hc (Fab-Dia)
MNLLLILTFVAAAVAEVQLVESGGGLVQPGGSLRLSCAASGFNIHSSSIHWVRQAPGKGLEWVAATYSSFGSITYADSVKGRFTISAD
TSKNTAYLQMNSLRAEDTAVYYCARYHHPFGYALDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVEPKSCDKTHTGGSGGEVQLVESGGGLVQPGG
SLRLSCAASGFTISYSSIHWVRQAPGKGLEWVASISPYYGYTYYADSVKGRFTISADTSKNTAYLQMNSLRAEDTAVYYCARSSYFPW
FSAMDYWGQGTLVTVSSGGGGSDIQMTQSPSSLSASVGDRVTITCRASQSVSSAVAWYQQKPGKAPKLLIYSASSLYSGVPSRFSGSR
SGTDFTLTISSLQPEDFATYYCQQYYWPITFGQGTKVEIK (SEQ ID NO: 1016)
Fab-Dia LC (Fab-Dia)
MNLLLILTFVAAAVADIQMTQSPSSLSASVGDRVTITCRASQSVSSAVAWYQQKPGKAPKLLIYSASSLYSGVPSRFSGSRSGTDFTL
TISSLQPEDFATYYCQQGVYLFTFGQGTKVEIKRTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGECGGGGSEVQLVESGGGLVQPGGSLRLSCAASGFTISS
YYIHWVRQAPGKGLEWVASIYSSYGYTSYADSVKGRFTISADTSKNTAYLQMNSLRAEDTAVYYCARTVRGSKKPYFSGWAMDYWGQG
TLVTVSSGGGGSDIQMTQSPSSLSASVGDRVTITCRASQSVSSAVAWYQQKPGKAPKLLIYSASSLYSGVPSRFSGSRSGTDFTLTIS
SLQPEDFATYYCQQYYFLITFGQGTKVEIK (SEQ ID NO: 1017)
CH3 (Dia-CH3-Dia)
MNLLLILTFVAAAVAEFEVQLVESGGGLVQPGGSLRLSCAASGFNIGSSSIHWVRQAPGKGLEWVASIYSAFASTSYADSVKGRFTIS
ADTSKNTAYLQMNSLRAEDTAVYYCARYHFPFGFALDYWGQGTLVTVSSGGGGSDIQMTQSPSSLSASVGDRVTITCRASQSVSSAVA
WYQQKPGKAPKLLIYSASSLYSGVPSRFSGSRSGTDFTLTISSLQPEDFATYYCQQGVYLFTFGQGTKVEIKDKTHTKVEPKTSDKTH
TCPPCPGQPREPQVYTLPPSREEMTKNQVSLTCLVKGFYPSDIAVEWESNGQPENNYKTTPPVLDSDGSFFLYSKLTVDKSRWQQGNV
FSCSVMHEALHNHYTQKSLSLSPGKGTTAASGSSGGSSSGAGRTEVQLVESGGGLVQPGGSLRLSCAASGFNISYSSIHWVRQAPGKG
LEWVAYISSYYGYTYYADSVKGRFTISADTSKNTAYLQMNSLRAEDTAVYYCARAHYFPWAGAMDYWGQGTLVTVSSGGGGSDIQMTQ
SPSSLSASVGDRVTITCRASQSVSSAVAWYQQKPGKAPKLLIYSASSLYSGVPSRFSGSRSGTDFTLTISSLQPEDFATYYCQQYSWG
PFTFGQGTKVEIK (SEQ ID NO: 1018)
CM0156
EVQLVESGGGLVQPGGSLRLSCAASGFNISYSYMHWVRQAPGKGLEWVASISPYYSYTSYADSVKGRFTISADTSKNTAYLQMNSLRA
EDTAVYYCARPSAWSHYYPSSSSSAFDYWGQGTLVTVSSGGGGSDIQMTQSPSSLSASVGDRVTITCRASQSVSSAVAWYQQKPGKAP
KLLIYSASSLYSGVPSRFSGSRSGTDFTLTISSLQPEDFATYYCQQSSYSLITFGQGTKVEIKLEDKTHTKVEPKSSDKTHTCPPCPA
PELLGGPSVFLFPPKPKDTLMISRTPEVTCVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGKSGSETPGTSESATPESGGGEVQLVESGGGLVQPGGSLRLSCAASGFNI
SYSYMHWVRQAPGKGLEWVASISPYYSYTSYADSVKGRFTISADTSKNTAYLQMNSLRAEDTAVYYCARPSAWSHYYPSSSSSAFDYW
GQGTLVTVSSGGGGSDIQMTQSPSSLSASVGDRVTITCRASQSVSSAVAWYQQKPGKAPKLLIYSASSLYSGVPSRFSGSRSGTDFTL
TISSLQPEDFATYYCQQSSYSLITFGQGTKVEIK (SEQ ID NO: 1019)

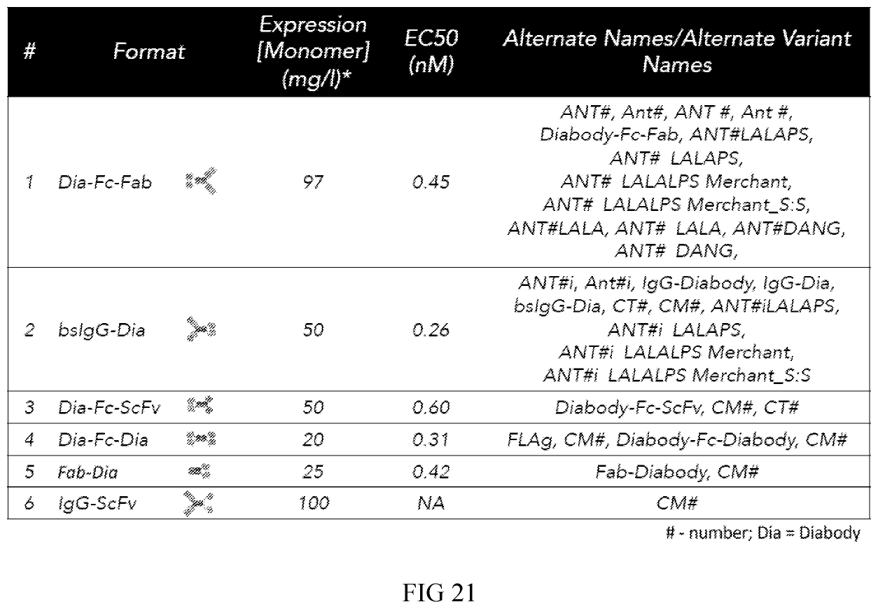

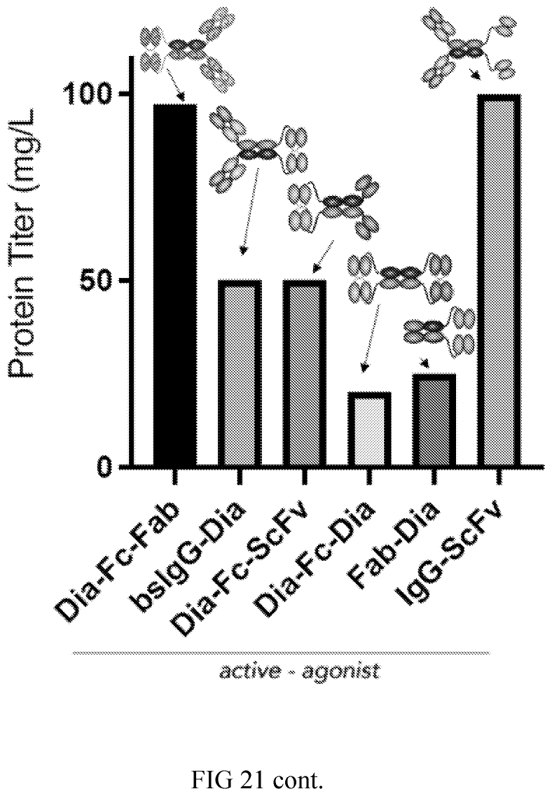

TABLE 14
Comparison of expression titers and monodispersity of FZD agonists
following Protein A purification. See corresponding FIG. 21.

| | | N-term | C-term | Expression | % | Monomer |
|---|---|---|---|---|---|---|
| Molecule | Format | Paratope | Paratope | Titer mg/l | Monomer | Yield (mg/l) |
| CM0042 | Dia - Fc- Dia | 5019 | 2539 | 8 ± 4 | 43 | 3 |
| CM0011 | Dia - Fc- Dia | 5019 | 2539/2542 | 12 ± 3 | 81 ± 5 | 10 |
| CM0126 | Dia - Fc- Dia | 5016 | 2539/2542 | 8 ± 0.7 | ND | ND |
| CM0107 | Dia - Fc - scFv | 5019 | 2539 | 90 | 87 | 80 |
| CM0108 | Dia - Fc - scFv | 5019 | 2542 | 47 | 89 | 40 |
| CM0109 | Dia - Fc - scFv | 5019 | 2539/2542 | 79 | 86 | 70 |
| CT003 | Dia - Fc - scFv | 2539/2542 | 5016 | 47 | 67 | 30 |
| CM0112 | scFv - Fc - Dia | 5019 | 2539/2542 | 15 | 79 | 10 |
| CM0110 | scFv - Fc - scFv | 5019 | 2539/2542 | 82 | 81 | 70 |
| CT001 | bslgG-dia | 5016 | 2539/2542 | 47 ± 21 | 44 ± 11 | 30 |
| CM0299 | lgG-dia | 5016 | 2539 | 20 ± 8 | ND | ND |
| CM0300 | lgG - scFv | 5016 | 2539 | 134 ± 38 | 66 ± 7 | 100 |
| CM0301 | lgG - scFv | 5016 | 2542 | 134 ± 27 | 71 ± 8 | 90 |
| ANT1 | Dia-Fc-Fab | 5016 | 2539/2542 | 294 ± 75 | 53 ± 9 | 150 |
| CH3 | Dia-CH3-Dia | 5019 | 2539 | <1 | ND | ND |
| Fab-Dia | FAb-Dia | 5016 | 2540/2542 | 1 | ND | ND |

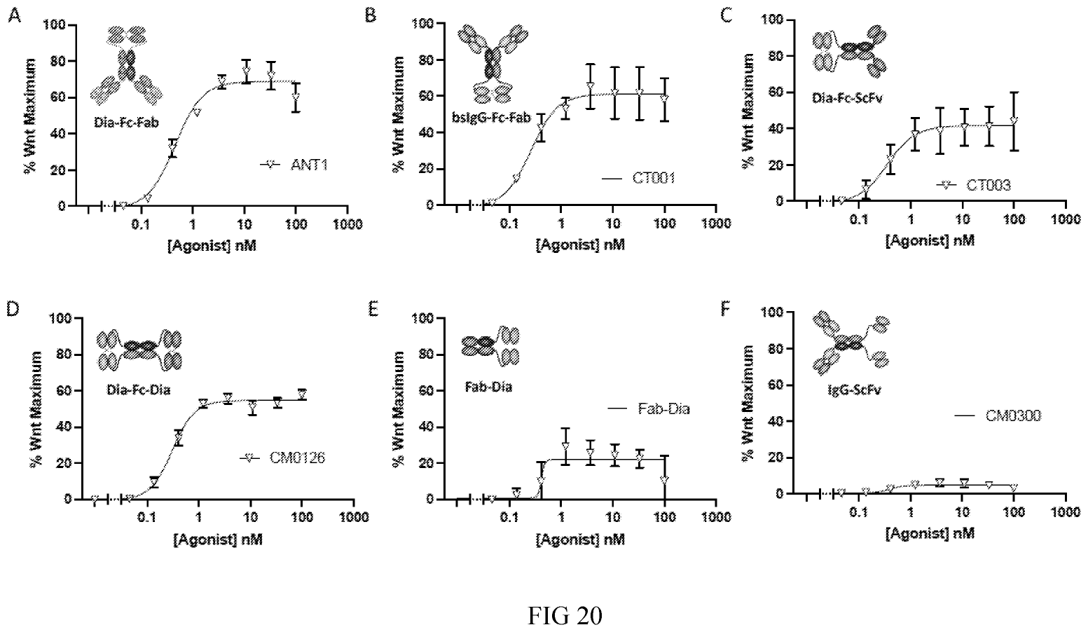

TABLE 15
Functional comparison of FZD agonists. Concentration-response curves of each
FZD agonist were generated using the TOPFLASH assay. FIG. 20 shows representative
traces of the A) Diabody-Fc-Diabody and B) Diabody-Fc-Fab format overlaid with
Wnt3a for comparison. Calculated EC50 and maximum efficacy relative to recombinant
Wnt3a control were derived and are presented as the average ± SD.

| | | | | | Maximal |
|---|---|---|---|---|---|
| | | | | | Signal |
| | | N-term | C-term | EC₅₀ | (% Wnt3a |
| Name | Format | Paratope | Paratope | (nM) | maxima) |
| Wnt3a | Recombinant Protein | NA | NA | 6 ± 3 | 100 |
| CM0042 | Dia - Fc - Dia | 5019 | 2539 | 0.9 ± 0.8 | 33 ± 22 |
| CM0011 | Dia - Fc - Dia | 5019 | 2539/2542 | 0.3 ± 0.1 | 63 ± 10 |
| CM0126 | Dia - Fc - Dia | 5016 | 2539/2542 | 0.3 ± 0.2 | 52 ± 1 |
| CM0107 | Dia-Fc-scFv | 5019 | 2539 | ND | ND |
| CM0108 | Dia-Fc-scFv | 5019 | 2542 | ND | ND |
| CM0109 | Dia-Fc-scFv | 5019 | 2539/2542 | ND | ND |
| CT003 | Dia-Fc-scFv | 2539/2542 | 5016 | 0.4 ± 0.1 | 42 ± 16 |
| CM0112 | scFv-Fc-Dia | 5019 | 2539/2542 | ND | ND |
| CM0110 | scFv-Fc-scFv | 5019 | 2539/2542 | ND | ND |
| CT001 | bslgG-dia | 5016 | 2539/2542 | 0.3 ± 0.1 | 57 ± 17 |
| CM0299 | lgG-dia | 5016 | 2539 | 0.1 ± 0.1 | 14 ± 5 |
| CM0300 | lgG-scFv | 5016 | 2539 | 0.3 ± 0.2 | 5 ± 2 |
| CM0301 | lgG-scFv | 5016 | 2542 | 0.2 ± 0.2 | 1 ± 1 |
| ANT1 | Dia-Fc-Fab | 5016 | 2539/2542 | 0.7 ± 0.5 | 50 ± 18 |
| CH3 | Dia-CH3-Dia | 5019 | 2539 | ND | 22 ± 10 |
| FAb-Dia | Fab-Dia | 5016 | 2540/2542 | 0.4 ± 0.2 | 42 ± 16 |

TABLE 16
FZD2-LRP6 CM0072

| Knob | MNLLLILTFVAAAVAEFEVQLVESGGGLVQPGGSLRLSCAASGFNIYYSSIHWVRQAPGKGLEWVASIYPYYGYTYYADSVKGR |
|---|---|
| construct | FTISADTSKNTAYLQMNSLRAEDTAVYYCARYYHYGLDYWGQGTLVTVSSGGGGSDIQMTQSPSSLSASVGDRVTITCRASQS |
| | VSSAVAWYQQKPGKAPKLLIYSASSLYSGVPSRFSGSRSGTDFTLTISSLQPEDFATYYCQQSYWHSYLITFGQGTKVEIKLEDKT |
| | HTKVEPKTSDKTHTCPPCPAPELLGGPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPMVFDLPPSREEMTKNQVSLWCMVKGFYPSDI |
| | AVEWESNGQPENNYKTTPPVLDSDGSFFLYSKLTVDKSRWQQGNVFSCSVMHEALHNHYTQKSLSLSPGKSGSETPGTSESAT |
| | PESGGGEVQLVESGGGLVQPGGSLRLSCAASGFNISYSSIHWVRQAPGKGLEWVAYISSYYGYTYYADSVKGRFTISADTSKNT |
| | AYLQMNSLRAEDTAVYYCARAHYFPWAGAMDYWGQGTLVTVSSGGGGSDIQMTQSPSSLSASVGDRVTITCRASQSVSSAVA |
| | WYQQKPGKAPKLLIYSASSLYSGVPSRFSGSRSGTDFTLTISSLQPEDFATYYCQQYYWPITFGQGTKVEIK (SEQ ID NO: 1020) |
| Hole | MNLLLILTFVAAAVAEFEVQLVESGGGLVQPGGSLRLSCAASGFNIYYSSIHWVRQAPGKGLEWVASIYPYYGYTYYADSVKGR |
| construct | FTISADTSKNTAYLQMNSLRAEDTAVYYCARYYHYGLDYWGQGTLVTVSSGGGGSDIQMTQSPSSLSASVGDRVTITCRASQS |
| | VSSAVAWYQQKPGKAPKLLIYSASSLYSGVPSRFSGSRSGTDFTLTISSLQPEDFATYYCQQSYWHSYLITFGQGTKVEIKLEDKT |
| | HTKVEPKSSDKTHNCPPCPAPELLGGPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPIRELMTSNQVSLSCAVKGFYPSDIAV |
| | EWESNGQPENNYKTTPPVLDSDGSFFLVSKLTVDKSRWQQGNVFSCSVMHEALHNHYTQKSLSLSPGKSGSETPGTSESATPES |
| | GGGEVQLVESGGGLVQPGGSLRLSCAASGFNISSYYIHWVRQAPGKGLEWVASIYSSYGYTSYADSVKGRFTISADTSKNTAYL |
| | QMNSLRAEDTAVYYCARTVRGSKKPYFSGWAMDYWGQGTLVTVSSGGGGSDIQMTQSPSSLSASVGDRVTITCRASQSVSSA |
| | VAWYQQKPGKAPKLLIYSASSLYSGVPSRFSGSRSGTDFTLTISSLQPEDFATYYCQQYSWGPFTFGQGTKVEIK (SEQ ID NO: |
| | 1021) |

FZD5-LRP6 CM0024

| | |
|---|---|
| Knob | MNLLLILTFVAAAVAEFEVQLVESGGGLVQPGGSLRLSCAASGFNISYSSIHWVRQAPGKGLEWVASIYPSYSSTYYADSVKGR |
| construct | FTISADTSKNTAYLQMNSLRAEDTAVYYCARYYAMDYWGQGTLVTVSSGGGGSDIQMTQSPSSLSASVGDRVTITCRASQSVS |
| | SAVAWYQQKPGKAPKLLIYSASSLYSGVPSRFSGSRSGTDFTLTISSLQPEDFATYYCQQAFYYPITFGQGTKVEIKLEDKTHTKV |
| | EPKTSDKTHTCPPCPAPELLGGPSVFLFPPKPKDTLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPMVFDLPPSREEMTKNQVSLWCMVKGFYPSDIAVEW |
| | ESNGQPENNYKTTPPVLDSDGSFFLYSKLTVDKSRWQQGNVFSCSVMHEALHNHYTQKSLSLSPGKSGSETPGTSESATPESGG |
| | GEVQLVESGGGLVQPGGSLRLSCAASGFNISYSSIHWVRQAPGKGLEWVAYISSYYGYTYYADSVKGRFTISADTSKNTAYLQM |
| | NSLRAEDTAVYYCARAHYFPWAGAMDYWGQGTLVTVSSGGGGSDIQMTQSPSSLSASVGDRVTITCRASQSVSSAVAWYQQKP |
| | GKAPKLLIYSASSLYSGVPSRFSGSRSGTDFTLTISSLQPEDFATYYCQQYYWPITFGQGTKVEIK (SEQ ID NO: 1022) |
| Hole | MNLLLILTFVAAAVAEFEVQLVESGGGLVQPGGSLRLSCAASGFNISYSSIHWVRQAPGKGLEWVASIYPSYSSTYYADSVKGR |
| construct | FTISADTSKNTAYLQMNSLRAEDTAVYYCARYYAMDYWGQGTLVTVSSGGGGSDIQMTQSPSSLSASVGDRVTITCRASQSVS |
| | SAVAWYQQKPGKAPKLLIYSASSLYSGVPSRFSGSRSGTDFTLTISSLQPEDFATYYCQQAFYYPITFGQGTKVEIKLEDKTHTKV |
| | EPKSSDKTHNCPPCPAPELLGGPSVFLFPPKPKDTLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQVYTLPPIRELMTSNQVSLSCAVKGFYPSDIAVEWES |
| | NGQPENNYKTTPPVLDSDGSFFLVSKLTVDKSRWQQGNVFSCSVMHEALHNHYTQKSLSLSPGKSGSETPGTSESATPESGGG |
| | EVQLVESGGGLVQPGGSLRLSCAASGFNISSYYIHWVRQAPGKGLEWVASIYSSYGYTSYADSVKGRFTISADTSKNTAYLQMN |
| | SLRAEDTAVYYCARTVRGSKKPYFSGWAMDYWGQGTLVTVSSGGGGSDIQMTQSPSSLSASVGDRVTITCRASQSVSSAVAWYQ |
| | QKPGKAPKLLIYSASSLYSGVPSRFSGSRSGTDFTLTISSLQPEDFATYYCQQYSWGPFTFGQGTKVEIK (SEQ ID NO: 1023) |

FZD7-LRP6 CM0172

| | |
|---|---|
| Knob | MNLLLILTFVAAAVAEFEVQLVESGGGLVQPGGSLRLSCAASGFNISSSSMHWVRQAPGKGLEWVASIYSYYGSTYYADSVKG |
| construct | RFTISADTSKNTAYLQMNSLRAEDTAVYYCARWYGMDYWGQGTLVTVSSGGGGSDIQMTQSPSSLSASVGDRVTITCRASQS |
| | VSSAVAWYQQKPGKAPKLLIYSASSLYSGVPSRFSGSRSGTDFTLTISSLQPEDFATYYCQQPGSWYFPPITFGQGTKVEIKLEDK |
| | THTKVEPKTSDKTHTCPPCPAPELLGGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPMVFDLPPSREEMTKNQVSLWCMVKGFYPSD |
| | IAVEWESNGQPENNYKTTPPVLDSDGSFFLYSKLTVDKSRWQQGNVFSCSVMHEALHNHYTQKSLSLSPGKSGSETPGTSESA |
| | TPESGGGEVQLVESGGGLVQPGGSLRLSCAASGFNISYSSIHWVRQAPGKGLEWVAYISSYYGYTYYADSVKGRFTISADTSKN |
| | TAYLQMNSLRAEDTAVYYCARAHYFPWAGAMDYWGQGTLVTVSSGGGGSDIQMTQSPSSLSASVGDRVTITCRASQSVSSAVA |
| | WYQQKPGKAPKLLIYSASSLYSGVPSRFSGSRSGTDFTLTISSLQPEDFATYYCQQYYWPITFGQGTKVEIK (SEQ ID NO: 1024) |
| Hole | MNLLLILTFVAAAVAEFEVQLVESGGGLVQPGGSLRLSCAASGFNISSSSMHWVRQAPGKGLEWVASIYSYYGSTYYADSVKG |
| construct | RFTISADTSKNTAYLQMNSLRAEDTAVYYCARWYGMDYWGQGTLVTVSSGGGGSDIQMTQSPSSLSASVGDRVTITCRASQS |
| | VSSAVAWYQQKPGKAPKLLIYSASSLYSGVPSRFSGSRSGTDFTLTISSLQPEDFATYYCQQPGSWYFPPITFGQGTKVEIKLEDK |
| | THTKVEPKSSDKTHNCPPCPAPELLGGPSVFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQPREPQVYTLPPIRELMTSNQVSLSCAVKGFYPSDI |
| | AVEWESNGQPENNYKTTPPVLDSDGSFFLVSKLTVDKSRWQQGNVFSCSVMHEALHNHYTQKSLSLSPGKSGSETPGTSESAT |
| | PESGGGEVQLVESGGGLVQPGGSLRLSCAASGFNISSYYIHWVRQAPGKGLEWVASIYSSYGYTSYADSVKGRFTISADTSKNTA |
| | YLQMNSLRAEDTAVYYCARTVRGSKKPYFSGWAMDYWGQGTLVTVSSGGGGSDIQMTQSPSSLSASVGDRVTITCRASQSVSSA |
| | VAWYQQKPGKAPKLLIYSASSLYSGVPSRFSGSRSGTDFTLTISSLQPEDFATYYCQQYSWGPFTFGQGTKVEIK (SEQ ID NO: |
| | 1025) |

PanFZD-LRP6 CM0011

| | |
|---|---|
| Knob | MNLLLILTFVAAAVAEFEVQLVESGGGLVQPGGSLRLSCAASGFNIGSSSIHWVRQAPGKGLEWVASIYSAFASTSYADSVKGR |
| construct | FTISADTSKNTAYLQMNSLRAEDTAVYYCARYHFPFGFALDYWGQGTLVTVSSGGGGSDIQMTQSPSSLSASVGDRVTITCRA |
| | SQSVSSAVAWYQQKPGKAPKLLIYSASSLYSGVPSRFSGSRSGTDFTLTISSLQPEDFATYYCQQGVYLFTFGQGTKVEIKLEDKT |
| | HTKVEPKTSDKTHTCPPCPAPELLGGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPMVFDLPPSREEMTKNQVSLWCMVKGFYPSD |
| | IAVEWESNGQPENNYKTTPPVLDSDGSFFLYSKLTVDKSRWQQGNVFSCSVMHEALHNHYTQKSLSLSPGKSGSETPGTSESA |
| | TPESGGGEVQLVESGGGLVQPGGSLRLSCAASGFNISYSSIHWVRQAPGKGLEWVAYISSYYGYTYYADSVKGRFTISADTSKN |
| | TAYLQMNSLRAEDTAVYYCARAHYFPWAGAMDYWGQGTLVTVSSGGGGSDIQMTQSPSSLSASVGDRVTITCRASQSVSSAVA |
| | WYQQKPGKAPKLLIYSASSLYSGVPSRFSGSRSGTDFTLTISSLQPEDFATYYCQQYYWPITFGQGTKVEIK (SEQ ID NO: 1026) |
| Hole | MNLLLILTFVAAAVAEFEVQLVESGGGLVQPGGSLRLSCAASGFNIGSSSIHWVRQAPGKGLEWVASIYSAFASTSYADSVKGR |
| construct | FTISADTSKNTAYLQMNSLRAEDTAVYYCARYHFPFGFALDYWGQGTLVTVSSGGGGSDIQMTQSPSSLSASVGDRVTITCRA |
| | SQSVSSAVAWYQQKPGKAPKLLIYSASSLYSGVPSRFSGSRSGTDFTLTISSLQPEDFATYYCQQGVYLFTFGQGTKVEIKLEDKT |
| | HTKVEPKSSDKTHNCPPCPAPELLGGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPPIRELMTSNQVSLSCAVKGFYPSDIA |
| | VEWESNGQPENNYKTTPPVLDSDGSFFLVSKLTVDKSRWQQGNVFSCSVMHEALHNHYTQKSLSLSPGKSGSETPGTSESATP |
| | ESGGGEVQLVESGGGLVQPGGSLRLSCAASGFNISSYYIHWVRQAPGKGLEWVASIYSSYGYTSYADSVKGRFTISADTSKNTA |
| | YLQMNSLRAEDTAVYYCARTVRGSKKPYFSGWAMDYWGQGTLVTVSSGGGGSDIQMTQSPSSLSASVGDRVTITCRASQSVSSA |
| | VAWYQQKPGKAPKLLIYSASSLYSGVPSRFSGSRSGTDFTLTISSLQPEDFATYYCQQYSWGPFTFGQGTKVEIK (SEQ ID NO: |
| | 1027) |

FZD4-LRP6 CM0016

| | |
|---|---|
| Knob | MNLLLILTFVAAAVAEFEVQLVESGGGLVQPGGSLRLSCAASGFNISYYYIHWVRQAPGKGLEWVASIYPSSGYTYYADSVKGR |
| construct | FTISADTSKNTAYLQMNSLRAEDTAVYYCARSSFYWAMDYWGQGTLVTVSSGGGGSDIQMTQSPSSLSASVGDRVTITCRASQ |
| | SVSSAVAWYQQKPGKAPKLLIYSASSLYSGVPSRFSGSRSGTDFTLTISSLQPEDFATYYCQQSYAAYLFTFGQGTKVEIKLEDKT |
| | HTKVEPKSSDKTHNCPPCPAPELLGGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPPIRELMTSNQVSLSCAVKGFYPSDIA |
| | VEWESNGQPENNYKTTPPVLDSDGSFFLVSKLTVDKSRWQQGNVFSCSVMHEALHNHYTQKSLSLSPGKSGSETPGTSESATP |
| | ESGGGEVQLVESGGGLVQPGGSLRLSCAASGFNISSYYIHWVRQAPGKGLEWVASIYSSYGYTSYADSVKGRFTISADTSKNTA |
| | YLQMNSLRAEDTAVYYCARTVRGSKKPYFSGWAMDYWGQGTLVTVSSGGGGSDIQMTQSPSSLSASVGDRVTITCRASQSVSSA |
| | VAWYQQKPGKAPKLLIYSASSLYSGVPSRFSGSRSGTDFTLTISSLQPEDFATYYCQQYSWGPFTFGQGTKVEIK (SEQ ID NO: |
| | 1028) |
| Hole | MNLLLILTFVAAAVAEFEVQLVESGGGLVQPGGSLRLSCAASGFNISYYYIHWVRQAPGKGLEWVASIYPSSGYTYYADSVKGR |
| construct | FTISADTSKNTAYLQMNSLRAEDTAVYYCARSSFYWAMDYWGQGTLVTVSSGGGGSDIQMTQSPSSLSASVGDRVTITCRASQ |
| | SVSSAVAWYQQKPGKAPKLLIYSASSLYSGVPSRFSGSRSGTDFTLTISSLQPEDFATYYCQQSYAAYLFTFGQGTKVEIKLEDKT |
| | HTKVEPKTSDKTHTCPPCPAPELLGGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPMVFDLPPSREEMTKNQVSLWCMVKGFYPSD |
| | IAVEWESNGQPENNYKTTPPVLDSDGSFFLYSKLTVDKSRWQQGNVFSCSVMHEALHNHYTQKSLSLSPGKSGSETPGTSESA |
| | TPESGGGEVQLVESGGGLVQPGGSLRLSCAASGFNISYSSIHWVRQAPGKGLEWVAYISSYYGYTYYADSVKGRFTISADTSKN |
| | TAYLQMNSLRAEDTAVYYCARAHYFPWAGAMDYWGQGTLVTVSSGGGGSDIQMTQSPSSLSASVGDRVTITCRASQSVSSAVA |
| | WYQQKPGKAPKLLIYSASSLYSGVPSRFSGSRSGTDFTLTISSLQPEDFATYYCQQYYWPITFGQGTKVEIK (SEQ ID NO: 1029) |

1. A tetravalent binding antibody molecule comprising:
(a) an Fc domain or fragment thereof comprising a constant heavy chain domain 3 (CH3),
(b) a bivalent low-density lipoprotein receptor-related protein (LRP) binding domain, and
(c) a bivalent Frizzled (FZD) binding domain,
wherein the LRP binding domain is attached to one terminus of the Fc domain and the FZD binding domain is attached to the other end of the Fc domain,
wherein the LRP binding domain is selected from the group consisting of: a diabody that binds LRP5, a diabody that binds LRP6, two scFv that bind LRP5, two scFv that bind LRP6, two Fab that bind LRP5, and two Fab that bind LRP6 and combinations thereof; and
wherein the FZD binding domain is selected from the group consisting of: a diabody that binds one or more FZD, two scFv that bind one or more FZD, two Fab that bind one or more FZD wherein the FZD is a Frizzled Class Receptor 1 (FZD1), Frizzled Class Receptor 2 (FZD2), Frizzled Class Receptor 3 (FZD3), Frizzled Class Receptor 5 (FZD4), Frizzled Class Receptor 5 (FZD5), Frizzled Class Receptor 6 (FZD6), Frizzled Class Receptor 7 (FZD7), Frizzled Class Receptor 8 (FZD8), Frizzled Class Receptor 9 (FZD9), or Frizzled Class Receptor 10 (FZD10)
wherein when the LRP binding domain binds only LRP5, the FZD binding domain binds a FZD selected from the group consisting of FZD1, FZD2, FZD3, FZD5, FZD6, FZD7, FZD8, FZD9, FZD10, and combinations thereof.

2. The tetravalent binding antibody molecule of claim 1, wherein
(a) the LRP binding domain diabody is attached to the N-terminal of the Fc domain, and
(b) the FZD binding domain is attached to the C-terminal of the Fc domain.

3. The tetravalent binding antibody molecule of claim 2, wherein
(a) the LRP binding domain diabody is attached to the N-terminal of the Fc domain via a VL or VH of the diabody, and
(b) the FZD binding domain comprises two FZD binding Fab fused to the C-terminal of the Fc, wherein each Fab is attached to the Fc domain via a heavy or light chain variable domain (VH or VL) of the Fab linked to the CH3 domain of the Fc domain.

4. The tetravalent binding antibody molecule of claim 1, wherein the FZD binding domain is attached to the N-terminal of the Fc domain and the LRP binding domain is attached to the C-terminal of the Fc domain.

5. The tetravalent binding antibody molecule of claim 4, wherein
(a) the FZD binding domain comprises two Fabs that bind the FZD, wherein each Fab is attached to N-terminal of the Fc domain via a heavy or light chain variable domain (VH or VL) of the Fab linked to the CH2 domain of the Fc domain, and
(b) the LRP binding domain comprises a diabody or two scFv that bind LRP5 and/or LRP6, wherein the diabody or two scFv are attached to the C-terminal of the Fc domain via a VL or VH of the diabody or scFv linked to the CH3 of the Fc domain.

6. The tetravalent binding antibody molecule of claim 1, wherein the LRP binding domain and/or the FZD binding domain is bispecific.

7. The tetravalent binding antibody molecule of claim 6 wherein the LPR binding domain binds to LRP5 and LRP6.

8. The tetravalent binding antibody molecule of claim 7 wherein the LPR binding domain binds to a Wnt3A site (E3E4 domains) on LRP5 and a Wnt3A site (E3E4 domains) on LRP6.

9. The tetravalent binding antibody molecule of claim 1, wherein
(a) the diabody of the LRP binding domain binds LRP5 and comprises heavy chain complementary determining regions CDR-H1, CDR-H2 and CDR-H3 and light chain complementary determining regions CDR-L1, CDR-L2 and CDR-L3, of an antibody of Table 3 or Table 6, or
(b) the diabody of the LRP binding domain binds LRP6 and comprises heavy chain complementary determining regions CDR-H1, CDR-H2 and CDR-H3 and light chain complementary determining regions CDR-L1, CDR-L2 and CDR-L3 of an antibody of Table 4 or Table 6, or
(c) the diabody of the LRP binding domain binds LRP5 and LRP6, the diabody comprising CDR-H1, CDR-H2 and CDR-H3 and CDR-L1, CDR-L2 and CDR-L3 of an antibody of Table 3 or Table 6 and CDR-H1, CDR-H2 and CDR-H3 and CDR-L1, CDR-L2 and CDR-L3 of an antibody of Table 4.

10. The tetravalent binding antibody molecule of claim 1 wherein the Fc domain or fragment thereof is in a knob-in-hole configuration.

11. The tetravalent binding antibody molecule of claim 1, wherein the Fc domain lacks one or more effector functions.

12. The tetravalent binding antibody molecule of claim 1, wherein the LRP binding domain and FZD binding domain are each attached to the Fc domain by a linker.

13. The tetravalent binding antibody molecule of claim 12, wherein the linker comprises 1 to 100, 1 to 50, 1-30, 1-25, 1-10, 1-6 amino acids, 1-5 amino acids, or 2-4 amino acids.

14. The tetravalent binding antibody molecule of claim 1, wherein the LRP binding domain binds LRP6 or binds LRP5 and LRP6, and the FZD binding domain binds a FZD selected from the group consisting of: FZD1, FZD2, FZD3, FZD4, FZD5, FZD6, FZD7, FZD8, FZD9, and FZD10.

15. The tetravalent binding antibody molecule of claim 14, wherein the FZD binding domain binds FZD4 or FZD5.

16. The tetravalent binding antibody molecule of claim 15, wherein the FZD binding domain comprises two Fabs that bind FZD4 or FZD5 or multiple FZD.

17. The tetravalent binding antibody molecule of claim 16, wherein the FZD-binding Fab comprise light chain complementary determining regions CDR-L1, CDR-L2 and CDR-L3 and heavy chain CDRs, CDR-H1, CDR-H2 and CDR-H3 of an antibody of Table 1, Table 2 or Table 6.

18. The tetravalent binding antibody molecule of claim 15, wherein the LRP binding diabody binds to a Wnt3A site on LRP5 and a Wnt 3A site on LRP6.

19. The tetravalent binding antibody molecule of claim 1, wherein
(a) the diabody of the LRP binding domain binds LRP5 comprises heavy chain complementary determining regions CDR-H1, CDR-H2 and CDR-H3 and light chain complementary determining regions CDR-L1, CDR-L2 and CDR-L3, of an antibody of Table 3 or Table 6, or
(b) the diabody of the LRP binding domain binds LRP6 and comprises heavy chain complementary determining regions CDR-H1, CDR-H2 and CDR-H3 and light chain complementary determining regions CDR-L1, CDR-L2 and CDR-L3 of an antibody of Table 4 or Table 6, or
(c) the diabody of the LRP binding domain binds LRP5 and LRP6, the diabody comprising
(i) CDR-H1, CDR-H2 and CDR-H3 and CDR-L1, CDR-L2 and CDR-L3 of an antibody of Table 3 or Table 6, and
(ii) CDR-H1, CDR-H2 and CDR-H3 and CDR-L1, CDR-L2 and CDR-L3 of an antibody of Table 4 or Table 6.

20. The tetravalent binding antibody molecule of claim 3 comprising,
(a) a dimer of a first and second heavy chain monomer, each monomer comprising a single-chain polypeptide comprising, from N-terminus to C-terminus:
(1) a peptide comprising a heavy chain variable domain (VH) that binds LRP5 and/or LRP6 and a light chain variable domain (VL) that binds LRP5 and/or LRP6
(2) an Fc region, or fragment thereof comprising the CH3,
(3) a VH that binds FZD4, FZD5, or FZD4 and/or FZD5 and one or more other FZD, and
(4) a constant heavy chain domain 1 (CH1),
wherein
(5) the VH that binds LRP5 comprises heavy chain CDRs (CDR-H1, CDR-H2 and CDR-H3), of an antibody of Table 3 or Table 6, or the VH that binds LRP6 comprises the CDR-H1, CDR-H2 and CDR-H3 of an antibody of Table 4 or Table 6, and
(b) the VL that binds LRP5 comprises light chain CDRs (CDR-L1, CDR-L2 and CDR-L3), of an antibody of Table 3 or Table 6, or the VL that binds LRP6 comprises the CDR-L1, CDR-L2 and CDR-L3 of an antibody of Table 4 or Table 6, and
(c) the VH that binds FZD4, FZD5, or FZD4 and/or FZD5 and one or more other FZD comprises the heavy chain CDRs (CDR-H1, CDR-H2 and CDR-H3), of an antibody of Table 1, Table 2 or Table 6, and
(d) a third and fourth light chain monomer each comprising from N terminus to C terminus a VL that binds FZD4, FZD5, or FZD4 and/or FZD5 and one or more other FZD and a constant light chain domain 1 (CL1), the VL that binds FZD4, FZD5, or FZD4 and/or FZD5 and one or more other FZD comprises the light chain CDRs (CDR-L1, CDR-L2 and CDR-L3), of an antibody of Table 1, Table 2 or Table 6,
wherein the first and second heavy chain monomer dimerize via their Fc regions and the VL and VH that bind LRP5 or LRP6 of the first monomer pair with the VH and VL that bind LRP5 or LRP6 of the second monomer forming a bivalent diabody that binds LRP5 or LRP6, and
the CL1 and VLs that bind FZD4, FZD5, or FZD4 and/or FZD5 and one or more other FZD of the third and fourth light chain monomers pair with the CH1 and VHs that bind FZD4, FZD5, or FZD4 and/or FZD5 and one or more other FZD of the first and second heavy chain monomers forming two Fabs that bind FZD4, FZD5, or FZD4 and/or FZD5 and one or more other FZD, wherein the diabody forms the N-terminal bivalent LRP5 or LRP6 binding domain and the two Fab form the C-terminal bivalent FZD4, FZD5, or FZD4 and/or FZD5 and one or more other FZD binding domain.

21. The tetravalent binding antibody molecule of claim 20, wherein the LRP binding diabody is bispecific, wherein
the CDRs of the VHs that bind LRP5 or LRP6 of the first heavy chain monomer and the CDRs of the VHs that bind LRP5 or LRP6 of the second heavy chain monomer are non-identical, and
the CDRs of the VLs that bind LRP5 and LRP6 of the first heavy chain monomer and the CDRs of the VLs that bind LRP5 and LRP6 second heavy chain monomer are non-identical.

22. The tetravalent binding antibody molecule of claim 20, wherein the first and second monomers dimerize via a knob-in-hole configuration of the Fc regions or fragments thereof.

23. The tetravalent binding antibody molecule of claim 20, wherein the Fc domain lacks one or more effector functions.

24. The tetravalent binding antibody molecule of claim 20, wherein the peptide of (a)(1) is linked to the Fc region by a first peptide linker, and the Fc region is linked to the VH that binds FZD4 or FZD5 or to multiple FZD (pan-FZD) by a second peptide linker and the VH that binds FZD4 or FZD5 or to multiple FZD (pan-FZD) is linked to the CH1 by a third peptide linker, and the VL that binds FZD4 or FZD5 or to multiple FZD (pan-FZD) is linked to the CL1 domain by a peptide linker.

25. The tetravalent binding antibody molecule of claim 24, wherein one or more of the second, third or fourth peptide linker comprises 1 to 100, 1 to 50, 1-30, 1-25, 1-10 or 1-5 amino acids.

26. The tetravalent binding antibody molecule of claim 20, wherein in each first and second heavy chain monomer, the VH that binds to LRP5 or LRP6 is linked to the VL that binds to LRP5 or LRP6 by a peptide linker.

27. The tetravalent binding antibody molecule of claim 26, wherein the peptide linker comprises 1-6 amino acids, 1-5 amino acids, or 2-4 amino acids.

28. The tetravalent binding antibody molecule of claim 20 wherein the tetravalent binding antibody molecule does not comprise a FZD binding domain comprising the CDRs of the FZD-binding antibody 5044 in combination with the Wnt co-receptor binding domain comprising the CDRs of LRP6-binding antibody 2542 and/or antibody 2539.

29. The tetravalent binding antibody molecule of claim 20, wherein the tetravalent binding antibody molecule comprises
(a) a first heavy chain comprising the amino acid sequence of the hole heavy chain construct of FZD5-LRP6 of Table 11, a second heavy chain comprising the amino acid sequence of the knob heavy chain construct of FZD5-LRP6 of Table 11 and a light chain comprising the amino acid sequence of the light chain construct of LRP6 of Table 11 wherein the amino acid sequence of the CDRs are the amino acid sequence of the CDRs of ANT59, or
(b) a first heavy chain comprising the amino acid sequence of the hole heavy chain construct of pFZD-LRP6 of Table 11, a second heavy chain comprising the amino acid a knob heavy chain construct of pFZD-LRP6 of Table 11 and a light chain comprising the amino acid sequence of the light chain construct of pFZD-LRP6 of Table 11 wherein the amino acid sequence of the CDRs are the amino acid sequence of the CDRs of ANT9.

30. A tetravalent binding antibody molecule having an N-terminal FZD4 or FZD5 binding domain comprising two FZD4-binding or two FZD5-binding Fabs and a C-terminal LRP binding domain comprising two LRP binding scFvs, the tetravalent binding antibody molecule comprising
(a) a dimer of a first and second heavy chain monomer, wherein each monomer comprises a single-chain polypeptide comprising, from N-terminus to C-terminus:
(i) a first heavy chain variable domain (VH) that binds FZD 4 or 5
(ii) a heavy chain constant region domain 1 (CH1 domain)
(iii) an Fc region or fragment thereof comprising a constant heavy chain domain 3 (CH3 domain),
(iv) a peptide comprising a VH that binds LRP5 or LRP6, and a first light chain variable domain (VL) that binds LRP5 or LRP6, and
(b) a third and fourth light chain monomer each monomer comprising from N terminus to C terminus a second VL that binds the FZD and a constant light chain domain 1 (CL1 domain),
wherein the first and second heavy chain monomers dimerize via the Fc regions or fragments thereof,
wherein the first VH that binds FZD4 or FZD5 and CH1 domain of the first and second monomer pairs with the second VL and CL1 domain of the third and fourth monomers forming two Fabs that bind FZD4 or FZD5, and the second VH and first VL of each heavy chain monomer pair to form a scFv that binds LRP5 or LRP6,
wherein the LRP binding scFvs form a C-terminal LRP binding domain of the tetravalent binding antibody molecule and the FZD4 or FZD5 binding Fabs form an N-terminus FZD4 or FZD5-binding domain of the tetravalent binding antibody molecule, wherein when the LRP binding domain binds only LRP5, the FZD binding domain binds FZD5.

31. The tetravalent binding antibody molecule of claim 30, wherein
the VH that binds FZD4 or FZD5 comprises the heavy chain CDRs (CDR-H1, CDR-H2 and CDR-H3), of an antibody of Table 1, Table 2 or Table 6,
the VH that binds LRP5 comprises heavy chain CDRs (CDR-H1, CDR-H2 and CDR-H3), of an antibody of Table 3 or Table 6, or the VH that binds LRP6 comprises the CDR-H1, CDR-H2 and CDR-H3 of an antibody of Table 4, and
the VL that binds LRP5 comprises light chain CDRs (CDR-L1, CDR-L2 and CDR-L3), of an antibody of Table 3 or Table 6, or the VL that binds LRP6 comprises the CDR-L1, CDR-L2 and CDR-L3 of an antibody of Table 4.

32. A tetravalent binding antibody molecule comprising an N-terminal LRP binding diabody and a C-terminal domain comprising two FZD4-binding or FZD5-binding scFv, the tetravalent binding antibody molecule comprising
a dimer of a first and second monomer, wherein each monomer comprises a single-chain polypeptide comprising, from N-terminus to C-terminus:
(a) a first peptide, said first peptide comprising a first heavy chain variable domain (VH) and a first light chain variable (VL) domain that bind LRP5 or LRP6,
(b) an Fc region, or fragment thereof comprising a constant heavy chain domain 3 (CH3), and
(c) a second peptide, said second peptide comprising a second VL and a second VH that bind a FZD4 or FZD5,
wherein the first and second monomers dimerize via the Fc regions or fragments thereof, the first VH and VL of each monomer pairs with the first VH and VL of the other monomer forming a diabody that binds LRP5 or LRP6 and the second VL and VH of each monomer pair to form a scFvs that bind FZD4 or FZD5,
wherein the LRP binding diabody forms the N-terminal LRP binding domain of the tetravalent binding antibody molecule and the two FZD4-binding or FZD5-binding scFvs form the C-terminal FZD-binding domain of the tetravalent binding antibody molecule wherein when the LRP binding domain binds only LRP5, the FZD binding domain binds FZD5.

33. The tetravalent binding antibody molecule of claim 32, wherein
the VH that binds LRP5 comprises heavy chain CDRs (CDR-H1, CDR-H2 and CDR-H3), of an antibody of Table 3 or Table 6, or the VH that binds LRP6 comprises the CDR-H1, CDR-H2 and CDR-H3 of an antibody of Table 4,
the VL that binds LRP5 comprises light chain CDRs (CDR-L1, CDR-L2 and CDR-L3), of an antibody of Table 3 or Table 6, or the VL that binds LRP6 comprises the CDR-L1, CDR-L2 and CDR-L3 of an antibody of Table 4, and
the VH that binds FZD comprises the heavy chain CDRs (CDR-H1, CDR-H2 and CDR-H3), of an antibody of Table 1, Table 2 or Table 6.

34. A tetravalent binding antibody molecule in a Diabody-Fc-scFv format comprising an Fc domain, a LRP5/6 co-receptor binding domain including a bispecific diabody that binds at least two sites on the co-receptor,
wherein the diabody is attached to the amino terminus of the Fc domain and a FZD binding domain is attached to the carboxy terminus of the Fc domain comprising two scFv fragments each binding FZD wherein the scFv is specific for FZD5, or pan-specific binding to FZD5 and one or more other FZD.

35. A tetravalent binding antibody molecule in an IgG-diabody format having an Fc domain, a FZD binding domain that comprises of two Fab fragments attached to the N-terminus of the Fc domain, each Fab binding to an FZD5, or FZD4 and FZD5 and a LRP5/6 co-receptor binding domain attached to the C-terminus of the Fc domain that is composed of a diabody that binds at least two sites on the co-receptor LRP5/6, wherein the Fabs are specific for FZD5 or pan-specific, binding to FZD5 and one or more FZD.

36. A tetravalent binding antibody molecule in an IgG-Diabody format comprising an Fc domain, an N-terminal binding domain for a FZD, wherein the FZD-binding Fabs bind FZD5 or FZD5 and at least one other FZD and a C-terminal binding domain for a LRP5/6 co-receptor and including a LRP5/6 coreceptor-binding diabody.

37. The tetravalent binding antibody molecule of claim 36, comprising:
a first and second heavy chain monomer, wherein each heavy chain monomer includes a single-chain polypeptide from N-terminus to C-terminus:
(a) a heavy chain variable domain (VH) that binds FZD5 or FZD4 and FZD5, linked to
(b) a heavy chain constant region domain 1 (CH1 domain), linked to
(c) an Fc region (or fragment thereof comprising a constant heavy chain domain 3 (CH3 domain)), linked to
(d) a peptide comprising a VH that binds a LRP5/6 co-receptor, linked to a light chain variable domain (VL) that binds a LRP5/6 co-receptor, and
a first and second light chain monomer, wherein each light chain monomer includes from N terminus to C terminus a VL that binds the FZD, linked to a constant light chain domain 1 (CL1 domain).

38. The tetravalent binding antibody molecule of claim 1, wherein the tetravalent binding antibody molecule comprises
(a) a first heavy chain comprising the amino acid sequence of the hole heavy chain construct of SEQ ID NO: 986, a second heavy chain comprising the amino acid a knob heavy chain construct of SEQ ID NO: 987 and a light chain comprising the amino acid sequence of the light chain construct of SEQ ID NO: 992 wherein the amino acid sequence of the CDRs are the amino acid sequence of the CDRs of ANT80; or
(b) a first heavy chain comprising the amino acid sequence of the hole heavy chain construct of SEQ ID NO: 988, a second heavy chain comprising the amino acid a knob heavy chain construct of SEQ ID NO: 989 and a light chain comprising the amino acid sequence of the light chain construct of SEQ ID NO: 992 wherein the amino acid sequence of the CDRs are the amino acid sequence of the CDRs of ANT 98; or
(c) a first heavy chain comprising the amino acid sequence of the hole heavy chain construct of SEQ ID NO: 990, a second heavy chain comprising the amino acid a knob heavy chain construct of SEQ ID NO: 991 and a light chain comprising the amino acid sequence of the light chain construct of SEQ ID NO: 992 wherein the amino acid sequence of the CDRs are the amino acid sequence of the CDRs of ANT99; or
(d) a first heavy chain comprising the amino acid sequence of the hole heavy chain construct selected from the group consisting of SEQ ID NO: 986, SEQ ID NO: 988, and SEQ ID NO: 990; a second heavy chain comprising the amino acid of a knob heavy chain construct selected from the group consisting of SEQ ID NO: 987, SEQ ID NO: 989, and SEQ ID NO: 991; and a light chain comprising the amino acid sequence of the light chain construct of SEQ ID NO: 992.

39. A pharmaceutical composition comprising a tetravalent binding antibody molecule of claim 1 and a pharmaceutically acceptable carrier.

40. A method for promoting endothelial cell barrier functions in a tissue comprising administering an effective amount of a tetravalent binding antibody molecule of claim 1 to a tissue.

41. The method of claim 40, wherein the tissue is brain, kidney or eye tissue.

42. The method of claim 41, wherein the tetravalent binding antibody molecule is administered to eye tissue by intravitreal injection.

43. A method for treating inflammatory bowel disease in a subject in need thereof comprising administering an effective amount of a tetravalent binding molecule of claim 1 to said subject.

44. A method for increasing retinal or brain endothelial cell barrier functions, decreasing endothelial cell permeability, enhancing or restoring blood retina and blood brain barrier maintenance in a subject in need thereof comprising contacting an endothelial cell comprising an FZD4 receptor and an LRP5 and/or LRP6 in a subject in need thereof with an effective amount of a tetravalent binding antibody molecule of claim 1.

45. The method of claim 43, wherein the tetravalent binding antibody molecule is administered to the subject in need thereof by injection, topically, or orally.

46. The method of claim 43, wherein the tetravalent binding antibody molecule is administered subcutaneously, intravenously, intraperitoneally, intrathecally, intravitreously or intraocularly.
